SEQUENCE LISTING (333 sequences)

SEQ ID NO: 1 - DNA, 330 bp, synthetic construct
  source 1..330: mol_type = other DNA
  misc_feature 1..330: note = BLV1H12 Light chain DNA
caggccgtcctgaaccagccaagcagcgtctccgggtctctggggcagcgggtctcaatcacctgtagcgggtcttcctccaatgtcggcaacggctacgtgtcttggtatcagctgatccctggcagtgccccacgaaccctgatctacggcgacacatccagagcttctggggtccccgatcggttctcagggagcagatccggaaacacagctactctgaccatcagctccctccaggctgaggacgaagcagattatttctgcgcgtctgccgaggactctagttcaaatgccgtgtttggaagcggcaccacactgacagtccta

SEQ ID NO: 2 - protein, 110 aa, synthetic construct
  REGION 1..110: note = BLV1H12 variable Light chain
QAVLNQPSSVSGSLGQRVSITCSGSSSNVGNGYVSWYQLIPGSAPRTLIYGDTSRASGVPDRFSGSRSGNTATLTISSLQAEDEADYFCASAEDSSSNAVFGSGTTLTVL

SEQ ID NO: 3 - DNA, 25 bp, synthetic construct
  source 1..25: mol_type = other DNA
  misc_feature 1..25: note = igGCDNA2REV
ccgctcttcagggcacccgagttcc

SEQ ID NO: 4 - DNA, 25 bp, synthetic construct
  source 1..25: mol_type = other DNA
  misc_feature 1..25: note = igMCDNA2REV
ctgactgtgctgttgttgaacttcc

SEQ ID NO: 5 - DNA, 25 bp, synthetic construct
  source 1..25: mol_type = other DNA
  misc_feature 1..25: note = igACDNA2REV
gacacgctgtcgccattctggttcc

SEQ ID NO: 6 - DNA, 32 bp, synthetic construct
  source 1..32: mol_type = other DNA
  misc_feature 1..32: note = igGCDNA1.7REV
cgggcacggtcaccatgctgctgagagagtag

SEQ ID NO: 7 - DNA, 56 bp, synthetic construct
  source 1..56: mol_type = other DNA
  misc_feature 1..56: note = BOVVHFR4REV
ttacctgcggccgctgaggagacggtgaccaggagtccaactggagctccatcaag

SEQ ID NO: 8 - DNA, 40 bp, synthetic construct
  source 1..40: mol_type = other DNA
  misc_feature 1..40: note = BOVSTALKFOR1
cagccggccatggccacatactacagtactactgtacacc

SEQ ID NO: 9 - DNA, 40 bp, synthetic construct
  source 1..40: mol_type = other DNA
  misc_feature 1..40: note = BOVSTALKFOR2
cagccggccatggccacatactacagtactactgtatacc

SEQ ID NO: 10 - DNA, 40 bp, synthetic construct
  source 1..40: mol_type = other DNA
  misc_feature 1..40: note = BOVSTALKFOR3
cagccggccatggccacatactacagtactactgtgctcc

SEQ ID NO: 11 - DNA, 40 bp, synthetic construct
  source 1..40: mol_type = other DNA
  misc_feature 1..40: note = BOVSTALKFOR4
cagccggccatggccacatactacagtggtactgtgcacc

SEQ ID NO: 12 - DNA, 37 bp, synthetic construct
  source 1..37: mol_type = other DNA
  misc_feature 1..37: note = BOVVHNCOFOR2 NotI restriction enzyme site (bold/underline)
aaaaagccatggtgcaggtgcagctgcgggagtcggg

SEQ ID NO: 13 - DNA, 36 bp, synthetic construct
  source 1..36: mol_type = other DNA
  misc_feature 1..36: note = BOVVHFR4XHOREV Xho I restriction enzyme site (bold/underline)
ttacctctcgagtgaggagacggtgaccaggagtcc

SEQ ID NO: 14 - DNA, 507 bp, synthetic construct
  source 1..507: mol_type = other DNA
  misc_feature 1..507: note = R2G3
caggtgcagctgcgggagtcgggccccagcctggtgaagccctcacagaccctctcgctcacctgcgcggcctctggattctcattgagcgacaaggctgtaggctgggtccgccgggctccagggaaggcgctggagtggctcggtagtatagacactggtggaagcacaggctataacccaggcctgaaatcccggctcagcatcaccaaggacaactccaagagccaagtctccctgtcaattagcagcgtaacgtctgaggactcggccacatactactgtgcaactgtacaccagaaaacagctgaaggagacaaaacgtgtcctgatggttacgagcatacttgtggttgcattgggggttgtggttgcaaaaggtctgcctgtataggtgcactttgttgccaagcgtcgttgggtggttggcttagtgacggtgaaacctacacttacgagttccacgtcgatacctggggccaaggactcgtggtcaccgtctcctca

SEQ ID NO: 15 - DNA, 507 bp, synthetic construct
  source 1..507: mol_type = other DNA
  misc_feature 1..507: note = SR3A3
caggtgcagctgcgggagtcgggccccagcctggtgaagccctcacagaccctctccctcacctgcacaatctctggattctcattgagtagctatgctgtactctgggtccgccaggctccagggaagccgctggagtggctcggtagtatagacactgcggaaaacacaggctataacccaggcctgaaatcccggctcagcatcaccaaggacaactccaagagccaagtctctctgtcagtgagcagcgtgacaaccgaggactcggccacatactactgtgctactgtacaccagaaaacgcgaaaagaaaaaaattgtcctgatggctatatctatagttctaatatcactagcggttttgattgtggtgtctggatttgtcgtcgcgtcggtagtgccttctgtagtcgtactggtgattatactagtcctactgaacttgacatttacgagttctacgtcgaagggtggggccagggagtcccggtcaccgtctcctca

SEQ ID NO: 16 - DNA, 498 bp, synthetic construct
  source 1..498: mol_type = other DNA
  misc_feature 1..498: note = R2F12
caggtgcagctgcgggagtcgggccccagcctggtgaagccctcacagaccctctccctcacctgcacggtctctggattctcattgagcgacaaggctgtaggctgggtccgccgggctccagggaaggcgctggagtggctcggtagtatagacactggtggaatgacaggctataacccaggcctgaaatcccggctcagcatcaccaaggacaactccaaaagccaagtctctctatcagtgaatagcgtgacaactgaggactcggccacgtactactgtgccactgtagaccagaaaacgaaaaatgcttgccctgatgatttcgattatcgttgttcgtgtatcggtggttgtggctgcgcccgtaaaggatgcgttggtcctctttgttgtcgttctgatttgggtggctatcttactgatagtcctgcttacatttacgaatggtatattgatctttggggccaaggactcctggtcaccgtctcctca

SEQ ID NO: 17 - DNA, 405 bp, synthetic construct
  source 1..405: mol_type = other DNA
  misc_feature 1..405: note = R5A3
caggtgcagctgcgggagtcgggccccagcctggtgaagccgtcacagaccctctcgctcacctgcacggcctctggattctcattgagcgacaaggctgtaggctgggtccgccaggctccagggaaggcgctggagtggctcggtagtatagacactggtggaagcacaggctataacccaggcctgaaatcccggctcagcatcaccaaggacaactccaagagccaagtctctctgtcagtgagcagcgtgacaactgaggactcggccacatactactgtactactgtgcactgtagtgatggtggttatgttgaggcgggttttggttgttggccttgggattatggttatccttacgtcgatgcctggggccaaggactcctggtcaccgtctcctca

SEQ ID NO: 18 - DNA, 396 bp, synthetic construct
  source 1..396: mol_type = other DNA
  misc_feature 1..396: note = R4G11
caggtgcagctgcgggagtcgggccccagcctggtgaagccctcacagaccctctccctcacctgcacggtctctggattctcattgagcagctatggtataacctgggtccgccaggctccagggaaggcgctggagtgcctcggtagtataagcagtggtggaaccacagactacaacccagccctgaaatcccggctcagcatcaccaaggacaactccaagagccaagtctctctgtcagtgagcagcgtgacacctgaggacacggccacatactactgttcgaagtggaatttagaatatacttggggtggtgttggttgcgctagttttgctgatgaggacacccacgttgatgcctggggccaaggactcctggtcaccgtctcctca

SEQ ID NO: 19 - DNA, 417 bp, synthetic construct
  source 1..417: mol_type = other DNA
  misc_feature 1..417: note = R4G3
caggtgcagctgcgggagtcgggccccagcctgatgaagccctcacagaccctctccctcacctgcacggtctctgggttctcattgagcgactatgctgtaggctgggtccgccaggccccagggaaggcgctggagtggctcggtggtatagacactggtggaagcacaggctataacccaggcctggaatcccggctcagcatcaccaaggacaactccaagagccaagtctctctgtcagtgagcagcgtgacaactgaggactcggccacatactactgtactactgtggtcctttgttattttaattatgttgttcgtcgttataattgtggtggtcttggttatgggcatggctttaatagtttctacgtcgatgcctggggccaaggactcctggtcaccgtctcctca

SEQ ID NO: 20 - DNA, 417 bp, synthetic construct
  source 1..417: mol_type = other DNA
  misc_feature 1..417: note = R4E5
caggtgcagctgcgggagtcgggccccagcctggtgaagccctcacagaccctctccctcacctgcacgacctctggattctcactgagaaactatgctgtaggctgggtccgccaggctccggggaaggcgctggagtggctcggtggtatagacactggtggaagcacaggctataacccaggcctggaatcccggctcagcatcaccaaggacaactccaagagccaagtctctctgtcagtgagcagcgtgacaactgaggactcggccacatactactgtactactgtggtcctttgttattttaattatgttgttcgtcgttataattgtggtggtcttggttatgggcatggctttaatagtttctacgtcgatgcctggggccaaggactcctggtcaccgtctcctca

SEQ ID NO: 21 - DNA, 507 bp, synthetic construct
  source 1..507: mol_type = other DNA
  misc_feature 1..507: note = R4C1
caggtgcagctgcgggagtcgggccccagcctggtgaagccgtcacagaccctctcgctcacctgcacggcctctggattctcattgagcgataaggctgtaggctgggtccgccaggctccagggaagccgctggagtggctcggtagtatagacactgcggaaaacacaggctataacccaggcctgaaatctcggctcagcatcaccaaggacaactccaagagccaagtctctctgtcagtgagcagcgtgacaactgaggactcggccacatactactgtgctactgtacaccagaaaacgcgaaaagaaaaaaattgtcctgatggctatatctatagttctaataccgccagcggttatgattgtggtgtctggatttgtcgtcgcgtcggtagtgccttctgtagtcgtactggtgattatactagtcctagtgaatttgacatttacgagttctacgtcgaagggtggggccagggactcctggtcaccgtctcctca

SEQ ID NO: 22 - DNA, 417 bp, synthetic construct
  source 1..417: mol_type = other DNA
  misc_feature 1..417: note = R4A10
caggtgcagctgcgggagtcgggccccagcctggtgaagccctcacagaccctctccctcacctgcacgacctctggattctcattgagcgactatgctgtaggctgggtccgccaggctccagggaaggcgctggagtggctcggtggtatagacactggtggaagcacaggctataacccaggcctgaaatcccggctcagcatcaccaaggacaactccaagagtcaagtctctctgtcagtgagcagcgtgacaactgaggattcggccacatactactgtactgccgtggtcctctgttattacaatcgggttgtgcgtcgtaataattgtggtgggcttggttatgattatggttttgatcatttctacgtcgatgcctggggccaaggactcctggtcaccgtctcctca

SEQ ID NO: 23 - DNA, 384 bp, synthetic construct
  source 1..384: mol_type = other DNA
  misc_feature 1..384: note = R2G1
caggtgcagctgcgggagtcgggccccagcctggtgaagccctcacagaccctctccctcacctgcacggtctctggattctcattgagcaactatgctgtaggctgggtccgccaggctccagggaaggcgctggagtgcctcggtgatgtagacagtagtggaggcacagcctataacccagccctgaaatcccggttcatcatcgccaaggacaactccaagaaccaagtctctctgtcagtccgcagcgtgacacctgaggacacggccacatactactgtgcgaagtttgctaagggtactacgagtgctggtgcttgtgattattcagaaagctacgtcgatgcctggggccagggactcctggtcaccgtctcctca

SEQ ID NO: 24 - DNA, 399 bp, synthetic construct
  source 1..399: mol_type = other DNA
  misc_feature 1..399: note = R2D6
caggtgcagctgcgggagtcgggccccagcctggtgaagccctcacagaccctctccctcacctgcacgacctctggattctcactgagcagctatgctgtaggctgggtccgccaggctccggggaaggcgctggagtgggttggtgatatagattatgtcggaaacacagactataacccagccctgaaatcccggctcagcatcaccaaggacaactccaagagccaagtctctctggtagtgagcagcgtgacagctgaggacgcggccacatactactgtgcgaaatattccggtgcttatgcttatgctgcttgcaattattatggttggcgttgtgcttgggaaagctacatcgatgcctggggccaaggactcctggtcaccgtctcctca

SEQ ID NO: 25 - DNA, 399 bp, synthetic construct
  source 1..399: mol_type = other DNA
  misc_feature 1..399: note = R2B1
caggtgcagctgcgggagtcgggccccagcctggtgaagccctcacagaccctctccctcacctgcacggtctctggattttcattaagcgataataatgtaggctgggtccgccaggctccaggaaaggcgctggagtggctcggtgtaatgcataatgatgggaacaaaggctataacccagccctgaaatcccggctcagcatcaccaaggacagctccaagagccaagtctctctatcactaagcagcgtgacaagtgaggacacggccacatactactgtacaagagacaatgcacgttgtgatagttggacgtatgacagctgtgatacttggtatcgcaattcgtggcacgttgatgcctggggccaaggactcctggtcaccgtctcctca

SEQ ID NO: 26 - DNA, 504 bp, synthetic construct
  source 1..504: mol_type = other DNA
  misc_feature 1..504: note = SKM-BLV1H12
caggtgcagctgcgcgagtcgggccccagcctggtgaagccgtcacagaccctctcgctcacctgcacggcctctggattctcattgagcgacaaggctgtaggctgggtccgccaggctccagggaaggcgctggagtggctcggtagtatagacactggtggaaacacaggctataacccaggcctgaaatcccggctcagcatcaccaaggacaactccaagagtcaagtctctctgtcagtgagcagcgtgacaactgaggactcggccacatactactgtactactgtgcaccaagagaccttacgtagttgtcctgatggttatattgataattctggatgcacggctgattggggttgtgcagctcttgattgttggcggcgtcgttttggttaccacagcactgatccttctcattatactggtgcgacgtatatttacacgtacagcttgcacatcgatgcctggggccaaggactcctggtcaccgtctcctca

SEQ ID NO: 27 - DNA, 507 bp, synthetic construct
  source 1..507: mol_type = other DNA
  misc_feature 1..507: note = SKD-BLV1H12
caggtgcagctgcgcgagtcgggccccagcctggtgaagccgtcacagaccctctcgctcacctgcacggcctctggattctcattgagcgacaaggctgtaggctgggtccgccaggctccagggaaggcgctggagtggctcggtagtatagacactggtggaaacacaggctataacccaggcctgaaatcccggctcagcatcaccaaggacaactccaagagtcaagtctctctgtcagtgagcagcgtgacaactgaggactcggccacatactactgtactactgtgcaccagcgtacaagcgaaaaaagaagttgtcctggcggtagtagtagacgttatcctagtggcgccagttgtgacgttagtgggggcgcttgtgcgtgttatgtttctaattgtagaggcgttttgtgtcctactcttaacgaaatcgttgcttatacctacgaatggcacgtcgacgcctggggccaaggactcctggtcaccgtctcctca

SEQ ID NO: 28 - DNA, 378 bp, synthetic construct
  source 1..378: mol_type = other DNA
  misc_feature 1..378: note = RBD F4
caggtgcagctgcgggagtcgggccccagcctggtgaagccctcacagaccctctccctcacctgcacggtctctggattctcattgagcagcaatggtgtggtctgggtccgccaggctccagggaaggcgctggagtggctcggtgatatatgcagtactggaggcacaagctttaacccagccctgaaatcccggctcagcatcgccaaggacaactccaagagccaagtctctctgtcagtgagaagcgtgacacctgaggacacggccacatattactgtgcaagaagtcgtggttatgattgttatgctaatgtggatgctttggactacgtcgatgcctggggccaaggactcctggtcaccgtctcctca

SEQ ID NO: 29 - DNA, 378 bp, synthetic construct
  source 1..378: mol_type = other DNA
  misc_feature 1..378: note = RBD C6
caggtgcagctgcgggagtcgggccccagcctggtgaagccctcacagaccctctccctcacctgcacggtctctggattctcattgagcagcaatggtgtagtctgggtccgccaggctccagggagaccactggagtggctcggtgatatatgcagtaatggaggcacaagctttaacccagccctgaaatcccggctcagcatcgccaaggacaactccgagagccaagtctctctgaccgtgagaagcgtgacacctgaggacacagccacatattactgtgcaagaagtcgtggttatgattgttatgcttatgtttatgctttggacaccgtcgatgcctggggccaaggactcctggtcaccgtctcctca

SEQ ID NO: 30 - DNA, 378 bp, synthetic construct
  source 1..378: mol_type = other DNA
  misc_feature 1..378: note = RBD A2
caggtgcagctgcgggagtcgggccccagcctggtgaagcccctacagatcctctccctcacctgcacggtctctggattctcattgagcagcaatggtgtggtctgggtccgccaggctccagggaaggcgctggagtggctcggtgatatatgcagtactggaggcacaagctttaacccagccctgaaatcccggctcagcatcgccaaggacaactccaagagccaagtctctctgtcagtgagaagcgtgacacctgaggacacggccacatattactgtgcaagaagtcgtggttatgattgttatgctaatgtggatgctttggactacgtcgatgcctggggccaaggactcctggtcaccgtctcctca

SEQ ID NO: 31 - DNA, 507 bp, synthetic construct
  source 1..507: mol_type = other DNA
  misc_feature 1..507: note = SA-R2C3
caggtgcagctgcgggagtcgggccccagcctggtgaagccgtcacagaccctctcgctcacctgcacggcctctggattctcattgagcgataagcctgtaggctgggtccgccaggctccagggaagccactggagtggctcggtagtatagacactgcggaaaacacaggctataacccaggcctgaaatctcggctcagcatcaccaaggacaactccaagagccaagtctctctgtcactgagcagcgtgacgactgaggactcggccacatactactgtgctactgtacaccagaaaacgcggaaggaaaaaagttgtcctgatggctatctctatagttctaataccggccgcggttatgattgtggtgtctggacttgtcgtcgcgtcggtggtgaattctgtagtgctactggtgattggactagtcctagtgaagaagacttttacgaattctacgtcgatacgtggggccagggagccccggtcaccgtctcctca

SEQ ID NO: 32 - DNA, 501 bp, synthetic construct
  source 1..501: mol_type = other DNA
  misc_feature 1..501: note = SA-R2D9
caggtgcagctgcgggagtcgggccccagcctggtgaagccgtcacagaccctctcgctcacctgcacggcctctggattctcattaagcgacaaggctattggctgggtccgccaggctccagggaaggcgctggagtggctcggtagtatagacacccgtggaaacacaggctataacccaggcctgaaatcccgactcagcatcaccaaggacagctccaagagccaagtctctctgtcagtgaacagcgtgacaactgaagactcggccacgtacctctgtgctattgtgcagcagatcacacacaaaacttgtcctaatggttacaattggtttgatcgttgttgttcttgggatggtacctgtggtgatggttgttgcagtaatcgtgcttggcctagtggtaatggtagagccgacagtagtattggtgaaacttatggttacgaatttcacgtggctgcctggggccaaggactcctggtcaccgtctcctca

SEQ ID NO: 33 - protein, 169 aa, synthetic construct
  REGION 1..169: note = R2G3
QVQLRESGPSLVKPSQTLSLTCAASGFSLSDKAVGWVRRAPGKALEWLGSIDTGGSTGYNPGLKSRLSITKDNSKSQVSLSISSVTSEDSATYYCATVHQKTAEGDKTCPDGYEHTCGCIGGCGCKRSACIGALCCQASLGGWLSDGETYTYEFHVDTWGQGLVVTVSS

SEQ ID NO: 34 - protein, 169 aa, synthetic construct
  REGION 1..169: note = SR3A3
QVQLRESGPSLVKPSQTLSLTCTISGFSLSSYAVLWVRQAPGKPLEWLGSIDTAENTGYNPGLKSRLSITKDNSKSQVSLSVSSVTTEDSATYYCATVHQKTRKEKNCPDGYIYSSNITSGFDCGVWICRRVGSAFCSRTGDYTSPTELDIYEFYVEGWGQGVPVTVSS

SEQ ID NO: 35 - protein, 166 aa, synthetic construct
  REGION 1..166: note = R2F12
QVQLRESGPSLVKPSQTLSLTCTVSGFSLSDKAVGWVRRAPGKALEWLGSIDTGGMTGYNPGLKSRLSITKDNSKSQVSLSVNSVTTEDSATYYCATVDQKTKNACPDDFDYRCSCIGGCGCARKGCVGPLCCRSDLGGYLTDSPAYIYEWYIDLWGQGLLVTVSS

SEQ ID NO: 36 - protein, 135 aa, synthetic construct
  REGION 1..135: note = R5A3
QVQLRESGPSLVKPSQTLSLTCTASGFSLSDKAVGWVRQAPGKALEWLGSIDTGGSTGYNPGLKSRLSITKDNSKSQVSLSVSSVTTEDSATYYCTTVHCSDGGYVEAGFGCWPWDYGYPYVDAWGQGLLVTVSS

SEQ ID NO: 37 - protein, 132 aa, synthetic construct
  REGION 1..132: note = R4G11
QVQLRESGPSLVKPSQTLSLTCTVSGFSLSSYGITWVRQAPGKALECLGSISSGGTTDYNPALKSRLSITKDNSKSQVSLSVSSVTPEDTATYYCSKWNLEYTWGGVGCASFADEDTHVDAWGQGLLVTVSS

SEQ ID NO: 38 - protein, 139 aa, synthetic construct
  REGION 1..139: note = R4G3
QVQLRESGPSLMKPSQTLSLTCTVSGFSLSDYAVGWVRQAPGKALEWLGGIDTGGSTGYNPGLESRLSITKDNSKSQVSLSVSSVTTEDSATYYCTTVVLCYFNYVVRRYNCGGLGYGHGFNSFYVDAWGQGLLVTVSS

SEQ ID NO: 39 - protein, 139 aa, synthetic construct
  REGION 1..139: note = R4E5
QVQLRESGPSLVKPSQTLSLTCTTSGFSLRNYAVGWVRQAPGKALEWLGGIDTGGSTGYNPGLESRLSITKDNSKSQVSLSVSSVTTEDSATYYCTTVVLCYFNYVVRRYNCGGLGYGHGFNSFYVDAWGQGLLVTVSS

SEQ ID NO: 40 - protein, 169 aa, synthetic construct
  REGION 1..169: note = R4C1
QVQLRESGPSLVKPSQTLSLTCTASGFSLSDKAVGWVRQAPGKPLEWLGSIDTAENTGYNPGLKSRLSITKDNSKSQVSLSVSSVTTEDSATYYCATVHQKTRKEKNCPDGYIYSSNTASGYDCGVWICRRVGSAFCSRTGDYTSPSEFDIYEFYVEGWGQGLLVTVSS

SEQ ID NO: 41 - protein, 139 aa, synthetic construct
  REGION 1..139: note = R4A10
QVQLRESGPSLVKPSQTLSLTCTTSGFSLSDYAVGWVRQAPGKALEWLGGIDTGGSTGYNPGLKSRLSITKDNSKSQVSLSVSSVTTEDSATYYCTAVVLCYYNRVVRRNNCGGLGYDYGFDHFYVDAWGQGLLVTVSS

SEQ ID NO: 42 - protein, 128 aa, synthetic construct
  REGION 1..128: note = R2G1
QVQLRESGPSLVKPSQTLSLTCTVSGFSLSNYAVGWVRQAPGKALECLGDVDSSGGTAYNPALKSRFIIAKDNSKNQVSLSVRSVTPEDTATYYCAKFAKGTTSAGACDYSESYVDAWGQGLLVTVSS

SEQ ID NO: 43 - protein, 133 aa, synthetic construct
  REGION 1..133: note = R2D6
QVQLRESGPSLVKPSQTLSLTCTTSGFSLSSYAVGWVRQAPGKALEWVGDIDYVGNTDYNPALKSRLSITKDNSKSQVSLVVSSVTAEDAATYYCAKYSGAYAYAACNYYGWRCAWESYIDAWGQGLLVTVSS

SEQ ID NO: 44 - protein, 133 aa, synthetic construct
  REGION 1..133: note = R2B1
QVQLRESGPSLVKPSQTLSLTCTVSGFSLSDNNVGWVRQAPGKALEWLGVMHNDGNKGYNPALKSRLSITKDSSKSQVSLSLSSVTSEDTATYYCTRDNARCDSWTYDSCDTWYRNSWHVDAWGQGLLVTVSS

SEQ ID NO: 45 - protein, 168 aa, synthetic construct
  REGION 1..168: note = SKM-BLV1H12
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCTTVHQETLRSCPDGYIDNSGCTADWGCAALDCWRRRFGYHSTDPSHYTGATYIYTYSLHIDAWGQGLLVTVSS

SEQ ID NO: 46 - protein, 169 aa, synthetic construct
  REGION 1..169: note = SKD-BLV1H12
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCTTVHQRTSEKRSCPGGSSRRYPSGASCDVSGGACACYVSNCRGVLCPTLNEIVAYTYEWHVDAWGQGLLVTVSS

SEQ ID NO: 47 - protein, 126 aa, synthetic construct
  REGION 1..126: note = RBD F4
QVQLRESGPSLVKPSQTLSLTCTVSGFSLSSNGVVWVRQAPGKALEWLGDICSTGGTSFNPALKSRLSIAKDNSKSQVSLSVRSVTPEDTATYYCARSRGYDCYANVDALDYVDAWGQGLLVTVSS

SEQ ID NO: 48 - protein, 126 aa, synthetic construct
  REGION 1..126: note = RBD C6
QVQLRESGPSLVKPSQTLSLTCTVSGFSLSSNGVVWVRQAPGRPLEWLGDICSNGGTSFNPALKSRLSIAKDNSESQVSLTVRSVTPEDTATYYCARSRGYDCYAYVYALDTVDAWGQGLLVTVSS

SEQ ID NO: 49 - protein, 126 aa, synthetic construct
  REGION 1..126: note = RBD A2
QVQLRESGPSLVKPLQILSLTCTVSGFSLSSNGVVWVRQAPGKALEWLGDICSTGGTSFNPALKSRLSIAKDNSKSQVSLSVRSVTPEDTATYYCARSRGYDCYANVDALDYVDAWGQGLLVTVSS

SEQ ID NO: 50 - protein, 169 aa, synthetic construct
  REGION 1..169: note = SA-R2C3
QVQLRESGPSLVKPSQTLSLTCTASGFSLSDKPVGWVRQAPGKPLEWLGSIDTAENTGYNPGLKSRLSITKDNSKSQVSLSLSSVTTEDSATYYCATVHQKTRKEKSCPDGYLYSSNTGRGYDCGVWTCRRVGGEFCSATGDWTSPSEEDFYEFYVDTWGQGAPVTVSS

SEQ ID NO: 51 - protein, 167 aa, synthetic construct
  REGION 1..167: note = SA-R2D9
QVQLRESGPSLVKPSQTLSLTCTASGFSLSDKAIGWVRQAPGKALEWLGSIDTRGNTGYNPGLKSRLSITKDSSKSQVSLSVNSVTTEDSATYLCAIVQQITHKTCPNGYNWFDRCCSWDGTCGDGCCSNRAWPSGNGRADSSIGETYGYEFHVAAWGQGLLVTVSS

SEQ ID NO: 52 - DNA, 144 bp, synthetic construct
  source 1..144: mol_type = other DNA
  misc_feature 1..144: note = R2G3
gaaggagacaaaacgtgtcctgatggttacgagcatacttgtggttgcattgggggttgtggttgcaaaaggtctgcctgtataggtgcactttgttgccaagcgtcgttgggtggttggcttagtgacggtgaaacctacact

SEQ ID NO: 53 - DNA, 153 bp, synthetic construct
  source 1..153: mol_type = other DNA
  misc_feature 1..153: note = SR3A3
aaagaaaaaaattgtcctgatggctatatctatagttctaatatcactagcggttttgattgtggtgtctggatttgtcgtcgcgtcggtagtgccttctgtagtcgtactggtgattatactagtcctactgaacttgacatttacgagttc

SEQ ID NO: 54 - DNA, 150 bp, synthetic construct
  source 1..150: mol_type = other DNA
  misc_feature 1..150: note = R2F12
aaaacgaaaaatgcttgccctgatgatttcgattatcgttgttcgtgtatcggtggttgtggctgcgcccgtaaaggatgcgttggtcctctttgttgtcgttctgatttgggtggctatcttactgatagtcctgcttacatttacgaa

SEQ ID NO: 55 - DNA, 147 bp, synthetic construct
  source 1..147: mol_type = other DNA
  misc_feature 1..147: note = R4C1
aaagaaaaaaattgtcctgatggctatatctatagttctaataccgccagcggttatgattgtggtgtctggatttgtcgtcgcgtcggtagtgccttctgtagtcgtactggtgattatactagtcctagtgaatttgacatttac

SEQ ID NO: 56 - DNA, 147 bp, synthetic construct
  source 1..147: mol_type = other DNA
  misc_feature 1..147: note = SKM-BLV1H12
ctgcgtagttgtcctgatggttatattgataattctggatgcacggctgattggggttgtgcagctcttgattgttggcggcgtcgttttggttaccacagcactgatccttctcattatactggtgcgacgtatatttacacgtac

SEQ ID NO: 57 - DNA, 150 bp, synthetic construct
  source 1..150: mol_type = other DNA
  misc_feature 1..150: note = SKD-BLV1H12
agcgaaaaaagaagttgtcctggcggtagtagtagacgttatcctagtggcgccagttgtgacgttagtgggggcgcttgtgcgtgttatgtttctaattgtagaggcgttttgtgtcctactcttaacgaaatcgttgcttatacctac

SEQ ID NO: 58 - DNA, 156 bp, synthetic construct
  source 1..156: mol_type = other DNA
  misc_feature 1..156: note = SA-R2C3
cggaaggaaaaaagttgtcctgatggctatctctatagttctaataccggccgcggttatgattgtggtgtctggacttgtcgtcgcgtcggtggtgaattctgtagtgctactggtgattggactagtcctagtgaagaagacttttacgaattc

SEQ ID NO: 59 - DNA, 156 bp, synthetic construct
  source 1..156: mol_type = other DNA
  misc_feature 1..156: note = SA-R2D9
atcacacacaaaacttgtcctaatggttacaattggtttgatcgttgttgttcttgggatggtacctgtggtgatggttgttgcagtaatcgtgcttggcctagtggtaatggtagagccgacagtagtattggtgaaacttatggttacgaattt

SEQ ID NO: 60 - protein, 51 aa, synthetic construct
  REGION 1..51: note = R2G3
EGDKTCPDGYEHTCGCIGGCGCKRSACIGALCCQASLGGWLSDGETYTYEF

SEQ ID NO: 61 - protein, 51 aa, synthetic construct
  REGION 1..51: note = SR3A3
KEKNCPDGYIYSSNITSGFDCGVWICRRVGSAFCSRTGDYTSPTELDIYEF

SEQ ID NO: 62 - protein, 50 aa, synthetic construct
  REGION 1..50: note = R2F12
KTKNACPDDFDYRCSCIGGCGCARKGCVGPLCCRSDLGGYLTDSPAYIYE

SEQ ID NO: 63 - protein, 50 aa, synthetic construct
  REGION 1..50: note = R4C1
RKEKNCPDGYIYSSNTASGYDCGVWICRRVGSAFCSRTGDYTSPSEFDIY

SEQ ID NO: 64 - protein, 49 aa, synthetic construct
  REGION 1..49: note = SKM-BLV1H12
LRSCPDGYIDNSGCTADWGCAALDCWRRRFGYHSTDPSHYTGATYIYTY

SEQ ID NO: 65 - protein, 50 aa, synthetic construct
  REGION 1..50: note = SKD-BLV1H12
SEKRSCPGGSSRRYPSGASCDVSGGACACYVSNCRGVLCPTLNEIVAYTY

SEQ ID NO: 66 - protein, 52 aa, synthetic construct
  REGION 1..52: note = SA-R2C3
RKEKSCPDGYLYSSNTGRGYDCGVWTCRRVGGEFCSATGDWTSPSEEDFYEF

SEQ ID NO: 67 - protein, 52 aa, synthetic construct
  REGION 1..52: note = SA-R2D9
ITHKTCPNGYNWFDRCCSWDGTCGDGCCSNRAWPSGNGRADSSIGETYGYEF

SEQ ID NO: 68 - protein, 75 aa, synthetic construct
  REGION 1..75: note = SKD
CTTVHQRTSEKRSCPGGSSRRYPSGASCDVSGGACACYVSNCRGVLCPTLNEIVAYTYEWHVDAWGQGLLVTVSS

SEQ ID NO: 69 - protein, 74 aa, synthetic construct
  REGION 1..74: note = SKM
CTTVHQETLRSCPDGYIDNSGCTADWGCAALDCWRRRFGYHSTDPSHYTGATYIYTYSLHIDAWGQGLLVTVSS

SEQ ID NO: 70 - protein, 75 aa, synthetic construct
  REGION 1..75: note = R4C1
CATVHQKTRKEKNCPDGYIYSSNTASGYDCGVWICRRVGSAFCSRTGDYTSPSEFDIYEFYVEGWGQGLLVTVSS

SEQ ID NO: 71 - protein, 75 aa, synthetic construct
  REGION 1..75: note = R5C1
CATVHQKTRKEKSCPDGYLYSSNTGRGYDCGVWTCRRVGGEFCSATGDWTSPSEEDFYEFYVDTWGQGLLVTVSS

SEQ ID NO: 72 - protein, 75 aa, synthetic construct
  REGION 1..75: note = SR3A3
CATVHQKTRKEKNCPDGYIYSSNITSGFDCGVWICRRVGSAFCSRTGDYTSPTELDIYEFYVEGWGQGVPVTVSS

SEQ ID NO: 73 - protein, 72 aa, synthetic construct
  REGION 1..72: note = RR2F12
CATVDQKTKNACPDDFDYRCSCIGGCGCARKGCVGPLCCRSDLGGYLTDSPAYIYEWYIDLWGQGLLVTVSS

SEQ ID NO: 74 - protein, 75 aa, synthetic construct
  REGION 1..75: note = RR2G3
CATVHQKTAEGDKTCPDGYEHTCGCIGGCGCKRSACIGALCCQASLGGWLSDGETYTYEFHVDTWGQGLVVTVSS

SEQ ID NO: 75 - protein, 69 aa, synthetic construct
  REGION 1..69: note = Germ
CTTVHQSCPDGYSYGYGCGYGYGCSGYDCYGYGGYGGYGGYGYSSYSYSYTYEYYVDAWGQGLLVTVSS

SEQ ID NO: 76 - protein, 1272 aa, synthetic construct
  REGION 1..1272: note = WT Wuhan-Hu-1 S protein NCBI Reference Sequence: YP_009724390.1 (RBD shown in bold, intravirion in underline)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELKYEQYIKWPWYIWLGFIAGLIAIVMVTIMLCCMTSCCSCLKGCCSCGSCCKFDEDDSEPVLKGVKLHYT

SEQ ID NO: 77 - protein, 223 aa, synthetic construct
  REGION 1..223: note = Wuhan-Hu-1 S protein NCBI Reference Sequence: YP_009724390.1 RBD AA 319-541
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 78 - protein, 1229 aa, synthetic construct
  REGION 1..1229: note = Wuhan-Hu-1 S protein with furin site removed (AA685-686) and K986P and V987P stabilizing mutations (bold) Extracellular domain only (AA1233-1273 removed) NCBI Reference Sequence: YP_009724390.1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELKYEQYIKWPWYIWLGFIAGLIAIVMVTI

SEQ ID NO: 79 - protein, 1288 aa, synthetic construct
  REGION 1..1288: note = 7LYN South African (B.1.351) SARS-CoV-2 spike protein variant (S-GSAS-B.1.351)
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 80 - protein, 1288 aa, synthetic construct
  REGION 1..1288: note = 7LYN South African (B.1.351) SARS-CoV-2 spike protein variant (S-GSAS-B.1.351) with furin site removed and K986P and V987P stabilizing mutations (bold) Extracellular domain only
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 81 - protein, 100 aa, synthetic construct
  REGION 1..100: note = IgHV1-7 V gene
QVQLRESGPSLVKPSQTLSLTCTVSGFSLSDKAVGWVRQAPGKALEWLGGIDTGGSTGYNPGLKSRLSITKDNSKSQVSLSVSSVTTEDSATYYCTTVHQ

SEQ ID NO: 82 - protein, 48 aa, synthetic construct
  REGION 1..48: note = IDHD8-2
SCPDGYSYGYGCGYGYGCSGYDCYGYGGYGGYGGYGYSSYSYSYTYEY

SEQ ID NO: 83 - protein, 15 aa, synthetic construct
  REGION 1..15: note = IGHJ2-4
YVDAWGQGLLVTVSS

SEQ ID NO: 84 - protein, 25 aa, synthetic construct
  REGION 1..25: note = Minimal BOVVHNCOFOR2 primer
TGCAGGTGCAGCTGCGGGAGTCGGG

SEQ ID NO: 85 - DNA, 24 bp, synthetic construct
  source 1..24: mol_type = other DNA
  misc_feature 1..24: note = Minimal BOVVHFR4XHOREV primer
tgaggagacggtgaccaggagtcc

SEQ ID NO: 86 - protein, 56 aa, synthetic construct
  REGION 1..56: note = R2G3 Parental
GGGGAMGSEGDKTCPDGYEHTCGCIGGCGCKRSACIGALCCQASLGGWLSDGETYT

SEQ ID NO: 87 - protein, 51 aa, synthetic construct
  REGION 1..51: note = R2G3 TRUNC1
GGSEGDKTCPDGYEHTCGCIGGCGCKRSACIGALCCQASLGGWLSDGETYT

SEQ ID NO: 88 - protein, 48 aa, synthetic construct
  REGION 1..48: note = R2G3 TRUNC2
GGSEGDKTCPDGYEHTCGCIGGCGCKRSACIGALCCQASLGGWLSDGE

SEQ ID NO: 89 - protein, 45 aa, synthetic construct
  REGION 1..45: note = R2G3 TRUNC3
GGSEGDKTCPDGYEHTCGCIGGCGCKRSACIGALCCQASLGGWLS

SEQ ID NO: 90 - protein, 44 aa, synthetic construct
  REGION 1..44: note = R2G3 TRUNC3A
GGSEGDKTCPDGYEHTCGCIGGCGCKRSACIGALCCQASLGGWL

SEQ ID NO: 91 - protein, 43 aa, synthetic construct
  REGION 1..43: note = R2G3 TRUNC3B
GGSEGDKTCPDGYEHTCGCIGGCGCKRSACIGALCCQASLGGW

SEQ ID NO: 92 - protein, 42 aa, synthetic construct
  REGION 1..42: note = R2G3 TRUNC4
GGSEGDKTCPDGYEHTCGCIGGCGCKRSACIGALCCQASLGG

SEQ ID NO: 93 - protein, 39 aa, synthetic construct
  REGION 1..39: note = R2G3 TRUNC5
GGSEGDKTCPDGYEHTCGCIGGCGCKRSACIGALCCQAS

SEQ ID NO: 94 - protein, 15 aa, synthetic construct
  REGION 1..15: note = Flexible Linker
GGGGSGGGGSGGGGS

SEQ ID NO: 95 - protein, 34 aa, synthetic construct
  REGION 1..34: note = Mcoti-I
GGVCPKILQRCRRDSDSPGACICRGNGYCGSGSD

SEQ ID NO: 96 - protein, 34 aa, synthetic construct
  REGION 1..34: note = Mcoti-II
GGVCPKILKKCRRDSDSPGACICRGNGYCGSGSD

SEQ ID NO: 97 - protein, 30 aa, synthetic construct
  REGION 1..30: note = Mcoti-III
ERACPRILKKCRRDSDSPGACICRGNGYCG

SEQ ID NO: 98 - protein, 6 aa, synthetic construct
  REGION 1..6: note = Base of Stalk A
CTTVHQ

SEQ ID NO: 99 - protein, 6 aa, synthetic construct
  REGION 1..6: note = Base of Stalk A
CATVHQ

SEQ ID NO: 100 - protein, 6 aa, synthetic construct
  REGION 1..6: note = Base of Stalk A
CAIVQQ

SEQ ID NO: 101 - protein, 6 aa, synthetic construct
  REGION 1..6: note = Base of Stalk A
CATVDQ

SEQ ID NO: 102 - skipped

SEQ ID NO: 103 - protein, 6 aa, synthetic construct
  REGION 1..6: note = Ascending Stalk Domain
  VARIANT 2: note = X is any amino acid
  VARIANT 5: note = X is any amino acid
CXTVXQ

SEQ ID NO: 104 - protein, 6 aa, synthetic construct
  REGION 1..6: note = Ascending Stalk Domain
  VARIANT 2: note = X is Ser, Thr, Gly, Asn, Ala, or Pro
  VARIANT 5: note = X is His, Gln, Arg, Lys, Gly, Thr, Tyr, Phe, Trp, Met, Ile, Val, or Leu
CXTVXQ

SEQ ID NO: 105 - protein, 6 aa, synthetic construct
  REGION 1..6: note = Ascending Stalk Domain
  VARIANT 2: note = X is Ser, Ala
  VARIANT 5: note = X is His or Tyr
CXTVXQ

SEQ ID NO: 106 - protein, 5 aa, synthetic construct
  REGION 1..5: note = Enterokinase Cleavage Tag
DDDDK

SEQ ID NO: 107 - protein, 113 aa, synthetic construct
  REGION 1..113: note = Humanized BLV1H12 Variable Light
QAVLNQPSSVSGSLGQKVTISCSGSSSNIGNNYVSWYQQLPGTAPKLLIYGDTKRPSGIPDRFSGSKSGTSATLGITGLQTGDEADYYCASAEDSSSNAVFGSGTTLTVLGQP

SEQ ID NO: 108 - protein, 8 aa, synthetic construct
  REGION 1..8: note = Flexible Linker
GGGGAMGS

SEQ ID NO: 109 - skipped

SEQ ID NO: 110 - protein, 113 aa, synthetic construct
  REGION 1..113: note = BLV5B8 Variable Light Region
QAVLNQPSSVSGSLGQRVSITCSGSSSNVGNGYVSWYQLIPGSAPRTLIYGDTSRASGVPDRFSGSRSGNTATLTISSLQAEDEADYFCASAEDSSSNAVFGSGTTLTVLGQP

SEQ ID NO: 111 - protein, 94 aa, synthetic construct
  REGION 1..94: note = Humanized V1 Region
QVQLREWGAGLLKPSETLSLTCAVYGGSFSDKYWSWIRQPPGKGLEWIGSINHSGSTNYNPSLKSRVTISVDTSKNQFSLKLSSVTAADTAVYY

SEQ ID NO: 112 - protein, 11 aa, synthetic construct
  REGION 1..11: note = V2 Region
WGQGLLVTVSS

SEQ ID NO: 113 - protein, 216 aa, synthetic construct
  REGION 1..216: note = BLV1H12 Light Chain
QAVLNQPSSVSGSLGQRVSITCSGSSSNVGNGYVSWYQLIPGSAPRTLIYGDTSRASGVPDRFSGSRSGNTATLTISSLQAEDEADYFCASAEDSSSNAVFGSGTTLTVLGQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 114 - protein, 216 aa, synthetic construct
  REGION 1..216: note = B15 Humanized Light Chain
QAVLNQPSSVSGSLGQKVTISCSGSSSNIGNNYVSWYQQLPGTAPKLLIYGDTKRPSGIPDRFSGSKSGTSATLGITGLQTGDEADYYCASAEDSSSNAVFGSGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 115 - protein, 216 aa, synthetic construct; identical to SEQ ID NO: 113
  REGION 1..216: note = BLV5B8 light chain

SEQ ID NO: 116 - protein, 216 aa, synthetic construct
  REGION 1..216: note = human VL1-51
QSVLTQPPSVSAAPGQKVTISCSGSSSNIGNNYVSWYQQLPGTAPKLLIYDNNKRPSGIPDRFSGSKSGTSATLGITGLQTGDEADYYCASAEDSSSNAVFGSGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 117 - protein, 98 aa, synthetic construct
  REGION 1..98: note = Human germline light chain variable region sequence VL1-47
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSG

SEQ ID NO: 118 - protein, 99 aa, synthetic construct
  REGION 1..99: note = Human germline light chain variable region sequence VL1-40*1
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSG

SEQ ID NO: 119 - protein, 98 aa, synthetic construct
  REGION 1..98: note = Human germline light chain variable region sequence VL1-51*01
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSA

SEQ ID NO: 120 - protein, 99 aa, synthetic construct
  REGION 1..99: note = Human germline light chain variable region sequence VL2-18*02
QSALTQPPSVSGSPGQSVTISCTGTSSDVGSYNRVSWYQQPPGTAPKLMIYEVSNRPSGVPDRFSGSKSGNTASLTISGLQAEDEADYYCSSYTSSSTF

SEQ ID NO: 121 - DNA, 38 bp, synthetic construct
  source 1..38: mol_type = other DNA
  misc_feature 1..38: note = BOVVHFR4REV
ttacctgcggccgctgaggagacggtgaccaggagtcc

SEQ ID NO: 122 - DNA, 35 bp, synthetic construct
  source 1..35: mol_type = other DNA
  misc_feature 1..35: note = ULp1
ttttttgcggccgcccaggcgctgacgtaccattc

SEQ ID NO: 123 - DNA, 35 bp, synthetic construct
  source 1..35: mol_type = other DNA
  misc_feature 1..35: note = ULp2
ttttttgcggccgcccaggcatcgacgtagaattc

SEQ ID NO: 124 - DNA, 35 bp, synthetic construct
  source 1..35: mol_type = other DNA
  misc_feature 1..35: note = ULp3
ttttttgcggccgcccagacatcgacgaaaaattc

SEQ ID NO: 125 - DNA, 35 bp, synthetic construct
  source 1..35: mol_type = other DNA
  misc_feature 1..35: note = ULp4
ttttttgcggccgcccaggcatggacgtaaaattg

SEQ ID NO: 126 - DNA, 35 bp, synthetic construct
  source 1..35: mol_type = other DNA
  misc_feature 1..35: note = ULp5
ttttttgcggccgcccaagtctcgacataaaattc

SEQ ID NO: 127 - DNA, 35 bp, synthetic construct
  source 1..35: mol_type = other DNA
  misc_feature 1..35: note = ULp6
ttttttgcggccgcccaggcatcgacgagccattg

SEQ ID NO: 128 - DNA, 35 bp, synthetic construct
  source 1..35: mol_type = other DNA
  misc_feature 1..35: note = ULp7
ttttttgcggccgcccaggcatcgacgtgccattc

SEQ ID NO: 129 - DNA, 35 bp, synthetic construct
  source 1..35: mol_type = other DNA
  misc_feature 1..35: note = ULp8
ttttttgcggccgcccaggcatcgacgtggaattc

SEQ ID NO: 130 - DNA, 35 bp, synthetic construct
  source 1..35: mol_type = other DNA
  misc_feature 1..35: note = ULp9
ttttttgcggccgcccaggcatcgacgtggaagct

SEQ ID NO: 131 - protein, 46 aa, synthetic construct
  REGION 1..46: note = G3 Parental
GGSEGDKTCPDGYEHTCGCIGGCGCKRSACIGALCCQASLGGWLSD

SEQ ID NO: 132 - protein, 45 aa, synthetic construct
  REGION 1..45: note = G3 NTRUNC1
GGSGDKTCPDGYEHTCGCIGGCGCKRSACIGALCCQASLGGWLSD

SEQ ID NO: 133 - protein, 44 aa, synthetic construct
  REGION 1..44: note = G3 NTRUNC2
GGSDKTCPDGYEHTCGCIGGCGCKRSACIGALCCQASLGGWLSD

SEQ ID NO: 134 - protein, 43 aa, synthetic construct
  REGION 1..43: note = G3 NTRUNC3
GGSKTCPDGYEHTCGCIGGCGCKRSACIGALCCQASLGGWLSD

SEQ ID NO: 135 - protein, 42 aa, synthetic construct
  REGION 1..42: note = G3 NTRUNC4
GGSTCPDGYEHTCGCIGGCGCKRSACIGALCCQASLGGWLSD

SEQ ID NO: 136 - protein, 41 aa, synthetic construct
  REGION 1..41: note = G3 NTRUNC5
GGSCPDGYEHTCGCIGGCGCKRSACIGALCCQASLGGWLSD

SEQ ID NO: 137 - DNA, 107 bp, synthetic construct
  source 1..107: mol_type = other DNA
  misc_feature 1..107: note = Ultralong CD3 Antibody 014
tttgttgccaagcgtcgttgggtggttggcttagtgacggtgaaacctacacttacgagttccacgtcgatacctggggccaaggactcgtggtcaccgtctcctca

SEQ ID NO: 138 - DNA, 107 bp, synthetic construct
  source 1..107: mol_type = other DNA
  misc_feature 1..107: note = Ultralong CD3 Antibody 015
gtgccttctgtagtcgtactggtgattatactagtcctactgaacttgacatttacgagttctacgtcgaagggtggggccagggagtcccggtcaccgtctcctca

SEQ ID NO: 139 - DNA, 107 bp, synthetic construct
  source 1..107: mol_type = other DNA
  misc_feature 1..107: note = Ultralong CD3 Antibody 032
cttggcctagtggtaatggtagagccgacagtagtattggtgaaacttatggttacgaatttcacgtggctgcctggggccaaggactcctggtcaccgtctcctca

SEQ ID NO: 140 - DNA, 107 bp, synthetic construct
  source 1..107: mol_type = other DNA
  misc_feature 1..107: note = Ultralong CD3 Antibody 016
tttgttgtcgttctgatttgggtggctatcttactgatagtcctgcttacatttacgaatggtatattgatctttggggccaaggactcctggtcaccgtctcctca

SEQ ID NO: 141 - DNA, 107 bp, synthetic construct
  source 1..107: mol_type = other DNA
  misc_feature 1..107: note = Ultralong CD3 Antibody 031
gtgaattctgtagtgctactggtgattggactagtcctagtgaagaagacttttacgaattctacgtcgatacgtggggccagggagccccggtcaccgtctcctca

SEQ ID NO: 142 - DNA, 107 bp, synthetic construct
  source 1..107: mol_type = other DNA
  misc_feature 1..107: note = Ultralong CD3 Antibody 027
ctaattgtagaggcgttttgtgtcctactcttaacgaaatcgttgcttatacctacgaatggcacgtcgacgcctggggccaaggactcctggtcaccgtctcctca

SEQ ID NO: 143 - DNA, 107 bp, synthetic construct
  source 1..107: mol_type = other DNA
  misc_feature 1..107: note = Ultralong CD3 Antibody 021
gtgccttctgtagtcgtactggtgattatactagtcctagtgaatttgacatttacgagttctacgtcgaagggtggggccagggactcctggtcaccgtctcctca

SEQ ID NO: 144 - DNA, 107 bp, synthetic construct
  source 1..107: mol_type = other DNA
  misc_feature 1..107: note = Ultralong CD3 Antibody 026
gttaccacagcactgatccttctcattatactggtgcgacgtatatttacacgtacagcttgcacatcgatgcctggggccaaggactcctggtcaccgtctcctca

SEQ ID NO: 145 - DNA, 93 bp, synthetic construct
  source 1..93: mol_type = other DNA
  misc_feature 1..93: note = Standard Short CDR3 Antibody 028
gcaagaagtcgtggttatgattgttatgctaatgtggatgctttggactacgtcgatgcctggggccaaggactcctggtcaccgtctcctca

SEQ ID NO: 146 - DNA, 107 bp, synthetic construct
  source 1..107: mol_type = other DNA
  misc_feature 1..107: note = Ultralong CD3 Antibody 018
agtggaatttagaatatacttggggtggtgttggttgcgctagttttgctgatgaggacacccacgttgatgcctggggccaaggactcctggtcaccgtctcctca

SEQ ID NO: 147 - DNA, 107 bp, synthetic construct
  source 1..107: mol_type = other DNA
  misc_feature 1..107: note = Ultralong CD3 Antibody 019
attatgttgttcgtcgttataattgtggtggtcttggttatgggcatggctttaatagtttctacgtcgatgcctggggccaaggactcctggtcaccgtctcctca

SEQ ID NO: 148 - DNA, 107 bp, synthetic construct; identical to SEQ ID NO: 147
  source 1..107: mol_type = other DNA
  misc_feature 1..107: note = Ultralong CD3 Antibody 020

SEQ ID NO: 149 - DNA, 107 bp, synthetic construct
  source 1..107: mol_type = other DNA
  misc_feature 1..107: note = Ultralong CD3 Antibody 022
atcgggttgtgcgtcgtaataattgtggtgggcttggttatgattatggttttgatcatttctacgtcgatgcctggggccaaggactcctggtcaccgtctcctca

SEQ ID NO: 150 - DNA, 99 bp, synthetic construct
  source 1..99: mol_type = other DNA
  misc_feature 1..99: note = Ultralong CD3 Antibody 023
gcgaagtttgctaagggtactacgagtgctggtgcttgtgattattcagaaagctacgtcgatgcctggggccagggactcctggtcaccgtctcctca

SEQ ID NO: 151 - DNA, 100 bp, synthetic construct
  source 1..100: mol_type = other DNA
  misc_feature 1..100: note = Ultralong CD3 Antibody 024
attccggtgcttatgcttatgctgcttgcaattattatggttggcgttgtgcttgggaaagctacatcgatgcctggggccaaggactcctggtcaccgt

SEQ ID NO: 152 - DNA, 107 bp, synthetic construct
  source 1..107: mol_type = other DNA
  misc_feature 1..107: note = Ultralong CD3 Antibody 025
acaatgcacgttgtgatagttggacgtatgacagctgtgatacttggtatcgcaattcgtggcacgttgatgcctggggccaaggactcctggtcaccgtctcctca

SEQ ID NO: 153 - DNA, 93 bp, synthetic construct
  source 1..93: mol_type = other DNA
  misc_feature 1..93: note = Standard Short CDR3 Antibody 029
gcaagaagtcgtggttatgattgttatgcttatgtttatgctttggacaccgtcgatgcctggggccaaggactcctggtcaccgtctcctca

SEQ ID NO: 154 - DNA, 93 bp, synthetic construct; identical to SEQ ID NO: 145
  source 1..93: mol_type = other DNA
  misc_feature 1..93: note = Standard Short CDR3 Antibody 030

SEQ ID NO: 155 - protein, 39 aa, synthetic construct
  REGION 1..39: note = R2G3
TCPDGYEHTCGCIGGCGCKRSACIGALCCQASLGGWLSD

SEQ ID NO: 156 - protein, 40 aa, synthetic construct
  REGION 1..40: note = Forward primer
GGAACCCATGGCCGGCNNKNNKACGTGTCCTGATGGTTAC

SEQ ID NO: 157 - protein, 37 aa, synthetic construct
  REGION 1..37: note = Reverse primer
GGAACGCGGCCGCMNNMNNGTCACTAAGCCAACCACC

SEQ ID NO: 158 - protein, 43 aa, synthetic construct
  REGION 1..43: note = G3-A knob
TITCPDGYEHTCGCIGGCGCKRSACIGALCCQASLGGWLSDQS

SEQ ID NO: 159 - protein, 43 aa, synthetic construct
  REGION 1..43: note = G3-B knob
CKTCPDGYEHTCGCIGGCGCKRSACIGALCCQASLGGWLSDQH

SEQ ID NO: 160 - protein, 43 aa, synthetic construct
  REGION 1..43: note = G3-C knob
DYTCPDGYEHTCGCIGGCGCKRSACIGALCCQASLGGWLSDMP

SEQ ID NO: 161 - protein, 43 aa, synthetic construct
  REGION 1..43: note = G3-D knob
LQTCPDGYEHTCGCIGGCGCKRSACIGALCCQASLGGWLSDPP

SEQ ID NO: 162 - protein, 43 aa, synthetic construct
  REGION 1..43: note = G3-E knob
SVTCPDGYEHTCGCIGGCGCKRSACIGALCCQASLGGWLSDYI

SEQ ID NO: 163 - protein, 43 aa, synthetic construct
  REGION 1..43: note = G3-F knob
NGTCPDGYEHTCGCIGGCGCKRSACIGALCCQASLGGWLSDMQ

SEQ ID NO: 164 - protein, 43 aa, synthetic construct
  REGION 1..43: note = G3-G knob
LVTCPDGYEHTCGCIGGCGCKRSACIGALCCQASLGGWLSDIP

SEQ ID NO: 165 - protein, 43 aa, synthetic construct
  REGION 1..43: note = G3-H knob
QGTCPDGYEHTCGCIGGCGCKRSACIGALCCQASLGGWLSDCQ

SEQ ID NO: 166 - protein, 43 aa, synthetic construct
  REGION 1..43: note = G3-I knob
LSTCPDGYEHTCGCIGGCGCKRSACIGALCCQASLGGWLSDQV

SEQ ID NO: 167 - protein, 43 aa, synthetic construct
  REGION 1..43: note = G3-K knob
MITCPDGYEHTCGCIGGCGCKRSACIGALCCQASLGGWLSDQV

SEQ ID NO: 168 - protein, 43 aa, synthetic construct
  REGION 1..43: note = G3-L knob
LVTCPDGYEHTCGCIGGCGCKRSACIGALCCQASLGGWLSDRQ

SEQ ID NO: 169 - protein, 43 aa, synthetic construct
  REGION 1..43: note = G3-M knob
QPTCPDGYEHTCGCIGGCGCKRSACIGALCCQASLGGWLSDKQ

SEQ ID NO: 170 - protein, 43 aa, synthetic construct
  REGION 1..43: note = G3-N
QQTCPDGYEHTCGCIGGCGCKRSACIGALCCQASLGGWLSDYL

SEQ ID NO: 171 - protein, 43 aa, synthetic construct
  REGION 1..43: note = G3-O
AQTCPDGYEHTCGCIGGCGCKRSACIGALCCQASLGGWLSDYH

SEQ ID NO: 172 - protein, 43 aa, synthetic construct
  REGION 1..43: note = G3-P
HLTCPDGYEHTCGCIGGCGCKRSACIGALCCQASLGGWLSDQQ

SEQ ID NO: 173 - protein, 43 aa, synthetic construct
  REGION 1..43: note = G3-Q
PQTCPDGYEHTCGCIGGCGCKRSACIGALCCQASLGGWLSDQF

SEQ ID NO: 174 - protein, 43 aa, synthetic construct
  REGION 1..43: note = G3-R
HWTCPDGYEHTCGCIGGCGCKRSACIGALCCQASLGGWLSDSF

SEQ ID NO: 175 - protein, 43 aa, synthetic construct
  REGION 1..43: note = G3-S
SCTCPDGYEHTCGCIGGCGCKRSACIGALCCQASLGGWLSDQP

SEQ ID NO: 176 - protein, 43 aa, synthetic construct
  REGION 1..43: note = G3-T
PFTCPDGYEHTCGCIGGCGCKRSACIGALCCQASLGGWLSDQH

SEQ ID NO: 177 - protein, 43 aa, synthetic construct
  REGION 1..43: note = G3-U
WMTCPDGYEHTCGCIGGCGCKRSACIGALCCQASLGGWLSDQQ

SEQ ID NO: 178 - protein, 43 aa, synthetic construct
  REGION 1..43: note = G3-V
SCTCPDGYEHTCGCIGGCGCKRSACIGALCCQASLGGWLSDDS

SEQ ID NO: 179 - protein, 43 aa, synthetic construct
  REGION 1..43: note = G3-W
LSTCPDGYEHTCGCIGGCGCKRSACIGALCCQASLGGWLSDVQ

SEQ ID NO: 180 - protein, 43 aa, synthetic construct
  REGION 1..43: note = G3-X
QLTCPDGYEHTCGCIGGCGCKRSACIGALCCQASLGGWLSDYV

SEQ ID NO: 181 - protein, 43 aa, synthetic construct
  REGION 1..43: note = G3-Y
LVTCPDGYEHTCGCIGGCGCKRSACIGALCCQASLGGWLSDQQ

SEQ ID NO: 182 - protein, 43 aa, synthetic construct
  REGION 1..43: note = G3-Z
GMTCPDGYEHTCGCIGGCGCKRSACIGALCCQASLGGWLSDRS

SEQ ID NO: 183 - protein, 43 aa, synthetic construct
  REGION 1..43: note = G3-AA
ISTCPDGYEHTCGCIGGCGCKRSACIGALCCQASLGGWLSDTV

SEQ ID NO: 184 - protein, 43 aa, synthetic construct
  REGION 1..43: note = G3-BB
SQTCPDGYEHTCGCIGGCGCKRSACIGALCCQASLGGWLSDHL

SEQ ID NO: 185 - protein, 43 aa, synthetic construct
  REGION 1..43: note = G3-CC
CDTCPDGYEHTCGCIGGCGCKRSACIGALCCQASLGGWLSDQR

SEQ ID NO: 186 - protein, 43 aa, synthetic construct
  REGION 1..43: note = G3-DD
SLTCPDGYEHTCGCIGGCGCKRSACIGALCCQASLGGWLSDQR

SEQ ID NO: 187 - protein, 40 aa, synthetic construct
  REGION 1..40: note = R4C1
NCPDGYIYSSNTASGYDCGVWICRRVGSAFCSRTGDYTSP

SEQ ID NO: 188 - protein, 44 aa, synthetic construct
  REGION 1..44: note = HW-SF modified R4C1
HWNCPDGYIYSSNTASGYDCGVWICRRVGSAFCSRTGDYTSPSF

SEQ ID NO: 189 - protein, 44 aa, synthetic construct
  REGION 1..44: note = IS-TV modified R4C1
ISNCPDGYIYSSNTASGYDCGVWICRRVGSAFCSRTGDYTSPTV

SEQ ID NO: 190 - protein, 42 aa, synthetic construct
  REGION 1..42: note = R2D9
TCPNGYNWFDRCCSWDGTCGDGCCSNRAWPSGNGRADSSIGE

SEQ ID NO: 191 - protein, 46 aa, synthetic construct
  REGION 1..46: note = HW-SF modified R2D9
HWTCPNGYNWFDRCCSWDGTCGDGCCSNRAWPSGNGRADSSIGESF

SEQ ID NO: 192 - protein, 46 aa, synthetic construct
  REGION 1..46: note = IS-TV modified R2D9
ISTCPNGYNWFDRCCSWDGTCGDGCCSNRAWPSGNGRADSSIGETV

SEQ ID NO: 193 - DNA, 327 bp, synthetic construct
  source 1..327: mol_type = other DNA
  rRNA 1..327: note = TrxA
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcc

SEQ ID NO: 194 - protein, 109 aa, synthetic construct
  REGION 1..109: note = TrxA
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 195 - protein, 92 aa, synthetic construct
  REGION 1..92: note = TrxA N-terminal portion before last engineered loop
MSDKIIHLTDDSFDTDVLKADGAILVDFWAEWCGPCKMIAPILDEIADEYQGKLTVAKLNIDQNPGTAPKYGIRGIPTLLLFKNGEVAATKV

SEQ ID NO: 196 - protein, 6 aa, synthetic construct
  REGION 1..6: note = SKM stalk A
HQETLR

SEQ ID NO: 197 - protein, 6 aa, synthetic construct
  REGION 1..6: note = SKM stalk B
YTYSLH

SEQ ID NO: 198 - protein, 44 aa, synthetic construct
  REGION 1..44: note = SKM knob
SCPDGYIDNSGCTADWGCAALDCWRRRFGYHSTDPSHYTGATYI

SEQ ID NO: 199 - protein, 17 aa, synthetic construct
  REGION 1..17: note = TrxA C-terminal portion after last engineered loop
GALSKGQLKEFLDANLA

SEQ ID NO: 200 - DNA, 549 bp, synthetic construct
  source 1..549: mol_type = other DNA
  misc_feature 1..549: note = TrxA Loop SKS-STM Nucleotide
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgcaccaagagaccttacgtagttgtcctgatggttatattgataattctggatgcacggctgattggggttgtgcagctcttgattgttggcggcgtcgttttggttaccacagcactgatccttctcattatactggtgcgacgtatatttacacgtacagcttgcacggtgcactgtctaaaggtcagttgaaagagttcctcgacgctaacctggccggtggccaggaaacctttagcgatctgtggaaactgctgccggaaaatctcgag

SEQ ID NO: 201 - protein, 183 aa, synthetic construct
  REGION 1..183: note = TrxA Loop SKS-STM Amino acid
MSDKIIHLTDDSFDTDVLKADGAILVDFWAEWCGPCKMIAPILDEIADEYQGKLTVAKLNIDQNPGTAPKYGIRGIPTLLLFKNGEVAATKVHQETLRSCPDGYIDNSGCTADWGCAALDCWRRRFGYHSTDPSHYTGATYIYTYSLHGALSKGQLKEFLDANLAGGQETFSDLWKLLPENLE

SEQ ID NO: 202 - protein, 23 aa, synthetic construct
  REGION 1..23: note = Coiled-coil ascending
GGSGAKLAALKAKLAALKGGGGS

SEQ ID NO: 203 - protein, 23 aa, synthetic construct
  REGION 1..23: note = Coiled-coil descending
GGGGSELAALEAELAALEAGGSG

SEQ ID NO: 204 - DNA, 693 bp, synthetic construct
  source 1..693: mol_type = other DNA
  misc_feature 1..693: note = TrxA_Loop_CKC-SKM nucleotide
tctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggcggcagcggcgcgaaactggcggcgctgaaagcgaaactggcggcgctgaaaggcggcggcggcagcagttgtcctgatggttatattgataattctggatgcacggctgattggggttgtgcagctcttgattgttggcggcgtcgttttggttaccacagcactgatccttctcattatactggtgcgacgtatattggcggcggcggcagcgaactggcggcgctggaagcggaactggcggcgctggaagcgggcggcagcggcggtgcactgtctaaaggtcagttgaaagagttcctcgacgctaacctggccggtggccaggaaacctttagcgatctgtggaaactgctgccggaaaatctcgag

SEQ ID NO: 205 - protein, 217 aa, synthetic construct
  REGION 1..217: note = TrxA_Loop_CKC-SKM Amino acid
MSDKIIHLTDDSFDTDVLKADGAILVDFWAEWCGPCKMIAPILDEIADEYQGKLTVAKLNIDQNPGTAPKYGIRGIPTLLLFKNGEVAATKVGGSGAKLAALKAKLAALKGGGGSSCPDGYIDNSGCTADWGCAALDCWRRRFGYHSTDPSHYTGATYIGGGGSELAALEAELAALEAGGSGGALSKGQLKEFLDANLAGGQETFSDLWKLLPENLE

SEQ ID NO: 206 - DNA, 576 bp, synthetic construct
  source 1..576: mol_type = other DNA
  misc_feature 1..576: note = TrxA_Loop_GKG-SKM Nucleotide
tctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggcggcggatccagttgtcctgatggttatattgataattctggatgcacggctgattggggttgtgcagctcttgattgttggcggcgtcgttttggttaccacagcactgatccttctcattatactggtgcgacgtatattggcggtaccggtgcactgtctaaaggtcagttgaaagagttcctcgacgctaacctggccggtggccaggaaacctttagcgatctgtggaaactgctgccggaaaatctcgag

SEQ ID NO: 207 - protein, 178 aa, synthetic construct
  REGION 1..178: note = TrxA_Loop_GKG-SKM Amino acid
MSDKIIHLTDDSFDTDVLKADGAILVDFWAEWCGPCKMIAPILDEIADEYQGKLTVAKLNIDQNPGTAPKYGIRGIPTLLLFKNGEVAATKVGGGSSCPDGYIDNSGCTADWGCAALDCWRRRFGYHSTDPSHYTGATYIGGTGALSKGQLKEFLDANLAGGQETFSDLWKLLPENLE

SEQ ID NO: 208 - protein, 14 aa, synthetic construct
  REGION 1..14: note = D01 tag
QETFSDLWKLLPEN

SEQ ID NO: 209 - protein, 8 aa, synthetic construct
  REGION 1..8: note = Flag/Enterokinase site
DYKDDDDK

SEQ ID NO: 210 - protein, 13 aa, synthetic construct
  REGION 1..13: note = Flag/Enterokinase site with GS linkers
GGSDYKDDDDKGS

SEQ ID NO: 211 - DNA, 633 bp, synthetic construct
  source 1..633: mol_type = other DNA
  misc_feature 1..633: note = TrxA_Cterm_SKS-SKM nucleotide
tctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggcggatccgattataaagatgacgacgataaaggcagccaccaagagaccttacgtagttgtcctgatggttatattgataattctggatgcacggctgattggggttgtgcagctcttgattgttggcggcgtcgttttggttaccacagcactgatccttctcattatactggtgcgacgtatatttacacgtacagcttgcacggcggtagccaggaaacctttagcgatctgtggaaactgctgccggaaaatctcgag

SEQ ID NO: 212 - protein, 197 aa, synthetic construct
  REGION 1..197: note = TrxA_Cterm_SKS-SKM Amino acid
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGSDYKDDDDKGSHQETLRSCPDGYIDNSGCTADWGCAALDCWRRRFGYHSTDPSHYTGATYIYTYSLHGGSQETFSDLWKLLPENLE

SEQ ID NO: 213 - DNA, 735 bp, synthetic construct
  source 1..735: mol_type = other DNA
  misc_feature 1..735: note = TrxA_Cterm_CKC-SKM Nucleotide
tctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggcggatccgattataaagatgacgacgataaaggcagcggcggcagcggcgcgaaactggcggcgctgaaagcgaaactggcggcgctgaaaggcggcggcggcagcagttgtcctgatggttatattgataattctggatgcacggctgattggggttgtgcagctcttgattgttggcggcgtcgttttggttaccacagcactgatccttctcattatactggtgcgacgtatattggcggcggcggcagcgaactggcggcgctggaagcggaactggcggcgctggaagcgggcggcagcggcggcggtagccaggaaacctttagcgatctgtggaaactgctgccggaaaatctcgag

SEQ ID NO: 214 - protein, 231 aa, synthetic construct
  REGION 1..231: note = TrxA_Cterm_CKC-SKM
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGSDYKDDDDKGSGGSGAKLAALKAKLAALKGGGGSSCPDGYIDNSGCTADWGCAALDCWRRRFGYHSTDPSHYTGATYIGGGGSELAALEAELAALEAGGSGGGSQETFSDLWKLLPENLE

SEQ ID NO: 215 - DNA, 600 bp, synthetic construct
  source 1..600: mol_type = other DNA
  misc_feature 1..600: note = TrxA_Cterm_GKG-SKM Nucleotide
tctagaaataattttgtttaactttaagaaggagatatacatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggcggtagcgattataaagatgacgacgataaaggcggatccagttgtcctgatggttatattgataattctggatgcacggctgattggggttgtgcagctcttgattgttggcggcgtcgttttggttaccacagcactgatccttctcattatactggtgcgacgtatattggcaccggtcaggaaacctttagcgatctgtggaaactgctgccggaaaatctcgag

SEQ ID NO: 216 - protein, 186 aa, synthetic construct
  REGION 1..186: note = TrxA_Cterm_GKG-SKM Amino acid
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGSDYKDDDDKGGSSCPDGYIDNSGCTADWGCAALDCWRRRFGYHSTDPSHYTGATYIGTGQETFSDLWKLLPENLE

SEQ ID NO: 217 - protein, 6 aa, synthetic construct
  REGION 1..6: note = 6xHis
HHHHHH

SEQ ID NO: 218 - protein, 6 aa, synthetic construct
  REGION 1..6: note = Factor Xa cleavage site
  VARIANT 6: note = X can be any amino acid except arginine and proline
IEDGRX

SEQ ID NO: 219 - skipped

SEQ ID NO: 220 - protein, 6 aa, synthetic construct
  REGION 1..6: note = Thrombin cleavage site
LVPRGS

SEQ ID NO: 221 - protein, 6 aa, synthetic construct
  REGION 1..6: note = Thrombin cleavage site
LVPRGF

SEQ ID NO: 222 - protein, 6 aa, synthetic construct
  REGION 1..6: note = Thrombin cleavage site
MYPRGN

SEQ ID NO: 223 - protein, 7 aa, synthetic construct
  REGION 1..7: note = TEV protease cleavage site
  VARIANT 2: note = X can be any amino acid
  VARIANT 3: note = X can be any amino acid
  VARIANT 5: note = X can be any amino acid
EXXYXQS

SEQ ID NO: 224-226 - skipped (3 entries)

SEQ ID NO: 227 - protein, 40 aa, synthetic construct
  REGION 1..40: note = 4C1 knob
CPDGYIYSSNTASGYDCGVWICRRVGSAFCSRTGDYTSPS

SEQ ID NO: 228 - protein, 41 aa, synthetic construct
  REGION 1..41: note = 4C1 knob
NCPDGYIYSSNTASGYDCGVWICRRVGSAFCSRTGDYTSPS

SEQ ID NO: 229 - protein, 40 aa, synthetic construct
  REGION 1..40: note = R2C3 (R5C1) knob
CPDGYLYSSNTGRGYDCGVWTCRRVGGEFCSATGDWTSPS

SEQ ID NO: 230 - protein, 41 aa, synthetic construct
  REGION 1..41: note = R2C3 (R5C1) knob
SCPDGYLYSSNTGRGYDCGVWTCRRVGGEFCSATGDWTSPS

SEQ ID NO: 231 - protein, 40 aa, synthetic construct
  REGION 1..40: note = SKD knob
CPGGSSRRYPSGASCDVSGGACACYVSNCRGVLCPTLNEI

SEQ ID NO: 232 - protein, 41 aa, synthetic construct
  REGION 1..41: note = SKD knob
SCPGGSSRRYPSGASCDVSGGACACYVSNCRGVLCPTLNEI

SEQ ID NO: 233 - protein, 43 aa, synthetic construct
  REGION 1..43: note = SKM knob
CPDGYIDNSGCTADWGCAALDCWRRRFGYHSTDPSHYTGATYI

SEQ ID NO: 234 - protein, 38 aa, synthetic construct
  REGION 1..38: note = 2G3 knob
CPDGYEHTCGCIGGCGCKRSACIGALCCQASLGGWLSD

SEQ ID NO: 235 - protein, 41 aa, synthetic construct
  REGION 1..41: note = R2F12 knob
CPDDFDYRCSCIGGCGCARKGCVGPLCCRSDLGGYLTDSPA

SEQ ID NO: 236 - protein, 42 aa, synthetic construct
  REGION 1..42: note = R2F12 knob
ACPDDFDYRCSCIGGCGCARKGCVGPLCCRSDLGGYLTDSPA

SEQ ID NO: 237 - protein, 40 aa, synthetic construct
  REGION 1..40: note = SR3A3 knob
CPDGYIYSSNITSGFDCGVWICRRVGSAFCSRTGDYTSPT

SEQ ID NO: 238 - protein, 41 aa, synthetic construct
  REGION 1..41: note = SR3A3 knob
NCPDGYIYSSNITSGFDCGVWICRRVGSAFCSRTGDYTSPT

SEQ ID NO: 239 - protein, 40 aa, synthetic construct
  REGION 1..40: note = R2D9 knob
CPNGYNWFDRCCSWDGTCGDGCCSNRAWPSGNGRADSSIG

SEQ ID NO: 240 - protein, 41 aa, synthetic construct
  REGION 1..41: note = R2D9 knob
TCPNGYNWFDRCCSWDGTCGDGCCSNRAWPSGNGRADSSIG

SEQ ID NO: 241 - protein, 53 aa, synthetic construct
  REGION 1..53: note = R4C1 knob
RKEKNCPDGYIYSSNTASGYDCGVWICRRVGSAFCSRTGDYTSPSEFDIYEFY

SEQ ID NO: 242 - protein, 43 aa, synthetic construct
  REGION 1..43: note = R4C1 knob
RKEKNCPDGYIYSSNTASGYDCGVWICRRVGSAFCSRTGDYTS

SEQ ID NO: 243 - protein, 44 aa, synthetic construct
  REGION 1..44: note = R4C1 knob
RKEKNCPDGYIYSSNTASGYDCGVWICRRVGSAFCSRTGDYTSP

SEQ ID NO: 244 - protein, 45 aa, synthetic construct
  REGION 1..45: note = R4C1 knob
RKEKNCPDGYIYSSNTASGYDCGVWICRRVGSAFCSRTGDYTSPS

SEQ ID NO: 245 - protein, 46 aa, synthetic construct
  REGION 1..46: note = R4C1 knob
RKEKNCPDGYIYSSNTASGYDCGVWICRRVGSAFCSRTGDYTSPSE

SEQ ID NO: 246 - protein, 47 aa, synthetic construct
  REGION 1..47: note = R4C1 knob
RKEKNCPDGYIYSSNTASGYDCGVWICRRVGSAFCSRTGDYTSPSEF

SEQ ID NO: 247 - protein, 48 aa, synthetic construct
  REGION 1..48: note = R4C1 knob
RKEKNCPDGYIYSSNTASGYDCGVWICRRVGSAFCSRTGDYTSPSEFD

SEQ ID NO: 248 - protein, 49 aa, synthetic construct
  REGION 1..49: note = R4C1 knob
RKEKNCPDGYIYSSNTASGYDCGVWICRRVGSAFCSRTGDYTSPSEFDI

SEQ ID NO: 249 - protein, 51 aa, synthetic construct
  REGION 1..51: note = R4C1 knob
RKEKNCPDGYIYSSNTASGYDCGVWICRRVGSAFCSRTGDYTSPSEFDIYE

SEQ ID NO: 250 - protein, 52 aa, synthetic construct
  REGION 1..52: note = R4C1 knob
RKEKNCPDGYIYSSNTASGYDCGVWICRRVGSAFCSRTGDYTSPSEFDIYEF

SEQ ID NO: 251 - protein, 43 aa, synthetic construct
  REGION 1..43: note = R2C3 knob
RKEKSCPDGYLYSSNTGRGYDCGVWTCRRVGGEFCSATGDWTS

SEQ ID NO: 252 - protein, 44 aa, synthetic construct
  REGION 1..44: note = R2C3 knob
RKEKSCPDGYLYSSNTGRGYDCGVWTCRRVGGEFCSATGDWTSP

SEQ ID NO: 253 - protein, 45 aa, synthetic construct
  REGION 1..45: note = R2C3 knob
RKEKSCPDGYLYSSNTGRGYDCGVWTCRRVGGEFCSATGDWTSPS

SEQ ID NO: 254 - protein, 46 aa, synthetic construct
  REGION 1..46: note = R2C3 knob
RKEKSCPDGYLYSSNTGRGYDCGVWTCRRVGGEFCSATGDWTSPSE

SEQ ID NO: 255 - protein, 47 aa, synthetic construct
  REGION 1..47: note = R2C3 knob
RKEKSCPDGYLYSSNTGRGYDCGVWTCRRVGGEFCSATGDWTSPSEE

SEQ ID NO: 256 - protein, 48 aa, synthetic construct
  REGION 1..48: note = R2C3 knob
RKEKSCPDGYLYSSNTGRGYDCGVWTCRRVGGEFCSATGDWTSPSEED

SEQ ID NO: 257 - protein, 49 aa, synthetic construct
  REGION 1..49: note = R2C3 knob
RKEKSCPDGYLYSSNTGRGYDCGVWTCRRVGGEFCSATGDWTSPSEEDF

SEQ ID NO: 258 - protein, 50 aa, synthetic construct
  REGION 1..50: note = R2C3 knob
RKEKSCPDGYLYSSNTGRGYDCGVWTCRRVGGEFCSATGDWTSPSEEDFY

SEQ ID NO: 259 - protein, 51 aa, synthetic construct
  REGION 1..51: note = R2C3 knob
RKEKSCPDGYLYSSNTGRGYDCGVWTCRRVGGEFCSATGDWTSPSEEDFYE

SEQ ID NO: 260 - protein, 55 aa, synthetic construct
  REGION 1..55: note = SKD knob
SEKRSCPGGSSRRYPSGASCDVSGGACACYVSNCRGVLCPTLNEIVAYTYEWHVD

SEQ ID NO: 261 - protein, 45 aa, synthetic construct
  REGION 1..45: note = SKD knob
SEKRSCPGGSSRRYPSGASCDVSGGACACYVSNCRGVLCPTLNEI

SEQ ID NO: 262 - protein, 46 aa, synthetic construct
  REGION 1..46: note = SKD knob
SEKRSCPGGSSRRYPSGASCDVSGGACACYVSNCRGVLCPTLNEIV

SEQ ID NO: 263 - protein, 47 aa, synthetic construct
  REGION 1..47: note = SKD knob
SEKRSCPGGSSRRYPSGASCDVSGGACACYVSNCRGVLCPTLNEIVA

SEQ ID NO: 264 - protein, 48 aa, synthetic construct
  REGION 1..48: note = SKD knob
SEKRSCPGGSSRRYPSGASCDVSGGACACYVSNCRGVLCPTLNEIVAY

SEQ ID NO: 265 - protein, 49 aa, synthetic construct
  REGION 1..49: note = SKD knob
SEKRSCPGGSSRRYPSGASCDVSGGACACYVSNCRGVLCPTLNEIVAYT

SEQ ID NO: 266 - protein, 51 aa, synthetic construct
  REGION 1..51: note = SKD knob
SEKRSCPGGSSRRYPSGASCDVSGGACACYVSNCRGVLCPTLNEIVAYTYE

SEQ ID NO: 267 - protein, 52 aa, synthetic construct
  REGION 1..52: note = SKD knob
SEKRSCPGGSSRRYPSGASCDVSGGACACYVSNCRGVLCPTLNEIVAYTYEW

SEQ ID NO: 268 - protein, 53 aa, synthetic construct
  REGION 1..53: note = SKD knob
SEKRSCPGGSSRRYPSGASCDVSGGACACYVSNCRGVLCPTLNEIVAYTYEWH

SEQ ID NO: 269 - protein, 54 aa, synthetic construct
  REGION 1..54: note = SKD knob
SEKRSCPGGSSRRYPSGASCDVSGGACACYVSNCRGVLCPTLNEIVAYTYEWHV

SEQ ID NO: 270 - protein, 53 aa, synthetic construct
  REGION 1..53: note = SKM knob
LRSCPDGYIDNSGCTADWGCAALDCWRRRFGYHSTDPSHYTGATYIYTYSHID

SEQ ID NO: 271 - protein, 44 aa, synthetic construct
  REGION 1..44: note = SKM knob
LRSCPDGYIDNSGCTADWGCAALDCWRRRFGYHSTDPSHYTGAT

SEQ ID NO: 272 - protein, 45 aa, synthetic construct
  REGION 1..45: note = SKM knob
LRSCPDGYIDNSGCTADWGCAALDCWRRRFGYHSTDPSHYTGATY

SEQ ID NO: 273 - protein, 46 aa, synthetic construct
  REGION 1..46: note = SKM knob
LRSCPDGYIDNSGCTADWGCAALDCWRRRFGYHSTDPSHYTGATYI

SEQ ID NO: 274 - protein, 47 aa, synthetic construct
  REGION 1..47: note = SKM knob
LRSCPDGYIDNSGCTADWGCAALDCWRRRFGYHSTDPSHYTGATYIY

SEQ ID NO: 275 - protein, 48 aa, synthetic construct
  REGION 1..48: note = SKM knob
LRSCPDGYIDNSGCTADWGCAALDCWRRRFGYHSTDPSHYTGATYIYT

SEQ ID NO: 276 - protein, 50 aa, synthetic construct
  REGION 1..50: note = SKM knob
LRSCPDGYIDNSGCTADWGCAALDCWRRRFGYHSTDPSHYTGATYIYTYS

SEQ ID NO: 277 - protein, 50 aa, synthetic construct; identical to SEQ ID NO: 276
  REGION 1..50: note = SKM knob

SEQ ID NO: 278 - protein, 51 aa, synthetic construct
  REGION 1..51: note = SKM knob
LRSCPDGYIDNSGCTADWGCAALDCWRRRFGYHSTDPSHYTGATYIYTYSH

SEQ ID NO: 279 - protein, 52 aa, synthetic construct
  REGION 1..52: note = SKM knob
LRSCPDGYIDNSGCTADWGCAALDCWRRRFGYHSTDPSHYTGATYIYTYSHI

SEQ ID NO: 280 - protein, 42 aa, synthetic construct
  REGION 1..42: note = R2G3 knob
EGDKTCPDGYEHTCGCIGGCGCKRSACIGALCCQASLGGWLS

SEQ ID NO: 281 - protein, 43 aa, synthetic construct
  REGION 1..43: note = R2G3 knob
EGDKTCPDGYEHTCGCIGGCGCKRSACIGALCCQASLGGWLSD

SEQ ID NO: 282 - protein, 44 aa, synthetic construct
  REGION 1..44: note = R2G3 knob
EGDKTCPDGYEHTCGCIGGCGCKRSACIGALCCQASLGGWLSDG

SEQ ID NO: 283 - protein, 45 aa, synthetic construct
  REGION 1..45: note = R2G3 knob
EGDKTCPDGYEHTCGCIGGCGCKRSACIGALCCQASLGGWLSDGE

SEQ ID NO: 284 - protein, 46 aa, synthetic construct
  REGION 1..46: note = R2G3 knob
EGDKTCPDGYEHTCGCIGGCGCKRSACIGALCCQASLGGWLSDGET

SEQ ID NO: 285 - protein, 47 aa, synthetic construct
  REGION 1..47: note = R2G3 knob
EGDKTCPDGYEHTCGCIGGCGCKRSACIGALCCQASLGGWLSDGETY

SEQ ID NO: 286 - protein, 48 aa, synthetic construct
  REGION 1..48: note = R2G3 knob
EGDKTCPDGYEHTCGCIGGCGCKRSACIGALCCQASLGGWLSDGETYT

SEQ ID NO: 287 - protein, 49 aa, synthetic construct
  REGION 1..49: note = R2G3 knob
EGDKTCPDGYEHTCGCIGGCGCKRSACIGALCCQASLGGWLSDGETYTY

SEQ ID NO: 288 - protein, 50 aa, synthetic construct
  REGION 1..50: note = R2G3 knob
EGDKTCPDGYEHTCGCIGGCGCKRSACIGALCCQASLGGWLSDGETYTYE

SEQ ID NO: 289 - protein, 42 aa, synthetic construct
  REGION 1..42: note = R2G3 knob
GDKTCPDGYEHTCGCIGGCGCKRSACIGALCCQASLGGWLSD

SEQ ID NO: 290 - protein, 41 aa, synthetic construct
  REGION 1..41: note = R2G3 knob
DKTCPDGYEHTCGCIGGCGCKRSACIGALCCQASLGGWLSD

SEQ ID NO: 291 - protein, 40 aa, synthetic construct
  REGION 1..40: note = R2G3 knob
KTCPDGYEHTCGCIGGCGCKRSACIGALCCQASLGGWLSD

SEQ ID NO: 292 - protein, 52 aa, synthetic construct
  REGION 1..52: note = R2F12 knob
KTKNACPDDFDYRCSCIGGCGCARKGCVGPLCCRSDLGGYLTDSPAYIYEWY

SEQ ID NO: 293 - protein, 44 aa, synthetic construct
  REGION 1..44: note = R2F12 knob
KTKNACPDDFDYRCSCIGGCGCARKGCVGPLCCRSDLGGYLTDS

SEQ ID NO: 294 - protein, 45 aa, synthetic construct
  REGION 1..45: note = R2F12 knob
KTKNACPDDFDYRCSCIGGCGCARKGCVGPLCCRSDLGGYLTDSP

SEQ ID NO: 295 - protein, 46 aa, synthetic construct
  REGION 1..46: note = R2F12 knob
KTKNACPDDFDYRCSCIGGCGCARKGCVGPLCCRSDLGGYLTDSPA

SEQ ID NO: 296 - protein, 47 aa, synthetic construct
  REGION 1..47: note = R2F12 knob
KTKNACPDDFDYRCSCIGGCGCARKGCVGPLCCRSDLGGYLTDSPAY

SEQ ID NO: 297 - protein, 48 aa, synthetic construct
  REGION 1..48: note = R2F12 knob
KTKNACPDDFDYRCSCIGGCGCARKGCVGPLCCRSDLGGYLTDSPAYI

SEQ ID NO: 298 - protein, 49 aa, synthetic construct
  REGION 1..49: note = R2F12 knob
KTKNACPDDFDYRCSCIGGCGCARKGCVGPLCCRSDLGGYLTDSPAYIY

SEQ ID NO: 299 - protein, 51 aa, synthetic construct
  REGION 1..51: note = R2F12 knob
KTKNACPDDFDYRCSCIGGCGCARKGCVGPLCCRSDLGGYLTDSPAYIYEW

SEQ ID NO: 300 - protein, 42 aa, synthetic construct
  REGION 1..42: note = SR3A3 knob
KEKNCPDGYIYSSNITSGFDCGVWICRRVGSAFCSRTGDYTS

SEQ ID NO: 301 - protein, 43 aa, synthetic construct
  REGION 1..43: note = SR3A3 knob
KEKNCPDGYIYSSNITSGFDCGVWICRRVGSAFCSRTGDYTSP

SEQ ID NO: 302 - protein, 44 aa, synthetic construct
  REGION 1..44: note = SR3A3 knob
KEKNCPDGYIYSSNITSGFDCGVWICRRVGSAFCSRTGDYTSPT

SEQ ID NO: 303 - protein, 45 aa, synthetic construct
  REGION 1..45: note = SR3A3 knob
KEKNCPDGYIYSSNITSGFDCGVWICRRVGSAFCSRTGDYTSPTE

SEQ ID NO: 304 - protein, 46 aa, synthetic construct
  REGION 1..46: note = SR3A3 knob
KEKNCPDGYIYSSNITSGFDCGVWICRRVGSAFCSRTGDYTSPTEL

SEQ ID NO: 305 - protein, 47 aa, synthetic construct
  REGION 1..47: note = SR3A3 knob
KEKNCPDGYIYSSNITSGFDCGVWICRRVGSAFCSRTGDYTSPTELD

SEQ ID NO: 306 - protein, 48 aa, synthetic construct
  REGION 1..48: note = SR3A3 knob
KEKNCPDGYIYSSNITSGFDCGVWICRRVGSAFCSRTGDYTSPTELDI

SEQ ID NO: 307 - protein, 49 aa, synthetic construct
  REGION 1..49: note = SR3A3 knob
KEKNCPDGYIYSSNITSGFDCGVWICRRVGSAFCSRTGDYTSPTELDIY

SEQ ID NO: 308 - protein, 50 aa, synthetic construct
  REGION 1..50: note = SR3A3 knob
KEKNCPDGYIYSSNITSGFDCGVWICRRVGSAFCSRTGDYTSPTELDIYE

SEQ ID NO: 309 - protein, 43 aa, synthetic construct
  REGION 1..43: note = R2D9 knob
ITHKTCPNGYNWFDRCCSWDGTCGDGCCSNRAWPSGNGRADSS

SEQ ID NO: 310 - protein, 44 aa, synthetic construct
  REGION 1..44: note = R2D9 knob
ITHKTCPNGYNWFDRCCSWDGTCGDGCCSNRAWPSGNGRADSSI

SEQ ID NO: 311 - protein, 45 aa, synthetic construct
  REGION 1..45: note = R2D9 knob
ITHKTCPNGYNWFDRCCSWDGTCGDGCCSNRAWPSGNGRADSSIG

SEQ ID NO: 312 - protein, 46 aa, synthetic construct
  REGION 1..46: note = R2D9 knob
ITHKTCPNGYNWFDRCCSWDGTCGDGCCSNRAWPSGNGRADSSIGE

SEQ ID NO: 313 - protein, 47 aa, synthetic construct
  REGION 1..47: note = R2D9 knob
ITHKTCPNGYNWFDRCCSWDGTCGDGCCSNRAWPSGNGRADSSIGET

SEQ ID NO: 314 - protein, 48 aa, synthetic construct
  REGION 1..48: note = R2D9 knob
ITHKTCPNGYNWFDRCCSWDGTCGDGCCSNRAWPSGNGRADSSIGETY

SEQ ID NO: 315 - protein, 49 aa, synthetic construct
  REGION 1..49: note = R2D9 knob
ITHKTCPNGYNWFDRCCSWDGTCGDGCCSNRAWPSGNGRADSSIGETYG

SEQ ID NO: 316 - protein, 50 aa, synthetic construct
  REGION 1..50: note = R2D9 knob
ITHKTCPNGYNWFDRCCSWDGTCGDGCCSNRAWPSGNGRADSSIGETYGY

SEQ ID NO: 317 - protein, 51 aa, synthetic construct
  REGION 1..51: note = R2D9 knob
ITHKTCPNGYNWFDRCCSWDGTCGDGCCSNRAWPSGNGRADSSIGETYGYE

SEQ ID NO: 318 - protein, 56 aa, synthetic construct
  REGION 1..56: note = SKM knob
HQETLRSCPDGYIDNSGCTADWGCAALDCWRRRFGYHSTDPSHYTGATYIYTYSLH

SEQ ID NO: 319 - protein, 133 aa, synthetic construct [sequence omitted]

SEQ ID NO: 320 - protein, 114 aa, synthetic construct
  REGION 1..114: note = IL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 321 - protein, 248 aa, synthetic construct
  REGION 1..248: note = sfGFP control
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGGLEHHHHHH

SEQ ID NO: 322 - protein, 296 aa, synthetic construct
  REGION 1..296: note = sfGFP-link-G3
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGGGSKTCPDGYEHTCGCIGGCGCKRSACIGALCCQASLGGWLSDGGGSGSVQLADHYQQNTPIGDGPVLLPDNHYLSTQSVLSKDPNEKRDHMVLLEFVTAAGITHGMDELYKGGLEHHHHHH

SEQ ID NO: 323 - protein, 313 aa, synthetic construct
  REGION 1..313: note = sfGFP-stalk-G3
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDCATVHQKTAEGDKTCPDGYEHTCGCIGGCGCKRSACIGALCCQASLGGWLSDGETYTYEFHVDTWGSVQLADHYQQNTPIGDGPVLLPDNHYLSTQSVLSKDPNEKRDHMVLLEFVTAAGITHGMDELYKGGLEHHHHHH

SEQ ID NO: 324 - protein, 335 aa, synthetic construct
  REGION 1..335: note = sfGFP-coiled coil-G3
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGGSGAKLAALKAKLAALKGGGGSKTCPDGYEHTCGCIGGCGCKRSACIGALCCQASLGGWLSDGGGGSELAALEAELAALEAGGSGGSVQLADHYQQNTPIGDGPVLLPDNHYLSTQSVLSKDPNEKRDHMVLLEFVTAAGITHGMDELYKGGSLEHHHHHH

SEQ ID NO: 325 - protein, 343 aa, synthetic construct
  REGION 1..343: note = Fc-G3 coiled coil (cc)
MRRMQLLLLIALSLALVTNSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGGGGSAKLAALKAKLAALKGGGGSKTCPDGYEHTCGCIGGCGCKRSACIGALCCQASLGGWLSDGGGGSELAALEAELAALEAGGGSQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 305 aa, synthetic construct
  REGION 1..305: note = Fc-G3-link
MRRMQLLLLIALSLALVTNSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGGGGSKTCPDGYEHTCGCIGGCGCKRSACIGALCCQASLGGWLSDGGGSQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 327 - protein, 122 aa, synthetic construct
  REGION 1..122: note = CKC-G3
MGGSGAKLAALKAKLAALKGGGGSDYKDDDDKGGGSKTCPDGYEHTCGCIGGCGCKRSACIGALCCQASLGGWLSDGGGSDYKDDDDKGGGSSELAALEAELAALEAGGSGGGSLEHHHHHH

SEQ ID NO: 328 - protein, 288 aa, synthetic construct
  REGION 1..288: note = TrxA_Cterm-cchIL15
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGSDYKDDDDKGGSGAKLAALKAKLAALKGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSELAALEAELAALEAGGSGLEHHHHHH

SEQ ID NO: 329 - protein, 120 aa, synthetic construct
  REGION 1..120: note = TrxA+flag
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGGSDYKDDDDK

SEQ ID NO: 330 - protein, 168 aa, synthetic construct
  REGION 1..168: note = ccIL15
GGSGAKLAALKAKLAALKGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSELAALEAELAALEAGGSGLEHHHHHH

SEQ ID NO: 331 - protein, 210 aa, synthetic construct
  REGION 1..210: note = cc-hIL15-TEV
MDAMKRGLCCVLLLCGAVFVSPSQEIHARFRRGARGGSGAKLAALKAKLAALKGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSELAALEAELAALEAGGSGENLYFQSAGHHHHHH

SEQ ID NO: 332 - protein, 232 aa, synthetic construct
  REGION 1..232: note = IgG Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 20 aa, synthetic construct
  REGION 1..20: note = IL-2 signal
MRRMQLLLLIALSLALVTNS

(19) United States
(12) Patent Application Publication
(10) Pub. No.: US 2026/0184766 A1
(43) Pub. Date: Jul. 2, 2026

(54) FUSION POLYPEPTIDES AND BINDING PEPTIDES AND METHODS FOR PRODUCING AND USING SAME

(71) Applicant: Applied Biomedical Science Institute, San Diego, CA (US)

(72) Inventors: Vaughn SMIDER, San Diego, CA (US); Ruiqi HUANG, San Diego, CA (US); Duncan MCGREGOR, San Diego, CA (US); Alexandra STAMBAUGH, San Diego, CA (US)

(73) Assignee: Applied Biomedical Science Institute, San Diego, CA (US)

(21) Appl. No.: 19/130,004

(22) PCT Filed: Nov. 16, 2023
(86) PCT No.: PCT/US2023/080158

§ 371 Date: May 14, 2025

Related U.S. Application Data
(60) Provisional application No. 63/426,016, filed on Nov. 16, 2022.

(51) Int. Cl. C07K 16/104; A61P 31/14; C07K 14/54; C07K 14/55

(52) U.S. Cl. CPC C07K 16/104; A61P 31/14; C07K 14/5443; C07K 14/55; C07K 2317/10; C07K 2317/20; C07K 2317/24; C07K 2317/565; C07K 2317/64; C07K 2317/76; C07K 2319/30; C07K 2319/60

(57) ABSTRACT

Provided herein are fusion polypeptides in which a small polypeptide is modified by fusion with N- and C-termini sequences that interact with one another when in proximity. In some embodiments, the small polypeptide is a cysteine motif binding peptide or a cytokine. Also provided herein are cysteine motif binding peptides, such as from the knob region of an ultralong CDR3, and methods of producing and using the same. Also provided herein are compositions comprising the binding peptides.

### CROSS-REFERENCE TO RELATED APPLICATIONS

This application claims priority from U.S. provisional application No. 63/426,016, filed Nov. 16, 2022, entitled “FUSION POLYPEPTIDES AND BINDING PEPTIDES AND METHODS FOR PRODUCING AND USING SAME”, the contents of which are incorporated by reference in their entirety.

### STATEMENT OF GOVERNMENT INTEREST

This invention was made with government support under R01GM105826 awarded by the National Institutes of Health. The government has certain rights in the invention.

### INCORPORATION BY REFERENCE OF SEQUENCE LISTING

The present application is being filed along with a Sequence Listing in electronic format. The Sequence Listing is provided as a file entitled 16577-20005.40.xml created Nov. 15, 2023, which is 449,050 bytes in size. The information in the electronic format of the Sequence Listing is incorporated by reference in its entirety.

### FIELD

The present disclosure provides fusion polypeptides in which a small polypeptide is modified by fusion with N- and C-termini sequences that interact with one another when in proximity. In some embodiments, the small polypeptide is a cysteine motif binding peptide or a cytokine. Among embodiments of the present disclosure are cysteine motif binding peptides, such as from the knob region of an ultralong CDR3, and methods of producing and using the same. Also provided herein are compositions comprising the fusion polypeptides and binding peptides.

### BACKGROUND

Antibodies are natural proteins that the vertebrate immune system forms in response to foreign substances (antigens), primarily for defense against infection. Antibodies contain complementarity determining regions (CDRs) that mediate binding to a target antigen. Some bovine antibodies have unusually long variable heavy (VH) CDR3 sequences compared to other vertebrates. These long CDR3s, which can be up to 70 amino acids long, can form unique domains that protrude from the antibody surface, thereby permitting a unique antibody platform. Improved methods are needed for screening for and producing antibodies or portions thereof containing long CDR3s, as well as for screening for and producing other disulfide-bonded polypeptides.

### SUMMARY

Provided herein is a modified fusion polypeptide containing the formula N-terminus to C-terminus: Y1-[polypeptide]-Y2, wherein the polypeptide is a protein in which the distance between the N-termini and C-termini is no more than 10 Angstroms and wherein Y1 and Y2 are amino acid peptide sequences that interact with one another when in proximity.

In some embodiments, the Y1 and Y2 are characterized by: (i) Y1 and Y2 are selected from (i) HW and SF, respectively; (ii) IS and TV, respectively; (iii) DY and MP, respectively; (iv) LV and IP, respectively; or (v) SV and YI, respectively; or (ii) Y1 and Y2 are sequences that are each 14-30 amino acids in length containing a heptad repeat, wherein Y1 and Y2 are able to interact to form an anti-parallel coiled-coil motif.

In some embodiments, the Y1 and Y2 are HW and SF, respectively. In some embodiments, Y1 and Y2 are IS and TV, respectively.

In some embodiments, Y1 and Y2 are sequences that are each 14-30 amino acids in length containing a heptad repeat, wherein Y1 and Y2 are able to interact to form an anti-parallel coiled-coil motif.

In some of any embodiments, Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203. In some of any embodiments, the Y1 is set forth in SEQ ID NO:202 and Y2 is set forth in SEQ ID NO:203, respectively.

In some of any embodiments, the distance between the N- and C-termini is no more than 5, 6, 7, 8 or 9 Angstoms. In some of any embodiments, the distance between the N- and C-termini is between 2 and 10 Angstroms. In some of any embodiments, the distance between the N- and C-termini is between 2 and 8 Angstroms.

In some of any embodiments, the polypeptide is selected from a cysteine motif peptide or a cytokine. In some embodiments, the polypeptide is a cysteine motif binding peptide and the cysteine motif binding peptide is 20-50 amino acids in length in which 2-12 amino acids are cysteine residues able to form 1-6 disulfide bonds.

In some embodiments, the cysteine motif binding peptide is a knob peptide from an ultralong CDR3 of a cow antibody.

In some of any embodiments, the cysteine motif binding peptide does not include an ascending or descending stalk domain or a contiguous portion thereof of more than 3 amino acids in length from the ultralong CDR3 of the cow antibody.

In some of any embodiments, the cysteine motif binding peptide is 22 to 43 amino acids in length.

In some of any embodiments, the cysteine motif binding peptide includes at least 4 cysteine residues. In some of any embodiments, the cysteine motif binding peptide contains 4 cysteine residues, 6 cysteine residues or 8 cysteine residues.

In some of any embodiments, the cysteine motif binding peptide is able to form at least 2 disulfide bonds. In some of any embodiments, the cysteine motif binding peptide is able to form 2 disulfide bonds, 3 disulfide bonds or 4 disulfide bonds.

In some of any embodiments, the cysteine motif binding peptides binds to a target antigen that is a nonvirulent bacteria, a virus, a viral protein, an immunomodulatory protein, a cancer antigen, a human IgG, or a recombinant protein thereof.

In some embodiments, the target antigen is a coronavirus, a coronavirus pseudovirus, a recombinant coronavirus Spike protein, or a receptor-binding domain (RBD) of a coronavirus Spike protein. In some embodiments, the coronavirus is selected from the group consisting of 229E, NL63, OC43, HKU1, MERS-CoV, SARS-CoV, and SARS-CoV2. In some of any embodiments, the coronavirus is a SARS-CoV2 selected from Wuhan-Hu-1 isolate, B.1.351 South African variant, B.1.1.7 UK variant, a beta variant, a delta variant, or a omicron variant.

In some of any embodiments, the cysteine motif binding peptide is set forth in any one of SEQ ID NOS: 155, 198, and 227-240.

In some of any embodiments, the cytokine is IL-2 or IL-15.

Provided herein is a fusion protein, comprising a modified fusion polypeptide as provided herein and a moiety selected from a half-life extending moiety or a detectable moiety. In some of any of the provided embodiments, the half-life extending moiety is an immunoglobulin Fc. In some of any of the provided embodiments, the detectable moiety is a fluorescent protein. In some of any of the provided embodiments, the fluorescent protein is a GFP, optionally sfGFP. In some of any of the provided embodiments, the modified fusion polypeptide is inserted within the half-life extending moiety or detectable moiety. In some of any of the provided embodiments, the modified fusion polypeptide is inserted within a loop of the half-life extending moiety or detectable moiety.

Provided herein is a nucleic acid encoding a modified fusion polypeptide of some of any embodiments.

Provided herein is an expression vector containing the nucleic acid molecule of some embodiments.

Provided herein is a composition containing the modified fusion polypeptide of some of any embodiments. In some embodiments, the composition is a pharmaceutical composition containing a pharmaceutically acceptable excipient.

Provided herein is a method of producing a modified fusion polypeptide, the method including adding an N-terminus sequence (Y1) and a C-terminus sequence (Y2) to a polypeptide, wherein the polypeptide is a protein in which the distance between the the N-termini and C-termini is no more than 10 Angstroms and wherein the modified fusion polypeptide has the formula N-terminus to C-terminus: Y1-[polypeptide]-Y2, wherein Y1 and Y2 are amino acid peptide sequences that interact with one another when in proximity.

In some embodiments, Y1 and Y2 are characterized by: (i) Y1 and Y2 are selected from (i) HW and SF, respectively; (ii) IS and TV, respectively; (iii) DY and MP, respectively; (iv) LV and IP, respectively; or (v) SV and YI, respectively; or (ii) Y1 and Y2 are sequences that are each 14-30 amino acids in length containing a heptad repeat, wherein Y1 and Y2 are able to interact to form an anti-parallel coiled-coil motif.

In some of any embodiments, the polypeptide is selected from a cysteine motif peptide or a cytokine, wherin the cysteine motif binding peptide is 20-50 amino acids in length in which 2-12 amino acids are cysteine residues able to form 1-6 disulfide bonds.

In some embodiments, the cytokine is IL-2 or IL-15.

Provided herein is a method of producing a modified binding peptide, the method including: obtaining a cysteine motif binding peptide of 20-50 amino acids in length in which 2-12 amino acids are cysteine residues able to form 1-6 disulfide bonds; and modifying the binding peptide by adding an N-terminus sequence (Y1) and a C-terminus sequence (Y2) to the cysteine motif binding peptide, wherein the modified binding peptide has the formula N-terminus to C-terminus: Y1-[cysteine motif binding peptide]-Y2, wherein Y1 and Y2 are characterized by: (i) Y1 and Y2 are selected from (i) HW and SF, respectively; (ii) IS and TV, respectively; (iii) DY and MP, respectively; (iv) LV and IP, respectively; or (v) SV and YI, respectively; or (ii) Y1 and Y2 are sequences that are each 14-30 amino acids in length containing a heptad repeat, wherein Y1 and Y2 are able to interact to form an anti-parallel coiled-coil motif.

In some of any embodiments, the Y1 and Y2 are HW and SF, respectively. In some of any embodiments, the Y1 and Y2 are IS and TV, respectively.

In some of any embodiments, Y1 and Y2 are sequences that are each 14-30 amino acids in length containing a heptad repeat, wherein Y1 and Y2 are able to interact to form an anti-parallel coiled-coil motif.

In some of any embodiments, Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203. In some of any embodiments, 1, 4 or 5, wherein the Y1 is set forth in SEQ ID NO:202 and Y2 is set forth in SEQ ID NO:203, respectively.

In some of any embodiments, the cysteine motif binding peptide is a knob peptide from an ultralong CDR3 of a cow antibody.

In some embodiments, the cysteine motif binding peptide does not include an ascending or descending stalk domain or a contiguous portion thereof of more than 3 amino acids in length from the ultralong CDR3 of the cow antibody.

In some of any embodiments, the cysteine motif binding peptide is 22 to 43 amino acids in length.

In some of any embodiments, the cysteine motif binding peptide includes at least 4 cysteine residues. In some of any embodiments, the cysteine motif binding peptide contains 4 cysteine residues, 6 cysteine residues or 8 cysteine residues.

In some of any embodiments, the cysteine motif binding peptide is able to form at least 2 disulfide bonds. In some of any embodiments, the cysteine motif binding peptide is able to form 2 disulfide bonds, 3 disulfide bonds or 4 disulfide bonds.

In some of any embodiments, the cysteine motif binding peptide is identified by a method including: (1) immunizing a cow with a target antigen or a sequence portion containing an epitope thereof; (2) identifying a knob peptide sequence from an antibody variable heavy chain (VH) sequence from peripheral blood mononuclear cells (PBMCs) from the immunized cow, wherein the knob peptide is a sequence between the ascending and descending stalk sequences of an ultralong CDR3, wherein the ultralong CDR3 is 40 to 70 amino acids in length, and wherein the knob peptide is a cysteine motif binding peptide of 20-50 amino acids in length in which 2-12 amino acids are cysteine residues able to form 1-6 disulfide bonds.

In some embodiments, the knob peptide is identified from the VH sequence by an algorithm including: identifying the conserved cysteine in framework 3 and the conserved tryptophan in framework 4; and determining the sequence of the knob, in which: the knob has the amino acid sequence length K; the sequence begins at position X+1 and ends at X+K; and K=L−2X; wherein L is the number of amino acids in an amino acid sequence starting at the conserved cysteine in framework 3 and ending at the conserved tryptophan in framework 4, and X is the number of amino acids from the first cysteine in framework 3 to the first conserved cysteine encoded by the D_{H} region in CDR H3.

In some of any embodiments, the cysteine motif binding peptide is extended by one, two, three, four, or five amino acids at the N and/or C termini of the ultralong CDR3 compared to the determined knob sequence.

In some of any embodiments, identifying the knob peptide includes: (a) amplifying sequences encoding a plurality of variable heavy (VH) regions of the IgHV1-7 family from a VH chain complementary DNA (cDNA) template library prepared from RNA isolated from the PBMCs from the immunized cow; (b) constructing a plurality of replicable expression vectors for the plurality of VH regions, wherein each replicable expression vector includes a nucleic acid sequence encoding a single chain variable fragment (scFv) including an amplified VH region joined to a variable lambda light (VL) region selected from the group consisting of VL regions of BLV1H12, BLV5D3, BLV8C11, BF1H1, BLV5B8, and F18, or a humanized variant thereof; (c) transforming suitable host cells with the plurality of replicable expression vectors under conditions suitable to produce amplified display particles; and (d) collecting the amplified display particles, wherein the amplified display particles include display particles displaying a fusion protein including an scFv; and (e) contacting the amplified display particles with a target antigen under conditions to allow binding of a display particle to the target antigen; and (f) selecting display particles including an antibody that binds to the target antigen by separating the display particles that bind from those that do not; and (g) sequencing the fusion gene in the selected display particles to identify the antibody with a VH sequence that contains or is suspected of containing an ultralong CDR3.

In some embodiments, the VL region is the BLV1H12 VL region. In some embodiments, the BLV1H12 lambda VL region is set forth in SEQ ID NO: 2.

In some embodiments, the BLV1H12 lambda VL region is a humanized variant of the lambda VL region of BLV1H12. In some embodiments, the humanized variant includes one or more of amino acid replacements S2A, T5N, P8S, A12G, A13S, and P14L based on Kabat numbering, amino acid replacements I29V and N32G in the CDR1 region, and/or amino acid substitution of DNN to GDT in the CDR2 region. In some of any embodiments, the humanized variant includes the sequence set forth in SEQ ID NO: 107.

In some of any embodiments, the cDNA template library is prepared from RNA isolated from peripheral blood mononuclear cells (PBMCs) from an immunized cow.

In some of any embodiments, the plurality of VH regions of the IgHV1-7 family from the cDNA template library are amplified with a forward primer containing the sequence set forth in SEQ ID NO: 84 and a reverse primer containing the sequence set forth in SEQ ID NO: 85.

In some of any embodiments, prior to the constructing, the method further includes performing a size separation on the sequences encoding the plurality of amplified VH regions to enrich for VH regions with an ultralong CDR3, optionally wherein the size separation is by gel electrophoresis. In some of any embodiments, the size separation includes separating sequences of, of about, or greater than 550 base pairs in length from the sequences encoding the plurality of amplified VH regions, wherein the sequences of, of about, or greater than 550 base pairs in length contain sequences encoding VH regions with an ultralong CDR3.

In some of any embodiments, identifying the knob peptide sequence includes amplification from a variable heavy chain cDNA template library from the immunized cow using primers specific for either side of the stalk domain of a cow ultralong CDR3 region.

In some of any embodiments, identifying the knob peptide includes: (a) amplifying sequences encoding a plurality of CDR3-knob only antibodies from a cow antibody variable heavy (VH) chain complementary DNA (cDNA) template library with forward and reverse primers specific for the ascending and descending stalk domains of a cow ultralong CDR3 region; (b) constructing a plurality of replicable expression vectors for the plurality of CDR3-knob only antibodies, wherein each replicable expression vector includes a nucleic acid sequence encoding an amplified CDR3 knob; (c) transforming suitable host cells with the plurality of replicable expression vectors under conditions suitable to produce amplified display particles; (d) collecting the amplified display particles, wherein the amplified display particles include display particles displaying a fusion protein containing an amplified CDR3 knob; (e) contacting the amplified display particles with a target antigen under conditions to allow binding of a display particle to the target antigen; (f) selecting display particles containing a CDR3-knob only antibody that binds to the target antigen by separating the display particles that bind from those that do not; and (g) sequencing the fusion gene in the selected display particles to identify the CDR3-knob antibody.

In some of any embodiments, the cDNA template library is prepared from RNA isolated from peripheral blood mononuclear cells (PBMCs) from an immunized cow.

In some of any embodiments, the primers are a pool of primers that contain or consist of any of the sequences set forth in SEQ ID NO: 7-11 and 121-130, optionally contain or consist of any of the sequences set forth in SEQ ID NO: 123, 127, and 128.

In some of any embodiments, the amplified display particles are phage display particles.

In some of any embodiments, the binding peptide binds to a target antigen. In some of any embodiments, the target antigen is a nonvirulent bacteria, a virus, a viral protein, an immunomodulatory protein, a cancer antigen, a human IgG, or a recombinant protein thereof.

In some of any embodiments, the target antigen is a coronavirus, a coronavirus pseudovirus, a recombinant coronavirus Spike protein, or a receptor-binding domain (RBD) of a coronavirus Spike protein. In some embodiments, the coronavirus is selected from the group consisting of 229E, NL63, OC43, HKU1, MERS-CoV, SARS-CoV, and SARS-CoV2. In some of any embodiments, the coronavirus is a SARS-CoV2 selected from Wuhan-Hu-1 isolate, B.1.351 South African variant, B.1.1.7 UK variant, a beta variant, a delta variant, or a omicron variant.

In some of any embodiments, Y1 and Y2 are added by synthetic methods or by recombinant DNA techniques.

Provided herein is a modified binding peptide produced by the methods of some of any embodiments.

Provided herein is a nucleic acid molecule encoding a modified binding peptide produced by some of any embodiments.

Provided herein is a method for producing a soluble binding peptide, including: (a) transforming E. coli with an expression vector encoding a fusion protein containing a binding peptide and thioredoxin A (TrxA) bacterial chaperone set forth in SEQ ID NO:194 or a sequence of amino acids that exhibits at least 85%, 86%, 87%, 88%, 89%, 90%, 91%, 92%, 92%, 94%, 95%, 96%, 97%, 98%, 99% sequence identity to SEQ ID NO:194, wherein the binding peptide is a cysteine modified binding peptide of 20-50 amino acids in length in which 2-12 amino acids are cysteine residues able to form 1-6 disulfide bonds; and; (b) culturing the bacteria under conditions permissive of expression of the fusion protein; and (c) isolating the fusion protein from supernatant of a bacterial cell lysate.

In some embodiments, the cysteine modified binding peptide binding peptide contains a knob peptide from an ultralong CDR3 of a cow antibody.

In some of any embodiments, the cysteine modified binding peptide is set forth in any one of SEQ ID NOS: 60, 61, 62, 63, 64, 65, 66, 67, 68, 155, 198 and 227-317.

Provided herein is a method for producing a soluble ultralong CDR3 knob, including: (a) transforming E. coli with an expression vector encoding a fusion protein containing a binding peptide and a bacterial chaperone, wherein the binding peptide is a modified binding peptide that has the formula N-terminus to C-terminus: Y1-[cysteine motif binding peptide]-Y2, wherein the cysteine motif binding peptide is a peptide sequence of 20-50 amino acids with a cysteine motif containing 2-12 cysteine residues able to form 1-6 disulfide bonds and Y1 and Y2 are characterized by: (i) Y1 and Y2 are selected from (i) HW and SF, respectively; (ii) IS and TV, respectively; (iii) DY and MP, respectively; (iv) LV and IP, respectively; or (v) SV and YI, respectively; or (ii) Y1 and Y2 are sequences that are each 14-30 amino acids in length containing a heptad repeat, wherein Y1 and Y2 are able to interact to form an anti-parallel coiled-coil motif; (b) culturing the bacteria under conditions permissive of expression of the fusion protein; and (c) isolating the fusion protein from supernatant of a bacterial cell lysate.

In some embodiments, the Y1 and Y2 are HW and SF, respectively. In some embodiments, the Y1 and Y2 are IS and TV, respectively. In some embodiments, the Y1 and Y2 are sequences that are each 14-30 amino acids in length containing a heptad repeat, wherein Y1 and Y2 are able to interact to form an anti-parallel coiled-coil motif.

In some of any embodiments, Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203. In some of any embodiments, the Y1 is set forth in SEQ ID NO:202 and Y2 is set forth in SEQ ID NO:203, respectively.

In some of any embodiments, the cysteine motif binding peptide is a knob peptide from an ultralong CDR3 of a cow antibody. In some embodiments, the cysteine motif binding peptide does not include an ascending or descending stalk domain or a contiguous portion thereof of more than 3 amino acids in length from the ultralong CDR3 of the cow antibody.

In some of any embodiments, the cysteine motif binding peptide is 22 to 43 amino acids in length.

In some of any embodiments, the cysteine motif binding peptide includes at least 4 cysteine residues. In some of any embodiments, the cysteine motif binding peptide contains 4 cysteine residues, 6 cysteine residues or 8 cysteine residues.

In some of any embodiments, the cysteine motif binding peptide is able to form at least 2 disulfide bonds. In some of any embodiments, the cysteine motif binding peptide is able to form 2 disulfide bonds, 3 disulfide bonds or 4 disulfide bonds.

Provided herein is a method for producing a soluble binding peptide, including: (a) transforming E. coli with an expression vector encoding a fusion protein containing a binding peptide and a bacterial chaperone, wherein the binding peptide is set forth in any one of SEQ ID NOS: 60, 61, 62, 63, 64, 65, 66, 67, 68, 155, 198 and 227-317 and; (b) culturing the bacteria under conditions permissive of expression of the fusion protein; (c) isolating the fusion protein from supernatant of a bacterial cell lysate.

In some of any embodiments, the binding peptide is set forth in any of SEQ ID NOS: 155, 198, and 227-240.

In some of any embodiments, the bacterial chaperone is thioredoxin A (TrxA). In some embodiments, TrxA has the sequence set forth in SEQ ID NO:194 or a sequence of amino acids that exhibits at least 85%, 86%, 87%, 88%, 89%, 90%, 91%, 92%, 92%, 94%, 95%, 96%, 97%, 98%, 99% sequence identity to SEQ ID NO:194. In some of any embodiments, TrxA has the sequence set forth in SEQ ID NO:194.

In some of any embodiments, the binding peptide and bacterial chaperone are joined by a cleavable linker. In some of any embodiments, the binding peptide is C-terminal to the bacterial chaperone.

In some of any embodiments, the method further includes (d) cleaving the cleavable linker of the fusion protein, thereby producing a soluble binding peptide containing 1-6 disulfide bonds free of the bacterial chaperone.

In some of any embodiments, the cleavable linker contains a cleavage site selected from: (i) a enterokinase cleavage site, optionally containing the amino acid sequence set forth by DDDDK (SEQ ID NO:106); (ii) a Factor Xa cleavage site, optionally containing the amino acid sequence set forth by SEQ ID NO: 218 or I-E/D-G-R; (iii) a thrombin cleavage site, optionally containing the amino acid sequence set forth by any one of SEQ ID NOS: 219-222, more optionally set forth by SEQ ID NO: 220; or (iv) a TEV protease cleavage site set forth by SEQ ID NO: 223 or SEQ ID NO:224.

In some of any embodiments, cleaving the cleavable linker includes contacting the fusion protein with the protease that recognizes the cleavage site.

In some embodiments, the cleavable linker is a enterokinase cleavable linker containing the amino acid sequence DDDDK (SEQ ID NO:106). In some of any embodiments, the enterokinase cleavable linker contains the sequence DYKDDDK (SEQ ID NO: 209) or GGSDYKDDDDKGS (SEQ ID NO:210). In some of any embodiments, cleaving the cleavable linker includes contacting the fusion protein with an enterokinase.

In some of any embodiments, the method further including removing the bacterial chaperone from the solution containing the soluble modified binding peptide.

In some of any embodiments, the method further including removing the protease, optionally the enterokinase, from the solution containing the soluble modified binding peptide.

In some of any embodiments, the binding is peptide is engineered into a loop of the bacterial chaperone. In some embodiments, the bacterial chaperone is TrxA and the loop is selected the catalytic loop corresponding to residues 31-35 of SEQ ID NO: 194, the first binding loop corresponding to residues 74-76 of SEQ ID NO:194 or the second binding loop corresponding to residues 91-93 of SEQ ID NO:194. In some of any embodiments, the bacterial chaperone is TrxA and the loop is the second binding loop corresponding to residues 91-93 of SEQ ID NO:194, optionally wherein the modified binding peptide is engineered between Val-92 and Gly-93 of the sequence set forth in SEQ ID NO:194.

Provided herein is a soluble modified binding peptide produced by the method of some of any embodiments.

In some of any embodiments, the binding peptide is engineered into the loop between a first and second cleavable linker positioned on the N-berminus and C-terminus of the binding polypeptide, respectively. In some embodiments, the first and second cleavable linker are the same.

In some of any embodiments, the first and second cleavable linker contains a cleavage site selected from: (i) a enterokinase cleavage site, optionally containing the amino acid sequence set forth by DDDDK (SEQ ID NO: 106); (ii) a Factor Xa cleavage site, optionally containing the amino acid sequence set forth by SEQ ID NO: 218 or I-E/D-G-R; (iii) a thrombin cleavage site, optionally containing the amino acid sequence set forth by any one of SEQ ID NOS: 219-222, more optionally set forth by SEQ ID NO: 220; or (iv) a TEV protease cleavage site set forth by SEQ ID NO: 223 or SEQ ID NO:224.

Provided herein is a fusion protein containing a modified binding peptide and a bacterial chaperone joined by a cleavable linker, wherein the modified binding peptide has the formula formula N-terminus to C-terminus: Y1-[cysteine motif binding peptide]-Y2, wherein: the cysteine motif binding peptide is 20-50 amino acids in length in which 2-12 amino acids are cysteine residues able to form 1-6 disulfide bonds; and Y1 and Y2 are amino acid peptide sequences that interact with one another when in proximity.

In some embodiments, Y1 and Y2 are characterized by: (i) Y1 and Y2 are selected from (i) HW and SF, respectively; (ii) IS and TV, respectively; (iii) DY and MP, respectively; (iv) LV and IP, respectively; or (v) SV and YI, respectively; or (ii) Y1 and Y2 are sequences that are each 14-30 amino acids in length containing a heptad repeat, wherein Y1 and Y2 are able to interact to form an anti-parallel coiled-coil motif.

In some of any embodiments, the Y1 and Y2 are HW and SF, respectively. In some of any embodiments, Y1 and Y2 are IS and TV, respectively.

In some of any embodiments, Y1 and Y2 are sequences that are each 14-30 amino acids in length containing a heptad repeat, wherein Y1 and Y2 are able to interact to form an anti-parallel coiled-coil motif.

In some of any embodiments, Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203. In some of any embodiments, the Y1 is set forth in SEQ ID NO:202 and Y2 is set forth in SEQ ID NO:203, respectively.

In some of any embodiments, the cysteine motif binding peptide is a knob peptide from an ultralong CDR3 of a cow antibody.

In some embodiments, the cysteine motif binding peptide does not contain an ascending or descending stalk domain or a contiguous portion thereof of more than 3 amino acids in length from the ultralong CDR3 of the cow antibody.

In some of any embodiments, the cysteine motif binding peptide is 22 to 43 amino acids in length.

In some of any embodiments, the cysteine motif binding peptide includes at least 4 cysteine residues. In some of any embodiments, the cysteine motif binding peptide contains 4 cysteine residues, 6 cysteine residues or 8 cysteine residues.

In some of any embodiments, the cysteine motif binding peptide is able to form at least 2 disulfide bonds. In some of any embodiments, the cysteine motif binding peptide is able to form 2 disulfide bonds, 3 disulfide bonds or 4 disulfide bonds.

In some of any embodiments, the cysteine motif binding peptides binds to a target antigen that is a nonvirulent bacteria, a virus, a viral protein, an immunomodulatory protein, a cancer antigen, a human IgG, or a recombinant protein thereof.

In some of any embodiments, the target antigen is a coronavirus, a coronavirus pseudovirus, a recombinant coronavirus Spike protein, or a receptor-binding domain (RBD) of a coronavirus Spike protein. In some embodiments, the coronavirus is selected from the group consisting of 229E, NL63, OC43, HKU1, MERS-CoV, SARS-CoV, and SARS-CoV2. In some of any embodiments, the coronavirus is a SARS-CoV2 selected from Wuhan-Hu-1 isolate, B.1.351 South African variant, B.1.1.7 UK variant, a beta variant, a delta variant, or a omicron variant.

In some of any embodiments, the cysteine motif binding peptide is set forth in any one of SEQ ID NOS: 155, 198, and 227-240.

In some of any embodiments, the cleavable linker includes a cleavage site selected from: (i) a enterokinase cleavage site, optionally containing the amino acid sequence set forth by DDDDK (SEQ ID NO:106); (ii) a Factor Xa cleavage site, optionally containing the amino acid sequence set forth by SEQ ID NO: 218 or I-E/D-G-R; (iii) a thrombin cleavage site, optionally containing the amino acid sequence set forth by any one of SEQ ID NOS: 219-222, more optionally set forth by SEQ ID NO: 220; or (iv) a TEV protease cleavage site set forth by SEQ ID NO: 223 or SEQ ID NO:224.

In some of any embodiments, the cleavable linker is an enterokinase cleavable linker containing the amino acid sequence DDDDK (SEQ ID NO:106). In some embodiments, the enterokinase cleavable linker containes the sequence DYKDDDK (SEQ ID NO: 209) or GGSDYKDDDDKGS (SEQ ID NO:210).

In some of any embodiments, the bacterial chaperone is thioredoxin A (TrxA).

In some embodiments, TrxA has the sequence set forth in SEQ ID NO:194 or a sequence of amino acids that exhibits at least 85%, 86%, 87%, 88%, 89%, 90%, 91%, 92%, 92%, 94%, 95%, 96%, 97%, 98%, 99% sequence identity to SEQ ID NO:194

Provided herein is a soluble modified binding peptide produced by the method of some of any embodiments.

Provided herein is a soluble peptide containing a modified binding peptide that is disulfide-bonded, wherein the modified binding peptide has the formula formula N-terminus to C-terminus: Y1-[cysteine motif binding peptide]-Y2, wherein: the cysteine motif binding peptide is 20-50 amino acids in length in which 2-12 amino acids are cysteine residues and wherein the soluble peptide contains 1 to 6 disulfide bonds; and Y1 and Y2 are amino acid peptide sequences that interact with one another when in proximity.

In some embodiments, Y1 and Y2 are characterized by: (i) Y1 and Y2 are selected from (i) HW and SF, respectively; (ii) IS and TV, respectively; (iii) DY and MP, respectively; (iv) LV and IP, respectively; or (v) SV and YI, respectively; or (ii) Y1 and Y2 are sequences that are each 14-30 amino acids in length containing a heptad repeat, wherein Y1 and Y2 are able to interact to form an anti-parallel coiled-coil motif.

In some of any embodiments, the Y1 and Y2 are HW and SF, respectively. In some of any embodiments, Y1 and Y2 are IS and TV, respectively.

In some of any embodiments, Y1 and Y2 are sequences that are each 14-30 amino acids in length containing a heptad repeat, wherein Y1 and Y2 are able to interact to form an anti-parallel coiled-coil motif.

In some of any embodiments, Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203. In some of any embodiments, the Y1 is set forth in SEQ ID NO:202 and Y2 is set forth in SEQ ID NO:203, respectively.

In some of any embodiments, the cysteine motif binding peptide is a knob peptide from an ultralong CDR3 of a cow antibody.

In some embodiments, the cysteine motif binding peptide does not include an ascending or descending stalk domain or a contiguous portion thereof of more than 3 amino acids in length from the ultralong CDR3 of the cow antibody.

In some of any embodiments, the cysteine motif binding peptide is 22 to 43 amino acids in length.

In some of any embodiments, the cysteine motif binding peptide contains at least 4 cysteine residues. In some of any embodiments, the cysteine motif binding peptide contains 4 cysteine residues, 6 cysteine residues or 8 cysteine residues.

In some of any embodiments, the soluble peptide has at least 2 disulfide bonds. In some of any embodiments, the soluble peptide has 2 disulfide bonds, 3 disulfide bonds or 4 disulfide bonds.

In some of any embodiments, the soluble peptide binds to a target antigen that is a nonvirulent bacteria, a virus, a viral protein, an immunomodulatory protein, a cancer antigen, a human IgG, or a recombinant protein thereof.

In some of any embodiments, the target antigen is a coronavirus, a coronavirus pseudovirus, a recombinant coronavirus Spike protein, or a receptor-binding domain (RBD) of a coronavirus Spike protein. In some embodiments, the coronavirus is selected from the group consisting of 229E, NL63, OC43, HKU1, MERS-CoV, SARS-CoV, and SARS-CoV2. In some of any embodiments, the coronavirus is a SARS-CoV2 selected from Wuhan-Hu-1 isolate, B.1.351 South African variant, B.1.1.7 UK variant, a beta variant, a delta variant, or an omicron variant.

In some of any embodiments, the cysteine motif binding peptide is set forth in any one of SEQ ID NOS: 155, 198, and 227-240.

Also provided herein is a composition comprising any of the provided soluble peptides. In some embodiments, the composition is a pharmaceutical composition comprising a pharmaceutically acceptable excipient.

Also provided herein is a method of administering to a subject any one of the modified fusion polypeptides, modified binding peptides, soluble peptide, or any composition containing same, for use in treating a disease or condition. In some embodiments, the disease or condition is a virus infection. In some embodiments, the virus infection is infection with a coronavirus.

### BRIEF DESCRIPTION OF FIGURES

FIG. 1 depicts a schematic of an exemplary Ultralong CDR3 cow antibody, including the “knob” peptide having a size between 4 and 6 KDa.

FIG. 2A depicts binding of immunized calf serum against the RBD domain of the SARS CoV-2 S protein via ELISA. Neutralization activity of the sera IgG against SARS-CoV-2 pseudovirus is shown in FIG. 2B.

FIG. 3A depicts the pIII phage fusion constructs in each display library (i.e., scFv and “knob” display).

FIG. 3B displays a schematic of pTAU1 phage vector multiple cloning site, used for direct cloning of bovine CDR3 knob DNA fragments as NcoI-NotI fragments. A schematic of pTAU1-BLV1H12(-VH) phage scFv vector multiple cloning site used for cloning of bovine VH DNA fragments as NcoI-XhoI fragments in-frame with BLV1H12 V-lambda DNA is shown in FIG. 3C. FIG. 3D depicts the separation between Ultralong VH fragments and shorter VH fragments without the Ultralong CDR3 region on an agarose gel.

Sequences alignments for exemplary Ultralong antibodies R2C1 (SKD, SEQ ID NO: 68), R2C3 (SKM, SEQ ID NO: 69), R4C1 (SEQ ID NO: 70), R5C1 (SEQ ID NO: 71), SR3A3 (SEQ ID NO: 72), RR2F12 (SEQ ID NO: 73), and RR2G3 (SEQ ID NO: 74) are shown in FIG. 4. A germline sequence is also shown (SEQ ID NO: 75).

FIG. 5A depicts binding of exemplary chimeric bovine-human IgG1 antibodies to spike protein, binding to the RBD is also shown in FIG. 5B. FIG. 5C shows ELISA binding of IgG antibodies to recombinant stabilized spike proteins derived from several SARS CoV strains.

FIG. 5D shows ELISA binding curves of select IgG antibodies against the omicron variant RBD (left) or recombinant stabilized spike trimer (right).

FIG. 5E reflects exemplary ELISA data of R4C1 and R2D9 on SARS-CoV-2 compared to SARS-CoV-1. FIG. 5F shows ELISA binding activity for three different exemplary antibody knob candidates against WT (Wuhan) SARS CoV-2 spike protein. FIG. 5G depicts a modified western blot using SDS and detected with biotinylated RBD.

FIG. 6A displays a schematic of the pET32b vector cloning site used for trxA-CDR3-knob fusion and CDR3-knob expression. A schematic of purification process from bacterial lysate is shown in FIG. 6B. FIG. 6C depicts CDR3-knob SDS-PAGE showing efficient purification of soluble CDR3-KNOB from E. coli lysate. FIG. 6D depicts an exemplary SDS-PAGE gel of several purified ultralong CDR H3 knob peptides.

FIG. 7A shows the results of a Wuhan-Hu-1 spike protein capture ELISA, using serial dilutions of IMAC purified trxA-fusions. Binding for the TrxA-R2G3 fusion protein is also shown in FIG. 7B.

FIG. 8A depicts a background-subtracted ELISA of soluble biotinylated RBD binding to exemplary purified R2-G3 CDR3-knob. Soluble R2G3 knob binding relative to a reference anti-spike antibody (CR3022) is shown in FIG. 8B.

Amino acid sequences of exemplary truncated R2G3 mutants are shown in FIG. 8C. Exemplary truncated R2G3 mutants include R2G3 TRUNCI (SEQ ID NO: 87), R2G3 TRUNC2 (SEQ ID NO: 88), R2G3 TRUNC3 (SEQ ID NO: 89), R2G3 TRUNC3A (SEQ ID NO: 90), R2G3 TRUNC3B (SEQ ID NO: 91), R2G3 TRUNC4 (SEQ ID NO: 92), and R2G3 TRUNC5 (SEQ ID NO: 93). The parental R2G3 variant from which the exemplary truncated mutants were derived is also shown (SEQ ID NO: 86).

FIG. 8D depicts a SDS-PAGE of R2G3 truncations after bacterial expression and purification. Results of an ELISA binding of biotinylated RBD by coated CDR3-knob truncation as shown in FIG. 8E.

FIG. 9A depicts a size exclusion chromatograph for purified R4C1 knobs. A gel electrophoresis gel of two fractions (A4 and A7) are shown in FIG. 9B.

FIG. 9C depicts a size exclusion chromatograph for purified R2G3 knobs. A gel electrophoresis gel of a fraction (A6) is shown in FIG. 9D. Results of a pseudovirus infection assay carried out on parental SARS-CoV-2 pseudovirus comparing 2G3 IgG, Fab fragment, and knob are shown in FIG. 9E.

Results of a pseudoviral luciferase assay are shown in FIG. 10 for four exemplary Ultralong CDR3 antibodies (F12, G3, SKD, and SKM) against wild-type (FIG. 10A), “UK” variant (FIG. 10B), “484K” variant (FIG. 10C), and “SA” variant (FIG. 10D) SARS CoV-2 spike protein expressing viruses.

FIG. 11A shows the IC50 values of different IgG antibodies against pseudoviruses from various coronavirus strains. FIG. 11B shows a comparison of the R2G3 IgG, Fab, and knob in neutralization of wild-type SARS-CoV-2 pseudovirus.

FIG. 12 is a depiction of multispecific knob peptide compositions and formats. A plurality of paratope knob peptides can be attached to an immunoglobulin, including as a homodimer or heterodimer, to provide a multispecific binding polypeptide. A plurality of paratope knob peptides also may be linked directly in tandem, such as via a linker. A plurality of knob peptides also may be combined as a mixture or cocktail to provide a combined polyclonal composition.

FIG. 13A depicts the crystal structure of BLV1H12 Fab (PDB 4k3d), an enlarged view of stalk and knob region, with framework 3 cysteine, knob position 1 cysteines, and the framework 4 tryptophan side chains is shown in FIG. 13B.

A sequence alignment of the stalk and knob regions for 12 exemplary antibodies is shown in FIG. 14, the knob regions are flanked by the ascending and descending stalk regions which are shown with white letters highlighted in black.

FIG. 15 is a schematic representation of the stalk and knob domain (L), containing the CDR H3 plus three residues on the N-terminal end.

Binding of biotinylated RBD by coated CDR3-knob truncations as assessed via ELISA are shown in FIG. 16A. An exemplary SDS-PAGE of R2G3 truncations after bacterial expression and purification is shown in FIG. 16B.

FIG. 17A shows ELISA binding of biotinylated RBD by coated CDR3-knob N-terminal truncations, and an exemplary SDS-PAGE of R2G3 N-terminal truncations after bacterial expression and purification is shown in FIG. 17B.

FIG. 18A shows a sequence alignment of primers specific for the ascending and descending stalk domains of a cow ultralong CDR3 region. FIG. 18B shows the PCR products obtained by amplification using the primers.

FIG. 19 shows a schematic of the strategy for randomization of two amino acids at each of the N- and C-termini of knobs using phage-displaying libraries.

FIG. 20A depicts size exclusion chromatographs for purified 2×NNK R2G3 knobs. A gel electrophoresis of elution fractions for HWSF (A10), ISTV (A8), DYMP (A8) and SVYI (A9), and G3 as a control are shown in FIG. 20B.

FIG. 21A depicts size exclusion chromatographs for R4C1 and modified R4C1 knobs. FIG. 21B depicts size exclusion chromatographs for R2D9 and modified R2D9 knobs. A gel electrophoresis with and without DTT of the elution fractions corresponding to R2D9HWSF (A10) and R2D9ISTV (A8) is shown in FIG. 21C.

FIG. 22 depicts results of a neutralization assay for 2×NNK R2G3 knobs against pseudotyped virus carrying SARS-CoV-2 (Wuhan-Hu-1) wild type (WT) spike protein.

FIG. 23A depicts ELISA data for binding of TrxA-SKM C-terminal fusions (from two different experiments) and TrxA-SKM Loop fusion constructs (from two different experiments) (FIG. 23B) to recombinant stabilized spike protein derived from the wild-type (WT) Wuhan-Hu-1 strain.

FIG. 24A shows an SDS-Page with linker, stalk, and coiled-coil (CC) knob fusion constructs. FIG. 24B shows an SDS-Page with linker and coiled-coil (CC) Fc knob fusion constructs. These linker and coiled-coil (CC) Fc knob fusion constructs are similarly shown in FIG. 24C in the presence and absence of a reducing agent (DTT). Coiled coil monomers and dimers along with TrxA and cc IL-15 constructs are shown in FIG. 24D.

Fc-G3 knob loop fusions were assessed for binding by ELISA in FIG. 25.

### DETAILED DESCRIPTION

Provided herein are modified fusion polypeptides that have a sequence with the formula N-terminus to C-terminus: Y1-[polypeptide]-Y2, wherein the polypeptide is a protein in which the distance between the N-termini and C-termini is no more than 10 Angstroms. In the provided embodiments, Y1 and Y2 are amino acid peptide sequences that interact with one another when in proximity. The ability of Y1 and Y2 to interact allows clamping of the polypeptide to thereby improvise its stability and activity. The polypeptide can be any small peptide or polypeptide, such as a cytokine or cysteine motif binding peptide (e.g. a knob peptide from an ultralong CDR3). Also provided are nucleic acid molecules encoding the modified fusion polypeptides, as well as expression vectors containing the nucleic acid molecules. Also provided herein are methods of making and using the modified fusion polypeptides.

Provided herein in some embodiments are binding peptides that are cysteine-motif binding peptides that contain 2 more more cysteines and that exhibit high affinity binding to a target antigen, including in some cases nanomolar or even picomolar binding affinity. In some embodiments, the binding peptide is based on or derived from an ultralong CDR3 of a cow antibody. Hence, in some aspects, the term binding peptide also is referred to as a cysteine motif binding peptide or knob peptide, which are terms that can be used interchangeably. In some embodiments, the binding peptide is a peptide of 20-50 amino acids in length in which 2-12 amino acids are cysteine residues able to form 1-6 disulfide bonds. In some embodiments, the binding peptide is or includes the knob portion of the ultralong CDR3 of a variable heavy (VH) chain sequence of an antibody present in a biological sample (e.g. peripheral blood) of a cow immunized with a target agent. Also provided herein are methods of improving the expression or activity of such binding peptides, as well as methods of producing soluble binding peptides. In some embodiments, the binding peptides are modified with N- and C-termini amino acid sequences which as described herein improve the expression, stability and/or activity of the binding peptide. Also provided herein are methods of producing the modified binding peptide by adding an N-terminus and C-terminus overhang sequence to the binding peptide, such as using recombinant DNA technology or by synthetic methods.

In some aspects, the provided embodiments allow for the efficient production of disulfide bonded knob peptides, including those derived from cow antibodies including an ultralong CDR3, that can be independently expressed and produced according to the provided methods as an independent binding unit. In some aspects, the provided embodiments also can relate to an immunization-based discovery platform for discovery and development of binding peptides that offers peptide structural diversity that is greater than that of in vitro display-based platforms, with each screened and produced knob peptide potentially having its own novel disulfide-bonded structure. This platform also allows for rapid hit discovery against target molecules.

Development of therapeutic molecules has seen remarkable advances as evidenced by the therapeutic response to emerging infectious diseases, including rapid identification of several classes of drugs, including biologics and small molecules. For the COVID-19 pandemic, initial efforts enabled rapid discovery of monoclonal antibodies, as well as eventually small molecule drug candidates. In any drug discovery program, the properties of the lead molecule (size, half-life, biodistribution, potential for formulation, routes of delivery, and manufacturing requirements) affect its potential for development into a therapeutic. Monoclonal antibodies can be readily discovered through several techniques, are usually highly specific to their cognate antigen, and have reproducible manufacturing and pharmacokinetic properties, making them suitable for a quick response to new microbial pathogens. Ideally, an expeditious response to an emerging disease threat would combine rapid discovery and deployment of antibodies with streamlined development of small molecules suitable for use as therapeutics. Although efforts of discovering and developing such therapeutic molecules are promising they are not always ideal. For example, it remains difficult to identify therapeutic molecules against cryptic epitopes, including cryptic viral epitopes. Further, efforts to manufacture and delivery such therapeutics may not be optimal. The provided embodiments address these needs.

Antibodies typically bind their antigen using up to six complementarity determining regions (CDRs), which are relatively short peptide loops found on the heavy and light chain variable regions. The most hypervariable CDR is H3, which in humans averages 12-15 amino acids but can be longer than 30 residues. The paradigm of using several CDR loops to bind antigen is conserved throughout vertebrates with the notable exception of bovines, which have a class of antibodies (about 10% of the repertoire) that appear to use primarily an exceptionally long (up to 70 residues) CDR H3. This ultralong CDR forms its own unique protruding minidomain comprising a β-ribbon “stalk” that supports a disulfide-bonded “knob” for antigen binding.

As described herein, cow antibodies have a unique structure containing an ultralong CDR3 sequence that forms a structure where a subdomain with an unusual architecture is formed from a “stalk”, composed of two 12-residue, anti-parallel β-strands (ascending and descending strands), and a longer, e.g., 39-residue, disulfide-rich “knob” that sits atop the stalk, far from the canonical antibody paratope. The knob region of the ultralong CDR3 confers antigen binding. Unlike antibodies from other species, such as human and mouse, the CDR regions L1, L2, L3, H1 and H2 of a bovine or bovine-derived antibody exhibit less sequence diversity as most of their sequence diversity is in CDR H3 (Stanfield et al. 2016 Sci. Immunol, 1(1): doi:10.1126/sciimmunol.aaf7962). Thus, for bovine or bovine-derived antibodies, antigen binding is mainly or only through CDR H3 and the other CDRs do not contribute to antigen binding.

This “knob” domain is analogous to natural cysteine-rich peptides such as knottins or cyclotides in that it is small, compact, and stable, but can accommodate diverse loop structures and disulfide bonding patterns.

Available methods of analysis and exploitation of the unique ultralong CDR H3 structure are not entirely satisfactory. In many cases, methods require excision and purification of the isolated knob domain (Macpherson et al. 2020 PLOS Biology, 18(9): e30000821). Such methods are not easily amenable to good manufacturing practices for generating therapeutic molecules and also are inefficient in terms of the amount of knob protein that can be produced. Further the use of enzymes for excision of the knob may also compromise the integrity of the isolated protein.

Remarkably, it is found herein that a disulfide bonded knob peptide derived from an ultralong CDR-H3 of a bovine antibody can be independently expressed and produced according to the provided methods as an independent binding unit and retains picomolar binding affinity and neutralizing activity against a target molecule, e.g., SARS-CoV2. The provided embodiments provide a platform for identifying unique disulfide-bonded binding peptide against any of a number of diverse antigens with binding characteristics that approximate a parent antibody.

Results herein also demonstrate knob stability and specific activity of the independent disulfide-bonded peptide can be further improved by including certain amino acid residue overhangs to the N- and C-termini of the knobs, in which it is believed these N- and C-termini are proximal to each other in the folded structure. Without wishing to be bound by theory, it is believed that the N- and C-terminal amino acid residues (also called Y1 and Y2 herein, respectively) can be used to clamp the ends of any small molecule polypeptide in which the N- and C-termini are normally within 10 Angstroms, such as 2 to 10 Angstroms. In some embodiments, the polypeptide is a cytokine, such as IL-2 or IL-15. In some embodiments, the polypeptide is a cysteine-motif binding peptide, such as a knob peptide derived from an ultralong CDR3 of a cow variable heavy chain of an antibody.

This knob peptide is only roughly 4-5 kDa in size, e.g., about 4.4 kDa, and represents the smallest independent antigen binding domain. It exhibits high affinity and epitope coverage, similar to a larger antibody. Its small size approaches the size of small molecules and thereby opens up the utility of the antigen binding domain as a new and novel therapeutic. For instance, its small size allows for better tissue penetration and also permits alveolar delivery. Further, the provided knob peptides are stable by virtue of their rigid disulfide-bonded small domain. This stable structure avoids aggregates seen in nanobodies and other immunoglobulin domain-based fragments. Also as demonstrated herein, findings also show that a knob peptide can be produced in high yield according to the provided methods in E. coli, making the knob peptide highly developable as a therapeutic molecule. Peptides generated according to the provided methods can target known viruses or viral classes. In some aspects, such knob peptides can be ready for rapid discovery and production in the event of pandemic outbreaks, and can be quickly pivoted in the case of new strains of disease. In some aspects, knob production according to the provided methods can move quickly to GMP standards. In some aspects, knobs can be used for “cocktails” of treatment regimens.

Embodiments provided herein demonstrate that immunization of cows may enable both discovery of potent anti-viral antibodies, and additionally allow identification of small disulfide-bonded knob domain peptides, which themselves could be therapeutic candidates with properties distinct from the parent antibody. Such small peptides may have enhanced tissue distribution, high stability, rapid manufacturing in microbial systems, or formulation and delivery advantages. For example, highly stable knob peptides may be exceptionally suitable for inhaled therapeutic delivery.

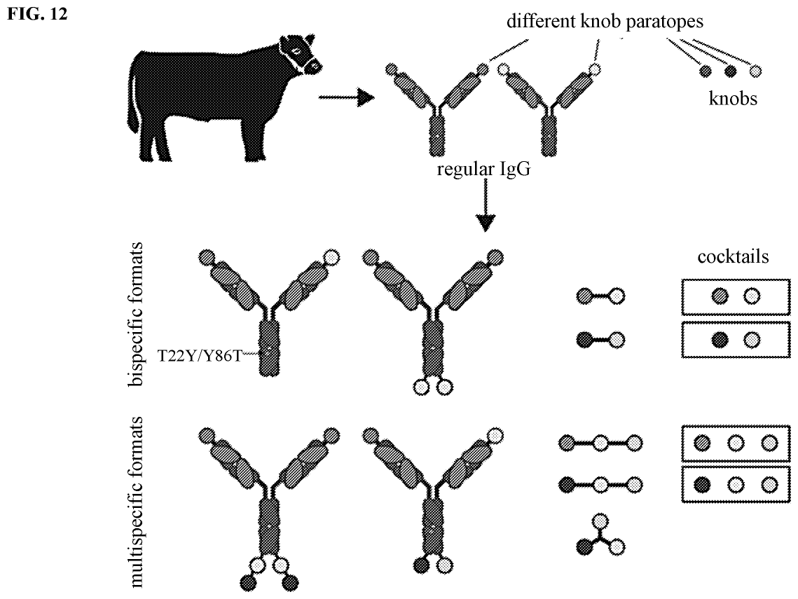

Also provided herein are compositions containing any of the knob peptides screened and produced according to the provided methods. In some embodiments, the compositions can be monoclonal providing a single knob peptide to provide a single paratope for binding a desired antigen, such as SARS-CoV2. In other embodiments, provided compositions are polyclonal and contain a mixture or cocktail of different knob peptides directed against different epitopes of an antigen or different antigens (FIG. 12).

Further, also provided herein are multispecific binding formats that exploit the small and unique size of the knob peptides (FIG. 12). For instance, different knob paratopes can be engineered into the backbone of a human or humanized ultralong CDR-H3 full length antibody in which dimerization of the Fc provides a bivalent or multivalent format. In some cases, “knobs into hole” Fc engineering strategy can be used to produce a heterodimeric bispecific or multispecific format containing two, three, four or more different knob peptides each providing a different paratope for binding to a desired antigen, such as Spike protein of SARS-CoV2.

Also provided herein are methods of treatment and uses of the provided binding peptides and compositions thereof.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the articles “a” and “an” refer to one or to more than one (i.e. to at least one) of the grammatical object of the article. By way of example, “an element” means one element or more than one element.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

An “ultralong CDR3” or an “ultralong CDR3 sequence”, used interchangeably herein, comprises a CDR3 or CDR3 sequence that is not derived from a human antibody sequence. An ultralong CDR3 may be 35 amino acids in length or longer, for example, 40 amino acids in length or longer, 45 amino acids in length or longer, 50 amino acids in length or longer, 55 amino acids in length or longer, or 60 amino acids in length or longer. In some embodiments, the ultralong CDR3 is 25-70 amino acids in length, such as 40-70 amino acids in length. Typically, the ultralong CDR3 is a heavy chain CDR3 (CDR-H3 or CDRH3). An ultralong CDR3H3 exhibits features of a CDRH3 of a ruminant (e.g., bovine) sequence. The structure of an ultralong CDR3 includes a “stalk”, composed of ascending and descending strands (e.g. each about 12 amino acids in length), and a disulfide-rich “knob” that sits atop the stalk. The unique “stalk and knob” structure of the ultralong CDR3 results in the two antiparallel β-strands (an ascending and descending stalk strand) supporting a disulfide bonded knob protruding out of the antibody surface to form a mini antigen binding domain. In some embodiments, the ultralong CDR3 antibodies comprise, in order, an ascending stalk region, a knob region, and a descending stalk region.

As used herein, a “knob peptide”, “CDR3-knob peptide” or “knob-only peptide,” which are terms used interchangeably, refers to peptide that is 20-50 amino acids in length, and contains cysteine motif made up of 6, 8, 10 or up to 12 non-canonical cysteine residues. A knob peptide may be derived from an ultralong CDR3 or can be produced synthetically. Typically, the first cysteine of the peptide sequences contains an initial cysteine residue with the amino acid motif cysteine-proline (CP). A knob peptide is a linear molecular that is not able to undergo cyclization to form a cyclic molecule. In some embodiments, a knob peptide can be produced as a soluble peptide that is an independently produced linear disulfide-bonded peptide containing 2-6 disulfide bonds formed by at least 4 non-canonical Cys residues.

“Substantially similar,” or “substantially the same”, refers to a sufficiently high degree of similarity between two numeric values (generally one associated with an antibody disclosed herein and the other associated with a reference/comparator antibody) such that one of skill in the art would consider the difference between the two values to be of little or no biological and/or statistical significance within the context of the biological characteristic measured by said values (e.g., Kd values). The difference between said two values is preferably less than about 50%, preferably less than about 40%, preferably less than about 30%, preferably less than about 20%, preferably less than about 10% as a function of the value for the reference/comparator antibody.

“Binding affinity” generally refers to the strength of the sum total of noncovalent interactions between a single binding site of a molecule (e.g., an antibody) and its binding partner (e.g., an antigen). Unless indicated otherwise, “binding affinity” refers to intrinsic binding affinity which reflects a 1:1 interaction between members of a binding pair (e.g., antibody and antigen). The affinity of a molecule X for its partner Y can generally be represented by the dissociation constant. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

“Percent (%) amino acid sequence identity” with respect to a peptide or polypeptide sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MegAlign (DNASTAR) software. Those skilled in the art can determine appropriate parameters for measuring alignment, including any algorithms needed to achieve maximal alignment over the full length of the sequences being compared.

“Polypeptide,” “peptide,” “protein,” and “protein fragment” may be used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

“Conservatively modified variants” applies to both amino acid and nucleic acid sequences. “Amino acid variants” refers to amino acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” including where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles disclosed herein. Typically conservative substitutions include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Humanized” or “Human engineered” forms of non-human (e.g., bovine) antibodies are chimeric antibodies that contain amino acids represented in human immunoglobulin sequences, including, for example, wherein minimal sequence is derived from non-human immunoglobulin. For example, humanized or human engineered antibodies may be non-human (e.g., bovine) antibodies in which some residues are substituted by residues from analogous sites in human antibodies (see, e.g., U.S. Pat. No. 5,766,886). A humanized antibody optionally may also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1: 105-115 (1998); Harris, Biochem. Soc. Transactions 23:1035-1038 (1995); Hurle and Gross, Curr. Op. Biotech. 5:428-433 (1994).

A “variable domain” with reference to an antibody refers to a specific Ig domain of an antibody heavy or light chain that contains a sequence of amino acids that varies among different antibodies. Each light chain and each heavy chain has one variable region domain (VL, and, VH). The variable domains provide antigen specificity, and thus are responsible for antigen recognition. Each variable region contains CDRs that are part of the antigen binding site domain and framework regions (FRs).

A “constant region domain” refers to a domain in an antibody heavy or light chain that contains a sequence of amino acids that is comparatively more conserved among antibodies than the variable region domain. Each light chain has a single light chain constant region (CL) domain and each heavy chain contains one or more heavy chain constant region (CH) domains, which include, CH1, CH2, CH3 and, in some cases, CH4. Full-length IgA, IgD and IgG isotypes contain CH1, CH2 CH3 and a hinge region, while IgE and IgM contain CH1, CH2 CH3 and CH4. CH1 and CL domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g. through interactions with various cells, biomolecules and tissues.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273,927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method. In other cases, the particular amino acid sequence of a CDR or FR is given.

An antibody containing an ultralong CDR3 is an antibody that contains a variable heavy (VH) chain with an ultralong CDR3. An antibody may further include pairing of the VH chain with a variable light (VL) chain. In some embodiments, the antibodies or antigen-binding fragments include a heavy chain variable region and a light chain variable region. Thus, the term antibody include full-length antibodies and portions thereof including antibody fragments, wherein such contain a heavy chain or portion thereof and/or a light chain or portion thereof. An antibody can contain two heavy chains (which can be denoted H and H′) and two light chains (which can be denoted L and L′), in which each L chain is linked to an H chain by a covalent disulfide bond and the two H chains are linked to each other by disulfide bonds. The terms “full-length antibody,” or “intact antibody” are used interchangeably to refer to an antibody in its substantially intact form, as opposed to an antibody fragment. A full-length antibody is an antibody typically having two full-length heavy chains (e.g., VH-CH1-CH2-CH3 or VH-CH1-CH2-CH3-CH4) and two full-length light chains (VL-CL) and hinge regions.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (VH) regions capable of specifically binding, and single chain variable fragments (scFv).

An “antibody fragment” comprises a portion of an intact antibody, the antigen binding and/or the variable region of the intact antibody. Antibody fragments, include, but are not limited to, Fab fragments, Fab′ fragments, F(ab′)₂ fragments, Fv fragments, disulfide-linked Fvs (dsFv), Fd fragments, Fd′ fragments; single-chain antibody molecules, including single-chain Fvs (scFv) or single-chain Fabs (scFab); antigen-binding fragments of any of the above and multispecific antibodies from antibody fragments.

A “Fab fragment” is an antibody fragment that results from digestion of a full-length immunoglobulin with papain, or a fragment having the same structure that is produced synthetically, e.g., by recombinant methods. A Fab fragment contains a light chain (containing a V_{L} and C_{L}) and another chain containing a variable domain of a heavy chain (V_{H}) and one constant region domain of the heavy chain (C_{H}1).

An “scFv fragment” refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “effective amount” or “therapeutically effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration and administration via inhalation.

As used herein, “disease or disorder” refers to a pathological condition in an organism resulting from cause or condition including, but not limited to, infections, acquired conditions, genetic conditions, and characterized by identifiable symptoms.

As used herein, the terms “treat,” “treating,” or “treatment” refer to ameliorating a disease or disorder, e.g., slowing or arresting or reducing the development of the disease or disorder, e.g., a root cause of the disorder or at least one of the clinical symptoms thereof.

As used herein, the term “subject” refers to an animal, including a mammal, such as a human being. The term subject and patient can be used interchangeably.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

### II. Modified Fusion Polypeptides and Binding Peptides and Methods of Producing Same

Provided herein is a modified fusion polypeptide comprising the formula N-terminus to C-terminus: Y1-[polypeptide]-Y2, wherein the polypeptide is a protein in which the distance between the N-termini and C-termini is no more than 10 Angstroms and wherein Y1 and Y2 are amino acid peptide sequences that interact with one another when in proximity. In some embodiments, the distance between the N- and C-termini is no more than 5, 6, 7, 8 or 9 Angstoms.

In some embodiments, the polypeptide is a cytokine. In some embodiments, the cytokine is IL-2. In some embodiments, the IL-2 cytokine sequence comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO:319. In some embodiments, the IL-2 cytokine sequence comprises the sequence of amino acids set forth in SEQ ID NO:319.

In some embodiments, the polypeptide is IL-15. In some embodiments, the IL-15 cytokine sequence comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO:320. In some embodiments, the IL-15 cytokine sequence comprises the sequence of amino acids set forth in SEQ ID NO:320.

In some embodiments, the polypeptide is a cysteine-motif binding peptide. Also provided herein are cysteine-motif binding peptides that are able to form disulfide bonds and bind to a target antigen, such as a viral antigen (e.g. spike protein of SARS-CoV-2 or a variant). The cysteine-motif binding peptides include peptides from the knob region derived from the ultralong CDR-H3 of a bovine or bovine-derived antibody. Among the provided binding peptides are neutralizing peptides, including broad spectrum neutralizing peptides (i.e. knob peptides), to SARS and related coronaviruses.

In some embodiments, the cysteine-motif binding peptide is a peptide of at least 20 amino acids in length in which 2-12 amino acids are cysteine residues able to form 1-6 disulfide bonds. In some embodiments, the binding peptide specifically binds to a target antigen. In some embodiments, the binding peptide is isolated or derived from a knob region of an ultralong CDR3 of a bovine antibody. Such bovine antibodies can be obtained by immunizing a cow with a target antigen of interest or an epitope-containing sequence thereof. Also provided herein are binding peptides of such knob peptides that are independently produced, such as synthetically or by recombinant DNA methods.

In some embodiments, any of such binding peptide is 20-50 amino acids in length in which 2-12 amino acids are cysteine residues able to form 1-6 disulfide bonds.

In some embodiments, the binding peptide is a binding peptide that is derived from an ultralong CDR3 of a VH chain of a cow antibody. Any of a variety of methods known to a skilled artisan can be used to obtain or identify a bovine antibody with an ultralong CDR3. In some embodiments, an antibody with an ultralong CDR3 is obtained by immunizing a cow and isolating antibodies with an ultralong CDR3. In some cases, display libraries of antibody sequences, or those enriched in antibodies with ultralong CDR3, can be prepared and screened for desired bind and activity to identify or obtain an antibody with an ultralong CDR3. Exemplary methods of immunization and screening are described in Sections III and IV.

In some embodiments, the binding peptide is the knob region of the ultralong CDR3 but may be extended 1-15 amino acids on the N- and/or C-terminus to include a contiguous portion of the ascending (stalk A) and/or descending (stalk B) amino acids of the ultralong CDR3. In some embodiments, the binding peptide is the knob region of the ultralong CDR3 but may be extended 1, 2, 3, 4 or 5 amino acids on the N- and/or C-terminus to include a contiguous portion of the ascending (stalk A) and/or descending (stalk B) amino acids of the ultralong CDR3.

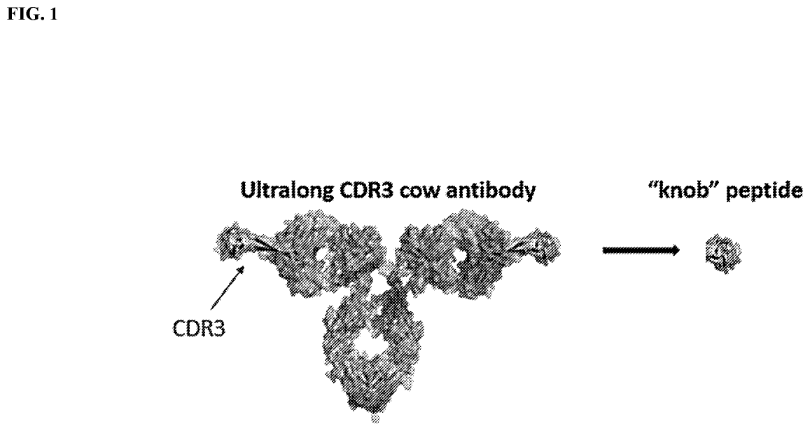

In some embodiments, the ultralong CDR-H3 includes an ascending stalk domain (Stalk A), a disulfide-rich knob region, and a descending stalk domain (Stalk B), in which the knob region is positioned between the ascending and descending stalk domains. In some embodiments, the sequence of the ultralong CDR-H3 provides a structure of an anti-parallel β-strands that protrude away from the antibody, in which the disulfide-rich knob region is positioned at the tip of the antibody (FIG. 1). Stalk A comprises mainly hydrophobic side chains and a relatively conserved motif at the base, which initiates the ascending strand. This conserved motif is typically found following the first cysteine residue in variable region sequences of the various bovine or cow sequences. In some embodiments, an ascending (stalk A) of an ultralong CDR3 includes the sequence CX₂TVX₅Q, wherein X₂ and X₅ are any amino acid. In some of any embodiments, X₂ is Ser, Thr, Gly, Asn, Ala, or Pro, and X₅ is His, Gln, Arg, Lys, Gly, Thr, Tyr, Phe, Trp, Met, Ile, Val, or Leu. In some of any embodiments, X₂ is Ser, Ala, or Thr, and X₅ is His or Tyr. In some embodiments, the base of Stalk A contains residues CTTVHQ (SEQ ID NO: 98), CATVHQ (SEQ ID NO: 99), CAIVQQ (SEQ ID NO: 100), or CATVDQ (SEQ ID NO: 101) that stabilizes the base by interacting with residues of the CDR-H1. The Stalk A further includes a variable number of connecting residues, e.g., 2 to 8 amino acid residues, before a first conserved cysteine residue that forms part of the disulfide-bonded knob region. In some embodiments, the knob region includes a first conserved amino acid motif Cys-Pro (CP), in which the initial cysteine residue forms the first disulfide bond with another cysteine residue in the knob. The knob may include 2-12 cysteine residues that are able to form 1-6 disulfide bonds. The stalk can be of variable length, and Stalk B may comprise alternating aromatics that form a ladder through stacking interactions, that may contribute to the stability of the long solvent-exposed, two stranded β-ribbon (Wang et al. Cell. 2013, 153 (6): 1379-1393). In some embodiments, the Stalk B contains a conserved pattern of alternating tyrosines, sometimes with the motif YX₁YX₂X₃(SEQ ID NO: 224), YX₁YX₂F (SEQ ID NO: 225), YX₁YX₂W (SEQ ID NO: 225) or YX₁YX₂Y (SEQ ID NO: 226), that support the knob structure. In some embodiments, the descending stalk portion of a knob may extend to include contiguous residues up to the conserved tryptophan in framework 4.

In some embodiments, the binding peptide is composed of the knob region of an ultralong CDR3 without any contiguous portion of the ascending (stalk A) and/or descending (stalk B) amino acids of the ultralong CDR3.

In some embodiments, the binding peptide is the knob region of the ultralong CDR3 and extends by 1, 2, 3, 4, 5, 6, 7, 8, 9, 10, 11, 12, 13 or 15 amino acids on the N- and/or C-terminus to include a contiguous portion of the ascending (stalk A) and/or descending (stalk B) amino acids of the ultralong CDR3. In some embodiments, the binding peptide is the knob region of the ultralong CDR3 and extends by 1, 2, 3, 4, 5 or 6 amino acids on the N-terminus and by 1, 2, 3, 4, 5 or 6 amino acids on the C-terminus to include a contiguous portion of the ascending (stalk A) and/or descending (stalk B) amino acids of the ultralong CDR3.

A knob region of an ultralong CDR3 of a cow antibody can be identified based on structural characteristics of the ultralong CDR3. In some embodiments, the CDR3-knob is identified from an antibody sequence by an algorithm comprising: identifying the conserved cysteine in framework 3 and the conserved tryptophan in framework 4; and determining the sequence of the CDR-3 knob, in which: the CDR-3 knob has the amino acid sequence length K; the sequence begins at position X+1 and ends at X+K; and K=L−2X; wherein L is the number of amino acids in an amino acid sequence starting at the conserved cysteine in framework 3 and ending at the conserved tryptophan in framework 4, and X is the number of amino acids from the first cysteine in framework 3 to the first conserved cysteine encoded by the DH region in CDR H3. The use of this formula to identify exemplary knob regions of ultralong CDRs is exemplified in Example 11.

In some embodiments, the binding peptide is 22 to 48 amino acids in length, such as 22 to 46 amino acids, 22 to 44 amino acids, 22 to 42 amino acids, 22 to 40 amino acids, 22 to 38 amino acids, 24 to 48 amino acids, 24 to 46 amino acids, 24 to 44 amino acids, 24 to 42 amino acids, 24 to 40 amino acids, 24 to 38 amino acids, 26 to 48 amino acids, 26 to 46 amino acids, 26 to 44 amino acids, 26 to 42 amino acids, 26 to 40 amino acids, 26 to 38 amino acids, 28 to 48 amino acids, 28 to 46 amino acids, 28 to 44 amino acids, 28 to 42 amino acids, 28 to 40 amino acids, 28 to 38 amino acids, 30 to 48 amino acids, 30 to 46 amino acids, 30 to 44 amino acids, 30 to 42 amino acids, 30 to 40 amino acids, 30 to 38 amino acids, 32 to 48 amino acids, 32 to 46 amino acids, 32 to 44 amino acids, 32 to 42 amino acids, 32 to 40 amino acids, 32 to 38 amino acids, 34 to 48 amino acids, 34 to 46 amino acids, 34 to 44 amino acids, 34 to 42 amino acids, 34 to 40 amino acids, 34 to 38 amino acids, 36 to 48 amino acids, 36 to 46 amino acids, 36 to 44 amino acids, 36 to 42 amino acids, 36 to 40 amino acids, 36 to 38 amino acids, 38 to 48 amino acids, 38 to 46 amino acids, 38 to 44 amino acids, 38 to 42 amino acids, 38 to 40 amino acids, 40 to 48 amino acids, 40 to 46 amino acids, 40 to 44 amino acids, 40 to 42 amino acids, 42 to 48 amino acids, 42 to 46 amino acids, 42 to 44 amino acids, 44 to 48 amino acis, 44 to 46 amino acids or 46 to 48 amino acids. In any of such embodiments, the binding peptide contains 2-12 amino acids are cysteine residues able to form 1-6 disulfide bonds.

In some embodiments, the binding peptide is about 22 amino acids in length. In some embodiments, the binding peptide is about 23 amino acids in length. In some embodiments, the binding peptide is about 24 amino acids in length. In some embodiments, the binding peptide is about 25 amino acids in length. In some embodiments, the binding peptide is about 26 amino acids in length. In some embodiments, the binding peptide is about 27 amino acids in length. In some embodiments, the binding peptide is about 28 amino acids in length. In some embodiments, the binding peptide is about 29 amino acids in length. In some embodiments, the binding peptide is about 30 amino acids in length. In some embodiments, the binding peptide is about 31 amino acids in length. In some embodiments, the binding peptide is about 32 amino acids in length. In some embodiments, the binding peptide is about 33 amino acids in length. In some embodiments, the binding peptide is about 34 amino acids in length. In some embodiments, the binding peptide is about 35 amino acids in length. In some embodiments, the binding peptide is about 36 amino acids in length. In some embodiments, the binding peptide is about 37 amino acids in length. In some embodiments, the binding peptide is about 38 amino acids in length. In some embodiments, the binding peptide is about 39 amino acids in length. In some embodiments, the binding peptide is about 40 amino acids in length. In some embodiments, the binding peptide is about 41 amino acids in length. In some embodiments, the binding peptide is about 42 amino acids in length. In some embodiments, the binding peptide is about 43 amino acids in length. In some embodiments, the binding peptide is about 44 amino acids in length. In some embodiments, the binding peptide is about 45 amino acids in length. In some embodiments, the binding peptide is about 46 amino acids in length. In some embodiments, the binding peptide is about 47 amino acids in length. In some embodiments, the binding peptide is about 48 amino acids in length. In any of such embodiments, the binding peptide contains 2-12 amino acids are cysteine residues able to form 1-6 disulfide bonds.

In some embodiments, the binding peptide has 2-12 cysteine (Cys) residues. In some embodiments, the binding peptide has 2, 4, 6, 8, 10 or 12 cysteine residues. In some embodiments, the binding peptide has 2-8 Cys residues. In some embodiments, the binding peptide has 2 Cys residues. In some embodiments, the binding peptide has 4 Cys residues. In some embodiments, the binding peptide has 6 Cys residues. In some embodiments, the binding peptide has 8 Cys residues.

In some embodiments, the binding peptide is capable of forming disulfide bonds, such as when expressed in a cell under conditions for folding. In some embodiments, the binding peptide is capable of forming 1 to 6 disulfide bonds. In some embodiments, the binding peptide is capable of forming 1, 2, 3, 4, 5 or 6 disulfide bonds. In some embodiments, the binding peptide is capable of forming 1 disulfide bond. In some embodiments, the binding peptide is capable of forming 2 disulfide bonds. In some embodiments, the binding peptide is capable of forming 3 disulfide bonds. In some embodiments, the binding peptide is capable of forming 4 disulfide bonds.

In some embodiments, the binding peptide is a disulfide-bonded peptide. In some embodiments, the binding peptide has 1 to 6 disulfide bonds. In some embodiments, the binding peptide has 1, 2, 3, 4, 5 or 6 disulfide bonds. In some embodiments, the binding peptide has 1 disulfide bond. In some embodiments, the binding peptide has 2 disulfide bonds. In some embodiments, the binding peptide has 3 disulfide bonds. In some embodiments, the binding peptide has 4 disulfide bonds.

In some embodiments, the binding peptide includes at least 6 amino acids following the most C-terminal cysteine residue present in the binding peptide. In some embodiments, the binding peptide includes 6-25 amino acids following the most C-terminal cysteine residue present in the binding peptide. In some embodiments, the binding peptide includes 6-20 amino acids following the most C-terminal cysteine residue present in the binding peptide. In some embodiments, the binding peptide includes 6-15 amino acids following the most C-terminal cysteine residue present in the binding peptide. In some embodiments, the soluble peptide includes 6, 7, 8, 9, 10, 11, 12, 13, 14, 15, 16, 17, 18, 19, 20, 21, 22, 23, 24 or 25 amino acids following the most C-terminal cysteine residue present in the binding peptide.

In some embodiments, the binding peptide retains at least a portion of the ascending and descending stalk domain on the N- and C-terminal ends, respectively, of the binding peptide. In some embodiments, the binding peptide includes adjacent N-terminal contiguous sequences of the ascending stalk most immediate to the knob region of 1, 2, 3, 4, 5, 6, 7, 8, 9 or 10 amino acids and the binding peptide includes adjacent most C-terminal contiguous sequences of the descending stalk most immediate to the knob region of 1, 2, 3, 4, 5, 6, 7, 8, 9 or 10 amino acids. In some embodiments, the binding peptide includes N-terminal contiguous sequences of the ascending stalk of 6 amino acids most immediate to the knob region and the binding peptide includes C-terminal contiguous sequences of the descending stalk of 6 amino acids most immediate to the knob region. In some embodiments, retaining residues from the ascending and descending stalk improves stability and activity of the knob peptide, including, in some cases, resulting in binding characteristics similar to Fab or full-length IgG forms of the ultralong CDR3 antibody from which the knob was derived.

In some embodiments, the binding peptide is modified with N-terminal (Y1) and C-terminal (Y2) sequences motifs that have been found herein to uniquely improve the stability and activity of knob binding peptides. Also provided herein are methods of producing a modified binding peptide by adding N- and C-terminal sequence motifs to a knob peptide sequence. In any of the provided embodiments, such N- and C-terminal sequences are added directly to the N- and C-terminal ends of the binding peptide sequence without any additional linker or other sequence between such sequence motifs and the binding peptide. In some embodiments, the knob binding peptide may be any as described above or identified using methods described herein. In some embodiments, the N- and C-terminal sequence motifs are non-bovine sequences. In some embodiments, the N- and C-terminal sequences are sequences that are not native to the ultralong CDR3 from which the knob peptide is derived. In some embodiments, the N- and C-terminal sequences are residues that are proximal to each other in the folded structure to “clamp” or stabilize the binding peptide. In some embodiments, the N- and C-terminal sequences are short peptide sequences. In some embodiments, the N- and C-terminal sequences are sequences able to form a coiled-coil motif.

In some embodiments, the binding peptide is modified by addition of two amino acids on the N-terminus and addition of two amino acids on the C-terminus that provide overhang for the knob peptide selected from: (i) HW and SF, respectively; (ii) IS and TV, respectively; (iii) DY and MP, respectively; (iv) LV and IP, respectively; or (v) SV and YI, respectively. In some embodiments, the Y1 and Y2 are selected from (i) HW and SF, respectively; (ii) IS and TV, respectively; (iii) DY and MP, respectively; (iv) LV and IP, respectively; or (v) SV and YI, respectively. In some embodiments, the N-terminus (Y1) and C-terminus (Y2) are modified with HW and SF, respectively. In some embodiments, th the N-terminus (Y1) and C-terminus (Y2) are modified with IS and TV, respectively.

In some embodiments, the binding peptide is modified by addition of N-terminus (Y1) and C-terminus (Y2) linker sequences able to interact to form an anti-parallel coiled-coil motif. In some embodiments, Y1 and Y2 are sequences that are each 14-30 amino acids in length comprising a heptad repeat, wherein Y1 and Y2 are able to interact to form an anti-parallel coiled-coil motif. In some embodiments, the binding peptide is modified by addition of N-terminus and C-terminus overhang sequences that include (i) the sequence set forth in SEQ ID NO:202 or a sequence having no or about 1, 2, 3 or 4 amino acid substitutions or deletions from SEQ ID NO:202, and (ii) the sequence set forth in SEQ ID NO:203 or a sequence having no or about 1, 2, 3 or 4 amino acid substitutions or deletions from SEQ ID NO:203. In some embodiments, the binding peptide is modified by addition of N-terminus and C-terminus overhang sequences that include (i) the sequence set forth in SEQ ID NO:202 and (ii) the sequence set forth in SEQ ID NO:203. In some embodiments, the modified binding peptide is produced by adding the sequence set forth in SEQ ID NO:202 to the N-terminus and the sequence set forth in SEQ ID NO:203 to the C-terminus of a knob peptide sequence.

In some embodiments, the modified binding peptide does not include an N-terminal or C-terminal GS linker sequence.

In some embodiments, the modified binding peptide does not contain a linker for cyclization of the peptide. In some embodiments, the modified binding peptide is not cyclized.

Any of a variety of methods known to a skilled artisan can be used to modify a binding peptide to add the above N-terminal and C-terminal residues. A variety of techniques including recombinant methods, chemical synthesis, or combinations thereof, may be employed. In some embodiments, methods for adding the N- and C-terminal amino acid residues to generate a modified binding peptide is by recombinant DNA methods. In some embodiments, methods for adding the N- and C-terminal amino acid residues to generate a modified binding peptide is by gene synthesis methods. Techniques for manipulating nucleic acids, such as those for generating mutation in sequences, subcloning, labeling, probing, sequencing, hybridization and so forth, are described in detail in scientific publications and patent documents. See, for example, Sambrook J, Russell D W (2001) Molecular Cloning: a Laboratory Manual, 3rd ed. Cold Spring Harbor Laboratory Press, New York; Current Protocols in Molecular Biology, Ausubel ed., John Wiley & Sons, Inc., New York (1997); Laboratory Techniques in Biochemistry and Molecular Biology: Hybridization With Nucleic Acid Probes, Part I, Theory and Nucleic Acid Preparation, Tijssen ed., Elsevier, N.Y. (1993). It is understood that the use of the term “added” or “addition” does not imply that the provided embodiments are limited to a particular method of making the modified binding peptides. The means by which the modified knob peptides are designed or created is not limited to any particular method.

In some embodiments, a modified binding peptide exhibits one or more improved activities or properties compared to a reference binding peptide that has not been modified by addition of N- and C-terminal amino acids. In some embodiments, the reference binding peptide is identical to the modified knob peptide, except that the added N- and C-terminal amino acids are absent. In some embodiments, the reference binding peptide is a binding peptide composed only of the knob region sequence. In some embodiments, the reference binding peptide is a knob region sequence that may be extended by 1, 2, 3, 4 or 5 amino acids on the N- and/or C-terminus to include a contiguous portion of the ascending (stalk A) and/or descending (stalk B) amino acids of the ultralong CDR3.

In some embodiments, the modified binding peptide is able to be expressed in soluble form, such as using a bacterial expression system described herein. In some embodiment, the degree or level of expression is increased or greater than the reference binding peptide that is similarly produced. In some embodiments, the degree or level of expression is increased by about 1.2-fold, 15.-fold, 2-fold, 3-fold, 4-fold, 5-fold, 10-fold, 20-fold, 10-fold or more.

In some embodiments, the modified binding peptide, including any produced by the provided methods, exhibits increased binding affinity the target antigen compared to the reference binding peptide. In some embodiments, the binding affinity is increased by about 1.2-fold, 15.-fold, 2-fold, 3-fold, 4-fold, 5-fold, 10-fold, 20-fold, 10-fold or more. Any of a variety of methods known to a skilled artisan can be used to assess binding affinity, including any of the methods described below. In some embodiments, the binding affinity dissociation constant (Kd) of binding is increased by about 1.2-fold, 15.-fold, 2-fold, 3-fold, 4-fold, 5-fold, 10-fold, 20-fold, 10-fold or more.

In some embodiments, the modified binding peptide, including any produced by the provided methods, exhibits increased activity or potency compared to the reference binding peptide against the target antigen. In some embodiments, the target antigen is a virus and the modified knob peptide exhibits improved neutralization activity compared to the reference binding peptide. In some embodiments, the neutralizing activity is an EC50 that is lower than the reference binding peptide. In some embodiments, the EC50 for neutralizing activity is about 1.2-fold, 15.-fold, 2-fold, 3-fold, 4-fold, 5-fold, 10-fold, 20-fold, 10-fold or more lower than the reference binding peptide. In some embodiments, the modified binding peptide has subnanomolar, such as picomolar, neutralizing activity.

Provided herein are fusion proteins, comprising a modified fusion polypeptide and a moiety. In some embodiments, the modified fusion polypeptide is any such as described herein, including in Section II. In some embodiments, the moiety is selected from a half-life extending moiety or a detectable moiety.

In some embodiments, provided are fusion proteins containing a modified fusion polypeptide and a half-life extending moiety. In some embodiments, half-life extending moieties of modified fusion polypeptides provide alterations in the pharmacodynamics and pharmacokinetics of the modified fusion polypeptide itself. As noted, in some embodiments, the half-life extending moiety extends the elimination half-life. In some embodiments, half-life extending moieties also modify the pharmacodynamic properties, including modifying the tissue distribution, penetration and diffusion of the modified fusion polypeptide. In some embodiments, the half-life extending moiety provides improved tissue (including tumor) targeting, tissue penetration, tissue distribution, diffusion within tissues, compared to polypeptides without the half-life extending binding moiety, and can provide enhanced efficacy.

Half-life extending moieties that extend the half-life of the modified fusion polypeptide are contemplated herein. Such domains are intended to include, but are not limited to immunogloulin domains, such as Fc domains; serum proteins, such as albumin; or transferrin. In some embodiments, the one or more half-life extending moiety comprise an Fc domain. In some embodiments, the half-life extending moiety can be by attachment to a PEG moiety or other polymer. In some embodiments, the half-life extending domain is at the N-terminus of the polypeptide, e.g., such as prior to protease cleavage. In some embodiments, the half-life extending moiety is at the C-terminus of the polypeptide, e.g., such as prior to protease cleavage. In some embodiments, the half-life extending moiety is neither C-terminal nor N-terminal to the polypeptide, e.g., such as prior to protease cleavage.

In some embodiments, provided are fusion proteins containing a modified fusion polypeptide and a detectable moiety. In some embodiments, a detectable moiety as recited herein relates to a moiety capable of being detected, e.g., primary labels and secondary labels. Primary labels, such as radioisotopes (e.g., tritium, 225Ac, 227Ac, 241Am, 72As, 74As, 211At, 198Au, 11B, 7Be, 212Bi, 213Bi, 75Br, 77Br, 11C, 14C, 48Ca, 109Cd, 139Ce, 141Ce, 252Cf, 55Co, 57Co, 60Co, 51Cr, 130Cs, 131Cs, 137Cs, 61Cu, 62Cu, 64Cu, 67Cu, 165Dy, 152Eu, 155Eu, 18F, 55Fe, 59Fe, 64Ga, 67Ga, 68Ga, 153Gd, 68Ge, 122I, 123I, 124I, 125I, 131I, 132I, 111In, 115mIn, 191mIr, 192Ir, 8lmKr, 177Lu, 51Mn, 52Mn, 99Mo, 13N, 95Nb, 150, 1910s, 1940s, 32P, 33P, 203Pb, 212Pb, 103Pd, 109Pd, 238Pu, 223Ra, 226Ra, 82Rb, 186Re, 188Re, 105Rh, 97Ru, 103Ru, 35S, 46Sc, 47Sc, 72Se, 75Se, 28Si, 145Sm, 153Sm, 117mSn, 85Sr, 89Sr, 90Sr, 178Ta, 179Ta, 182Ta, 149Tb, 96Tc, 99mTc, 228Th, 229Th, 201T1, 170Tm, 171Tm, 188W, 127Xe, 133Xe, 88Y, 90Y91Y, 169Yb, 62Zn, 65Zn, 89Zr or 95Zr, wherein a superscripted m denotes a meta-state), mass-tags, and fluorescent labels are signal generating reporter groups which can be detected without further modifications. Detectable moieties also include luminescent and phosphorescent groups or fluorescent molecules.

In some embodiments, the detectable moiety is a fluorescent molecule. In some embodiments, such a detectable moiety may be an optically detectable polypeptide. In some embodiments, the flurorescent molecule is a green fluorescent protein (GFP), red fluorescent protein (RFP), yellow fluorescent protein (YFP) and or cyan fluorescent protein (CFP), or any of the modified variants of these proteins, which are commercially available. For example, in some embodiments the detectable moiety may be sfGFP, super folded GFP. The detectable moiety may also be an enzyme such as luciferase orp-galactosidase, which can be reacted to give a detectable end point such as light emission or colour change. Nucleotide sequences encoding such polypeptides are readily available, for example from Clontech.

In some embodiments, the modified fusion polypeptide is linked to the moiety. In some embodiments, the linkage can be direct or indirect, such as via a peptide linker. In some embodiments, the modified fusion polypeptide is linked to the N-terminus of a moiety. In some embodiments, the modified fusion polypeptide is linked to the C-terminus of a moiety. In some embodiments, the modified fusion polypeptide is inserted within the moiety. For instance, in some embodiments the modified fusion polypeptide is inserted within a loop domain of the moiety.

In various other embodiments, the moiety is placed at a position within the molecule that is separated from the modified fusion polypeptide by a cleavable linker. Thus, for example, upon reaching a desired target within an agent that cleaves the linker (e.g., a protease, esterase, reducing or oxidizing microenvironment), the moiety is cleaved from the modified fusion polypeptide, reducing the size of the modified fusion polypeptide. facilitating its penetration into tissues or uptake by cells.

### A. Exemplary Binding Peptides Targeting SARS and Related Coronaviruses

Provided herein is an isolated binding peptide of between 20 and 60 amino acids in length containing 2-12 cysteine residues in which the binding peptide is directed against SARS-CoV-2. In some embodiments, binding peptide is a disulfide-bonded peptide of between 20 and 60 amino acids in length containing 1-6 disulfide bonds in which the binding peptide is directed against SARS-CoV-2. In some embodiments, the binding peptide is isolated or derived from the knob region of an ultralong CDR3 of a bovine antibody. Also provided are modified binding peptides of any of the provided binding peptides that are modified by addition of N- and C-terminal amino acid residues.

In some embodiments, the binding peptide is derived from an ultralong CDR3 of any of antibodies R4C1, R2C3, SKD, SKM, R2G3, R2F12, SR3A3 or R2D9. In some embodiments, the binding peptide is composed of the knob sequence of the ultralong CDR3 as contained in the heavy chain variable region set forth in any of SEQ ID NOS: 33, 34, 35, 40, 45, 46, 50 or 51. In some embodiments, the binding peptide has the sequence of any of the binding peptides in Table 2A. In some embodiments, the binding peptide has the sequence set forth in any one of SEQ ID NOS: 155, 198, and 227-240. In some embodiments, the binding petide has the sequence set forth in SEQ ID NO: 155. In some embodiments, the binding peptide has the sequence set forth in SEQ ID NO:198.

TABLE 2A
Exemplary CDR H3-Knob Sequences

| | SEQ | |
|---|---|---|
| Name | ID NO | Knob Sequence |
| 4C1 knob | 227 | CPDGYIYSSNTASGYDCGVWICRRVGSAFCSRTGDYTSPS |
| | 228 | NCPDGYIYSSNTASGYDCGVWICRRVGSAFCSRTGDYTSPS |
| R2C3 | 229 | CPDGYLYSSNTGRGYDCGVWTCRRVGGEFCSATGDWTSPS |
| (R5C1) | 230 | SCPDGYLYSSNTGRGYDCGVWTCRRVGGEFCSATGDWTSPS |
| knob | | |
| SKD | 231 | CPGGSSRRYPSGASCDVSGGACACYVSNCRGVLCPTLNEI |
| knob | 232 | SCPGGSSRRYPSGASCDVSGGACACYVSNCRGVLCPTLNEI |
| SKM | 233 | CPDGYIDNSGCTADWGCAALDCWRRRFGYHSTDPSHYTGATYI |
| knob | 198 | SCPDGYIDNSGCTADWGCAALDCWRRRFGYHSTDPSHYTGATYI |
| R2G3 | 234 | CPDGYEHTCGCIGGCGCKRSACIGALCCQASLGGWLSD |
| knob | 155 | TCPDGYEHTCGCIGGCGCKRSACIGALCCQASLGGWLSD |
| R2F12 | 235 | CPDDFDYRCSCIGGCGCARKGCVGPLCCRSDLGGYLTDSPA |
| knob | 236 | ACPDDFDYRCSCIGGCGCARKGCVGPLCCRSDLGGYLTDSPA |
| SR3A3 | 237 | CPDGYIYSSNITSGFDCGVWICRRVGSAFCSRTGDYTSPT |
| knob | 238 | NCPDGYIYSSNITSGFDCGVWICRRVGSAFCSRTGDYTSPT |
| R2D9 | 239 | CPNGYNWFDRCCSWDGTCGDGCCSNRAWPSGNGRADSSIG |
| knob | 240 | TCPNGYNWFDRCCSWDGTCGDGCCSNRAWPSGNGRADSSIG |

In some embodiments, the binding peptide may include a contiguous portion of amino acids of the ascending (Stalk A) or descending (Stalk B) as contained in the ultralong CDR3 of the heavy chain variable region set forth in any of SEQ ID NOS: 33, 34, 35, 40, 45, 46, 50 or 51. In some embodiments, the binding peptide includes a portion of the ascending stalk, knob region and a portion of the descending stalk of any of SEQ ID NOS: 33, 34, 35, 40, 45, 46, 50 or 51. In some embodiments, the binding peptide includes 1-15 amino acids N- and/or C-terminal amino acids that are contiguous residues of the ascending strain and/or contiguous residues of the descending strand. In some embodiments, the binding peptide has the sequence of any of the binding peptides in Table 2B. In some embodiments, the binding peptide has the sequence set forth in any one of SEQ ID NOS: 60, 61, 62, 63, 64, 65, 66, 67, 68 and 241-317.

In some embodiments, the binding peptide includes 1-7 amino acids of the ascending stalk, the knob sequence and 1-7 (e.g. 1, 2, 3, 4, 5, 6, or 7) amino acids of the descending stalk of an ultralong CDR3 of any of SEQ ID NOS: 33, 34, 35, 40, 45, 46, 50 or 51. In some embodiments, the binding peptide includes 1-7 (e.g. 1, 2, 3, 4, 5, 6, or 7) amino acids of the ascending stalk, the knob sequence and 4-7 amino acids of the descending stalk of an ultralong CDR3 set forth in SEQ ID NOS: 33. In some embodiments, the binding peptide includes 4-7 amino acids of the ascending stalk, the knob sequence and 4-7 amino acids of the descending stalk of an ultralong CDR3 set forth in SEQ ID NO: 34. In some embodiments, the binding peptide includes 4-7 amino acids of the ascending stalk, the knob sequence and 4-7 amino acids of the descending stalk of an ultralong CDR3 set forth in SEQ ID NO: 35. In some embodiments, the binding peptide includes 4-7 amino acids of the ascending stalk, the knob sequence and 4-7 amino acids of the descending stalk of an ultralong CDR3 set forth in SEQ ID NO: 40. In some embodiments, the binding peptide includes 4-7 amino acids of the ascending stalk, the knob sequence and 1-7 amino acids of the descending stalk of an ultralong CDR3 set forth in SEQ ID NO: 45. In some embodiments, the binding peptide includes 4-7 amino acids of the ascending stalk, the knob sequence and 4-7 amino acids of the descending stalk of an ultralong CDR3 set forth in SEQ ID NO: 46. In some embodiments, the binding peptide includes 4-7 amino acids of the ascending stalk, the knob sequence and 4-7 amino acids of the descending stalk of an ultralong CDR3 set forth in SEQ ID NO: 50. In some embodiments, the binding peptide includes 1-7 amino acids of the ascending stalk, the knob sequence and 4-7 amino acids of the descending stalk of an ultralong CDR3 set forth in SEQ ID NO: 51.

In some embodiments, the the binding peptide is set forth in SEQ ID NO: 318:

HQETLRSCPDGYIDNSGCTADWGCAALDCWRRRFGYHSTDPSHYTGATYI YTYSLH

TABLE 2B
Exemplary CDR H3-Knob Sequences

| | SEQ | |
|---|---|---|
| Name | ID NO | Knob Sequence |
| R4C1 | 241 | RKEKNCPDGYIYSSNTASGYDCGVWICRRVGSAFCSRTGDYTSPSEFDIYEFY |
| knob | 242 | RKEKNCPDGYIYSSNTASGYDCGVWICRRVGSAFCSRTGDYTS |
| | 243 | RKEKNCPDGYIYSSNTASGYDCGVWICRRVGSAFCSRTGDYTSP |
| | 244 | RKEKNCPDGYIYSSNTASGYDCGVWICRRVGSAFCSRTGDYTSPS |
| | 245 | RKEKNCPDGYIYSSNTASGYDCGVWICRRVGSAFCSRTGDYTSPSE |
| | 246 | RKEKNCPDGYIYSSNTASGYDCGVWICRRVGSAFCSRTGDYTSPSEF |
| | 245 | RKEKNCPDGYIYSSNTASGYDCGVWICRRVGSAFCSRTGDYTSPSEFD |
| | 248 | RKEKNCPDGYIYSSNTASGYDCGVWICRRVGSAFCSRTGDYTSPSEFDI |
| | 63 | RKEKNCPDGYIYSSNTASGYDCGVWICRRVGSAFCSRTGDYTSPSEFDIY |
| | 249 | RKEKNCPDGYIYSSNTASGYDCGVWICRRVGSAFCSRTGDYTSPSEFDIYE |
| | 250 | RKEKNCPDGYIYSSNTASGYDCGVWICRRVGSAFCSRTGDYTSPSEFDIYEF |
| R2C3 | 66 | RKEKSCPDGYLYSSNTGRGYDCGVWTCRRVGGEFCSATGDWTSPSEEDFYEF |
| knob | 251 | RKEKSCPDGYLYSSNTGRGYDCGVWTCRRVGGEFCSATGDWTS |
| | 252 | RKEKSCPDGYLYSSNTGRGYDCGVWTCRRVGGEFCSATGDWTSP |
| | 253 | RKEKSCPDGYLYSSNTGRGYDCGVWTCRRVGGEFCSATGDWTSPS |
| | 254 | RKEKSCPDGYLYSSNTGRGYDCGVWTCRRVGGEFCSATGDWTSPSE |
| | 255 | RKEKSCPDGYLYSSNTGRGYDCGVWTCRRVGGEFCSATGDWTSPSEE |
| | 256 | RKEKSCPDGYLYSSNTGRGYDCGVWTCRRVGGEFCSATGDWTSPSEED |
| | 257 | RKEKSCPDGYLYSSNTGRGYDCGVWTCRRVGGEFCSATGDWTSPSEEDF |
| | 258 | RKEKSCPDGYLYSSNTGRGYDCGVWTCRRVGGEFCSATGDWTSPSEEDFY |
| | 259 | RKEKSCPDGYLYSSNTGRGYDCGVWTCRRVGGEFCSATGDWTSPSEEDFYE |
| SKD | 260 | SEKRSCPGGSSRRYPSGASCDVSGGACACYVSNCRGVLCPTLNEIVAYTYEWH |
| | | VD |
| knob | 261 | SEKRSCPGGSSRRYPSGASCDVSGGACACYVSNCRGVLCPTLNEI |
| | 262 | SEKRSCPGGSSRRYPSGASCDVSGGACACYVSNCRGVLCPTLNEIV |
| | 263 | SEKRSCPGGSSRRYPSGASCDVSGGACACYVSNCRGVLCPTLNEIVA |
| | 264 | SEKRSCPGGSSRRYPSGASCDVSGGACACYVSNCRGVLCPTLNEIVAY |
| | 265 | SEKRSCPGGSSRRYPSGASCDVSGGACACYVSNCRGVLCPTLNEIVAYT |
| | 65 | SEKRSCPGGSSRRYPSGASCDVSGGACACYVSNCRGVLCPTLNEIVAYTY |
| | 266 | SEKRSCPGGSSRRYPSGASCDVSGGACACYVSNCRGVLCPTLNEIVAYTYE |
| | 267 | SEKRSCPGGSSRRYPSGASCDVSGGACACYVSNCRGVLCPTLNEIVAYTYEW |
| | 268 | SEKRSCPGGSSRRYPSGASCDVSGGACACYVSNCRGVLCPTLNEIVAYTYEWH |
| | 269 | SEKRSCPGGSSRRYPSGASCDVSGGACACYVSNCRGVLCPTLNEIVAYTYEWH |
| | | V |
| SKM | 270 | LRSCPDGYIDNSGCTADWGCAALDCWRRRFGYHSTDPSHYTGATYIYTYSHID |
| knob | 271 | LRSCPDGYIDNSGCTADWGCAALDCWRRRFGYHSTDPSHYTGAT |
| | 272 | LRSCPDGYIDNSGCTADWGCAALDCWRRRFGYHSTDPSHYTGATY |
| | 273 | LRSCPDGYIDNSGCTADWGCAALDCWRRRFGYHSTDPSHYTGATYI |
| | 274 | LRSCPDGYIDNSGCTADWGCAALDCWRRRFGYHSTDPSHYTGATYIY |
| | 275 | LRSCPDGYIDNSGCTADWGCAALDCWRRRFGYHSTDPSHYTGATYIYT |
| | 64 | LRSCPDGYIDNSGCTADWGCAALDCWRRRFGYHSTDPSHYTGATYIYTY |
| | 276 | LRSCPDGYIDNSGCTADWGCAALDCWRRRFGYHSTDPSHYTGATYIYTYS |
| | 277 | LRSCPDGYIDNSGCTADWGCAALDCWRRRFGYHSTDPSHYTGATYIYTYS |
| | 278 | LRSCPDGYIDNSGCTADWGCAALDCWRRRFGYHSTDPSHYTGATYIYTYSH |
| | 279 | LRSCPDGYIDNSGCTADWGCAALDCWRRRFGYHSTDPSHYTGATYIYTYSHI |
| R2G3 | 60 | EGDKTCPDGYEHTCGCIGGCGCKRSACIGALCCQASLGGWLSDGETYTYEF |
| knob | 280 | EGDKTCPDGYEHTCGCIGGCGCKRSACIGALCCQASLGGWLS |
| | 281 | EGDKTCPDGYEHTCGCIGGCGCKRSACIGALCCQASLGGWLSD |
| | 282 | EGDKTCPDGYEHTCGCIGGCGCKRSACIGALCCQASLGGWLSDG |
| | 283 | EGDKTCPDGYEHTCGCIGGCGCKRSACIGALCCQASLGGWLSDGE |
| | 284 | EGDKTCPDGYEHTCGCIGGCGCKRSACIGALCCQASLGGWLSDGET |
| | 285 | EGDKTCPDGYEHTCGCIGGCGCKRSACIGALCCQASLGGWLSDGETY |
| | 286 | EGDKTCPDGYEHTCGCIGGCGCKRSACIGALCCQASLGGWLSDGETYT |
| | 287 | EGDKTCPDGYEHTCGCIGGCGCKRSACIGALCCQASLGGWLSDGETYTY |
| | 288 | EGDKTCPDGYEHTCGCIGGCGCKRSACIGALCCQASLGGWLSDGETYTYE |
| | 289 | GDKTCPDGYEHTCGCIGGCGCKRSACIGALCCQASLGGWLSD |
| | 290 | DKTCPDGYEHTCGCIGGCGCKRSACIGALCCQASLGGWLSD |
| | 291 | KTCPDGYEHTCGCIGGCGCKRSACIGALCCQASLGGWLSD |
| R2F12 | 292 | KTKNACPDDFDYRCSCIGGCGCARKGCVGPLCCRSDLGGYLTDSPAYIYEWY |
| knob | 293 | KTKNACPDDFDYRCSCIGGCGCARKGCVGPLCCRSDLGGYLTDS |
| | 294 | KTKNACPDDFDYRCSCIGGCGCARKGCVGPLCCRSDLGGYLTDSP |
| | 295 | KTKNACPDDFDYRCSCIGGCGCARKGCVGPLCCRSDLGGYLTDSPA |
| | 296 | KTKNACPDDFDYRCSCIGGCGCARKGCVGPLCCRSDLGGYLTDSPAY |
| | 297 | KTKNACPDDFDYRCSCIGGCGCARKGCVGPLCCRSDLGGYLTDSPAYI |
| | 298 | KTKNACPDDFDYRCSCIGGCGCARKGCVGPLCCRSDLGGYLTDSPAYIY |
| | 62 | KTKNACPDDFDYRCSCIGGCGCARKGCVGPLCCRSDLGGYLTDSPAYIYE |
| | 299 | KTKNACPDDFDYRCSCIGGCGCARKGCVGPLCCRSDLGGYLTDSPAYIYEW |
| SR3A3 | 61 | KEKNCPDGYIYSSNITSGFDCGVWICRRVGSAFCSRTGDYTSPTELDIYEF |
| knob | 300 | KEKNCPDGYIYSSNITSGFDCGVWICRRVGSAFCSRTGDYTS |
| | 301 | KEKNCPDGYIYSSNITSGFDCGVWICRRVGSAFCSRTGDYTSP |
| | 302 | KEKNCPDGYIYSSNITSGFDCGVWICRRVGSAFCSRTGDYTSPT |
| | 303 | KEKNCPDGYIYSSNITSGFDCGVWICRRVGSAFCSRTGDYTSPTE |
| | 304 | KEKNCPDGYIYSSNITSGFDCGVWICRRVGSAFCSRTGDYTSPTEL |
| | 305 | KEKNCPDGYIYSSNITSGFDCGVWICRRVGSAFCSRTGDYTSPTELD |
| | 306 | KEKNCPDGYIYSSNITSGFDCGVWICRRVGSAFCSRTGDYTSPTELDI |
| | 307 | KEKNCPDGYIYSSNITSGFDCGVWICRRVGSAFCSRTGDYTSPTELDIY |
| | 308 | KEKNCPDGYIYSSNITSGFDCGVWICRRVGSAFCSRTGDYTSPTELDIYE |
| R2D9 | 68 | ITHKTCPNGYNWFDRCCSWDGTCGDGCCSNRAWPSGNGRADSSIGETYGYEF |
| knob | 309 | ITHKTCPNGYNWFDRCCSWDGTCGDGCCSNRAWPSGNGRADSS |
| | 310 | ITHKTCPNGYNWFDRCCSWDGTCGDGCCSNRAWPSGNGRADSSI |
| | 311 | ITHKTCPNGYNWFDRCCSWDGTCGDGCCSNRAWPSGNGRADSSIG |
| | 312 | ITHKTCPNGYNWFDRCCSWDGTCGDGCCSNRAWPSGNGRADSSIGE |
| | 313 | ITHKTCPNGYNWFDRCCSWDGTCGDGCCSNRAWPSGNGRADSSIGET |
| | 314 | ITHKTCPNGYNWFDRCCSWDGTCGDGCCSNRAWPSGNGRADSSIGETY |
| | 315 | ITHKTCPNGYNWFDRCCSWDGTCGDGCCSNRAWPSGNGRADSSIGETYG |
| | 316 | ITHKTCPNGYNWFDRCCSWDGTCGDGCCSNRAWPSGNGRADSSIGETYGY |
| | 317 | ITHKTCPNGYNWFDRCCSWDGTCGDGCCSNRAWPSGNGRADSSIGETYGYE |

Also provided herein are modified binding peptides of any of the above binding peptides in Tables 6A and 6B that are modified by the addition of N- and C-terminal amino acids sequences that are not present in the respective ultralong CDR3 set forth in any one of SEQ ID NOS: 33, 34, 35, 40, 45, 46, 50 or 51.

In some embodiments, a modified binding peptide herein is composed of a binding peptide set forth in Tables 6A or 6B with the addition of two amino acids on the N-terminus and addition of two amino acids on the C-terminus that provide overhang for the knob peptide selected from: (i) HW and SF, respectively; (ii) IS and TV, respectively; (iii) DY and MP, respectively; (iv) LV and IP, respectively; or (v) SV and YI, respectively. In some embodiments, the N- and C-terminus are modified with N-terminus and C-terminus overhang sequences HW and SF, respectively. In some embodiments, the binding peptide is modified with N-terminus and C-terminus overhang sequences IS and TV, respectively.

In some embodiments, provided herein is a modified binding peptide containing the sequence from N-terminus to C-terminus: HW-[binding peptide]-SF, in which the binding peptide is the sequence of amino acids set forth in any one of SEQ ID NOS: 60, 61, 62, 63, 64, 65, 66, 67, 68, 155, 198 and 227-317. In some embodiments, provided herein is a modified binding peptide containing the sequence from N-terminus to C-terminus: HW-[binding peptide]-SF, in which the binding peptide is the sequence of amino acids set forth in any one of SEQ ID NOS: 155, 198, and 227-240. In some embodiments, the modified binding peptide contains the sequence from N-terminus to C-terminus: HW-TCPDGYEHTCGCIGGCGCKRSACIGALCCQASLGGWLSD (SEQ ID NO:155)-SF. In some embodiments, the modified binding peptide contains the sequence from N-terminus to C-terminus: HW-SCPDGYIDNSGCTADWGCAALDCWRRRFGYHSTDPSHYTGATYI (SEQ ID NO:198)-SF.

In some embodiments, provided herein is a modified binding peptide containing the sequence from N-terminus to C-terminus IS-[binding peptide]-TV, in which the binding peptide is the sequence of amino acids set forth in any one of SEQ ID NOS: 60, 61, 62, 63, 64, 65, 66, 67, 68, 155, 198 and 227-317. In some embodiments, provided herein is a modified binding peptide containing the sequence from N-terminus to C-terminus: IS-[binding peptide]-TV, in which the binding peptide is the sequence of amino acids set forth in any one of SEQ ID NOS: 155, 198, and 227-240. In some embodiments, the modified binding peptide contains the sequence from N-terminus to C-terminus: IS-TCPDGYEHTCGCIGGCGCKRSACIGALCCQASLGGWLSD (SEQ ID NO:155)-TV. In some embodiments, the modified binding peptide contains the sequence from N-terminus to C-terminus: IS-SCPDGYIDNSGCTADWGCAALDCWRRRFGYHSTDPSHYTGATYI (SEQ ID NO:198)-TV.

In some embodiments, provided herein is a modified peptide containing the sequence from N-terminus to C-terminus: DY-[binding peptide]-MP, in which the binding peptide is the sequence of amino acids set forth in any one of SEQ ID NOS: 60, 61, 62, 63, 64, 65, 66, 67, 68, 155, 198 and 227-317. In some embodiments, provided herein is a modified binding peptide containing the sequence from N-terminus to C-terminus: DY-[binding peptide]-MP, in which the binding peptide is the sequence of amino acids set forth in any one of SEQ ID NOS: 155, 198, and 227-240. In some embodiments, the modified binding peptide contains the sequence from N-terminus to C-terminus: DY-TCPDGYEHTCGCIGGCGCKRSACIGALCCQASLGGWLSD (SEQ ID NO:155)-MP. In some embodiments, the modified binding peptide contains the sequence from N-terminus to C-terminus: DY-SCPDGYIDNSGCTADWGCAALDCWRRRFGYHSTDPSHYTGATYI (SEQ ID NO:198)-MP.

In some embodiments, provided herein is a modified binding peptide containing the sequence from N-terminus to C-terminus: LV-[binding peptide]-IP, in which the binding peptide is the sequence of amino acids set forth in any one of SEQ ID NOS: 60, 61, 62, 63, 64, 65, 66, 67, 68, 155, 198 and 227-317. In some embodiments, provided herein is a modified binding peptide containing the sequence from N-terminus to C-terminus: LV-[binding peptide]-IP, in which the binding peptide is the sequence of amino acids set forth in any one of SEQ ID NOS: 155, 198, and 227-240. In some embodiments, the modified binding peptide contains the sequence from N-terminus to C-terminus: LV-TCPDGYEHTCGCIGGCGCKRSACIGALCCQASLGGWLSD (SEQ ID NO:155)-IP. In some embodiments, the modified binding peptide contains the sequence from N-terminus to C-terminus: LV-SCPDGYIDNSGCTADWGCAALDCWRRRFGYHSTDPSHYTGATYI (SEQ ID NO:198)-IP.

In some embodiments, provided herein is a modified binding peptide containing the sequence from N-terminus to C-terminus: SV-[binding peptide]-YI, in which the binding peptide is the sequence of amino acids set forth in any one of SEQ ID NOS: 60, 61, 62, 63, 64, 65, 66, 67, 68, 155, 198 and 227-317. In some embodiments, provided herein is a modified binding peptide containing the sequence from N-terminus to C-terminus: SV-[binding peptide]-YI, in which the binding peptide is the sequence of amino acids set forth in any one of SEQ ID NOS: 155, 198, and 227-240. In some embodiments, the modified binding peptide contains the sequence from N-terminus to C-terminus: SV-TCPDGYEHTCGCIGGCGCKRSACIGALCCQASLGGWLSD (SEQ ID NO:155)-YI. In some embodiments, the modified binding peptide contains the sequence from N-terminus to C-terminus: SV-SCPDGYIDNSGCTADWGCAALDCWRRRFGYHSTDPSHYTGATYI (SEQ ID NO:198)-YI.

In some embodiments, the binding peptide is modified by addition of N- and C-terminal linker sequences able to interact to form an anti-parallel coiled-coil motif. In some eembodiments, a binding peptide set forth in Table 2A or 2B is modified by addition of N-terminus and C-terminus overhang sequences that include (i) the sequence set forth in SEQ ID NO:202 or a sequence having no or about 1, 2, 3 or 4 amino acid substitutions or deletions from SEQ ID NO:202, and (ii) the sequence set forth in SEQ ID NO:203 or a sequence having no or about 1, 2, 3 or 4 amino acid substitutions or deletions from SEQ ID NO:203. In some embodiments, the binding peptide is modified by addition of N-terminus and C-terminus overhang sequences that include (i) the sequence set forth in SEQ ID NO:202 and (ii) the sequence set forth in SEQ ID NO:203, respectively. In some embodiments, the modified binding peptide is produced by adding the sequence set forth in SEQ ID NO:202 to the N-terminus and the sequence set forth in SEQ ID NO:203 to the C-terminus of a knob peptide sequence.

In some embodiments, provided herein is a modified binding peptide containing the sequence from N-terminus to C-terminus: SEQ ID NO:202; a binding peptide set forth in any one of SEQ ID NOS: 60, 61, 62, 63, 64, 65, 66, 67, 68, 155, 198 and 227-317; and the sequence set forth in SEQ ID NO:203. In some embodiments, provided herein is a modified binding peptide containing the sequence from N-terminus to C-terminus: SEQ ID NO:202; a binding peptide set forth in any one of SEQ ID NOS: 155, 198, and 227-240; and the sequence set forth in SEQ ID NO:203. In some embodiments, provided herein is a modified binding peptide containing the sequence from N-terminus to C-terminus: SEQ ID NO:202; a binding peptide set forth in any one of SEQ ID NOS: 155, 198, and 227-240; and the sequence set forth in SEQ ID NO:203. In some embodiments, provided herein is a modified binding peptide containing the sequence from N-terminus to C-terminus: SEQ ID NO:202, SEQ ID NO: 155 and SEQ ID NO:203. In some embodiments, provided herein is a modified binding peptide containing the sequence from N-terminus to C-terminus: SEQ ID NO:202, SEQ ID NO: 198 and SEQ ID NO:203.

In some embodiments, any of the above binding peptides, including modified binding peptides provided herein binds to a coronavirus. In some embodiments, the binding peptides, including modified binding peptides, provided herein can bind to and/or neutralize an alphacoronavirus, a betacoronavirus, a gammacoronavirus, and/or a deltacoronavirus. In certain embodiments, this binding and/or neutralization can be specific for a particular genus of coronavirus or for a particular subgroup of a genus. In some embodiments, the coronavirus is SARS CoV-2. In some embodiments, any of the binding peptides provided herein, including aa modified binding peptide, binds to a SARS CoV-2 spike (S) protein.

In some embodiments, the binding peptide, including a modified binding peptide, binds to an epitope within SARS-CoV-2 spike protein. In some embodiments, the binding peptide, including a modified binding peptide, binds to the receptor binding domain (RBD) of SARS-CoV-2 spike protein. In some embodiments, the epitope recognized by a provided binding peptide, including a modified binding peptide, is within RBD of SARS-CoV-2 spike protein.

In another aspect, provided herein are binding peptides, including modified binding peptides, that bind the same or an overlapping epitope of S (e.g., an epitope of SARS CoV-2 S) as a binding peptide described herein.

In some embodiments, the epitope of a binding peptide can be determined by, e.g; NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al, (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251 6300-6303). Antibody: antigen crystals may be studied using well known X-ray diffraction techniques and may be refined using computer software such as X-PLOR (Yale University, 1992, distributed by Molecular Simulations, Inc.; see, e.g., Meth Enzymol (1985) volumes 114 & 1 15, eds Wyckoff H W et al; U.S. Patent Application No. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al, (2000) Acta Crystallogr D Biol Crystallogr 56(Pt 10): 1316-1323). Mutagenesis mapping studies may be accomplished using any method known to one of skill in the art. See, e.g., Champe M et al, (1995) supra and Cunningham B C & Wells J A (1989) supra for a description of mutagenesis techniques, including alanine scanning mutagenesis techniques.

In some embodiments, the epitope of a binding peptide is determined using alanine scanning mutagenesis studies. Usually, binding to the antigen is reduced or disrupted when a residue within the epitope is substituted to alanine. In one embodiment, the Kd of binding to the antigen is increased by about 5-fold, 10-fold, 20-fold, 10-fold or more when a residue within the epitope is substituted for alanine. In one embodiment, binding affinity is determined by ELISA. In addition, binding peptides that recognize and bind to the same or overlapping epitopes of S (e.g., an epitope of SASRS CoV-2 S) can be identified using routine techniques such as an immunoassay, for example, by showing the ability of one binding peptide to block the binding of anotherbinding peptide to a target antigen, i.e., a competitive binding assay.

In some embodiments, the binding peptides, including a modified binding peptide, provided herein neutralize a coronavirus. In some embodiments, the binding peptide, including a modified binding peptide, neutralize SARS-COV-2 including isolates or variants thereof. In some embodiments, the binding peptide, including a modified binding peptide, are pan-neutralizing to two or more coronaviruses.

In some embodiments, the provided binding peptide, including a modified binding peptide, are capable of binding CoV-2 spike protein, such as SARS-CoV-2 spike protein, with at least a certain affinity, as measured by any of a number of known methods. In some embodiments, the affinity is represented by an equilibrium dissociation constant (K_{D}); in some embodiments, the affinity is represented by EC₅₀.

A variety of assays are known for assessing binding affinity and/or determining whether a binding molecule (e.g., an antibody or fragment thereof or knob peptide) specifically binds to a particular antigen (e.g., spike protein). It is within the level of a skilled artisan to determine the binding affinity of a binding molecule, such as by using any of a number of binding assays that are well known in the art. For example, in some embodiments, a BIAcore® instrument can be used to determine the binding kinetics and constants of a complex between two proteins, using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed nucleic acids or binding of proteins.

In some embodiments, the binding peptide, such as a modified binding peptide, binds, such as specifically binds, to an antigen, e.g., a spike protein or receptor binding domain or an epitope therein, with an affinity or K_{A} (i.e., an equilibrium association constant of a particular binding interaction with units of 1/M; equal to the ratio of the on-rate [kₒₙ or kₐ] to the off-rate [k_{off} or k_{d}] for this association reaction, assuming bimolecular interaction) equal to or greater than 10⁸ M⁻¹. In some embodiments, the binding peptide, such as a modified binding peptide, exhibits a binding affinity for the peptide epitope with a K_{D} (i.e., an equilibrium dissociation constant of a particular binding interaction with units of M; equal to the ratio of the off-rate [k_{off} or k_{d}] to the on-rate [kₒₙ or kₐ] for this association reaction, assuming bimolecular interaction) of equal to or less than 10⁻⁸ M. For example, the equilibrium dissociation constant K_{D} ranges from 10⁻⁸ M to 10⁻¹³ M, such as 10⁻⁹ M to 10⁻¹³ M, 10⁻⁹ M to 10⁻¹² M, or 10⁻⁹ M to 10⁻¹¹ M, 10⁻⁹ M to 10⁻¹⁰ M, 10⁻¹⁰ M to 10⁻¹³ M, 10⁻¹⁰ M to 10⁻¹² M, or 10⁻¹⁰ M to 10⁻¹¹ M, 10⁻¹¹ M to 10⁻¹³ M, 10⁻¹¹ M to 10⁻¹² M or 10⁻¹¹ M to 10⁻¹³ M. In some embodiments, the K_{D} is less than at or about 10⁻⁹ M, less than at or about 10⁻¹⁰ M, less than at or about 10⁻¹¹ M or less than at or about 10⁻¹² M. Among provided binding binding peptides, including a modified binding peptide, the K_{D} is less than at or about 10⁻¹² M. The on-rate (association rate constant; kₒₙ or kₐ; units of 1/Ms) and the off-rate (dissociation rate constant; k_{off} or k_{d}; units of 1/s) can be determined using any of the assay methods known in the art, for example, surface plasmon resonance (SPR).

In some embodiments, a provided binding peptide, including a modified binding peptide, exhibits neutralizing activity. The term “neutralizing acitivity” refers to activity of a molecule, such as a binding peptide, to reduce at least one activity of a polypeptide comprising the epitope to which the molecule specifically binds. In some aspects, a neutralizing activity of a binding molecule prevents a structure (i.e., organism, virus, particle, etc.) comprising the epitope to which it binds from entering a cell, thus protecting that cell from infection. In certain embodiments, a binding molecule exhibits neutralizing activity if it reduces an activity in vitro and/or in vivo, such as viral entry into a host cell. In some aspects, a binding molecule that exhibits neutralizing activity can target an epitope of an infectious agent, such as a virus. In some aspects, a binding molecule that exhibits neutralizing activity can target an epitope of a host cell to which an infectious agent binds, such as a viral receptor glycoprotein.

In some aspects, the epitope bound by a binding peptide, such as one with neutralizing activity, can be any protein that includes a receptor binding domain (RBD) that mediates binding to a cognate receptor on a target host cell that can be infected by the virus. In some embodiments, the first step in any viral life cycle is contact and attachment with a target host cell, which can be mediated by structural proteins or viral membrane proteins via a surface exposed receptor binding domain (RBD). Antibodies or other binding domains which target the RBD or subvert its function are among the class of antibodies known as neutralizing, as their binding occludes virus-host receptor interaction and therefore “neutralizes” the ability to gain entry into a host cell.

In some embodiments, methods that calculate the neutralizing activity of a binding molecule include the plaque assay or plaque reduction and neutralization test (PRNT). Titrations of the virus are grown on cell monolayers that are overlaid with agarose in the presence of serial dilatation concentration of binding molecule. After incubation for a time period to achieve a cytopathic effect, such as for about 3 to 28 days, generally 7 to 10 days, the cells can be fixed and foci of absent cells visualized as plaques are determined. The neutralizing activity can be quantified via measurement of plaque number, plaque size, and plaque morphology. Percent neutralization, such as Percent Maximal Neutralization (PMN), can be calculated. Neutralizing efficacy of a binding molecule can be reported as 50%, 60%, 70%, 80%, or 90% neutralization, which represent the last dilution concentration of the binding molecule capable of inhibiting 50%, 60%, 70%, 80%, or 90% of total plaques.

In some embodiments, the binding peptide, including modified binding peptide, is capable of neutralizing a coronavirus, such as SARS-CoV-2, with an EC50 equal to or less than about 100 ng/mL, 10 ng/mL, 1 ng/mL, 0.1 ng/mL, 0.01 ng/mL, 0.001 ng/mL, or less. In some embodiments, the EC50 is less than about 100 nM, 10 nM, 1 nM, 0.1 nM, 0.01 nM or 0.001 nM or less. In some embodiments, the neutralizing activity is subnanomolar, such as picomolar. In some embodiments, the neutralizing activity is less than 1 nM. In some embodiments, the neutralizing activity is less than at or about 500 pM, 250 pM, 100 pM, 50 pM, 25 pM, 10 pM, 5 pM, 2.5 pM, 1 pM, 0.5 pM or less.

In some embodiments, the binding peptide, including modified binding peptide, is capable of neutralizing at least two cross-lineage SARS CoV-2 isolates. In some embodiments, a broadly neutralizing antibody or binding polypeptide (e.g. knob peptide) described herein specifically binds to S and is capable of neutralizing at least two isolates of SARS CoV-2. In some embodiments, the two isolates are two cross-lineage isolates. In some embodiments, the binding peptide, such as modified binding peptide, is capable of neutralizing at least about 50%, 60%, 70%, 80%, 90%, or 100% of cross-lineage SARS CoV-2 isolates. In one embodiment, the binding peptide, such as modified binding peptide, is capable of neutralizing at least about 90% of cross-lineage SARS CoV-2 isolates. In some embodiments, the binding peptide, such as modified binding peptide, is capable of neutralizing the cross-lineage SARS CoV-2 isolates with a median IC50 equal to or less than about 100 ng/mL, 10 ng/mL, 1 ng/mL, 0.1 ng/mL, 0.01 ng/mL, 0.001 ng/mL, or less.

The affinity or avidity of an antibody or fusion polypeptide for an antigen can be determined experimentally using any suitable method well known in the art, e.g., flow cytometry, enzyme-linked immunosorbent assay (ELISA), or radioimmunoassay (RIA), or kinetics (e.g., BIACORE™ analysis). Direct binding assays as well as competitive binding assay formats can be readily employed. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., KD or Kd, Kon, Koff) are made with standardized solutions of antibody and antigen, and a standardized buffer, as known in the art and such as the buffer described herein.

In some embodiments, any of the above modified binding peptides exhibit one or more improved activities or properties compared to a reference binding peptide that has not been modified by addition of N- and C-terminal amino acids. In some embodiments, the reference binding peptide is identical to the modified knob peptide, except that the added N- and C-terminal amino acids are absent. In some embodiments, the reference binding peptide is a binding peptide composed only of the knob region sequence. In some embodiments, the reference binding peptide is a knob region sequence that may be extended by 1, 2, 3, 4 or 5 amino acids on the N- and/or C-terminus to include a contiguous portion of the ascending (stalk A) and/or descending (stalk B) amino acids of the ultralong CDR3.

In some embodiments, the modified binding peptide is able to be expressed in soluble form, such as using a bacterial expression system described herein. In some embodiment, the degree or level of expression is increased or greater than the reference binding peptide that is similarly produced. In some embodiments, the degree or level of expression is increased by about 1.2-fold, 15.-fold, 2-fold, 3-fold, 4-fold, 5-fold, 10-fold, 20-fold, 10-fold or more.

In some embodiments, the modified binding peptide, including any produced by the provided methods, exhibits increased binding affinity the target antigen compared to the reference binding peptide. In some embodiments, the binding affinity is increased by about 1.2-fold, 15.-fold, 2-fold, 3-fold, 4-fold, 5-fold, 10-fold, 20-fold, 10-fold or more. Any of a variety of methods known to a skilled artisan can be used to assess binding affinity, including any of the methods described below. In some embodiments, the binding affinity dissociation constant (Kd) of binding is increased by about 1.2-fold, 15.-fold, 2-fold, 3-fold, 4-fold, 5-fold, 10-fold, 20-fold, 10-fold or more.

In some embodiments, the modified binding peptide, including any produced by the provided methods, exhibits increased activity or potency compared to the reference binding peptide against the target antigen. In some embodiments, the target antigen is a virus and the modified knob peptide exhibits improved neutralization activity compared to the reference binding peptide. In some embodiments, the neutralizing activity is an EC50 that is lower than the reference binding peptide. In some embodiments, the EC50 for neutralizing activity is about 1.2-fold, 15.-fold, 2-fold, 3-fold, 4-fold, 5-fold, 10-fold, 20-fold, 10-fold or more lower than the reference binding peptide. In some embodiments, the modified binding peptide has subnanomolar, such as picomolar, neutralizing activity.

### B. Multispecific Binding Peptides

Also provided is a multispecific binding protein, comprising a plurality of any of the provided binding peptides. In some embodiments, the plurality of binding peptides are paratopes. In some embodiments, the plurality of knob peptides are 2, 3, or 4 peptides. Exemplary formats for generating a multispecific polypeptide are depicted in FIG. 12.

In some embodiments, one or more binding peptides, including one or more modified binding peptide, are linked in tandem in a single polypeptide chain separated with a flexible linker (e.g. GGGS or other similar flexible linker, including longer linkers of (GGGS)n where n is 1-3). In some embodiments, the tandem single polypeptide may include 2, 3, 4 or more binding peptides to produce a bivalent, trivalent, tetravalent or other multivalent molecule.

In some embodiments, the binding peptides are re-formatted by replacement of a knob region of an ultralong CDR-H3 scaffold, including any of the humanized ultralong heavy chain molecules described herein. The heavy chain can be complexed with a light chain, such as any of the light chain molecules described herein. In some embodiment, when produced in a cell, a two chain polypeptide is formed by dimerization resulting from disulfide formation between two heavy chain molecules. In some embodiments, the modified immunoglobulin containing a provided binding peptide is a homodimer containing the binding peptide. In other embodiments, two different heavy chains may be co-expressed in a cell using knobs-into-hole engineering strategy or other strategy to produce a heterodimer in which two different heavy chains, each carrying a different binding peptide, may interact to form a heterodimer. In some embodiments, residues of the constant chain are modified by amino acid substitution to promote the heterodimer formation. In some of any embodiments, the one more amino acid modifications are selected from a knob-into-hole modification and a charge mutation to reduce or prevent self-association due to charge repulsion. The heterodimer can be formed by transforming into a cell both a first nucleic acid molecule encoding a first polypeptide subunit and a second nucleic acid molecule encoding a second different polypeptide subunit. In some aspects, the heterodimer is produced upon expression and secretion from a cell as a result of covalent or non-covalent interaction between residues of the two polypeptide subunits to mediate formation of the dimer. In such processes, generally a mixture of dimeric molecules is formed, including homodimers and heterodimers. For the generation of heterodimers, additional steps for purification can be necessary. For example, the first and second polypeptide can be engineered to include a tag with metal chelates or other epitope, where the tags are different. The tagged domains can be used for rapid purification by metal-chelate chromatography, and/or by antibodies, to allow for detection by western blots, immunoprecipitation, or activity depletion/blocking in bioassays. Methods include those described in U.S. Pat. No. 10,995,127. In some embodiments, a human IgG1 includes a T22Y amino acid substitution in the CH3 domain and a second IgG1 heavy chain includes a Y86T amino acid substitution in the heavy chain.

### C. Nucleic Acids and Vectors

Also provided are nucleic acids, e.g., polynucleotides, encoding any of the binding peptides, such as any of the modified binding peptides, provided herein. The nucleic acids may include those encompassing natural and/or non-naturally occurring nucleotides and bases, e.g., including those with backbone modifications. The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

Also provided are vectors containing the nucleic acids, e.g., polynucleotides, and host cells containing the vectors, e.g., for producing the binding peptides. In some embodiments, one or more vectors (e.g., expression vectors) comprising such nucleic acids are provided. In a further embodiment, a host cell comprising such nucleic acids is provided. In some embodiments, a host cell comprises (e.g., has been transformed with) one or more vectors comprising one or more nucleic acid that encodes one or more an amino acid sequence of a providing binding peptide. In some embodiments, one or more such host cells are provided. In some embodiments, a composition containing one or more such host cells are provided. In some embodiments, the one or more host cells can express different binding peptides or the same binding peptide.

Also provided are methods for making or producing the provided binding peptides using any of the provided nucleic acids, vectors or host cells. For recombinant production of the binding peptide, a nucleic acid sequence or a polynucleotide encoding a binding peptide, e.g., as described above, may be isolated and inserted into one or more vectors for further cloning and/or expression in a host cell. Such nucleic acid sequences may be readily isolated and sequenced using conventional procedures (e.g., by using oligonucleotide probes specific a region of the sequence or by synthetic methods). In some embodiments, a method of making the binding peptide is provided, wherein the method comprises culturing a host cell comprising a nucleic acid sequence encoding the binding peptide, any as described herein, under conditions suitable for expression of the binding peptide, and optionally recovering the binding peptide from the host cell (or host cell culture medium). Methods for producing binding peptides are described. The provided embodiments further include vectors and host cells and other expression systems for expressing and producing the binding peptides, including eukaryotic and prokaryotic host cells, including bacteria, filamentous fungi, and yeast, as well as mammalian cells such as human cells, as well as cell-free expression systems. Exemplary methods for generating soluble binding peptides is described in Section V.

### III. Immunization

In some embodiments, a cow antibody with specificity for a target antigen and containing an ultralong CDR3 can be obtained from a biological sample, such as a blood sample, of an immunized cow. In some embodiments, the methods include immunizing a cow with a target antigen. In some embodiments, the antibody sequence can be obtained by amplification of antibody sequences from RNA isolated from the biological sample. In some embodiments, a cDNA template library that is prepared from RNA isolated from an immunized cow can be generated and used to create a display library for screening of antibodies with the desired activity.

In some embodiments, a bovine is immunized by administering at least one dose of an antigenic composition comprising a target antigen or a group of related target antigens. In some embodiments, the target antigen is a nonvirulent bacteria, a virus, a viral protein, a cancer antigen, a human IgG, or a recombinant protein thereof. In some embodiments, the target antigen, includes, for example, any virus, bacterial, other pathogenic, an immunomodulatory protein (e.g. a checkpoint molecule), or cancer antigen. In some embodiments, the target antigen is associated with a virus or variants of a virus.

In some embodiments, the cow is immunized with a target antigen. In some embodiments, the target antigen is a nonvirulent bacteria, a virus, a viral protein, an immunomodulatory protein (e.g. a checkpoint molecule), a cancer antigen, a human IgG, or a recombinant protein thereof. In some embodiments, the target antigen is a viral protein. In some embodiments, the cow is immunized with multiple target antigens, for instance different viral antigens. In some embodiments, the different viral antigens are proteins associated with different variants, clades, or strains of a virus.

In some embodiments, the target antigen is a coronavirus, a coronavirus pseudovirus, or an antigen of such virus, such as a a recombinant coronavirus Spike protein, or a receptor-binding domain (RBD) of a coronavirus Spike protein. Coronaviruses may be from the subfamily Orthocoronavirinae, which is one of two sub-families in the family Coronaviridae, order Nidovirales, and realm Riboviria. There are four genera: Alphacoronavirus, Betacoronavirus, Gammacoronavirus and Deltacoronavirus. SARS CoV2 is a Betacoronavirus, belonging to the subgenus Sarbecovirus. In some embodiments, the coronavirus is selected from the group consisting of 229E, NL63, OC43, HKU1, MERS-CoV, SARS-CoV, and SARS-CoV2. In some embodiments, the coronavirus is a SARS-CoV2 selected from Wuhan-Hu-1 isolate, B.1.351 South African variant, or B.1.1.7 UK variant. In some embodiments, the SARS CoV-2 specific antigen comprises a S trimer polypeptide. In some embodiments, the SARS CoV-2 specific antigen comprises a S monomer polypeptide. In some embodiments, the SARS CoV-2 specific antigen comprises a polynucleotide encoding a S trimer or monomer polypeptide. In some embodiments, the cow is immunized with multiple target antigens associated with any combination of coronaviruses 229E, NL63, OC43, HKU1, MERS-CoV, SARS-CoV, and SARS-CoV2. In some embodiments, the cow is immunized with multiple target antigens associated with any combination of SARS-CoV2 variants selected from Wuhan-Hu-1 isolate, B.1.351 South African variant or B.1.1.7 UK variant.

In some embodiments, the target antigen is a virus or viral protein, e.g., that is associated with a coronavirus, e.g., SARS CoV-2. In some embodiments, a cow can be immunized with a SARS CoV-2 specific antigen. In some embodiments, the SARS CoV-specific antigen comprises a S trimer polypeptide. In some embodiments, the SARS CoV-specific antigen comprises a S monomer polypeptide. In some embodiments, the SARS CoV-specific antigen comprises a polynucleotide encoding a S trimer or monomer polypeptide.

In some embodiments, the SARS CoV-2 specific antigen comprises a virus, pseudovirus, or virus-like particle comprising a S trimer polypeptide. In some embodiments, the SARS CoV-2 specific antigen comprises an isolated S trimer polypeptide. A S trimer polypeptide may be produced by a number of different means, for example, as described in US Patent Appl. Pub. No. 2014/0212458, Sanders, R. W. et al, PLoS Pathog. 9, e1003618 (2013) and Guenaga J., et al, Immunity 46(5):792-803.e3 (2017), each of which is incorporated by reference herein in its entirety. For example, HEK293F cells can be co-transfected with a pg140 encoding plasmid and a furin encoding plasmid. Supernatants comprising the antigen are purified using a lectin column. The affinity-purified antigen can be further purified to size homogeneity using size exclusion chromatography.

In some embodiments, a trimer is formed from a wildtype SARS CoV-2 S protein. In some embodiments, a trimer is formed from a variant SARS CoV-2 S protein.

In some embodiments, the antigen is a cancer antigen. In some embodiments, the antigen is selected from among ACTHR, endothelial cell Anxa-1, aminopetidase N, anti-IL-6R, alpha-4-integrin, alpha-5-beta-3 integrin, alpha-5-beta-5 integrin, alpha-fetoprotein (AFP), ANPA, ANPB, APA, APN, APP, 1AR, 2AR, AT1, B1, B2, BAGE1, BAGE2, B-cell receptor BB1, BB2, BB4, calcitonin receptor, cancer antigen 125 (CA 125), CCK1, CCK2, CD5, CD10, CD11a, CD13, CD14, CD19, CD20, CD22, CD25, CD30, CD33, CD38, CD45, CD52, CD56, CD68, CD90, CD133, CD7, CD15, CD34, CD44, CD206, CD271, CEA (CarcinoEmbryonic Antigen), CGRP, chemokine receptors, cell-surface annexin-1, cell-surface plectin-1, Cripto-1, CRLR, CXCR2, CXCR4, DCC, DLL3, E2 glycoprotein, EGFR, EGFRvIII, EMR1, Endosialin, EP2, EP4, EpCAM, EphA2, ET receptors, Fibronectin, Fibronectin ED-B, FGFR, frizzled receptors, GAGE1, GAGE2, GAGE3, GAGE4, GAGE5, GAGE6, GLP-1 receptor, G-protein coupled receptors of the Family A (Rhodopsin-like), G-protein coupled receptors of the Family B (Secretin receptor-like) like), G-protein coupled receptors of the Family C (Metabotropic Glutamate Receptor-like), GD2, GP100, GP120, Glypican-3, hemagglutinin, Heparin sulfates, HER1, HER2, HER3, HER4, HMFG, HPV 16/18 and E6/E7 antigens, hTERT, IL11-R, IL-13R, ITGAM, Kalikrien-9, Lewis Y, LH receptor, LHRH-R, LPA1, MAC-1, MAGE 1, MAGE 2, MAGE 3, MAGE 4, MART1, MC1R, Mesothelin, MUC1, MUC16, Neu (cell-surface Nucleolin), Neprilysin, Neuropilin-1, Neuropilin-2, NG2, NK1, NK2, NK3, NMB-R, Notch-1, NY-ESO-1, OT-R, mutant p53, p⁹⁷ melanoma antigen, NTR2, NTR3, p32 (p32/gC1q-R/HABP1), p75, PAC1, PAR1, Patched (PTCH), PDGFR, PDFG receptors, PDT, Protease-cleaved collagen IV, proteinase 3, prohibitin, protein tyrosine kinase 7, PSA, PSMA, purinergic P2X family (e.g., P2X1-5), mutant Ras, RAMP1, RAMP2, RAMP3 patched, RET receptor, plexins, smoothened, sst1, sst2A, sst2B, sst3, sst4, sst5, substance P, TEMs, T-cell CD3 Receptor, TAG72, TGFBR1, TGFBR2, Tie-1, Tie-2, Trk-A, Trk-B, Trk-C, TR1, TRPA, TRPC, TRPV, TRPM, TRPML, TRPP (e.g., TRPV1-6, TRPA1, TRPC1-7, TRPM1-8, TRPP1-5, TRPML1-3), TSH receptor, VEGF receptors (VEGFR1 or Flt-1, VEGFR2 or FLK-1/KDR, and VEGF-3 or FLT-4), voltage-gated ion channels, VPAC1, VPAC2, Wilms tumor 1, Y1, Y2, Y4, and Y5.

In some embodiments, the antigen is HER1/EGFR, HER2/ERBB2, CD20, CD25 (IL-2Ra receptor), CD33, CD52, CD133, CD206, CEA, CEACAM1, CEACAM3, CEACAM5, CEACAM6, cancer antigen 125 (CA125), alpha-fetoprotein (AFP), Lewis Y, TAG72, Caprin-1, mesothelin, PDGF receptor, PD-1, PD-L1, CTLA-4, IL-2 receptor, vascular endothelial growth factor (VEGF), CD30, EpCAM, EphA2, Glypican-3, gpA33, mucins, CAIX, PSMA, folate-binding protein, gangliosides (such as GD2, GD3, GM1 and GM2), VEGF receptor (VEGFR), integrin αVβ3, integrin α5β1, ERBB3, MET, IGF1R, EPHA3, TRAILR1, TRAILR2, RANKL, FAP, tenascin, AFP, BCR complex, CD3, CD18, CD44, CTLA-4, gp72, HLA-DR 10R, HLA-DR antigen, IgE, MUC-1, nuC242, PEM antigen, metalloproteinases, Ephrin receptor, Ephrin ligands, HGF receptor, CXCR4, CXCR4, Bombesin receptor, and SK-1 antigen.

In some embodiments, the antigen is CD25, PD-1 (CD279), PD-L1 (CD274, B7-H1), PD-L2 (CD273, B7-DC), CTLA-4, LAG3 (CD223), TIM3 (HAVCR2), 4-iBB (CD137, TNFRSF9), CXCR2, CXCR4 (CD184), CD27, CEACAM1, Galectin 9, BTLA, CD160, VISTA (PD1 homologue), B7-H4 (VCTN1), CD80 (B7-1), CD86 (B7-2), CD28, HHLA2 (B7-H7), CD28H, CD155, CD226, TIGIT, CD96, Galectin 3, CD40, CD40L, CD70, LIGHT (TNFSF14), HVEM (TNFRSFi4), B7-H3 (CD276), 0x40L (TNFSF4), CD137L (TNFSF9, GITRL), B7RP1, ICOS (CD278), ICOSL, KIR, GAL9, NKG2A (CD94), GARP, TLiA, TNFRSF25, TMIGD2, BTNL2, Butyrophilin family, CD48, CD244, Siglec family, CD30, CSF1R, MICA (MHC class I polypeptide-related sequence A), MICB (MHC class I polypeptide-related sequence B), NKG2D, KIR family (Killer-cell immunoglobulin-like receptor, LILR family (Leukocyte immunoglobulin-like receptors, CD85, ILTs, LIRs), SIRPA (Signal regulatory protein alpha), CD47 (IAP), Neuropilin 1 (NRP-1), a VEGFR, and VEGF.

In some embodiments, the antigen is an immunomodulatory protein (e.g. a checkpoint molecule). In some embodiments, the antigen is an immune checkpoint receptor ligands. Illustrative immune checkpoint molecules that may be targeted for blocking or inhibition include, but are not limited to, PD1 (CD279), PDL1 (CD274, B7-H1), PDL2 (CD273, B7-DC), CTLA-4, LAG3 (CD223), TIM3, 4-1BB (CD137), 4-1BBL (CD137L), GITR (TNFRSFi8, AITR), CD40, 0×40 (CD134, TNFRSF4), CXCR2, tumor associated antigens (TAA), B7-H3, B7-H4, BTLA, HVEM, GAL9, B7H3, B7H4, VISTA, KIR, 2B4 (belongs to the CD2 family of molecules and is expressed on all NK, T6, and memory CD8+(ap) T cells), CD160 (also referred to as BY55) and CGEN-15049. In some embodiments, the immune checkpoint molecule is CD25, PD-1, PD-L1, PD-L2, CTLA-4, LAG-3, TIM-3, 4-1BB, GITR, CD40, CD40L, OX40, OX40L, CXCR2, B7-H3, B7-H4, BTLA, HVEM, CD28 and VISTA.

In some embodiments, the antigenic composition further comprises an adjuvant. The skilled person is familiar with many potentially useful adjuvants, such as Freund's complete adjuvant, alum, and squalene. See, e.g., US Patent Appl. Pub. No. 20150361160, which is incorporated by reference herein in its entirety for all purposes. Adjuvants which may be used in compositions of the invention include, but are not limited to oil emulsion compositions (oil-in-water emulsions and water-in-oil emulsions), complete Freund's adjuvant (CFA) and incomplete Freund's adjuvant (IFA). In one embodiment, the adjuvant comprises RIBI, Iscomatrix, or ENABL CI (VaxLiant). Adjuvants suitable for use in the invention include bacterial or microbial derivatives such as derivatives of enterobacterial lipopolysaccharide (LPS), Lipid A derivatives, immunostimulatory oligonucleotides and ADP-ribosylating toxins and detoxified derivatives thereof.

In some embodiments, the bovine is domestic cattle, bison, African buffalo, water buffalo, or yak. In some embodiments, the bovine is domestic cattle. In one embodiment, the domestic cattle is a dairy cow. In some embodiments, the cow is pregnant.

Methods for immunizing a bovine, such as a cattle, to produce, for example, high titer colostrum, milk, serum, or immune tissues (e.g., PBMC), are known in in the art. Such methods are disclosed, for example, in US Patent Appl. Pub. Nos US20070053917 and US20130022619, each of which is incorporated by reference herein in its entirety for all purposes.

In some embodiments, the immunizing comprises administering a priming dose and at least one booster dose of the antigenic composition. In some embodiments, the immunizing comprises administering more than one booster doses of the antigenic composition. In one embodiment, the priming dose and at least one booster dose comprise the same antigenic composition. In some embodiments, the more than one booster doses comprise the same antigenic composition. The animal may be dosed with the immunogenic composition at intervals over a period of days, weeks or months. At the conclusion of the immunization regime, the hyperimmune material such as blood, milk or colostrum is harvested. In one embodiment, the hyperimmune material is collected less than 2 months, less than 3 months, less than 4 months, less than 5 months, less than 6 months, less than 9 months, or less than 12 months after administering the priming dose. In one embodiment, the hyperimmune material is collected between about 3 months and about 6 months after administering the priming dose. In one embodiment, the hyperimmune material is collected between about 3 months and about 9 months after administering the priming dose. In some embodiments, the hyperimmune material is collected between about 3 months and about 12 months after administering the priming dose. In one embodiment, the hyperimmune material is collected between about 6 months and about 12 months after administering the priming dose.

In some embodiments, the methods further comprise isolating from the bovine a biological sample. In some embodiments, the biological sample is milk, blood, serum, colostrum, or peripheral blood mononuclear cells (PBMC). In one embodiment, the biological sample is collected less than 2 months, less than 3 months, less than 4 months, less than 5 months, less than 6 months, less than 9 months, or less than 12 months after administering the priming dose. In one embodiment, the biological sample is collected between about 3 months and about 6 months after administering the priming dose. In some embodiments, the biological sample is collected between about 3 months and about 9 months after administering the priming dose. In some embodiments, the biological sample is collected between about 3 months and about 12 months after administering the priming dose. In some embodiments, the biological sample is collected between about 6 months and about 12 months after administering the priming dose.

In some embodiments, a biological sample from the immunized cow can be obtained and the antibody can be purified from the bovine biological sample. The biological sample can be a peripheral blood mononuclear cell (PBMC) sample.

In some embodiments, the methods include isolating a biological sample from the bovine, and cloning a polynucleotide that encodes a candidate binding peptide, e.g., containing an ultralong CDR3. In one embodiment, the cloning the polynucleotide includes performing single-cell RT-PCR amplification. The biological sample can be a peripheral blood mononuclear cell (PBMC) sample. The cloned polynucleotide then can be used to prepare a display library for further screening, as described below.

### IV. Display Libraries and Selection Methods

In some embodiments, an ultralong CDR3 antibody can be identified or obtained from an ultralong CDR3 antibody display library. In some embodiments, the display library is a phage display library. In some embodiments, the ultralong CDR3 antibodies or knobs are derived from cow antibodies, for instance based on antibodies produced by a cow immunized with a target antigen. In some embodiments, the ultralong CDR3 antibodies or knobs are synthetic.

### A. Library Production Methods

Techniques for manipulating nucleic acids, such as those for generating mutation in sequences, subcloning, labeling, probing, sequencing, hybridization and so forth, are described in detail in scientific publications and patent documents. See, for example, Sambrook J, Russell D W (2001) Molecular Cloning: a Laboratory Manual, 3rd ed. Cold Spring Harbor Laboratory Press, New York; Current Protocols in Molecular Biology, Ausubel ed., John Wiley & Sons, Inc., New York (1997); Laboratory Techniques in Biochemistry and Molecular Biology: Hybridization With Nucleic Acid Probes, Part I, Theory and Nucleic Acid Preparation, Tijssen ed., Elsevier, N.Y. (1993).

Any known methods for generating libraries containing variant polynucleotides and/or polypeptides can be used with the provided methods and vectors to generate display libraries, e.g. phage display libraries, and to select binding proteins from the libraries. The libraries can be used in screening assays to select binding proteins from the library for any antigen, including, for example, any virus, bacterial, other pathogenic, an immunomodulatory protein (e.g. a checkpoint molecule), or cancer antigen. To facilitate screening, antibody libraries typically are screened using a display technique, such that there is a physical link between the individual molecules of the library (phenotype) and the genetic information encoding them (genotype). These methods include, but are not limited to, cell display, including bacterial display, yeast display, mammalian display, phage display (Smith, G. P. (1985) Science 228:1315-1317), mRNA display, ribosome display and DNA display.

In some embodiments, the provided libraries are phage display libraries. In some embodiments, the display library is a phage display library. In some embodiments, the phage display library is produced through use of a phagemid encoding at least a portion of a phage coat protein, in addition to encoding the polypeptide for display. In some embodiments, the phagemid particles are derived from M13 phage. In some embodiments, the coat protein is the M13 phage gene III coat protein (pIII).

In some embodiments, a phage display library is produced by fusion of a candidate binding polypeptide as described herein, such as an ultralong CDR3 scFv antibody fragment or an ultralong CDR3 knob peptide, with a gene III minor coat protein of an F-specific filamentous phage of Escherichia coli (Ff: f1, M13, or fd). Alternatively, other bacterial species can be used to produce the phage display library, including Pseudomonas fluorescens. In some embodiments, the gene III is a minor coat protein of M13 phage (also called pIII). The gene III minor coat protein (present in about 5 copies at one end of the virion) is involved in proper phage assembly and for infection by attachment to the pili of E. coli. Methods of phage display are known.

In some embodiments, a nucleic acid encoding a candidate binding polypeptide as described herein, such as an ultralong CDR3 scFv antibody fragment or an ultralong CDR3 knob peptide, is inserted into or constructed as part of a replicable expression vector, in which the nucleic acid is fused to a nucleic acid encoding at least a portion of a phage coat protein, such as pIII. In some embodiments, the nucleic acid encoding a candidate binding polypeptide as described herein, such as an ultralong CDR3 scFv antibody fragment or an ultralong CDR3 knob peptide, is fused to pIII.

In some embodiments, the replicable expression vector is a plasmid vector that generally contains a variety of components, including promoters, signal sequences, phenotypic selection genes, origin of replication sites, and other necessary components as are known to those of ordinary skill in the art. Promoters most commonly used in prokaryotic vectors include the lac Z promoter system, the alkaline phosphatase pho A promoter, the bacteriophage λPL promoter (a temperature sensitive promoter), the tac promoter (a hybrid trp-lac promoter that is regulated by the lac repressor), the tryptophan promoter, the bacteriophage T7 promoter, or other suitable microbial promoters. Examples of promoter systems include Lac Z, λPL, TAC, T 7 polymerase, tryptophan, and alkaline phosphatase promoters and combinations thereof. Suitable prokaryotic signal sequences may be obtained from genes encoding, for example, LamB or OmpF (Wong et al., Gene, 68:193 1983), MalE, PhoA, the E. coli heat-stable enterotoxin II (STII) signal sequence, or a Pel B secretory signal sequence. In some embodiments, the expression vector will further contain a secretory signal sequences operably fused to the nucleic acid encoding the polypeptide. In some embodiments, the secretory sequence is a Pel B secretory signal sequence. In some embodiments, the replicable expression vector also may contain a phenotypic selection genes. Typical phenotypic selection genes are those encoding proteins that confer antibiotic resistance upon the host cell. By way of illustration, the ampicillin resistance gene (amp), the tetracycline resistance gene (tet), or carbenicillen resistance gene may be used.

Construction of suitable vectors containing the nucleic acid encoding the desired polypeptide are prepared using standard recombinant DNA procedures. Isolated DNA fragments to be combined to form the vector are cleaved, tailored, and ligated together in a specific order and orientation to generate the desired vector. In some embodiments, the DNA is cleaved using the appropriate restriction enzyme or enzymes in a suitable buffer. Appropriate buffers, DNA concentrations, and incubation times and temperatures are specified by the manufacturers of the restriction enzymes. Generally, incubation times of about one or two hours at 37° C. are adequate, although several enzymes require higher temperatures. After incubation, the enzymes and other contaminants are removed by extraction of the digestion solution with a mixture of phenol and chloroform, and the DNA is recovered from the aqueous fraction by precipitation with ethanol.

To ligate the DNA fragments together to form a functional vector, the ends of the DNA fragments must be compatible with each other. In some cases, the ends will be directly compatible after endonuclease digestion. However, it may be necessary to first convert the sticky ends commonly produced by endonuclease digestion to blunt ends to make them compatible for ligation. To blunt the ends, the DNA is treated in a suitable buffer for at least 15 minutes at 15° C. with 10 units of the Klenow fragment of DNA polymerase I (Klenow) in the presence of the four deoxynucleotide triphosphates. The DNA is then purified by phenol-chloroform extraction and ethanol precipitation.

The DNA fragments that are to be ligated together (previously digested with the appropriate restriction enzymes such that the ends of each fragment to be ligated are compatible) are put in solution. In some embodiments, the DNA fragments are provided in about equimolar amounts. In some embodiments, the solution will also contain ATP, ligase buffer, and a ligase such as T4 DNA ligase, such as at or about 10 units per 0.5 μg of DNA. If the DNA fragment is to be ligated into a vector, the vector is first linearized by cutting with the appropriate restriction endonuclease(s). The linearized vector is then treated with alkaline phosphatase or calf intestinal phosphatase. The phosphatasing prevents self-ligation of the vector during the ligation step.

In some embodiments, a plurality of constructed replicable expression vectors are transformed into suitable host cells. Suitable host cells include prokaryotes host cells. In some embodiments, the host cell used for expressing or producing the display libraries are E. coli cells. Suitable prokaryotic host cells include E. coli strain JM101, E. coli K12 strain 294 (ATCC number 31,446), E. coli strain W3110 (ATCC number 27,325), E. coli X1776 (ATCC number 31,537), E. coli XL-1Blue (stratagene), and E. coli B; however, many other strains of E. coli, such as HB101, NM522, NM538, NM539, and many other species and genera of prokaryotes may be used as well. In addition to the E. coli strains listed above, bacilli such as Bacillus subtilis, other enterobacteriaceae such as Salmonella typhimurium or Serratia marcesans, and various Pseudomonas species may all be used as hosts. In some embodiments, the host cell is a protease deficient strain of E. coli. In some embodiments, the host cells are TG1 electrocompetent cells.

Transformation of prokaryotic cells is readily accomplished using the calcium chloride method as described in section 1.82 of Sambrook et al., supra. Alternatively, electroporation (Neumann et al., EMBO J., 1:841 1982) may be used to transform these cells. In some embodiments, the methods further include infecting the transformed host cells with a helper phage having a gene encoding the phage coat protein. In some embodiments, the methods further include the use of a helper phage in order to promote sufficient expression of the phagemid particles. In some embodiments, the helper phage is selected from the group consisting of M13K07, M13R408, M13-VCS, and Phi X 174. In some embodiments, the helper phage is M13K07. The transformed infected host cells are then cultured under conditions suitable for forming recombinant phagemid particles containing at least a portion of the plasmid and capable of transforming the host. The transformed cells are selected by growth on an antibiotic, for example tetracycline (tet) or ampicillin (amp), carbenicillin or other antibiotic depending on the particular expression vector, to which they are rendered resistant due to the presence of resistance genes on the vector.

After selection of the transformed cells, these cells are grown in culture and the plasmid DNA (or other vector with the foreign gene inserted) is then isolated. Plasmid DNA can be isolated using methods known in the art. The isolated DNA can be purified by methods known in the art. This purified plasmid DNA is then analyzed by restriction mapping and/or DNA sequencing.

### 1. Polypeptides for Display

In some embodiments, the polypeptides for display include an ultralong CDR3.

In some embodiments, the ultralong CDR3 includes or is a peptide sequence of 25-70 amino acids. In some embodiments, the ultralong CDR3 is a peptide sequence that is between or between about 35 and 70 amino acids in length, 40 and 70 amino acids in length, 45 and 70 amino acids in length, 50 and 70 amino acids in length, 55 and 70 amino acids in length, or 60 and 70 amino acids in length.

In some embodiments, the ultralong CDR3 includes a cysteine motif. In some embodiments, the cysteine motif includes 2-20 cysteine residues, for instance between or between about 2 and 18, 2 and 16, 2 and 14, 2 and 12, 2 and 10, 2 and 8, 2 and 6, 2 and 4, 4 and 20, 4 and 18, 4 and 16, 4 and 14, 4 and 12, 4 and 10, 4 and 8, 4 and 6, 6 and 20, 6 and 18, 6 and 16, 6 and 14, 6 and 12, 6 and 10, 6 and 8, 8 and 20, 8 and 18, 8 and 16, 8 and 14, 8 and 12, 8 and 10, 10 and 20, 10 and 18, 10 and 16, 10 and 14, 10 and 12, 12 and 20, 12 and 18, 12 and 16, 12 and 14, 14 and 20, 14 and 18, 14 and 16, 16 and 20, 16 and 18, or 18 and 20 cysteine residues, each inclusive. In some embodiments, the cysteine motif includes 2-12 cysteine residues.

In some embodiments, the ultralong CDR3 knob includes 1-10 disulfide bonds, for instance between or between about 1 and 9, 1 and 8, 1 and 7, 1 and 6, 1 and 5, 1 and 4, 1 and 3, 1 and 2, 2 and 10, 2 and 9, 2 and 8, 2 and 7, 2 and 6, 2 and 5, 2 and 4, 2 and 3, 3 and 10, 3 and 9, 3 and 8, 3 and 7, 3 and 6, 3 and 5, 3 and 4, 4 and 10, 4 and 9, 4 and 8, 4 and 7, 4 and 6, 4 and 5, 5 and 10, 5 and 9, 5 and 8, 5 and 7, 5 and 6, 6 and 10, 6 and 9, 6 and 8, 6 and 7, 7 and 10, 7 and 9, 7 and 8, 8 and 10, 8 and 9, or 9 and 10 disulfide bonds, each inclusive. In some embodiments, the ultralong CDR3 knob includes 1-6 disulfide bonds.

In some embodiments, the ultralong CDR3 includes an ascending stalk domain. In some embodiments, the ultralong CDR3 includes a descending stalk domain. In some embodiments, the cysteine motif (knob region) is between the ascending and descending stalk domains. In some embodiments, the ascending stalk domain includes the sequence CX₂TVX₅Q (SEQ ID NO: 103), wherein X₂ and X₅ are any amino acid. In some embodiments, X₂ is Ser, Thr, Gly, Asn, Ala, or Pro, and X₅ is His, Gln, Arg, Lys, Gly, Thr, Tyr, Phe, Trp, Met, Ile, Val, or Leu (SEQ ID NO: 104). In some embodiments, X₂ is Ser, Ala, or Thr, and X₅ is His or Tyr (SEQ ID NO: 105). In some embodiments, the ascending stalk includes residues CTTVHQ (SEQ ID NO: 98), CATVHQ (SEQ ID NO: 99), CAIVQQ (SEQ ID NO: 100), or CATVDQ (SEQ ID NO: 101). In some embodiments, the ascending stalk further includes a variable number of connecting residues, e.g., 2 to 8 amino acid residues, before a first conserved cysteine residue that forms part of the disulfide-bonded knob region. In some embodiments, the descending stalk includes alternating aromatics that form a ladder through stacking interactions, that may contribute to the stability of the long solvent-exposed, two stranded β-ribbon (Wang et al. Cell. 2013, 153 (6): 1379-1393). In some embodiments, the ascending stalk contains a conserved pattern of alternating tyrosines, sometimes with the motif YX₁YX₂X₃(SEQ ID NO: 224), YX₁YX₂F (SEQ ID NO: 225), YX₁YX₂W (SEQ ID NO: 225) or YX₁YX₂Y (SEQ ID NO: 226),

In some embodiments, the polypeptides for display, e.g., polypeptides including the ultralong CDR3, are derived from bovine antibodies. In some embodiments, the polypeptides for display are produced by amplifying sequences from a cow complementary DNA (cDNA) library. In some embodiments, the cDNA template library is prepared from RNA isolated from peripheral blood mononuclear cells (PBMCs) from a cow, generally a cow that has been immunized with a target antigen, e.g. as described in Section III. In some embodiments, the cDNA template library is synthesized using a pool of immunoglobulin-specific primers. In some embodiments, the cDNA template library is synthesized using a pool of IgM, IgA, and IgG-specific primers. Exemplary primers for use include those with sequences set forth in SEQ ID NO: 3 (IgG), SEQ ID NO: 4 (IgM), 5 (IgA), and SEQ ID NO: 6 (IgG).

In some embodiments, the polypeptides for display are synthetic. In some embodiments, the synthetic polypeptides include all or a portion of a bovine antibody, e.g., an ultralong CDR3 knob. In some embodiments, the synthetic polypeptide is a modified cyclotide. In some embodiments, the modified cyclotide includes an ultralong CDR3 knob sequence, e.g., of a cow.

In some embodiments, the polypeptides for display contain a variable heavy region containing the ultralong CDR-H3 and a variable light region. Particular formats include single chain formats, such as a single chain variable fragment (scFv). In other embodiments, the polypeptides for display is a smaller peptide of 25-70 amino acids, such as 40-70 amino acids, that is a knob peptide. Exemplary molecules for display and display libraries are described. a. scFv Peptides for Display

In some embodiments, the polypeptide for display is a single-chain variable fragment (scFv). In some embodiments, the scFv includes a VH region having a cow ultralong CDR3. In some embodiments, the VH region is encoded by a sequence that has been amplified from a cow cDNA template library, e.g., one prepared from RNA isolated from peripheral blood mononuclear cells (PBMCs) from an immunized cow. In some embodiments, the amplifying is by amplifying sequences encoding VH regions of bovine antibody families known or suspected to contain ultralong CDR3s. In some embodiments, sequences of VH regions of the IgHV1-7 family are amplified to produce sequences encoding the VH region of the scFv. In some embodiments, the VH regions of the IgHV1-7 family are amplified with a forward primer that includes the sequence set forth in SEQ ID NO: 84 and a reverse primer that includes the sequence set forth in SEQ ID NO: 85. In some embodiments, the forward primer and/or the reverse primer further include sequences specific to restriction enzyme sites in order to facilitate cloning. In some embodiments, the VH regions of the IgHV1-7 family are amplified with a forward primer set forth in SEQ ID NO: 12 and a reverse primer set forth in SEQ ID NO: 13.

In some embodiments, preparation of sequences for the VH regions of the polypeptides for display also includes a size separation step. In some embodiments, following amplification of VH region sequences, e.g., of the IgHV1-7 family, such as from a cow cDNA template library, sequences encoding VH regions with an ultralong CDR3 are separated from shorter sequences encoding VH regions without an ultralong CDR3. In some embodiments, the size separation step further enriches for amplified sequences encoding VH regions with an ultralong CDR3.

In some embodiments, the size separation step involves separating, from sequences encoding a plurality of amplified VH regions, sequences of, of about, or greater than 425, 450, 475, 500, 525, or 550 base pairs in length, wherein the sequences of, of about, or greater than 425, 450, 475, 500, 525, or 550 base pairs in length include the sequences encoding VH regions with an ultralong CDR3. In some embodiments, sequences of, of about, or greater than 550 base pairs in length are separated from the remaining sequences.

In some embodiments, the size separation is performed by agarose gel electrophoresis. In some embodiments, a 1.2%, 1.5%, or 2% agarose gel is used. In some embodiments, a 2% agarose gel is used.

In some embodiments, the scFv includes a VL region that is fixed across polypeptides of the display library. In some aspects, the use of a fixed VL region improves selection and/or screening for scFvs including a VH region with an ultralong CDR3. In some embodiments, the VL region is a variable lambda light (VL) region selected from the group consisting of BLV1H12, BLV5D3, BLV8C11, BF1H1, BLV5B8, and F18, or is a humanized variant thereof. In some embodiments, the VL region is the BLV5B8 lambda VL region (SEQ ID NO: 110) or a humanized variant thereof. In some embodiments, the VL region is the BLV1H12 lambda VL region or a humanized variant thereof. In some embodiments, the BLV1H12 VL region is set forth in SEQ ID NO: 2. In some embodiments, the humanized variant comprises one or more of amino acid replacements S2A, T5N, P8S, A12G, A13S, and P14L based on Kabat numbering, amino acid replacements I29V and N32G in the CDR1 region, and/or amino acid substitution of DNN to GDT in the CDR2 region. In some embodiments, the humanized variant of BLV1H12 comprises the sequence set forth in SEQ ID NO: 107.

In some embodiments, at least or at least about 20%, 30%, 35%, 40%, 45%, 50%, 60%, 70%, 80%, 85%, 90%, or 95% of the displayed scFvs include a VH region comprising an ultralong CDR3 region. In some embodiments, at least or at least about 30% of the displayed scFvs include a VH region comprising an ultralong CDR3 region. In some embodiments, at least or at least about 40% of the displayed scFvs include a VH region comprising an ultralong CDR3 region. In some embodiments, at least or at least about 50% of the displayed scFvs include a VH region comprising an ultralong CDR3 region. In some embodiments, at least or at least about 60% of the displayed scFvs include a VH region comprising an ultralong CDR3 region. In some embodiments, at least or at least about 70% of the displayed scFvs include a VH region comprising an ultralong CDR3 region. In some embodiments, at least or at least about 80% of the displayed scFvs include a VH region comprising an ultralong CDR3 region. In some embodiments, at least or at least about 90% of the displayed scFvs include a VH region comprising an ultralong CDR3 region. In some embodiments, at least or at least about 95% of the displayed scFvs include a VH region comprising an ultralong CDR3 region.

In some embodiments, the VH and VL regions of the scFv are joined directly. In some embodiments, the VH and VL regions of the scFv are joined indirectly, e.g., via a peptide linker. In some embodiments, the peptide linker is a flexible linker. In some embodiments, the peptide linker is (Gly4 Ser)3 (SEQ ID NO: 94).

b. Knob Peptides for Display

In some embodiments, the polypeptide for display is an ultralong CDR3 knob, e.g., a cow ultralong CDR3. In some embodiments, the ultralong CDR3 knob is encoded by a sequence that has been amplified from a cow cDNA template library, e.g., one prepared from RNA isolated from peripheral blood mononuclear cells (PBMCs) from an immunized cow.

In some embodiments, the amplifying is by amplifying sequences encoding ultralong CDR3 knobs. In some embodiments, primers specific for the ascending and descending stalk domains of a cow ultralong CDR3 region are used to amplify the sequences encoding ultralong CDR3 knobs. In some embodiments, the ultralong CDR3 knob comprises a portion of the ascending stalk domain, such as 1, 2, 3, 4, 5 or 6 amino acids. In some embodiments, the ultralong CDR3 knob comprises a portion of the descending stalk domain, such as 1, 2, 3, 4, 5, 6, 7, 8, or 9 amino acids. In some embodiments, the ascending stalk domain includes the sequence CX₂TVX₅Q, wherein X₂ and X₅ are any amino acid. In some embodiments, X₂ is Ser, Thr, Gly, Asn, Ala, or Pro, and X₅ is His, Gln, Arg, Lys, Gly, Thr, Tyr, Phe, Trp, Met, Ile, Val, or Leu. In some embodiments, X₂ is Ser, Ala, or Thr, and X₅ is His or Tyr. In some embodiments, the primers used for amplifying include or consist of the sequences set forth in SEQ ID NO: 7-11. In some embodiments, the primers used for amplifying include or consist of the sequences set forth in SEQ ID NO: 8-11. In some embodiments, the primers used for amplifying include or consist of the sequences set forth in SEQ ID NO: 121-130. In some embodiments, the primers used for amplifying include or consist of the sequences set forth in SEQ ID NO: 123, 127, and 128.

In some embodiments, the primers used for amplifying are a pool of different primers specific for the ascending and descending stalk domains. In some embodiments, the pool of primers contains at least two, three, four, five, six, seven, eight, nine, or 10 different primers. In some embodiments, the pool of primers contains at least two, three, four, five, six, seven, eight, nine, or 10 different primers from the primers set forth in SEQ ID NO: 7-11 and 121-130. In some embodiments, the pool of primers contains at least two, three, four, five, six, or seven different primers from the primers set forth in SEQ ID NO: 8-11, 123, 127, and 128. In some embodiments, the pool of primers contains the primers set forth in SEQ ID NO: 8-11. In some embodiments, the pool of primers contains the primers set forth in SEQ ID NO: 123, 127, and 128. In some embodiments, the pool of primers contains the primers set forth in SEQ ID NO: 8-11, 23, 27, and 28.

Once identified, the knob peptide sequences can be amplified using methods known to a skilled artisan. In other embodiment, the knob peptide may be synthetically generated. A variety of techniques including recombinant methods, chemical synthesis, or combinations thereof, may be employed. In some embodiments, chemical synthesis methods amy include known chemical synthesis techniques, such as the phosphoramidite method. In some instances, a recombinant or synthetic nucleic acid may be generated through polymerase chain reaction (PCR).

### B. Display Libraries

Also provided herein are libraries of display particles, e.g., phagemid particles, including any that are produced by any the provided methods.

Also provided herein is a phagemid that comprises or is a replicable expression vector containing a gene fusion encoding a fusion protein that includes a first nucleic acid sequence encoding a single chain variable fragment with a cow variable heavy (VH) region that includes an ultralong CDR3 joined to a variable lambda light (VL) region selected from the group consisting of BLV1H12, BLV5D3, BLV8C11, BF1H1, BLV5B8, and F18, or a humanized variant thereof, and a second nucleic acid sequence encoding at least a portion of a phage coat protein. In some embodiments, the VL region is the VL region of BLV1H12.

Also provided herein is a phagemid that comprises or is a replicable expression vector containing a gene fusion encoding a fusion protein that includes a first nucleic acid sequence encoding a cow ultralong CDR3 knob and a second nucleic acid sequence encoding at least a portion of a phage coat protein.

Also provided herein is a phagemid that comprises or is a replicable expression vector containing a gene fusion encoding a fusion protein that includes a first nucleic acid sequence encoding a peptide sequence of 25-70 amino acids with a cysteine motif that includes 2-12 cysteine residues able to form disulfide bonds joined and a second nucleic acid sequence encoding at least a portion of a phage coat protein.

In some embodiments, also provided herein are libraries of display particles, e.g., phagemid particles, that are encoded by any of the phagemids described herein.

In some embodiments, the display particles include an ultralong CDR3 knob, e.g., any as described herein.

In some embodiments, the display particles include a synthetic or semisynthetic ultralong CDR3 knob, e.g., any as described herein.

In some embodiments, the display particles include an scFv with a VH containing an ultralong CDR3 region. In some embodiments, at least or at least about 20%, 30%, 35%, 40%, 45%, 50%, 60%, 70%, 80%, 85%, 90%, or 95% of the display particles, e.g., phagemid particles, in the library include an scFv that includes a VH region with an ultralong CDR3 region. In some embodiments, at least or at least about 30% of the display particles, e.g., phagemid particles, in the library include an scFv that includes a VH region with an ultralong CDR3 region. In some embodiments, at least or at least about 35% of the display particles, e.g., phagemid particles, in the library include an scFv that includes a VH region with an ultralong CDR3 region. In some embodiments, at least or at least about 40% of the display particles, e.g., phagemid particles, in the library include an scFv that includes a VH region with an ultralong CDR3 region. In some embodiments, at least or at least about 45% of the display particles, e.g., phagemid particles, in the library include an scFv that includes a VH region with an ultralong CDR3 region. In some embodiments, at least or at least about 50% of the display particles, e.g., phagemid particles, in the library include an scFv that includes a VH region with an ultralong CDR3 region. In some embodiments, at least or at least about 60% of the display particles, e.g., phagemid particles, in the library include an scFv that includes a VH region with an ultralong CDR3 region. In some embodiments, at least or at least about 70% of the display particles, e.g., phagemid particles, in the library include an scFv that includes a VH region with an ultralong CDR3 region. In some embodiments, at least or at least about 80% of the display particles, e.g., phagemid particles, in the library include an scFv that includes a VH region with an ultralong CDR3 region. In some embodiments, at least or at least about 90% of the display particles, e.g., phagemid particles, in the library include an scFv that includes a VH region with an ultralong CDR3 region. In some embodiments, at least or at least about 95% of the display particles, e.g., phagemid particles, in the library include an scFv that includes a VH region with an ultralong CDR3 region

### C. Library Selection Methods

Also provided herein are methods for selecting, from any of the display libraries described herein, an antibody binding protein that is specific for a target antigen. These display libraries are then contacted with a target antigen and those members of the library having the highest affinity for the target are separated from those of lower affinity. These display libraries, are then contacted with a target antigen and those members of the library having the highest affinity for the target are separated from those of lower affinity. The high affinity binders are then amplified by any suitable system. This process is reiterated until polypeptides of the desired affinity are obtained.

For instance, the display library is a phage display library as described herein in which an ultralong CDR3 scFv polypeptide or a CDR3-knob peptide, is fused to a phage coat protein and displayed, usually on average as a single copy of each related polypeptide, on the surface of a phagemid particle containing DNA encoding that polypeptide. These phagemid particles are then contacted with a target antigen and those particles having the highest affinity for the target are separated from those of lower affinity. The high affinity binders are then amplified by infection of a bacterial host and the competitive binding step is repeated. This process is reiterated until polypeptides of the desired affinity are obtained.

In some embodiments, the provided methods include contacting any of the display libraries provided herein with a target antigen under conditions to allow binding of a display particle, e.g., a phagemid particle, to the target molecule. In some embodiments, the methods further include separating the display particles, e.g., the phagemid particles, that bind from those that do not, thereby selecting display particles, e.g., the phagemid particles, that include an antibody binding protein that binds to the target antigen. In some embodiments, the methods include sequencing the fusion gene in the selected particles to identify the antibody binding protein.

Target antigens may be isolated from natural sources or prepared by recombinant methods by procedures known in the art. The purified target molecule can be attached to a suitable matrix such as agarose beads, acrylamide beads, glass beads, cellulose, various acrylic copolymers, hydroxyalkyl methacrylate gels, polyacrylic and polymethacrylic copolymers, nylon, neutral and ionic carriers, and the like. Attachment of the target protein to the matrix may be accomplished by methods described in Methods in Enzymology, 44 1976, or by other means known in the art.

After attachment of the target antigen to the matrix, the immobilized target can be contacted with the library of display particles, e.g., phagemid particles, under conditions suitable for binding of at least a portion of the display particles with the immobilized target molecules. Normally, the conditions, including pH, ionic strength, temperature and the like will mimic physiological conditions. Exemplary “contacting” conditions may comprise incubation for 15 minutes to 4 hours, e.g. one hour, at 4°−37° C., e.g. at room temperature. However, these may be varied as appropriate depending on the nature of the interacting binding partners, etc. The mixture can be subjected to gentle rocking, mixing, or rotation. In addition, other appropriate reagents such as blocking agents to reduce nonspecific binding may be added. For example 1-4% BSA or other suitable blocking agent (e.g. milk) may be used. It will be appreciated however that the contacting conditions can be varied and adapted by a skilled person depending on the aim of the screening method. For example, if the incubation temperature is, for example, room temperature or 37° C., this may increase the possibility of identifying binders which are stable under these conditions, e.g., in the case of incubation at 37° C., are stable under conditions found in the human body. Such a property might be extremely advantageous if one or both of the binding partners was a candidate to be used in some sort of therapeutic application, e.g. an antibody. Again such adaptations to the conditions are within the ambit of the skilled person

Bound display particles (“binders”) having high affinity for the immobilized target antigen can be separated from those having a low affinity (and thus do not bind to the target) by washing. Binders can be dissociated from the immobilized target molecules by a variety of methods. These methods include competitive dissociation using the wild-type ligand, altering pH and/or ionic strength, and methods known in the art.

In some embodiments, the target antigen is a nonvirulent bacteria, a virus, a viral protein, a cancer antigen, a human IgG, or a recombinant protein thereof. In some embodiments, the target antigen is a viral protein. In some embodiments, the target antigen is a coronavirus, a coronavirus pseudovirus, a recombinant coronavirus Spike protein, or a receptor-binding domain (RBD) of a coronavirus Spike protein. In some embodiments, the coronavirus is selected from the group consisting of 229E, NL63, OC43, HKU1, MERS-CoV, SARS-CoV and SARS-CoV2. In some embodiments, the coronavirus is a SARS-CoV2 selected from Wuhan-Hu-1 isolate, B.1.351 South African variant or B.1.1.7 UK variant.

In some embodiments, the methods include steps wherein previously selected display particles are re-expressed and subjected to further selection steps, including with the same or a different target molecule. In some embodiments, the selection steps are repeated one or more times. In some embodiments, the further selection steps include infecting suitable host cells with replicable expression vectors encoding the previously selected display particles; collecting additional amplified display particles; and contacting the additional amplified display particles with the same or a different target antigen. In some embodiments, the different target molecule is related to the target antigen and is the same type of pathogen, the same group of pathogen, or a variant of the target antigen. In some embodiments, the target antigen and different target antigen are associated with any combination of coronaviruses 229E, NL63, OC43, HKU1, MERS-CoV, SARS-CoV, and SARS-CoV2. In some embodiments, the target antigen and different target antigen are associated with any combination of SARS-CoV2 variants selected from Wuhan-Hu-1 isolate, B.1.351 South African variant, and B.1.1.7 UK variant.

Once one or more sets of binders have been selected or isolated in accordance with the provided methods, these can be subjected to further analysis. In some embodiments, the further analysis involves the isolation of binders by infection of bacteria as an amplification step, isolating the phage or phagemid DNA, and cloning the DNA sequence encoding the candidate binders contained in said phage or phagemid DNA into a suitable expression vector. Such an infection step can also allow the amplification of the binders. Alternatively, binders can be amplified at this stage by other appropriate methods, for example by PCR of the nucleic acids encoding said binders or the transformation of said nucleic acid into an appropriate host cell (in the context of a suitable expression vector).

Once the DNA encoding the binders are cloned in a suitable expression vector, the DNA encoding the binders can be sequenced or the protein can be expressed in a soluble form, e.g., including according to the methods provided herein, and subjected to appropriate binding studies to further characterize the candidates at the protein level. Appropriate binding studies will depend on the nature of the binders, and include, but are not limited to ELISA, filter screening assays, FACS, or immunofluorescence assays, BiaCore affinity measurements or other methods to quantify binding constants, staining tissue slides or cells and other immunohistochemistry methods. One or more of these binding studies can be used to analyze the binders.

Also provided herein are methods for identifying an ultralong CDR H3 knob, such as a bovine CDR H3 knob, by amino acid sequence, including from a sequence library. In some aspects, methods for identifying an ultralong CDR H3 knob include defining the region of the knob domain, such as by reference to the formula described herein, e.g. set forth below.

In some embodiments, a method for identifying an ultralong CDR H3 knob, includes defining the knob region N-terminal boundary as the first D_{H} cysteine in the “CPDG” motif. In some embodiments, the method further includes defining the C-terminal boundary as the position located by subtracting number of ascending stalk residues from the framework 4 tryptophan position. In some aspects, the method can be used for identifying an ultralong CDR H3 knob from any antibody sequence. In particular embodiments, the antibody sequence is a bovine antibody, such as any of the antibodies described herein.

An expression of this embodiment of the method is shown below:
Knob boundary position (C-terminal end)=Position of conserved framework 4 tryptophan −X; wherein X=number of amino acids, starting at the framework 3 canonical cysteine that defines the ascending stalk, and ending at the amino acid preceding the conserved first D region cysteine in the “CPDG” motif;
Number of residues in the knob (K)=L−2X; wherein L=number of amino acids encompassing stalk and knob domains, starting at canonical framework 3 cysteine and ending at canonical framework 4 tryptophan;

$$K position = ( X + 1 ) to ( X + K )$$

### V. Soluble Peptide Expression

Also provided herein in some embodiments are methods of producing soluble disulfide bond-containing peptides, including methods of producing any of the antibody binding proteins (also referred to as binders) identified by any of the methods described herein. The soluble peptides produced by the provided methods are binding peptides as described herein that contain 2 or more cysteine residues from which it is desired to produce a disulfide-bonded soluble protein. In some embodiments, the provided methods include transforming a host cell, e.g., E. coli, with an expression vector encoding the soluble peptide. In some embodiments, the expression vector encodes a fusion protein that includes the soluble peptide and a chaperone, e.g., a bacterial chaperone. In some embodiments, the soluble peptide and the chaperone, e.g., bacterial chaperone, are joined by a linker. In some embodiments, the linker is a cleavable linker.

Techniques for manipulating nucleic acids, such as those for generating mutation in sequences, subcloning, labeling, probing, sequencing, hybridization and so forth, are described in detail in scientific publications and patent documents. See, for example, Sambrook J, Russell D W (2001) Molecular Cloning: a Laboratory Manual, 3rd ed. Cold Spring Harbor Laboratory Press, New York; Current Protocols in Molecular Biology, Ausubel ed., John Wiley & Sons, Inc., New York (1997); Laboratory Techniques in Biochemistry and Molecular Biology: Hybridization With Nucleic Acid Probes, Part I, Theory and Nucleic Acid Preparation, Tijssen ed., Elsevier, N.Y. (1993).

In some embodiments, the fusion protein has increased solubility relative to the soluble protein alone. In some aspects, this increased solubility is conferred at least in part by the inclusion of the chaperone, e.g., bacterial chaperone. In some aspects, the inclusion of the chaperone, e.g., bacterial chaperone, promotes solubility of the fusion protein while permitting disulfide bond formation in the soluble peptide, including in host cell environments that have been engineered or modified to promote disulfide bond formation.

In some embodiments, the chaperone, e.g., bacterial chaperone, is thioredoxin A (TrxA). In some embodiments, TrxA is set forth in SEQ ID NO:194 or a sequence of amino acids that exhibits at least 85%, 86%, 87%, 88%, 89%, 90%, 91%, 92%, 92%, 94%, 95%, 96%, 97%, 98%, 99% sequence identity to SEQ ID NO:194. In some embodiments, TrxA has the sequence set forth in SEQ ID NO:194. In some embodiments, TrxA is encoded by a nucleotide sequence set forth in SEQ ID NO: 193 or a sequence of nuceotides that exhibits at least 85%, 86%, 87%, 88%, 89%, 90%, 91%, 92%, 92%, 94%, 95%, 96%, 97%, 98%, 99% sequence identity to SEQ ID NO:193.

In some embodiments, the the binding peptide is C-terminal to the bacterial chaperone (e.g. TrxA). In some embodiments, the binding peptide and bacterial chaperone are joined by a cleavable linker. In some embodiments, the cleavable linker comprises a cleavage site selected from: (i) a enterokinase cleavage site, optionally comprising the amino acid sequence set forth by DDDDK (SEQ ID NO:106); (ii) a Factor Xa cleavage site, optionally comprising the amino acid sequence set forth by SEQ ID NO: 218 or I-E/D-G-R; (iii) a thrombin cleavage site, optionally comprising the amino acid sequence set forth by any one of SEQ ID NOS: 219-222, more optionally set forth by SEQ ID NO: 220; or (iv) a TEV protease cleavage site set forth by SEQ ID NO: 223 or SEQ ID NO:224. In some embodiments, the cleavable linker is a enterokinase cleavable linker comprising the amino acid sequence DDDDK (SEQ ID NO:106). In some embodiments, the enterokinase cleavable linker comprises the sequence DYKDDDK (SEQ ID NO: 209) or GGSDYKDDDDKGS (SEQ ID NO:210).

In some embodiments, the binding peptide is engineered into a loop of the bacterial chaperone. In some embodiments, the bacterial chaperone is TrxA and the loop is selected the catalytic loop corresponding to residues 31-35 of SEQ ID NO:194, the first binding loop corresponding to residues 74-76 of SEQ ID NO:194 or the second binding loop corresponding to residues 91-93 of SEQ ID NO:194. In some embodiments, the loop is the second binding loop corresponding to residues 91-93 of SEQ ID NO:194. In some embodiments, the modified binding peptide is engineered between Val-92 and Gly-93 of the sequence set forth in SEQ ID NO:194. In some embodiments, the binding peptide is engineered into the loop between a first and second cleavable linker positioned on the N-berminus and C-terminus of the binding polypeptide, respectively. In some embodiments, the first and second cleavable linker are the same. In some embodiments, the first and second cleavable linker comprise a cleavage site selected from: (i) a enterokinase cleavage site, optionally comprising the amino acid sequence set forth by DDDDK (SEQ ID NO:106); (ii) a Factor Xa cleavage site, optionally comprising the amino acid sequence set forth by SEQ ID NO: 218 or I-E/D-G-R; (iii) a thrombin cleavage site, optionally comprising the amino acid sequence set forth by any one of SEQ ID NOS: 219-222, more optionally set forth by SEQ ID NO: 220; or (iv) a TEV protease cleavage site set forth by SEQ ID NO: 223 or SEQ ID NO:224. In some embodiments, the first and second cleavable linker is a enterokinase cleavable linker comprising the amino acid sequence DDDDK (SEQ ID NO:106). In some embodiments, the enterokinase cleavable linker comprises the sequence DYKDDDK (SEQ ID NO: 209) or GGSDYKDDDDKGS (SEQ ID NO:210).

In some embodiments, the provided methods further include culturing the host cell, e.g., the bacteria, such as E. coli, under conditions permissive of expression of the fusion protein. In some embodiments, the provided methods further include, following the culturing, isolating the expressed fusion protein from supernatant of a lysate of the host cell, e.g., the bacteria, such as E. coli. In some embodiments, the provided methods further include cleaving the cleavable linker, thereby producing the soluble peptide that is free of the bacterial chaperone.

In some embodiments, the cleavable linker is an enterokinase cleavage tag. In some embodiments, the cleavable linker includes the amino acid sequence DD D D K (SEQ ID NO: 106). In some embodiments, the cleaving of the cleavable linker includes adding enterokinase. In some embodiments, enterokinase is added to the supernatant of the host cell lysate. In some embodiments, the provided methods further include, following cleaving the cleavable linker, removing the enterokinase and/or the bacterial chaperone from the solution containing the soluble peptide.

In some embodiments, the provided methods further include steps for enriching for the soluble peptide. In some embodiments, the provided methods further include separating the soluble peptide from any soluble aggregates present in solution, including soluble aggregates of the soluble peptide. In some embodiments, the separating involves the active soluble peptide from the larger, inactive or less active soluble aggregates thereof. In some embodiments, the separating is achieved using chromatographic methods. In some embodiments, the enriching or separating is by size exclusion chromatography. In some embodiments, the separating involves collecting one or more elution fractions containing the soluble peptide, but not the soluble aggregates thereof, thereby producing an enriched or purified composition of soluble peptides.

In some embodiments, the provided methods further include producing a multispecific binding molecule that includes the soluble peptide. In some embodiments, the multispecific binding molecule includes multiple copies of the soluble peptide. In some embodiments, the multispecific binding molecule includes different soluble peptides. In some embodiments, the multispecific binding molecule includes a flexible linker (e.g., Gly-Gly-Gly-Ser) between the soluble peptides (e.g., between the C-terminus of one soluble peptide copy and the N-Terminus of the other soluble peptide copy). In some embodiments, one soluble peptide is present in a VH region that is expressed with a light chain as an IgG, and the second soluble peptide is fused to the heavy chain constant region. In some embodiments, the multispecific binding molecule includes two VH regions with the same soluble peptide. In some embodiments, the multispecific binding molecule includes VH regions that include different soluble peptides, for instance using heavy chains with constant region mutations such that only the heterologous heavy chains effectively pair with one another to form a dimer. In some embodiments, these mutations are ‘knobs-into-holes’ mutations, such as T22Y on one chain and Y86T on the other chain in the CH3 domain of Fc.

In some embodiments, the expression vector further includes an inducible promoter sequence to control the expression of the fusion protein. The term “promoter sequence” as used herein refers to a DNA sequence, which is generally located upstream of a gene present in a DNA polymer, and provides a site for initiation of the transcription of said gene into mRNA. Promoter sequences suitable for use in this invention may be derived from viruses, bacteriophages, prokaryotic cells or eukaryotic cells, and may be a constitutive promoter or an inducible promoter.

In some embodiments, the inducible promoter sequence is operably linked to the sequence encoding the fusion protein. The term “operatively linked” as used herein means that a first sequence is disposed sufficiently close to a second sequence such that the first sequence can influence the second sequence or regions under the control of the second sequence. For instance, a promoter sequence may be operatively linked to a gene sequence, and is normally located at the 5′-terminus of the gene sequence such that the expression of the gene sequence is under the control of the promoter sequence. In addition, a regulatory sequence may be operatively linked to a promoter sequence so as to enhance the ability of the promoter sequence in promoting transcription. In such case, the regulatory sequence is generally located at the 5′-terminus of the promoter sequence.

Promoter sequences suitable for use in this invention are preferably derived from any one of the following: viruses, bacterial cells, yeast cells, fungal cells, algal cells, plant cells, insect cells, animal cells, and human cells. For example, a promoter useful in bacterial cells includes, but is not limited to, tac promoter, T7 promoter, T7 A1 promoter, lac promoter, trp promoter, trc promoter, araBAD promoter, and XPRPL promoter. A promoter useful in plant cells includes, e.g., 35S CaMV promoter, actin promoter, ubiquitin promoter, etc. Regulatory elements suitable for use in mammalian cells include CMV-HSV thymidine kinase promoters, SV40, RSV-promoters, CMV enhancers, or SV40 enhancers.

Vectors suitable for use in this invention include those commonly used in genetic engineering technology, such as bacteriophages, plasmids, cosmids, viruses, or retroviruses.

Vectors suitable for use in this invention may include other expression control elements, such as a transcription starting site, a transcription termination site, a ribosome binding site, a RNA splicing site, a polyadenylation site, a translation termination site, etc. Vectors suitable for use in this invention may further include additional regulatory elements, such as transcription/translation enhancer sequences, and at least a marker gene or reporter gene allowing for the screening of the vectors under suitable conditions. Marker genes suitable for use in this invention include, for instance, dihydrofolate reductase gene and G418 or neomycin resistance gene useful in eukaryotic cell cultures, and ampicillin, streptomycin, tetracycline or kanamycin resistance gene useful in E. coli and other bacterial cultures. Vectors suitable for use in this invention may further include a nucleic acid sequence encoding a secretion signal. These sequences are well known to those skilled in the art.

Depending on the vector and host cell system used, the recombinant gene product (protein) produced according to this invention may either remain within the recombinant cell, be secreted into the culture medium, be secreted into periplasm, or be retained on the outer surface of a cell membrane. The recombinant gene product (protein) produced by the method of this invention can be purified by using a variety of standard protein purification techniques, including, but not limited to, affinity chromatography, ion exchange chromatography, gel filtration, electrophoresis, reverse phase chromatography, chromatofocusing and the like. The recombinant gene product (protein) produced by the method of this invention is preferably recovered in “substantially pure” form. As used herein, the term “substantially pure” refers to a purity of a purified protein that allows for the effective use of said purified protein as a commercial product.

### A. Host Cells

The term “host cell” is used to refer to a cell which has been transformed, transfected or infected or is capable of being transformed, transfected or infected with a nucleic acid sequence and then of expressing a selected gene of interest to recombinantly produce a protein of interest. The term includes the progeny of the parent cell, whether or not the progeny is identical in morphology or in genetic make-up to the original parent, so long as the selected gene or genetic modification is present.

The provided methods for producing a soluble peptide or a fusion protein containing the soluble peptide and a chaperone, e.g., bacterial chaperone, can be performed using any host organism which is capable of expressing heterologous polypeptides, and is capable of being genetically modified. A host organism is preferably a unicellular host organism, however, the use of multicellular organisms is also encompassed by the provided methods, provided the organism can be modified as described herein and a polypeptide of interest expressed therein. For purposes of clarity, the term “host cell” will be used herein throughout, but it should be understood, that a host organism can be substituted for the host cell, unless unfeasible for technical reasons.

In some embodiments, the host cell is a prokaryotic cell, such as a bacterial cell. The host cell may be a gram positive bacterial cells, such as Bacillus or gram negative bacteria such as E. coli. The host organisms may be aerobic or anaerobic organisms. In some embodiments, host cells are those which have characteristics which are favorable for expressing polypeptides, such as host cells having fewer proteases than other types of cells. Suitable bacteria for this purpose include archaebacteria and eubacteria, for example, Enterobacteriaceae. Other examples of useful bacteria include Escherichia, Enterobacter, Azotobacter, Erwinia, Bacillus, Pseudomonas, Klebsiella, Proteus, Salmonella, Serratia, Shigella, Rhizobia, Vitreoscilla, and Paracoccus. Additional examples of useful bacteria include Corynebacterium, Lactococcus, Lactobacillus, and Streptomyces species, in particular Corynebacterium glutamicum, Lactococcus lactis, Lactobacillus plantarum, Streptomyces coelicolor, Streptomyces lividans. Suitable E. coli hosts include E. coli DHB4, E. coli BL-21 (which are deficient in both lon (Phillips et al. J. Bacteriol. 159: 283, 1984) and ompT proteases), E. coli AD494, E. coli W3110 (ATCC 27,325), E. coli 294 (ATCC 31,446), E. coli B, and E. coli X1776 (ATCC 31,537). Other strains include E. coli B834 which are methionine deficient and, therefore, enables high specific activity labeling of target proteins with ³⁵S-methionine or selenomethionine (Leahy et al. Science 258: 987, 1992). Yet other strains of interest include the BLR strain, and the K-12 strains HMS174 and NovaBlue, which are recA-derivative that improve plasmid monomer yields and may help stabilize target plasmids containing repetitive sequences.

In some embodiments, the E. coli host cell used in the provided methods is engineered or modified to improve soluble expression of disulfide-bonded proteins in the E. coli cytosol. In some embodiments, the cytoplasmic thiol-redox equilibrium environment is changed via alteration in reducing pathways, such as thioredoxin reductase. In some embodiments, the E. coli host cell has an oxidizing cytoplasm that is permissive of disulfide bond formation. Various types of mutant strains, including SHuffle (New England Biolabs) and Origami™ (DE3) (Novagen, Germany), which lack glutathione reductase Agor, thioredoxin reductase, and/or glutathione biosynthesis pathways, are commercially available. In some embodiments, the E. coli strain transformed as part of the provided methods is the Origami™ (DE3) (Novagen, Germany) mutant strain.

Suitable Bacillus strains include Bacillus subtilis, Bacillus anzyloliguelaciens, Bacillus licheniformis, Bacillus brevis, Bacillus alcalophilus, Bacillus clauseii, Bacillus cereus, Bacillus pumilus, Bacillus thuringiensis, or Bacillus halodurans. The Gram-positive bacterium B. subtilis is a preferred organism for secretory protein production in the biotechnological industry. Its popularity is primarily based on the fact that B. subtilis lacks an outer membrane, which retains many proteins in the periplasm of Gram-negative bacteria such as Escherichia coli. Accordingly, the majority of B. subtilis proteins that are transported across the cytoplasmic membrane end up directly in the growth medium. Additionally, the lack of an outer membrane implies that proteins produced with B. subtilis are free from lipopolysaccharide (endotoxin). Other advantages of using B. subtilis as a protein production host are its high genetic amenability, the availability of strains with mutations in nearly all of the ˜4100 genes, a toolbox with strains and vectors for gene expression, and the fact that this bacterium is generally recognized as safe (Braun et al., Curr. Opin. Biotechnol. 10:376-381, 1999; Kobayashi et al., Proc. Natl. Acad. Sci. U.S.A 100:4678-4683, 2003; Kunst et al. Nature 390:249-256, 1997; Zeigler et al., In E. Goldman and L. Green (ed.), Practical Handbook of Microbiology. CRC Press, Boca Raton, Fla., 2008).

In another embodiment, the host cell is a eukaryotic cell, such as a yeast cell or a mammalian cell. Examples of mammalian cells include, but are not limited to Chinese hamster ovary cells (CHO) (ATCC No. CCL61), CHO DHFR-cells (Urlaub et al., Proc. Natl. Acad. Sci. USA, 97:4216-4220 (1980)), human embryonic kidney (HEK) 293 or 293T cells (ATCC No. CRL1573), or 3T3 cells (ATCC No. CCL92). The selection of suitable mammalian host cells and methods for transformation, culture, amplification, screening and product production and purification are known in the art. Other suitable mammalian cell lines, are the monkey COS-1 (ATCC No. CRL1650) and COS-7 cell lines (ATCC No. CRL1651), and the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include but are not limited to, mouse neuroblastoma N2A cells, HeLa, mouse L-929 cells, 3T3 lines derived from Swiss, Balb-c or NIH mice, BHK or HaK hamster cell lines, which are available from the ATCC. Each of these cell lines is known by and available to those skilled in the art of protein expression.

Many strains of yeast cells known to those skilled in the art are also available as host cells for the expression of the polypeptides described herein. Exemplary yeast cells include, for example, Saccharomyces cerivisae and Pichia pastoris. Fungi, such as Aspergillum, are also available as host cells for the expression of the polypeptides described herein.

Additionally, where desired, insect cell systems may be utilized in the provided methods. Such systems are described for example in Kitts et al., Biotechniques, 14:810-817 (1993); Lucklow, Curr. Opin. Biotechnol., 4:564-572 (1993); and Lucklow et al. (J. Virol., 67:4566-4579 (1993). Exemplary insect cells are Sf-9 and Hi5 (Invitrogen, Carlsbad, Calif.).

### B. Soluble Peptides

In some embodiments, the soluble peptide produced in the provided methods is a soluble binding peptide. In some embodiments, the soluble peptide produced in the provided methods is a soluble synthetic or semisynthetic peptide. In some embodiments, the soluble peptide produced in the provided methods is a cyclotide. In some embodiments, the soluble peptide produced in the provided methods is a modified cyclotide. In some embodiments, the soluble peptide produced in the provided methods is a semisynthetic or modified ultralong CDR3 knob.

In some embodiments, the soluble peptide produced in the provided methods is a soluble ultralong CDR3 knob. In some embodiments, the soluble ultralong CDR3 knob is a cow ultralong CDR3. In some embodiments, the soluble ultralong CDR3 knob is encoded by a sequence that has been amplified from a cow cDNA template library, e.g., one prepared from RNA isolated from peripheral blood mononuclear cells (PBMCs) from an immunized cow. In some embodiments, the soluble ultralong CDR3 knob includes all or a portion of sequences that have been amplified from a cow cDNA template library according to any of the methods provided herein (see, e.g., Sections II-A-1-a and II-A-1-b). In some embodiments, the soluble ultralong CDR3 knob is any that has been identified or selected as a binder of a target molecule. In some embodiments, the soluble ultralong CDR3 knob is or is a portion of any ultralong CDR3 knob that has been identified or selected as a binder of a target molecule according to any of the methods provided herein (see, e.g., Sections II-C).

In some embodiments, the binding peptide also can include an N-terminal and/or C-terminal flexible linker at the N-terminus and/or C-terminus of the binding peptide In some embodiments, the flexible linker is included at the N-terminus of the soluble peptide. In some embodiments, the flexible linker is in addition to the 3-6 amino acids preceding the most N-terminal cysteine residue present in the soluble peptide. In some embodiments, the flexible linker is included in the 3-6 amino acids preceding the most N-terminal cysteine residue present in the soluble peptide. In some embodiments, the flexible linker is included at the C-terminus of the soluble peptide. In some embodiments, the flexible linker is in addition to the at least 6 amino acids following the most C-terminal cysteine residue present in the soluble peptide. In some embodiments, the flexible linker is included in the at least 6 amino acids following the most C-terminal cysteine residue present in the soluble peptide.

In some embodiments, the flexible linker is GGGGAMGS (SEQ ID NO: 108). In some embodiments, the flexible linker is GGS (SEQ ID NO: 109). In some embodiments, the flexible linker (e.g., GGGGAMGS, SEQ ID NO: 108) allows for cyclization of the soluble peptide. In some embodiments, the cyclization is via chemical or enzymatic methods. In some embodiments, the flexible linker (e.g., GGGGAMGS, SEQ ID NO: 108) allows for sortase-mediated cyclization of the soluble peptide. In some embodiments, the provided methods further include a step of cyclizing the soluble peptide, e.g., via chemical or enzymatic methods.

### VI. Compositions and Formulations

Also provided are compositions comprising the binding polypeptides, such as antibodies or antigen-binding fragments or knob peptides, described herein, including pharmaceutical compositions and formulations. In one embodiment, a composition comprises a soluble peptide produced as described herein. In one embodiment, a composition comprises a fusion protein containing a soluble peptide, produced as described herein. In one embodiment, a composition comprises a soluble peptide identified for binding ability to a target molecule, e.g., identified as described herein. In some embodiments, a composition comprises a knob polypeptide or a synthetic peptide comprising an ultralong CDR3. The pharmaceutical compositions and formulations generally include one or more optional pharmaceutically acceptable carrier or excipient.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Formulations of the antibodies described herein can include lyophilized formulations and aqueous solutions.

In some embodiments, an antibody described herein may be administered within a pharmaceutically-acceptable diluent, carrier, or excipient, in unit dose form. Conventional pharmaceutical practice may be employed to provide suitable formulations or compositions to administer to individuals being treated for SARS CoV-2 infection. In some embodiments, the administration is prophylactic. Any appropriate route of administration may be employed, for example, administration may be parenteral, intravenous, intra-arterial, subcutaneous, intramuscular, intraperitoneal, intranasal, aerosol, suppository, oral administration, or via inhalation.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Pharmaceutical compositions according to the invention may be, for example, in unit dose form, such as in the form of ampoules, vials, suppositories, tablets, pills, or capsules. The formulations can be administered to human individuals in therapeutically or prophylactic effective amounts (e.g., amounts which prevent, eliminate, or reduce a pathological condition) to provide therapy for a disease or condition. The preferred dosage of therapeutic agent to be administered is likely to depend on such variables as the type and extent of the disorder, the overall health status of the particular patient, the formulation of the compound excipients, and its route of administration.

In certain embodiments, the compositions described herein can be formulated for pneumonal administration, and in certain embodiments the composition is formulated for administration via inhalation (e.g., intrabronchial, intranasal or oral inhalation, intranasal drops). The composition may be administered with the use of a nebulizer, inhaler, atomizer, aerosolizer, mister, dry powder inhaler, metered dose inhaler, metered dose sprayer, metered dose mister, metered dose atomizer, or other suitable delivery device.

In some embodiments, the composition is a lyophilized composition. In some embodiments, the composition is formulated for aerosol administration, and in certain embodiments the composition is formulated for oral administration or administration via inhalation.

The pharmaceutical compositions described herein are prepared in a manner known per se, for example, by means of conventional dissolving, lyophilizing, mixing, granulating or confectioning processes. The pharmaceutical compositions may be formulated according to conventional pharmaceutical practice (see for example, in Remington: The Science and Practice of Pharmacy (21st ed.), ed. A. R. Gennaro, 2005, Lippincott Williams & Wilkins, Philadelphia, PA, and Encyclopedia of Pharmaceutical Technology, eds. J. Swarbrick and J. C. Boylan, 2013, Marcel Dekker, New York, NY).

In instances where aerosol administration is appropriate, the squalamine or a derivative thereof can be formulated as aerosols using standard procedures. The term “aerosol” includes any gas-borne suspended phase of a squalamine or a derivative thereof which is capable of being inhaled into the bronchioles or nasal passages, and includes dry powder and aqueous aerosol, and pulmonary and nasal aerosols. Specifically, aerosol includes a gas-bome suspension of droplets of squalamine or a derivative thereof, as may be produced in a metered dose inhaler or nebulizer, or in a mist sprayer. Aerosol also includes a dry powder composition of a compound of the invention suspended in air or other carrier gas, which may be delivered by insufflation from an inhaler device, for example. See Ganderton & Jones, Drug Delivery to the Respiratory Tract (Ellis Horwood, 1987); Gonda, Critical Reviews in therapeutic Drug Carrier Systems, 6:273-313 (1990); and Raeburn et al. Pharmacol. Toxicol. Methods, 27:143-159 (1992).

The formulations to be used for in vivo administration are generally sterile. The injection compositions are prepared in customary manner under sterile conditions; the same applies also to introducing the compositions into ampoules or vials and sealing the containers. Sterility may be readily accomplished, e.g., by filtration through sterile filtration membranes.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the binding polypeptides, such as antibodies or antigen binding fragments, in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition

### VII. Methods of Use and Treatment

Provided herein are methods of treatment and uses for treating a disease or condition in a subject. In some embodiments, the methods and uses include administering a provided binding peptide, such as a provided soluble binding peptide, into a subject (e.g. a human). In some embodiments, the binding binding peptide, such as a soluble binding peptide, or a composition containing same is administered to the subject by a parenteral administration. In some embodiments, the binding peptide, such as a soluble binding peptide, or a composition containing same is administered by intramuscularly, subcutaneously, intravenously, topically, orally or by inhalation. In particular embodiments, the administration is by inhalation. In some embodiments, a provided binding peptide, such as a soluble binding peptide, or composition containing same may be administered by aerosol administration, such as by delivery using an inhaler or nebulizer or a mist sprayer.

In some embodiments, the provided embodiments relate to methods for treating or preventing a disease or condition associated with the target antigen recognized by the binding peptide. In some embodiments, provided embodiments relate to methods for treating or preventing a cancer or proliferative disease in a subject. In some embodiments, provided embodiments relate to methods for treating or preventing a viral infection in a subject. Such compositions include those that contain any of the exemplary binding peptides, including a modified binding peptide, directed against SARS-CoV-2 as described in Section II.A. In provided aspects, the binding peptide is a soluble binding peptide as described in Section V.

In some embodiments, provided embodiments relate to methods for treating or preventing a coronavirus infection in a subject. In some embodiments, the methods are for prophylactic treatment of a viral infection in a subject at risk of a viral infection. In some embodiments, the methods are for treating a subject known or suspected of having a viral infection. In some embodiments, the methods may prevent a viral infection, such as a coronavirus infection, in a subject. In some embodiments, the methods may reduce signs of symptoms of the coronavirus infection in the subject, such as mitigate the presence or severity of one or more signs or symptoms. In some embodiments, the binding peptides, such as any of the provided soluble binding peptides, or compositions containing the same are administered to a subject in an effective amount to effect treatment of the infection. Also provided herein are uses of the binding peptides, such as any of the soluble binding peptides, or compositions containing the same in such methods and treatments, and in the preparation of a medicament in order to carry out such therapeutic methods. In some embodiments, the methods are carried out by administering the binding peptides, such as any of the soluble binding peptides, or compositions comprising the same, to the subject having, having had, or suspected of having the disease or condition. In some embodiments, the methods thereby treat the disease or condition or disorder in the subject.

In some embodiments, the subject may have a viral infection, e.g., an influenza infection, or be predisposed to developing an infection. Subjects predisposed to developing an infection, or subjects who may be at elevated risk for contracting an infection (e.g., of coronavirus or influenza virus), include subjects with compromised immune systems because of autoimmune disease, subjects receiving immunosuppressive therapy (for example, following organ transplant), subjects afflicted with human immunodeficiency syndrome (HIV) or acquired immune deficiency syndrome (AIDS), subjects with forms of anemia that deplete or destroy white blood cells, subjects receiving radiation or chemotherapy, or subjects afflicted with an inflammatory disorder. Additionally, subjects of very young (e.g., 5 years of age or younger) or old age (e.g., 65 years of age or older) are at increased risk. Moreover, a subject may be at risk of contracting a viral infection due to proximity to an outbreak of the disease, e.g. subject resides in a densely-populated city or in close proximity to subjects having confirmed or suspected infections of a virus, or choice of employment, e.g. hospital worker, pharmaceutical researcher, traveler to infected area, or frequent flier.

In aspects of any of the provided embodiments, the methods include a method for treating or preventing viral infection (e.g., coronavirus infection) or for inducing the regression or elimination or inhibiting the progression of at least one sign or symptom of viral infection.

Also provided herein are of use of any of the compositions, such as pharmaceutical compositions provided herein, for the treatment of a disease or disorder associated with a coronavirus infection, for example, due to SARS-CoV-2. Such compositions include those that contain any of the exemplary binding peptides, including a modified binding peptide, directed against SARS-CoV-2 as described in Section II.A. In provided aspects, the binding peptide is a soluble binding peptide as described in Section V.

In some embodiments, the provided methods and uses include prophylactic methods and uses. In some embodiments, provided herein are methods for prophylactically administering a provided binding peptide, such as a soluble binding peptide, to a subject having or who is at risk of viral infection so as to prevent such infection. In some embodiments, the amount administered is an effective or therapeutically effective amount or dose. In some embodiments, the provided methods and uses prevent a viral infection in the subject. In some embodiments, preventing a viral infection by a provided methods involves administering a provided binding peptide, such as a soluble binding peptide, to a subject to inhibit the manifestation of a disease or infection (e.g., viral infection) in the body of a subject. In some embodiments, the methods reduce one or more sign or symptom of a viral infection.

The coronavirus infection typically involves respiratory tract infections, often in the lower respiratory tract. Symptoms can include high fever, dry cough, shortness of breath, pneumonia, gastro-intestinal symptoms such as diarrhea, organ failure (kidney failure and renal dysfunction), septic shock, and death in severe cases. The coronavirus infection may include any virus infection in the body of a subject that is treatable or preventable by administration of a binding polypeptide directed against a coronavirus spike protein wherein infectivity of the virus is at least partially dependent on the coronavirus spike protein.

In particular, the virus is a virus that infects the respiratory tissue of a subject (e.g., upper and/or lower respiratory tract, trachea, bronchi, lungs) and is treatable or preventable by administration of an binding polypeptide against the coronavirus spike protein. Coronaviruses can include the genera of alphacoronaviruses, betacoronaviruses, gammacoronaviruses, and deltacoronaviruses. For example, the virus includes coronavirus, such as SARS-CoV-2 (severe acute respiratory syndrome coronavirus 2), SARS-CoV (severe acute respiratory syndrome coronavirus), and MERS-CoV (Middle East respiratory syndrome (MERS) coronavirus). In some embodiments, the coronavirus infection is an infection of a subject with a coronavirus such as SARS-CoV-2, MERS-CoV, or SARS-CoV. In some embodiments, the coronavirus infection is an infection of a subject with SARS-CoV-2.

In some embodiments, a provided binding binding peptide, such as a soluble binding peptide, is administered to the subject in an effective or therapeutically effective amount. An effective or therapeutically effective dose of a provided binding peptide, such as a soluble binding peptide, for treating or preventing a viral infection is an amount sufficient to alleviate one or more signs and/or symptoms of the disease or condition (e.g. infection) in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment, an effective or therapeutically effective dose of a provided binding peptide, such as a soluble binding peptide, for treating or preventing viral infection, e.g., in an adult human subject, is about 0.001 mg/kg to about 200 mg/kg, such as 0.01 mg/kg to 200 mg/kg or 0.1 mg/kg to 200 mg/kg. Depending on the severity of the infection, the frequency and the duration of the treatment can be adjusted.

The provided methods and uses include methods and uses for treating a viral infection in a subject. For instance, methods of treating include administering a provided binding polypeptide, such as an antibody or antigen-binding fragment or a knob peptide, to a subject having one or more signs or symptoms of a disease or infection, e.g., viral infection, at an effective or therapeutically effective amount or dose.

In aspects of the provided methods or uses, a sign or symptom of a viral infection in a subject is survival or proliferation of virus in the body of the subject, e.g., as determined by viral titer assay (e.g., coronavirus propagation in embryonated chicken eggs or coronavirus spike protein assay). In some embodiments, a sign or symptom of viral infection includes, for example, fever or feeling feverish/chills; cough; sore throat; runny or stuffy nose; sneezing; muscle or body aches; headaches; fatigue (tiredness); vomiting; diarrhea; respiratory tract infection; chest discomfort; shortness of breath; bronchitis; and/or pneumonia.

In further embodiments of the present disclosure, a composition comprising a binding peptide, such as a soluble binding peptide, can additionally be combined with other compositions for the treatment of a coronavirus infection, such as SARS CoV-2 infection or the prevention of SARS CoV-2 transmission.

### VIII. Exemplary Embodiments

Among the provided embodiments are:

1. A modified fusion polypeptide comprising the formula N-terminus to C-terminus: Y1-[polypeptide]-Y2, wherein the polypeptide is a protein in which the distance between the N-termini and C-termini is no more than 10 Angstroms and wherein Y1 and Y2 are amino acid peptide sequences that interact with one another when in proximity.

2. The modified fusion polypeptide of embodiment 1, wherein Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif.

3. The modified fusion polypeptide of embodiment 2, wherein the Y1 and Y2 are HW and SF, respectively.

4. The modified fusion polypeptide of embodiment 2, wherein Y1 and Y2 are IS and TV, respectively.

5. The modified fusion polypeptide of embodiment 2, wherein Y1 and Y2 are sequences that are each 14-30 amino acids in length comprising a heptad repeat, wherein Y1 and Y2 are able to interact to form an anti-parallel coiled-coil motif.

6. The modified fusion polypeptide of embodiment 2 and 5, wherein Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203.

7. The modified fusion polypeptide of embodiment 2, 5 or 6, wherein the Y1 is set forth in SEQ ID NO:202 and Y2 is set forth in SEQ ID NO:203, respectively.

8. The modified fusion polypeptide of any of embodiments 1-7, wherein the distance between the N- and C-termini is no more than 5, 6, 7, 8 or 9 Angstoms.

9. The modified fusion polypeptide of any of embodiments 1-8, wherein the distance between the N- and C-termini is between 2 and 10 Angstroms.

10. The modified fusion polypeptide of any of embodiments 1-9, wherein the distance between the N- and C-termini is between 2 and 8 Angstroms.

11. The modified fusion polypeptide of any of embodiments 1-10, wherein the polypeptide is selected from a cysteine motif peptide or a cytokine, wherein the cysteine motif binding peptide is 20-50 amino acids in length in which 2-12 amino acids are cysteine residues able to form 1-6 disulfide bonds.

12. The modified fusion polypeptide of embodiment 11, wherein the cysteine motif binding peptide is a knob peptide from an ultralong CDR3 of a cow antibody.

13. The modified fusion polypeptide of embodiment 11 or embodiment 12, wherein the cysteine motif binding peptide does not comprise an ascending or descending stalk domain or a contiguous portion thereof of more than 3 amino acids in length from the ultralong CDR3 of the cow antibody.

14. The modified fusion polypeptide of any of embodiments 11-13, wherein the cysteine motif binding peptide is 22 to 43 amino acids in length.

15. The modified fusion polypeptide of any of embodiments 11-14, wherein the cysteine motif binding peptide comprises at least 4 cysteine residues.

16. The modified fusion polypeptide of any of embodiments 11-15, wherein the cysteine motif binding peptide contains 4 cysteine residues, 6 cysteine residues or 8 cysteine residues.

17. The modified fusion polypeptide of any of embodiments 11-16, wherein the cysteine motif binding peptide is able to form at least 2 disulfide bonds.

18. The modified fusion polypeptide of any of embodiments 11-17, wherein the cysteine motif binding peptide is able to form 2 disulfide bonds, 3 disulfide bonds or 4 disulfide bonds.

19. The modified fusion polypeptide of any of embodiment 11-18, wherein the cysteine motif binding peptides binds to a target antigen that is a nonvirulent bacteria, a virus, a viral protein, an immunomodulatory protein, a cancer antigen, a human IgG, or a recombinant protein thereof.

20. The modified fusion polypeptide of embodiment 19, wherein the target antigen is a coronavirus, a coronavirus pseudovirus, a recombinant coronavirus Spike protein, or a receptor-binding domain (RBD) of a coronavirus Spike protein.

21. The modified fusion polypeptide of embodiment 20, wherein the coronavirus is selected from the group consisting of 229E, NL63, OC43, HKU1, MERS-CoV, SARS-CoV, and SARS-CoV2.

22. The modified fusion polypeptide of embodiment 20 or embodiment 21, wherein the coronavirus is a SARS-CoV2 selected from Wuhan-Hu-1 isolate, B.1.351 South African variant, B.1.1.7 UK variant, a beta variant, a delta variant, or a omicron variant.

23. The modified fusion polypeptide of any of embodiments 11-22, wherein the cysteine motif binding peptide is set forth in any one of SEQ ID NOS: 155, 198, and 227-240.

24. The modified polypeptide of any of embodiments 1-18, wherein the cytokine is IL-2 or IL-15.

25. A fusion protein, comprising a modified fusion polypeptide of any of embodiments 1-24 and a moiety selected from a half-life extending moiety or a detectable moiety.

26. The fusion protein of embodiment 25, wherein the half-life extending moiety is an immunoglobulin Fc.

27. The fusion protein of embodiment 25, wherein the detectable moiety is a fluorescent protein.

28. The fusion protein of embodiment 27, wherein the fluorescent protein is a GFP, optionally sfGFP.

29. The fusion protein of any of embodiments 25-28, wherein the modified fusion polypeptide is inserted within the half-life extending moiety or detectable moiety.

30. The fusion protein of any of embodiments 25-29, wherein the modified fusion polypeptide is inserted within a loop of the half-life extending moiety or detectable moiety.

31. A nucleic acid encoding a modified fusion polypeptide of any of embodiments 1-24 or the fusion protein of any of embodiments 25-30.

32. An expression vector comprising the nucleic acid molecule of embodiment 31.

33. A composition comprising the modified fusion polypeptide of any of embodiments 1-24 or the fusion protein of any of embodiments 25-30.

34. The composition of embodiment 33 that is a pharmaceutical composition comprising a pharmaceutically acceptable excipient.

35. A method of producing a modified fusion polypeptide, the method comprising adding an N-terminus sequence (Y1) and a C-terminus sequence (Y2) to a polypeptide, wherein the polypeptide is a protein in which the distance between the N-termini and C-termini is no more than 10 Angstroms and wherein the modified fusion polypeptide has the formula N-terminus to C-terminus: Y1-[polypeptide]-Y2, wherein Y1 and Y2 are amino acid peptide sequences that interact with one another when in proximity.

36. The method of embodiment 35, wherein Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif.

37. The method of embodiment 35 or embodiment 36, wherein the polypeptide is selected from a cysteine motif peptide or a cytokine, wherein the cysteine motif binding peptide is 20-50 amino acids in length in which 2-12 amino acids are cysteine residues able to form 1-6 disulfide bonds.

38. The method of embodiment 37, wherein the cytokine is IL-2 or IL-15.

39. A method of producing a modified binding peptide, the method comprising:
(a) obtaining a cysteine motif binding peptide of 20-50 amino acids in length in which 2-12 amino acids are cysteine residues able to form 1-6 disulfide bonds; and
(b) modifying the binding peptide by adding an N-terminus sequence (Y1) and a C-terminus sequence (Y2) to the cysteine motif binding peptide, wherein the modified binding peptide has the formula N-terminus to C-terminus: Y1-[cysteine motif binding peptide]-Y2, wherein Y1 and Y2 are characterized by:
   (i) Y1 and Y2 are selected from (i) HW and SF, respectively; (ii) IS and TV, respectively; (iii) DY and MP, respectively; (iv) LV and IP, respectively; or (v) SV and YI, respectively; or
   (ii) Y1 and Y2 are sequences that are each 14-30 amino acids in length comprising a heptad repeat, wherein Y1 and Y2 are able to interact to form an anti-parallel coiled-coil motif.

40. The method of any of embodiments 35-39, wherein the Y1 and Y2 are HW and SF, respectively.

41. The method of any of embodiments 35-39, wherein Y1 and Y2 are IS and TV, respectively.

42. The method of any of embodiments 35-39, wherein Y1 and Y2 are sequences that are each 14-30 amino acids in length comprising a heptad repeat, wherein Y1 and Y2 are able to interact to form an anti-parallel coiled-coil motif.

43. The method of any of embodiments 35-39 and 42, wherein Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203.

44. The method of any of embodiments 35-39, 42 and 43, wherein the Y1 is set forth in SEQ ID NO:202 and Y2 is set forth in SEQ ID NO:203, respectively.

45. The method of any of embodiments 37 and 39-44, wherein the cysteine motif binding peptide is a knob peptide from an ultralong CDR3 of a cow antibody.

46. The method of embodiment 45, wherein the cysteine motif binding peptide does not comprise an ascending or descending stalk domain or a contiguous portion thereof of more than 3 amino acids in length from the ultralong CDR3 of the cow antibody.

47. The method of any of embodiments 37 and 39-46, wherein the cysteine motif binding peptide is 22 to 43 amino acids in length.

48. The method of any of embodiments 37 and 39-47, wherein the cysteine motif binding peptide comprises at least 4 cysteine residues.

49. The method of any of embodiments 37 and 39-48, wherein the cysteine motif binding peptide contains 4 cysteine residues, 6 cysteine residues or 8 cysteine residues.

50. The method of any of embodiments 37 and 39-49, wherein the cysteine motif binding peptide is able to form at least 2 disulfide bonds.

51. The method of any of embodiments 37 and 39-50, wherein the cysteine motif binding peptide is able to form 2 disulfide bonds, 3 disulfide bonds or 4 disulfide bonds.

52. The method of any of embodiments 37 and 39-51, wherein the cysteine motif binding peptide is identified by a method comprising:
(1) immunizing a cow with a target antigen or a sequence portion comprising an epitope thereof;
(2) identifying a knob peptide sequence from an antibody variable heavy chain (VH) sequence from peripheral blood mononuclear cells (PBMCs) from the immunized cow, wherein the knob peptide is a sequence between the ascending and descending stalk sequences of an ultralong CDR3, wherein the ultralong CDR3 is 40 to 70 amino acids in length, and wherein the knob peptide is a cysteine motif binding peptide of 20-50 amino acids in length in which 2-12 amino acids are cysteine residues able to form 1-6 disulfide bonds.

53. The method of embodiment 52, wherein the knob peptide is identified from the VH sequence by an algorithm comprising:
identifying the conserved cysteine in framework 3 and the conserved tryptophan in framework 4; and
determining the sequence of the knob, in which:
   the knob has the amino acid sequence length K;
   the sequence begins at position X+1 and ends at X+K; and
   K=L−2X; wherein L is the number of amino acids in an amino acid sequence starting at the conserved cysteine in framework 3 and ending at the conserved tryptophan in framework 4, and X is the number of amino acids from the first cysteine in framework 3 to the first conserved cysteine encoded by the D_{H} region in CDR H3.

54. The method of embodiment 52 or embodiment 53, wherein the cysteine motif binding peptide is extended by one, two, three, four, or five amino acids at the N and/or C termini of the ultralong CDR3 compared to the determined knob sequence.

55. The method of any of embodiments 52-54, wherein identifying the knob peptide comprises:
(a) amplifying sequences encoding a plurality of variable heavy (VH) regions of the IgHV1-7 family from a VH chain complementary DNA (cDNA) template library prepared from RNA isolated from the PBMCs from the immunized cow;
(b) constructing a plurality of replicable expression vectors for the plurality of VH regions, wherein each replicable expression vector comprises a nucleic acid sequence encoding a single chain variable fragment (scFv) comprising an amplified VH region joined to a variable lambda light (VL) region selected from the group consisting of VL regions of BLV1H12, BLV5D3, BLV8C11, BF1H1, BLV5B8, and F18, or a humanized variant thereof;
(c) transforming suitable host cells with the plurality of replicable expression vectors under conditions suitable to produce amplified display particles; and
(d) collecting the amplified display particles, wherein the amplified display particles comprise display particles displaying a fusion protein comprising an scFv; and
(e) contacting the amplified display particles with a target antigen under conditions to allow binding of a display particle to the target antigen; and
(f) selecting display particles comprising an antibody that binds to the target antigen by separating the display particles that bind from those that do not; and
(g) sequencing the fusion gene in the selected display particles to identify the antibody with a VH sequence that comprises or is suspected of comprising an ultralong CDR3.

56. The method of embodiment 55, wherein the VL region is the BLV1H12 VL region.

57. The method of embodiment 56, wherein the BLV1H12 lambda VL region is set forth in SEQ ID NO: 2.

58. The method of embodiment 55, wherein the BLV1H12 lambda VL region is a humanized variant of the lambda VL region of BLV1H12.

59. The method of embodiment 58, wherein the humanized variant comprises one or more of amino acid replacements S2A, T5N, P8S, A12G, A13S, and P14L based on Kabat numbering, amino acid replacements I29V and N32G in the CDR1 region, and/or amino acid substitution of DNN to GDT in the CDR2 region.

60. The method of any of embodiment 58 or embodiment 59, wherein the humanized variant comprises the sequence set forth in SEQ ID NO: 107.

61. The method of any of embodiments 55-60, wherein the cDNA template library is prepared from RNA isolated from peripheral blood mononuclear cells (PBMCs) from an immunized cow.

62. The method of any of embodiments 55-61, wherein the plurality of VH regions of the IgHV1-7 family from the cDNA template library are amplified with a forward primer comprising the sequence set forth in SEQ ID NO: 84 and a reverse primer comprising the sequence set forth in SEQ ID NO: 85.

63. The method of any of embodiments 55-62, wherein prior to the constructing, the method further comprises performing a size separation on the sequences encoding the plurality of amplified VH regions to enrich for VH regions with an ultralong CDR3, optionally wherein the size separation is by gel electrophoresis.

64. The method of embodiment 63, wherein the size separation comprises separating sequences of, of about, or greater than 550 base pairs in length from the sequences encoding the plurality of amplified VH regions, wherein the sequences of, of about, or greater than 550 base pairs in length comprise sequences encoding VH regions with an ultralong CDR3.

65. The method of any of embodiments 52-54, wherein identifying the knob peptide sequence comprises amplification from a variable heavy chain cDNA template library from the immunized cow using primers specific for either side of the stalk domain of a cow ultralong CDR3 region.

66. The method of any of embodiments 52-54 and 65, wherein identifying the knob peptide comprises:
(a) amplifying sequences encoding a plurality of CDR3-knob only antibodies from a cow antibody variable heavy (VH) chain complementary DNA (cDNA) template library with forward and reverse primers specific for the ascending and descending stalk domains of a cow ultralong CDR3 region;
(b) constructing a plurality of replicable expression vectors for the plurality of CDR3-knob only antibodies, wherein each replicable expression vector comprises a nucleic acid sequence encoding an amplified CDR3 knob;
(c) transforming suitable host cells with the plurality of replicable expression vectors under conditions suitable to produce amplified display particles;
(d) collecting the amplified display particles, wherein the amplified display particles comprise display particles displaying a fusion protein comprising an amplified CDR3 knob;
(e) contacting the amplified display particles with a target antigen under conditions to allow binding of a display particle to the target antigen;
(f) selecting display particles comprising a CDR3-knob only antibody that binds to the target antigen by separating the display particles that bind from those that do not; and
(g) sequencing the fusion gene in the selected display particles to identify the CDR3-knob antibody.

67. The method of embodiment 65 or embodiment 66, wherein the cDNA template library is prepared from RNA isolated from peripheral blood mononuclear cells (PBMCs) from an immunized cow.

68. The method of any of embodiments 65-67, wherein the primers are a pool of primers that comprise or consist of any of the sequences set forth in SEQ ID NO: 7-11 and 121-130, optionally comprise or consist of any of the sequences set forth in SEQ ID NO: 123, 127, and 128.

69. The method of any of embodiments 55-68, wherein the amplified display particles are phage display particles.

70. The method of any of embodiments 39-69, wherein the cysteine motif binding peptide binds to a target antigen.

71. The method of any of embodiments 52-70, wherein the target antigen is a nonvirulent bacteria, a virus, a viral protein, an immunomodulatory protein, a cancer antigen, a human IgG, or a recombinant protein thereof.

72. The method of any of embodiments 52-71, wherein the target antigen is a coronavirus, a coronavirus pseudovirus, a recombinant coronavirus Spike protein, or a receptor-binding domain (RBD) of a coronavirus Spike protein.

73. The method of embodiment 72, wherein the coronavirus is selected from the group consisting of 229E, NL63, OC43, HKU1, MERS-CoV, SARS-CoV, and SARS-CoV2.

74. The method of embodiment 72 or embodiment 73, wherein the coronavirus is a SARS-CoV2 selected from Wuhan-Hu-1 isolate, B.1.351 South African variant, B.1.1.7 UK variant, a beta variant, a delta variant, or a omicron variant.

75. The method of any of embodiments 35-74, wherein Y1 and Y2 are added by synthetic methods or by recombinant DNA techniques.

76. A modified binding peptide produced by the methods of any of embodiments 35-75.

77. A nucleic acid molecule encoding a modified binding peptide produced by the methods of any of embodiments 35-75.

78. A method for producing a soluble binding peptide, comprising:
(a) transforming E. coli with an expression vector encoding a fusion protein comprising a binding peptide and thioredoxin A (TrxA) bacterial chaperone set forth in SEQ ID NO:194 or a sequence of amino acids that exhibits at least 85%, 86%, 87%, 88%, 89%, 90%, 91%, 92%, 92%, 94%, 95%, 96%, 97%, 98%, 99% sequence identity to SEQ ID NO:194, wherein the binding peptide is a cysteine modified binding peptide of 20-50 amino acids in length in which 2-12 amino acids are cysteine residues able to form 1-6 disulfide bonds; and;
(b) culturing the bacteria under conditions permissive of expression of the fusion protein; and
(c) isolating the fusion protein from supernatant of a bacterial cell lysate.

79. The method of embodiment 78, wherein the cysteine modified binding peptide binding peptide comprises a knob peptide from an ultralong CDR3 of a cow antibody.

80. The method of embodiment 78 or embodiment 79, wherein the cysteine modified binding peptide is set forth in any one of SEQ ID NOS: 60, 61, 62, 63, 64, 65, 66, 67, 68, 155, 198 and 227-317.

81. A method for producing a soluble ultralong CDR3 knob, comprising:
(a) transforming E. coli with an expression vector encoding a fusion protein comprising a binding peptide and a bacterial chaperone, wherein the binding peptide is a modified binding peptide that has the formula N-terminus to C-terminus: Y1-[cysteine motif binding peptide]-Y2, wherein the cysteine motif binding peptide is a peptide sequence of 20-50 amino acids with a cysteine motif comprising 2-12 cysteine residues able to form 1-6 disulfide bonds and Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif;
(b) culturing the bacteria under conditions permissive of expression of the fusion protein; and
(c) isolating the fusion protein from supernatant of a bacterial cell lysate.

82. The method of embodiment 81, wherein the Y1 and Y2 are HW and SF, respectively.

83. The method of embodiment 81, wherein Y1 and Y2 are IS and TV, respectively.

84. The method of embodiment 81, wherein Y1 and Y2 are sequences that are each 14-30 amino acids in length comprising a heptad repeat, wherein Y1 and Y2 are able to interact to form an anti-parallel coiled-coil motif.

85. The method of embodiment 81 or embodiment 84, wherein Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203.

86. The method of embodiment 81, 84 or 85, wherein the Y1 is set forth in SEQ ID NO:202 and Y2 is set forth in SEQ ID NO:203, respectively.

87. The method of any of embodiments 81-86, wherein the cysteine motif binding peptide is a knob peptide from an ultralong CDR3 of a cow antibody.

88. The method of embodiment 87, wherein the cysteine motif binding peptide does not comprise an ascending or descending stalk domain or a contiguous portion thereof of more than 3 amino acids in length from the ultralong CDR3 of the cow antibody.

89. The method of any of embodiments 81-88, wherein the cysteine motif binding peptide is 22 to 43 amino acids in length.

90. The method of any of embodiments 81-89, wherein the cysteine motif binding peptide comprises at least 4 cysteine residues.

91. The method of any of embodiments 81-90, wherein the cysteine motif binding peptide contains 4 cysteine residues, 6 cysteine residues or 8 cysteine residues.

92. The method of any of embodiments 81-91, wherein the cysteine motif binding peptide is able to form at least 2 disulfide bonds.

93. The method of any of embodiments 81-92, wherein the cysteine motif binding peptide is able to form 2 disulfide bonds, 3 disulfide bonds or 4 disulfide bonds.

94. A method for producing a soluble binding peptide, comprising:
(a) transforming E. coli with an expression vector encoding a fusion protein comprising a binding peptide and a bacterial chaperone, wherein the binding peptide is set forth in any one of SEQ ID NOS: 60, 61, 62, 63, 64, 65, 66, 67, 68, 155, 198 and 227-317 and;
(b) culturing the bacteria under conditions permissive of expression of the fusion protein;
(c) isolating the fusion protein from supernatant of a bacterial cell lysate.

95. The method of embodiments 78-94, wherein the binding peptide is set forth in any of SEQ ID NOS: 155, 198, and 227-240.

96. The method of any of embodiments 81-95, wherein the bacterial chaperone is thioredoxin A (TrxA).

97. The method of embodiment 96, wherein TrxA has the sequence set forth in SEQ ID NO:194 or a sequence of amino acids that exhibits at least 85%, 86%, 87%, 88%, 89%, 90%, 91%, 92%, 92%, 94%, 95%, 96%, 97%, 98%, 99% sequence identity to SEQ ID NO:194.

98. The method of any of embodiments 78-80, 96 and 97, wherein TrxA has the sequence set forth in SEQ ID NO:194.

99. The method of any of embodiments 78-98, wherein the binding peptide and bacterial chaperone are joined by a cleavable linker.

100. The method of any of embodiments 78-99, wherein the binding peptide is C-terminal to the bacterial chaperone.

101. The method of embodiment 99 or 100, wherein the method further comprises (d) cleaving the cleavable linker of the fusion protein, thereby producing a soluble binding peptide comprising 1-6 disulfide bonds free of the bacterial chaperone.

102. The method of any of embodiments 99-101, wherein the cleavable linker comprises a cleavage site selected from:
(i) an enterokinase cleavage site, optionally comprising the amino acid sequence set forth by DDDDK (SEQ ID NO:106);
(ii) a Factor Xa cleavage site, optionally comprising the amino acid sequence set forth by SEQ ID NO: 218 or I-E/D-G-R;
(iii) a thrombin cleavage site, optionally comprising the amino acid sequence set forth by any one of SEQ ID NOS: 219-222, more optionally set forth by SEQ ID NO: 220; or
(iv) a TEV protease cleavage site set forth by SEQ ID NO: 223 or SEQ ID NO:224.

103. The method of embodiment 101 or embodiment 102, wherein cleaving the cleavable linker comprises contacting the fusion protein with the protease that recognizes the cleavage site.

104. The method of any of embodiments 99-103, wherein the cleavable linker is a enterokinase cleavable linker comprising the amino acid sequence DDDDK (SEQ ID NO:106).

105. The method of embodiment 104, wherein the enterokinase cleavable linker comprises the sequence DYKDDDK (SEQ ID NO: 209) or GGSDYKDDDDKGS (SEQ ID NO:210).

106. The method of any of embodiments 101-105, wherein cleaving the cleavable linker comprises contacting the fusion protein with an enterokinase.

107. The method of any of embodiments 101-106, further comprising removing the bacterial chaperone from the solution comprising the soluble modified binding peptide.

108. The method of any of embodiments 101-107, further comprising removing the protease, optionally the enterokinase, from the solution comprising the soluble modified binding peptide.

109. The method of any of embodiments 78-99, wherein the binding is peptide is engineered into a loop of the bacterial chaperone.

110. The method of embodiment 109, wherein the bacterial chaperone is TrxA and the loop is selected the catalytic loop corresponding to residues 31-35 of SEQ ID NO:194, the first binding loop corresponding to residues 74-76 of SEQ ID NO:194 or the second binding loop corresponding to residues 91-93 of SEQ ID NO:194.

111. The method of embodiment 109 or embodiment 110, wherein the bacterial chaperone is TrxA and the loop is the second binding loop corresponding to residues 91-93 of SEQ ID NO:194, optionally wherein the modified binding peptide is engineered between Val-92 and Gly-93 of the sequence set forth in SEQ ID NO:194.

112. The method of any of embodiments 109-111, wherein the binding peptide is engineered into the loop between a first and second cleavable linker positioned on the N-terminus and C-terminus of the binding polypeptide, respectively.

113. The method of embodiment 112, wherein the first and second cleavable linker are the same.

114. The method of embodiment 112 or embodiment 113, wherein the first and second cleavable linker comprises a cleavage site selected from:
(i) a enterokinase cleavage site, optionally comprising the amino acid sequence set forth by DDDDK (SEQ ID NO:106);
(ii) a Factor Xa cleavage site, optionally comprising the amino acid sequence set forth by SEQ ID NO: 218 or I-E/D-G-R;
(iii) a thrombin cleavage site, optionally comprising the amino acid sequence set forth by any one of SEQ ID NOS: 219-222, more optionally set forth by SEQ ID NO: 220; or
(iv) a TEV protease cleavage site set forth by SEQ ID NO: 223 or SEQ ID NO:224.

115. A fusion protein comprising a modified binding peptide and a bacterial chaperone joined by a cleavable linker, wherein the modified binding peptide has the formula N-terminus to C-terminus: Y1-[cysteine motif binding peptide]-Y2, wherein:
the cysteine motif binding peptide is 20-50 amino acids in length in which 2-12 amino acids are cysteine residues able to form 1-6 disulfide bonds; and
Y1 and Y2 are amino acid peptide sequences that interact with one another when in proximity.

116. The fusion protein of embodiment 115, wherein Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif.

117. The fusion protein of embodiment 115 or embodiment 116, wherein the Y1 and Y2 are HW and SF, respectively.

118. The fusion protein of embodiment 115 or embodiment 116, wherein Y1 and Y2 are IS and TV, respectively.

119. The fusion protein of embodiment 115 or embodiment 116, wherein Y1 and Y2 are sequences that are each 14-30 amino acids in length comprising a heptad repeat, wherein Y1 and Y2 are able to interact to form an anti-parallel coiled-coil motif.

120. The fusion protein of embodiment 115 or 116, wherein Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203.

121. The fusion protein of embodiment 115, 116 or 120, wherein the Y1 is set forth in SEQ ID NO:202 and Y2 is set forth in SEQ ID NO:203, respectively.

122. The fusion protein of any of embodiments 115-121, wherein the cysteine motif binding peptide is a knob peptide from an ultralong CDR3 of a cow antibody.

123. The fusion protein of embodiment 122, wherein the cysteine motif binding peptide does not comprise an ascending or descending stalk domain or a contiguous portion thereof of more than 3 amino acids in length from the ultralong CDR3 of the cow antibody.

124. The fusion protein of any of embodiments 115-123, wherein the cysteine motif binding peptide is 22 to 43 amino acids in length.

125. The fusion protein of any of embodiments 115-124, wherein the cysteine motif binding peptide comprises at least 4 cysteine residues.

126. The fusion protein of any of embodiments 115-125, wherein the cysteine motif binding peptide contains 4 cysteine residues, 6 cysteine residues or 8 cysteine residues.

127. The fusion protein of any of embodiments 115-126, wherein the cysteine motif binding peptide is able to form at least 2 disulfide bonds.

128. The fusion protein of any of embodiments 115-127, wherein the cysteine motif binding peptide is able to form 2 disulfide bonds, 3 disulfide bonds or 4 disulfide bonds.

129. The fusion protein of any of embodiments 115-128, wherein the cysteine motif binding peptides binds to a target antigen that is a nonvirulent bacteria, a virus, a viral protein, an immunomodulatory protein, a cancer antigen, a human IgG, or a recombinant protein thereof.

130. The fusion protein of any of embodiments 115-129, wherein the target antigen is a coronavirus, a coronavirus pseudovirus, a recombinant coronavirus Spike protein, or a receptor-binding domain (RBD) of a coronavirus Spike protein.

131. The fusion protein of embodiment 130, wherein the coronavirus is selected from the group consisting of 229E, NL63, OC43, HKU1, MERS-CoV, SARS-CoV, and SARS-CoV2.

132. The fusion protein of embodiment 130 or embodiment 131, wherein the coronavirus is a SARS-CoV2 selected from Wuhan-Hu-1 isolate, B.1.351 South African variant, B.1.1.7 UK variant, a beta variant, a delta variant, or a omicron variant.

133. The fusion protein of any of embodiments 115-132, wherein the cysteine motif binding peptide is set forth in any one of SEQ ID NOS: 155, 198, and 227-240.

134. The fusion protein of any of embodiments 115-133, wherein the cleavable linker comprises a cleavage site selected from:
(i) a enterokinase cleavage site, optionally comprising the amino acid sequence set forth by DDDDK (SEQ ID NO:106);
(ii) a Factor Xa cleavage site, optionally comprising the amino acid sequence set forth by SEQ ID NO: 218 or I-E/D-G-R;
(iii) a thrombin cleavage site, optionally comprising the amino acid sequence set forth by any one of SEQ ID NOS: 219-222, more optionally set forth by SEQ ID NO: 220; or
(iv) a TEV protease cleavage site set forth by SEQ ID NO: 223 or SEQ ID NO:224.

135. The fusion protein of any of embodiments 115-134, wherein the cleavable linker is a enterokinase cleavable linker comprising the amino acid sequence DDDDK (SEQ ID NO:106).

136. The fusion protein of embodiment 135, wherein the enterokinase cleavable linker comprises the sequence DYKDDDK (SEQ ID NO: 209) or GGSDYKDDDDKGS (SEQ ID NO:210).

137. The fusion protein of any of embodiments 115-136, wherein the bacterial chaperone is thioredoxin A (TrxA).

138. The fusion protein of embodiment 137, wherein TrxA has the sequence set forth in SEQ ID NO:194 or a sequence of amino acids that exhibits at least 85%, 86%, 87%, 88%, 89%, 90%, 91%, 92%, 92%, 94%, 95%, 96%, 97%, 98%, 99% sequence identity to SEQ ID NO:194.

139. A soluble modified binding peptide produced by the method of any of embodiments 78-114.

140. A soluble peptide comprising a modified binding peptide that is disulfide-bonded, wherein the modified binding peptide has the formula N-terminus to C-terminus: Y1-[cysteine motif binding peptide]-Y2, wherein:
the cysteine motif binding peptide is 20-50 amino acids in length in which 2-12 amino acids are cysteine residues and wherein the soluble peptide contains 1 to 6 disulfide bonds; and
Y1 and Y2 are amino acid peptide sequences that interact with one another when in proximity.

141. The soluble peptide of embodiment 140, wherein Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif.

142. The soluble peptide of embodiment 140 or embodiment 141, wherein the Y1 and Y2 are HW and SF, respectively.

143. The soluble peptide of embodiment 140 or embodiment 141, wherein Y1 and Y2 are IS and TV, respectively.

144. The soluble peptide of embodiment 140 or embodiment 141, wherein Y1 and Y2 are sequences that are each 14-30 amino acids in length comprising a heptad repeat, wherein Y1 and Y2 are able to interact to form an anti-parallel coiled-coil motif.

145. The soluble peptide of embodiment 140 or embodiment 141, wherein Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203.

146. The soluble peptide of embodiment 140, 141 or 145, wherein the Y1 is set forth in SEQ ID NO:202 and Y2 is set forth in SEQ ID NO:203, respectively.

147. The soluble peptide of any of embodiments 140-146, wherein the cysteine motif binding peptide is a knob peptide from an ultralong CDR3 of a cow antibody.

148. The soluble peptide of embodiment 147, wherein the cysteine motif binding peptide does not comprise an ascending or descending stalk domain or a contiguous portion thereof of more than 3 amino acids in length from the ultralong CDR3 of the cow antibody.

149. The soluble peptide of any of embodiments 140-148, wherein the cysteine motif binding peptide is 22 to 43 amino acids in length.

150. The soluble peptide of any of embodiments 140-149, wherein the cysteine motif binding peptide comprises at least 4 cysteine residues.

151. The soluble peptide of any of embodiments 140-150, wherein the cysteine motif binding peptide contains 4 cysteine residues, 6 cysteine residues or 8 cysteine residues.

152. The soluble peptide of any of embodiments 140-151, wherein the soluble peptide has at least 2 disulfide bonds.

153. The soluble peptide of any of embodiments 140-152, wherein the soluble peptide has 2 disulfide bonds, 3 disulfide bonds or 4 disulfide bonds.

154. The soluble peptide of any of embodiments 140-153, wherein the soluble peptide binds to a target antigen that is a nonvirulent bacteria, a virus, a viral protein, an immunomodulatory protein, a cancer antigen, a human IgG, or a recombinant protein thereof.

155. The soluble peptide of any of embodiments 140-154, wherein the target antigen is a coronavirus, a coronavirus pseudovirus, a recombinant coronavirus Spike protein, or a receptor-binding domain (RBD) of a coronavirus Spike protein.

156. The soluble peptide of embodiment 155, wherein the coronavirus is selected from the group consisting of 229E, NL63, OC43, HKU1, MERS-CoV, SARS-CoV, and SARS-CoV2.

157. The soluble peptide of embodiment 155 or embodiment 156, wherein the coronavirus is a SARS-CoV2 selected from Wuhan-Hu-1 isolate, B.1.351 South African variant, B.1.1.7 UK variant, a beta variant, a delta variant, or a omicron variant.

158. The soluble peptide of any of embodiments 140-157, wherein the cysteine motif binding peptide is set forth in any one of SEQ ID NOS: 155, 198, and 227-240.

159. A composition comprising the soluble peptide of any of embodiments 139-158.

160. The composition of embodiment 159, that is a pharmaceutical composition comprising a pharmaceutically acceptable excipient.

161. A method of administering to a subject the modified fusion polypeptide of any of embodiments 1-24, the fusion peptide of any of embodiments 25-31, the composition of embodiment 33 or embodiment 34, the modified binding peptide of any of embodiments 77, the soluble peptide of any of embodiments 139-158, or the composition of embodiment 159 or embodiment 160 for use in treating a disease or condition.

162. The method of embodiment 161, wherein the disease or condition is a virus infection.

163. The method of embodiment 162, wherein the virus infection is infection with a coronavirus.

### EXAMPLES

The following examples are included for illustrative purposes only and are not intended to limit the scope of the invention.

### Example 1: Generation of Anti-SARS CoV-2 Antibodies

Cows were immunized with SARS CoV-2 Spike protein or receptor binding domain (RBD) portion thereof and sera was collected to assess binding activity.

### A. Spike Protein and Receptor Binding Domain Expression and Purification

SARS CoV-2 spike trimer protein from the parental Wuhan-Hu-1 isolate (NCBI YP_009724390.1) or the B.1.351 “South African” variant with the mutation E484K (and K417N and N501Y), or the parental receptor binding domain (RBD) protein (amino acids 319 to 541 of the spike protein), were produced by transfection of HEK293 cells. Approximately 120×10⁶ HEK293 Freestyle cells with 293fectin (Invitrogen) were combined with 120 μg of pCAGGS-based vector containing (1) the sequence encoding the extracellular domain of the Spike protein with furin-cleavage site removed and K986P and V987P stabilizing mutations, T4-fibritin trimerization domain and c-terminal 6×His-tag, or (2) spike RBD domain (amino acids 319 to 541 of the spike protein) with c-terminal 6×His-tag.

Cells were shaken at 37° C. for 4 days with 8% CO2 with 150 μl TCM-ProteaseArrest tissue culture protease inhibitor (G-Biosciences) added on day 3. The supernatant containing secreted spike or RBD protein was clarified from the supernatant by centrifugation at 4000 RPM for 5 minutes followed by filtration through a 0.45 m PES filter. The supernatant was concentrated and buffer-exchanged into PBS using Amicon Ultra Centrifugal Filter units (MWCO=50,000 for S protein preparation and 10,000 for the RBD protein) (EMD-Millipore) at 4° C. The concentrated supernatant was then purified using TALON cobalt metal affinity resin (Takara Bio) following the manufacturer's protocol, except that 50 mM, 100 mM, 200 mM, 300 mM and 400 mM imidazole gradient elution fractions (1 column volume of each) collected. Each elution fraction was resolved on an SDS-PAGE gel stained with InstantBlue Coomassie Protein Stain (Abcam). Fractions containing a single spike protein band or a single RBD band were pooled, buffer-exchanged into PBS as described above, and the concentration of protein quantified using Nanodrop One (Thermo Scientific) based on the extinction coefficient and molecular weight of the spike or RBD protein, respectively.

### B. Immunization Protocol

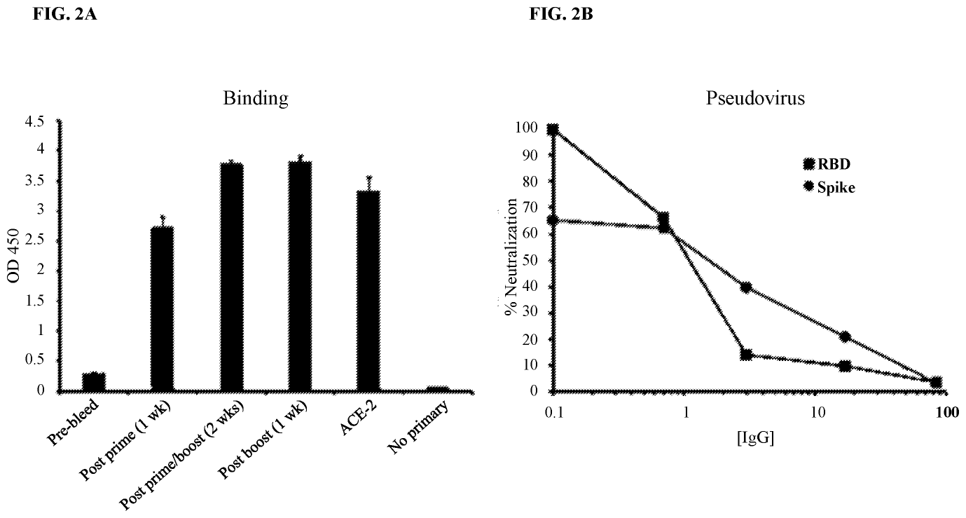

Two calves were immunized with purified Wuhan-Hu-1 spike protein or RBD protein variant with 200 μg/dose spread over 5 neck locations and boosted according to published methods (Sok et al. Nature 2017, 548(7665):108-111; Wang et al. Cell 2013, 153(6):1379-1393). Serum was collected and IgG ELISAs performed against the RBD domain of the SARS-CoV-2 spike on serum from the RBD immunized calf at a serum dilution range from 1:100 to 1:10,000. Spike protein reactivity was observed 7-21 days post-immunizations. As shown in FIG. 2A, binding activity for the RBD domain was significant after the first immunization.

Serum IgG was also assessed for neutralization of Spike protein and virus using a plaque reduction and neutralization test (PRNT). In this in vitro assay, virus and serum IgG are pre-incubated together before being concomitantly applied to permissive cells such that virus successfully bound by antibody can no longer penetrate cells and/or can no longer further propagate infection. As a result, foci of infection and cell damage called “plaques” appear to be smaller in size and/or number when the cellular monolayer is stained.

A pseudovirus expressing the SARS CoV-2 Spike protein was used as a model virus to assay percent neutralization of serum IgG from both parental Spike protein and RBD immunized cows in Vero6 cells. Compared with natural virus, the pseudovirus can be handled with BSL-2 considerations at high titer and can only infect cells in a single round. As shown in FIG. 2B, IgG obtained from cows in either of the immunization protocols was able to successfully neutralize the pseudovirus in a dose dependent manner. At higher concentrations, serum IgG (ng/mL) from cows immunized with the RBD alone was observed to neutralize 100% of pseudovirus.

Taken together, these results support that immunized cow serum, and antibodies contained therein, can neutralize SARS-CoV-2.

### Example 2: Generation of Ultralong CDR3 scFv Antibody or CDR3-Knob Only Phase Display Libraries for Antibody Discovery

Peripheral Blood Mononuclear cells (PBMCs) were collected from the immunized cows described in Example 1 and RNA was extracted to use to generate two phage display libraries as described below. Specifically, approximately 1-5×10⁷ PBMCs were collected after 14-64 days post-immunization and stored prior to RNA extraction and cDNA synthesis.

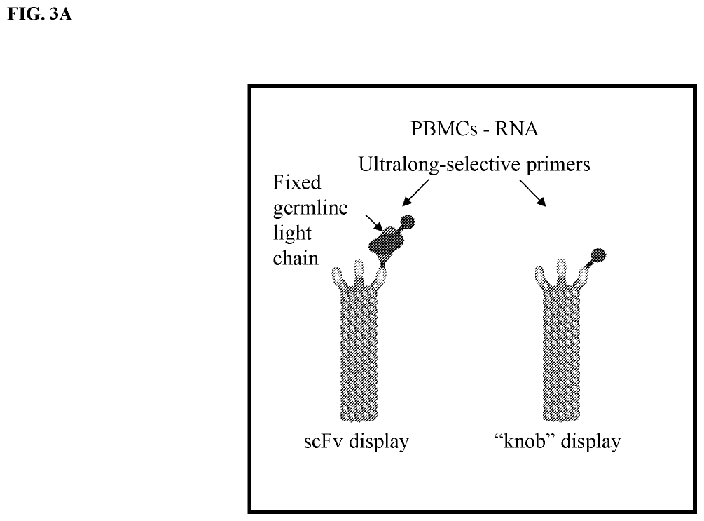

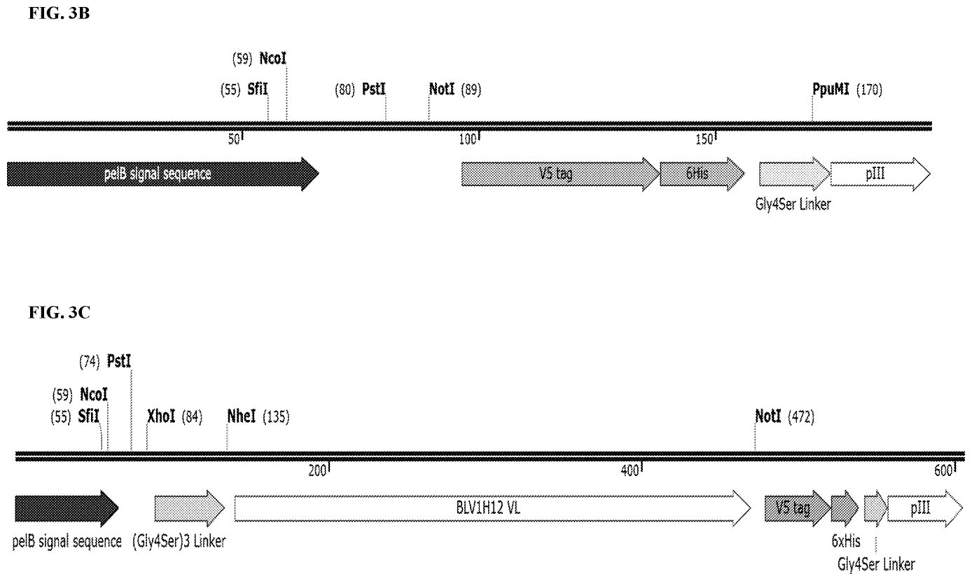

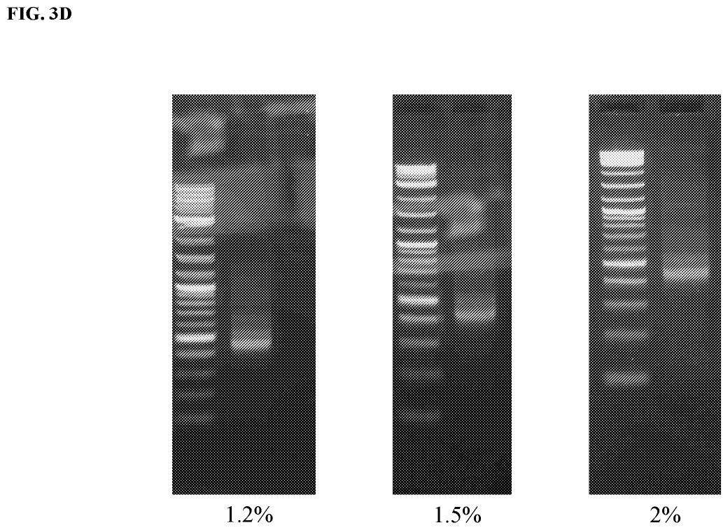

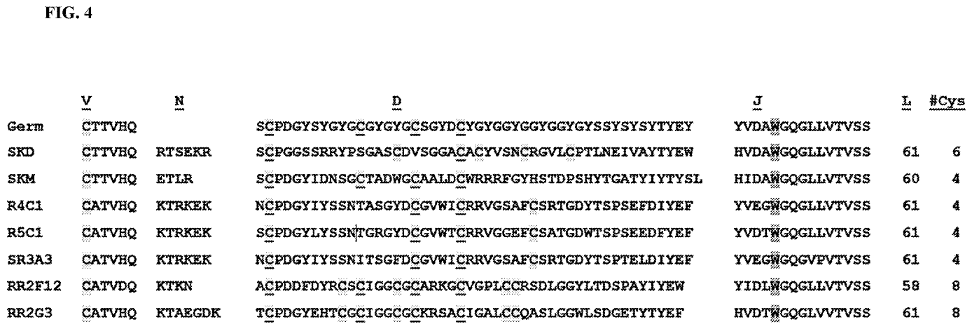

Two library strategies were employed, either using the antibodies in an scFv format with variable heavy chain (VH) and variable light chain (VL) fragments joined by a flexible linker peptide ((Gly₄Ser)₃ 15 amino acid linker, SEQ ID NO: 94), or using independent CDR3-knobs. In both approaches, the scFv or CDR3-knobs were fused to pIII via a flexible Gly₄Ser linker. FIG. 3A depicts the pIII fusion constructs in each display library. The generation of the display libraries are summarized below.

### A. ScFv Library Construction

In the first strategy, immune cow derived VH DNA fragments were combined with a fixed light chain BLV1H12 (Stanfield et al. Science immunology 2016, 1(1):aaf7962). RBD and full length spike protein immune libraries were constructed for different immunization time points.

RNA was isolated from 5×10⁶-10⁷ bovine PBMC's using an RNAeasy kit (Qiagen). Immune cow antibody VH repertoires were obtained through cDNA synthesis from 5 μg total RNA using Superscript IV First-Strand cDNA synthesis kit (ThermoFisher, #18091050), followed by PCR amplification. To generate a VH template library, the cDNA template for VHs were synthesized using a pool of IgM (SEQ ID NO: 4), IgA (SEQ ID NO: 5) and IgG-specific (SEQ ID NO: 3 and 6) primers.

In these hybrid libraries, full length donor ultra-long VHs were amplified from the VH template library with a VH family specific primer pair. Specifically, both V_{H} regions were amplified with FR1 and FR4 primers specific for the bovine IgHV1-7 family (SEQ ID NO: 12 and 13, respectively) in order to enrich for VH regions with ultralong CDR3 regions. The amplified products were combined with Linker-BLV1H12 lambda light chain variable region (BLV1H12 light chain set forth in SEQ ID NO: 2 and encoded by a DNA sequence set forth in 1) by cloning into pre-cloned pTAU1 pIII fusion phage display vector (pTAU1-BLV1H12(-VH) (see FIG. 3C). The amplified products were subjected to 2 hours digestion with NcoI and XhoI (NEB) and subcloned into pTAU1-BLV1H12(-VH) as NcoI-XhoI fragments for separation of the VH and VL by the flexible linker peptide ((Gly₄Ser)₃, SEQ ID NO: 94). In a further step, some ultra-long VH fragments were additionally enriched by separation from shorter VH fragments using agarose gel electrophoresis, prior to digestion with NcoI and XhoI restriction enzymes. As shown in FIG. 3D, a 2% agarose gel achieved the most separation between ultra-long VH fragments (˜550 base pairs in length) and shorter VH fragments without ultralong CDR3 regions (˜400 base pairs in length).

Next, this was ligated overnight with T4 DNA ligase at 16° C. Final libraries were obtained by electroporation of electrocompetent TG1 cells (Lucigen) with the purified ligation products. Each library was a minimum of 10⁷ clones with >90% with inserts.

### B. CDR3-Knob Library Construction

In a second strategy, a library of VH templates were generated substantially as described in the first strategy. Then, ultra-long VH only, immune cow derived CRD3-knob (also called “CDR3-knob only”) libraries were built by amplifying stalk-knob CDRs from the VH template library using conserved primers and cloning as pIII fusions into the pTAU1 phage display pIII fusion vector.

Specifically, RNA was isolated from 5×10⁶-10⁷ bovine PBMCs using an RNAeasy kit (Qiagen). Immune cow antibody CDR3-knob repertoires were obtained through cDNA synthesis from 5 μg total RNA using Superscript IV First-Strand cDNA synthesis kit (ThermoFisher), followed by PCR amplification. To generate the VH template library, the cDNA template for CDR3-knobs was synthesized using a pool of IgM (SEQ ID NO: 4), IgA (SEQ ID NO: 5) and IgG-specific (SEQ ID NO: 3 and 6) primers.

Primary stalk-knob CDR3 were amplified from 1^{st} strand cDNA, with IgHV1-7 family specific primers specific for either side of the stalk domain of the CDR3 region (SEQ ID NO: 7-11). These were then cloned into pTAU1 phage vector as NcoI-NotI fragments following 2 hours digestion with the NcoI and NotI (NEB), and ligated overnight with T4 DNA ligase at 16° C. (see FIG. 3B). Final libraries were obtained by electroporation of electrocompetent TG1 cells (Lucigen) with the purified ligation products. Each library was a minimum of 10⁷ clones with >90% with inserts.

### Example 3: Screening of Phage Display Libraries and Selection of Ultra-Long VH or CDR3-Knob Domains Against SARS Cov-2

The VH ultra-long CDR3 scFv antibody or CDR-knob only libraries generated as described in Example 2 were subjected to two-five rounds of phage display selections against SARS CoV-2 target proteins (both parental Wuhan Hu-1 or “South African” B.1.351 variant Spike proteins or parental Wuhan Hu-1 RBD). Spike protein from either viral isolate or parental RBD were coated onto NUNC immunotube with 1 mL of 10 μg/mL of target protein in PBS overnight at 4° C. Tubes were then blocked for 1 hour at room temperature on a blood mixer with 3-4 mL 2% Milk powder dissolved in PBS, and washed 3 times with PBS.

For each selection, approximately 10¹² phage particles from different immunized scFv or CDR3 knob libraries generated as described in Example 2 were added to 1 mL 4% milk powder dissolved in PBS, and made up to 2 mL total volume with PBS, and then added to the tubes with target protein and incubated on the blood mixer for 2 hours at room temperature. Tubes were then washed 10×PBS/0.1% Tween 20, and 10×PBS.

Bound phage were recovered with lmL fresh 0.1M triethylamine for 10 minutes on the blood mixer and neutralized with 0.5 mL 1M tris (pH 7.0) on ice. Log-phase TG1 Phage-Competent™ cells were infected with eluted phage for 1 hour at 37° C./200 rpm, and then grown at 30° C. overnight on 2×TY agar supplemented with 2% glucose/50 μg/mL carbenicillin.

After each round of selection described above, TG1 bacteria were scraped off the master plates into 20 mL 2×TY media supplemented with 20% glycerol/2% glucose/50 μg/mL carbenicillin. Approximately 4-5 mL of this solution was added to 20 mL of 2×TY media supplemented with 2% glucose/50 μg/mL carbenicillin containing 100 μl M13K07 helper phage (MOI=10). This suspension was incubated at 37° C./200 rpm for 1 hour, and added to 200 mL 2×TY/0.2M sucrose/50 μg/mL carbenicillin/25 μg/mL kanamycin/20 μm IPTG before incubating overnight at 30° C./200 rpm. Amplified phage were precipitated from cleared culture supernatants with 1/5 volume 2.5M NaCl, 20% PEG 8000 in a 250 mL Oakridge centrifuge tube after incubation on ice for 1 hour. The phage containing material was pelleted at 14,000g in a Sorvall centrifuge for 20 minutes, resuspended in 2 mL PBS, and lmL reserved for use in the next round of selection. Between 2-5 rounds of selection were carried out for each library, with phage ELISA carried out for each round beginning at Round 2.

From each selection, individual colonies were picked into 600PL 2×TY media supplemented with 50 μg/mL carbenicillin and 2% w/v glucose in 96-deepwell culture plates and incubated at 37° C. (with shaking) at 200 rpm overnight. For each culture, 50 μL was transferred to a fresh 96-deepwell plate containing 200 μL/well of the same medium and grown for 3 hours. Approximately 10⁸ kanamycin resistance units (k.r.u.) of M13K07 kanamycin-resistant helper phage was added to each well, and plates incubated at 37° C. for 1h. Expression medium (800 μL/well 2×TY media supplemented with 0.2M sucrose,100 μg/mL carbenicillin, 25 μg/mL kanamycin, and 20 μM IPTG) was added to each well and amplification continued overnight at 30° C.

Culture plates were centrifuged at 2000g for 10 mins at 4° C., and 25 L of culture supernatant per well was used for ELISA. Half-area Costar ELISA plates were coated overnight at 4° C. with 50 μL/well RBD or Spike target protein at 1 μg/mL in PBS, blocked for 1 hour at room temperature with 100 μL/well of 2% milk powder dissolved in PBS, and then washed 2×100 μL/well PBS. Approximately 25 μL phage culture supernatant per well was added to each target plate or negative control plate containing 25 μL/well 4% milk powder/PBS, and allowed to bind for 1 hour at room temperature. Each plate was washed two times with 200 μL/well PBS with 0.1% Tween 20, then two times with 200 μL/well PBS. Bound phage were detected with 50μ:/well, 1:5000 diluted anti-M13-HRP conjugate (Sinobiologicals) in 2% milk powder/PBS for 1 hour at room temperature. The plates were washed and developed for 5-10 minutes at room temperature with 50 μL/well TMB (3,3′, 5,5′-Tetramethylbenzidine) substrate buffer (Thermofisher). The reaction was stopped with 100 μL/well 0.5N H₂SO₄ per manufacture protocol and optical density read at 450 nm.

Positive clones from screening the scFv libraries were sequenced and both short and ultra-long VH sequences were transferred to the pFUSE human IgG1 Fc heavy chain expression vector for co-expression in mammalian HEK293 cells with chimeric BLV1H12 lambda light chain-human lambda light chain constant region. Positive clones from screening the knob-CDR3 only libraries were synthesized as full VH gene fragments and cloned into pFUSE human IgG1 Fc vector, and similarly expressed with the chimeric BLV1H12 lambda light chain as described above. Specifically, each V_{H} was PCR-amplified, from 10 ng phage plasmid miniprep (Qiagen), in a 50 μL reaction with 2× Phusion Hot Start II High-Fidelity PCR Master Mix (Thermo Scientific) and primers specific for V_{H} framework 1 (forward) and JH framework 4 (reverse). The PCR-generated insert was cloned into pFUSE mammalian expression vector at a 5′ EcoRI and 3′ NheI site on the 5′ end of a human IgG1 Fc gene. This was paired with a second pFUSE plasmid, containing bovine V_{L} (BLV1H12) and human λC_{L} sequences, for transfection in HEK 293F cells. Cells were seeded at a density of 1×10⁶ cells/mL in 30-60 mL Freestyle 293 Expression Medium (Gibco), then incubated in a humidified environment at 37° C. and 8% CO₂. Heavy and light chain plasmids were combined 1:1 to a total amount of 1 μg DNA per mL of 293F culture, then diluted in Opti MEM I media (Gibco) to a final volume of 1 mL per 30 mL of 293F culture. Approximately 60 L 293fectin Transfection Reagent (Gibco) and 940 L Opti MEM I were combined, for each 30 mL of 293F culture, then gently mixed and incubated for 5 minutes at room temperature before addition to diluted DNA. This mixture was incubated at room temperature for 30 minutes and then transferred to the 293F culture.

Medium was harvested 5 days after transfection and expressed chimeric bovine human IgG1 antibodies were purified by immobilized Protein A Sepharose (Cytiva Life Sciences) chromatography, then tested for antigen binding and neutralization of live and pseudovirus.

Selected candidate antibodies from the library screening were identified and sequenced (Table E1). A number of selected antibodies contained an ultralong CDR3 domain. Thus, despite ultralong CDR3 antibodies representing only about 10% of naturally occurring cow antibodies, candidate antibodies from the immunization described in Example 1 that were generated and screened by the above phage display approach were highly enriched for cow antibodies with an ultralong CDR3 (i.e., over 40% of candidates feature a CDR3 of at least 50 amino acids).

Exemplary antibodies SA-R2C3 and SA-R2D9 antibodies were derived from Ultra-long scFv library (immunization with parental Wuhan-Hu1 S protein), and identified by a screen involving selection on South African variant Spike protein. Exemplary SKM and SKD antibodies were identified from a screen from a phage library derived directly from CDR3-knob libraries as described.

Sequences alignments for exemplary ultralong antibodies SKD (SEQ ID NO: 68), SKM (SEQ ID NO: 69), R4C1 (SEQ ID NO: 70), R5C1 (SEQ ID NO: 71), SR3A3 (SEQ ID NO: 72), R2F12 (SEQ ID NO: 73), and R2G3 (SEQ ID NO: 74) are shown in FIG. 4 along

with a germline reference sequence (SEQ ID NO: 75). The length of the CDR3 and number of cysteine residues are also shown for each.

TABLE E1
Exemplary Candidate SARS CoV-2 Antibodies

| | | VH Sequence | | CDR3 Sequence | |
|---|---|---|---|---|---|
| | | Amino | Nucleic | Amino | Nucleic |
| Name | CDR3 Length | Acid | Acid | Acid | Acid |

Antibody (VH) Candidates

| | | | | | |
|---|---|---|---|---|---|
| RBD A2 | 18 | 49 | 30 | — | — |
| RBD C6 | 18 | 48 | 29 | — | — |
| RBD F4 | 18 | 47 | 28 | — | — |
| R2B1 | 25 | 44 | 25 | — | — |
| R2D6 | 25 | 43 | 24 | — | — |
| R2G1 | 20 | 42 | 23 | — | — |
| R4A10 | 31 | 41 | 22 | — | — |
| R4E5 | 31 | 39 | 20 | — | — |
| R4G3 | — | 38 | 19 | — | — |
| R4G11 | 24 | 37 | 18 | — | — |
| R5A3 | 27 | 36 | 17 | — | — |

Ultralong CDR3 Antibody (VH) Candidates

| | | | | | |
|---|---|---|---|---|---|
| R4C1 | 61 | 40 | 21 | 63 | 55 |
| R2C3 | 61 | 50 | 31 | 66 | 58 |
| SKD | 61 | 46 | 27 | 65 | 57 |
| SKM | 60 | 45 | 26 | 64 | 56 |
| R2G3 | 61 | 33 | 14 | 60 | 52 |
| R2F12 | 58 | 35 | 16 | 62 | 54 |
| SR3A3 | 61 | 34 | 15 | 61 | 53 |
| R2D9 | 52 | 51 | 32 | 67 | 59 |

### Example 4: Assessment of Binding to Spike Protein and RBD

Selected clones, expressed and purified as chimeric bovine-human IgG1 antibodies as described in Example 3, were then assayed for their ability to bind RBD and Spike protein.

### A. SARS CoV-2

RBD and spike binding of chimeric bovine-human IgG1 antibodies was assessed by ELISA. Approximately 50 μL of RBD or Spike protein, at 1 μg/mL in PBS, was added to each well of a half-area Costar ELISA plate (Corning) and coated overnight at 4° C. The plate was blocked with 180 L/well 2% milk powder/TBS/0.1% Tween20 at room temperature for 2 hours. Purified chimeric bovine-human IgG1 antibodies were diluted 5-fold from 20 nM-0.00129 nM in 2% milk powder/TBS/0.1% Tween20, and 50 L/well of each dilution was added in duplicate to coated/uncoated wells. The plate was incubated at room temperature for 1 hour, then washed four times with 180 L of TBS/0.1% Tween20, and bound IgG was detected with 50 L/well of anti-human Fc-HRP (Jackson ImmunoResearch Laboratories, Inc.) diluted 1:5000 in 2% milk powder/TBS/0.1% Tween20 at room temperature for 30 minutes. The plate was then washed five times with 180 L of TBS/0.1% Tween20 before 50 L/well of TMB (3,3′, 5,5′-Tetramethylbenzidine) substrate buffer (Thermo Scientific) was added. After 1-2 minutes at room temperature, the reaction was stopped with 50 L/well 1N H₂SO₄, and OD 450 nm values were recorded.

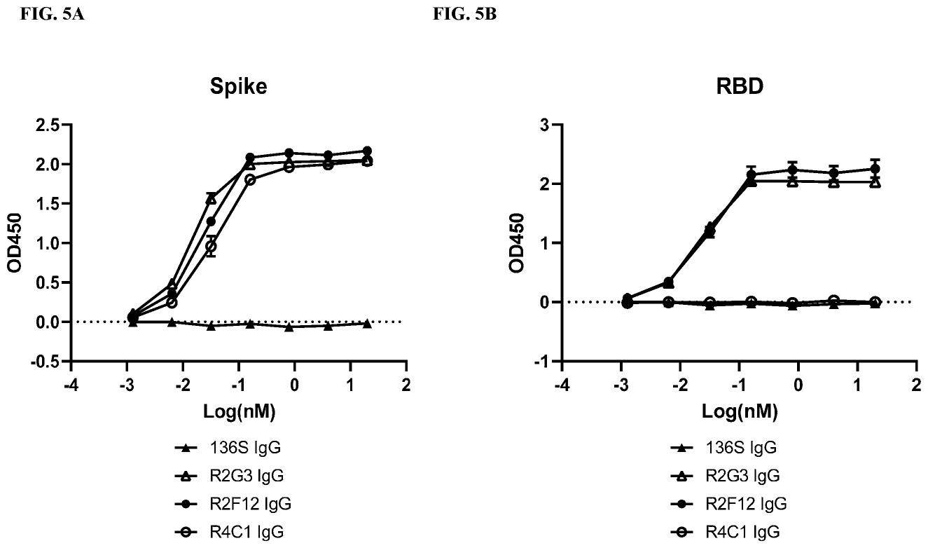

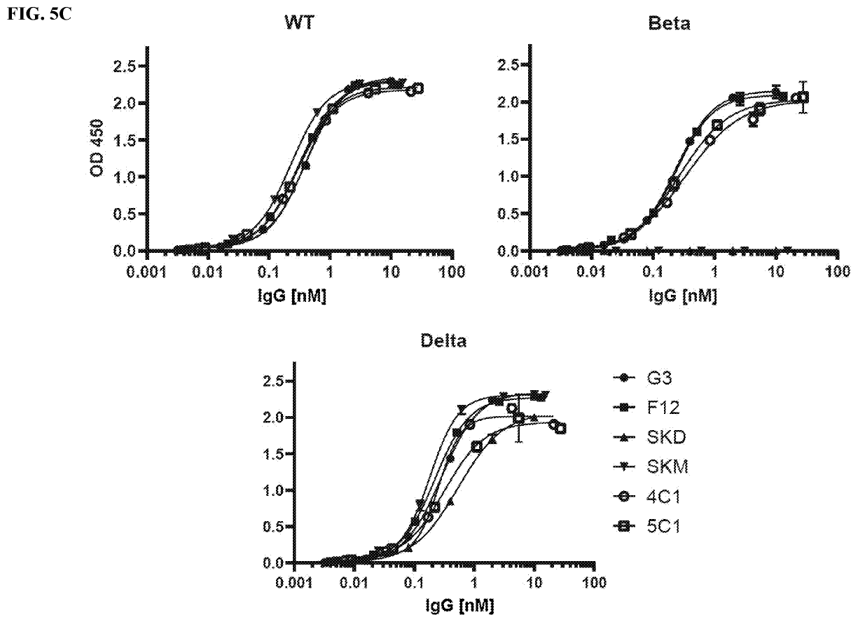

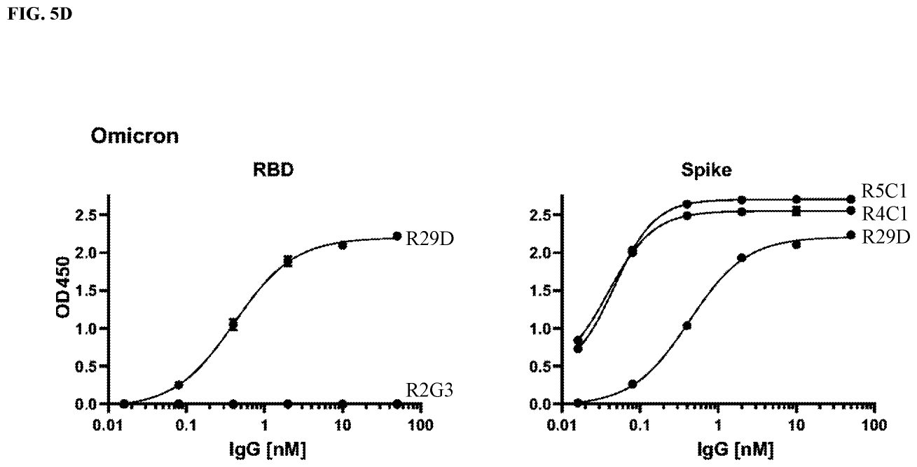

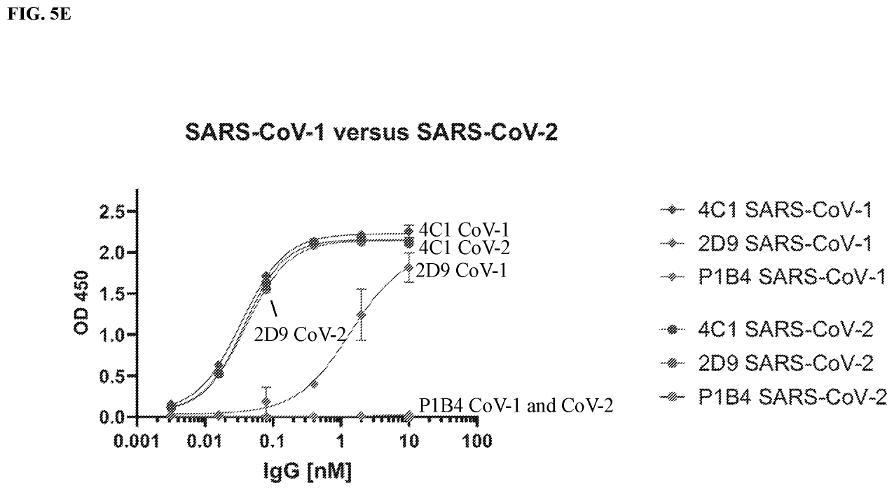

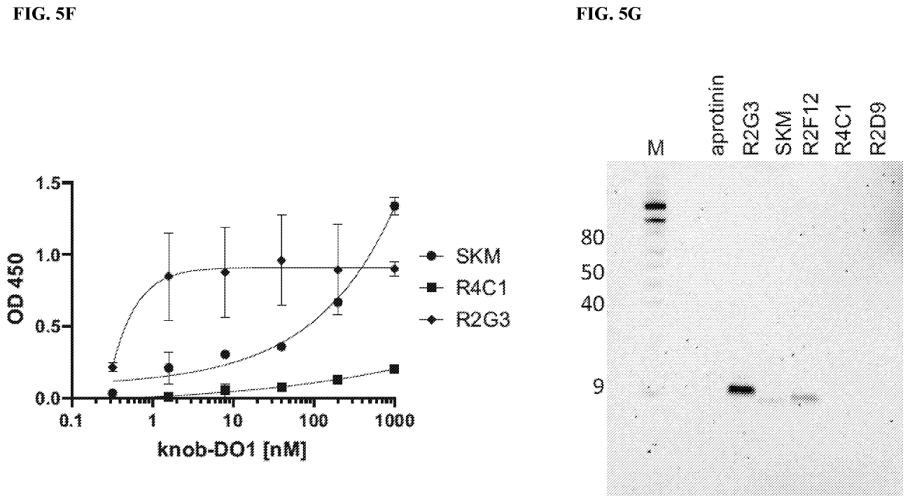

Representative results for three tested clones are shown in FIG. 5A and FIG. 5B. As shown in FIG. 5A, each of the purified chimeric bovine-human IgG1 antibodies (R2G3, R2F12, and R4C1) showed binding to the spike protein. An unrelated bovine-human IgG1 (136S IgG) did not show binding to the spike protein. As shown in FIG. 5B, purified chimeric bovine-human IgG1 antibodies with the V_{H} of clones R2G3 and R2F12 showed binding to the RBD. The unrelated bovine-human IgG1 (136S IgG), as well as the chimeric antibody with the V_{H} of clone R4C1, did not show binding to the RBD protein. These results are consistent with a finding that antibody R4C1 binds to a non-RBD epitope in the Spike protein, whereas R2G3 and R2F12 binding to a RBD epitope.

TABLE E2
Binding Activity of Exemplary Candidate SARS CoV-2 Antibodies

| | | | | |
|---|---|---|---|---|
| | | | | Spike |
| | | RBD | | Protein |
| | | Binding | Bind Spike | Binding |
| Name | Bind RBD | EC50 (nM) | Protein | EC50 (nM) |

Antibody (VH) Candidates

| | | | | |
|---|---|---|---|---|
| RBD A2 | Yes | 0.03 | Yes | 0.024 |
| RBD C6 | Yes | 0.03 | Yes | 0.03 |
| RBD F4 | Yes | 0.03 | Yes | 0.03 |
| R2B1 | Yes | 0.52 | Yes | 0.37 |
| R2D6 | Yes | 0.57 | Yes | 0.41 |

Ultralong CDR3 Antibody (VH) Candidates

| | | | | |
|---|---|---|---|---|
| R4C1 | Yes | — | Yes | 0.20 |
| R2C3 (R5C1) | Yes | — | Yes | 0.39 |
| SKD | Yes | 0.19 | Yes | 0.16 |
| SKM | Yes | 0.24 | Yes | 0.19 |
| R2G3 | Yes | 0.056 | Yes | 0.032 |
| R2F12 | Yes | 0.085 | Yes | 0.050 |
| SR3A3 | Yes | — | Yes | 0.037 |

### B. SARS CoV-2 Variants

RBD and spike binding of chimeric bovine-human IgG1 antibodies was assessed by ELISA against further isolates of SARS CoV-2, including variants from the beta, delta, and omicron lineages as well as a SARS CoV-1 virus. As described in Example 4, approximately 50 L of RBD or Spike protein, at 1 μg/ml in PBS, was added to each well and coated overnight at 4° C. The plate was blocked at room temperature for 2 hours. Purified chimeric bovine-human IgG1 antibodies were diluted 5-fold from 20 nM-0.00129 nM, and 50 μL/well of each dilution was added in duplicate to coated/uncoated wells. The plate was incubated at room temperature for 1 hour, then washed four times, and bound IgG was detected with anti-human Fc-HRP (Jackson ImmunoResearch Laboratories, Inc.). The plate was then washed five times before TMB substrate buffer was added. After 1-2 minutes at room temperature, the reaction was stopped with H2SO4, and OD 450 nm values were recorded.

FIG. 5C shows ELISA binding of IgG antibodies to recombinant stabilized spike proteins derived from the wild-type (WT) Wuhan-Hu-1 strain, beta strain (formerly described as the South African strain), or delta strain. It was observed that exemplary antibodies SKD and SKM appear to lose detectable binding to beta, but maintain binding to WT and delta SARS CoV-2. The other antibodies are shown to bind across the range of concentrations tested for each S protein.

In a complementary set of experiments performed with RBD, FIG. 5D shows ELISA binding curves of select IgG antibodies against the omicron variant RBD (left) or recombinant stabilized spike trimer (right). Of the exemplary RBD binders tested, only R2D9 was observed to maintain binding to an omicron variant spike RBD. R4C1, R5C1 and R2D9 were also observed to bind to full-length omicron spike with EC50s in the subnanomolar range.

FIG. 5E reflects exemplary ELISA data of R4C1 and R2D9 on SARS-CoV-2 compared to SARS-CoV-1. P1B4, also known as NC-Cowl, was used as a negative control, see Sok, et. al. Nature 2017. These data show that R4C1 maintains complete binding activity to SARS-CoV-1, whereas alternative exemplary antibody R2D9 loses >10× binding. However it was observed that R2D9 still maintains some binding activity in the low nanomolar range to SARS-CoV-1.

Finally, FIG. 5F shows ELISA binding activity (top) for three different exemplary antibody knob candidates against WT (Wuhan) SARS CoV-2 spike protein. For this experiment, each exemplary knob was expressed with a DO1 epitope tag, which was detected with an anti-DO1 antibody reflected on the X axis. FIG. 5G further depicts a modified western blot. Here, the indicated exemplary antibody knobs were heated to 70° C. in the presence of SDS, then resolved by SDS-PAGE before transferred to nitrocellulose membrane and detected with biotinylated RBD. RBD was biotinylated using EZ-Link NHS-LC-LC-biotin (Thermo Fisher). The NHS-LC-LC-biotin was reconstituted in DMF and combined with purified RBD at a 1:5 (RBD: biotin) molar ratio, then incubated at room temperature for 30 minutes. The reaction was then applied to a Pierce polyacrylamide spin desalting column 7K MWCO, equilibrated in PBS. A protonin was selected as a similar size control. It was observed that the R2G3 knob maintained binding to RBD despite heat and SDS treatment.

### Example 5: Virus Neutralization

In some aspects, binding of an antibody to a viral antigenic protein is insufficient to mitigate cell entry or infectious propagation. Whereas some antibodies, known as neutralizing antibodies, have the ability to inhibit virus in vitro and/or in vivo and are thus considered more relevant for therapeutic applications. Therefore, candidate antibodies as described above were tested for their ability to neutralize infection of cells with a SARS CoV-2 pseudovirus, a model virus to assay neutralization capacity of candidate antibodies. Compared with natural occurring isolates of SARS virus, the pseudovirus can be handled with BSL-2 considerations at high titer and is therefore appropriate for screening, such as in a pseudovirus luciferase assay (PVLA).

A pseudovirus expressing the SARS CoV-2 S protein of the parental Wuhan-Hu-1 Spike protein sequence in its vial envelope was engineered such that the gene for luciferase expression was carried as its cargo. Upon successful penetration into the cell, luciferase is expressed such that the pseudovirus neutralization inhibition rate is inversely proportional to luciferase activity expressed as relative light units (RLUs). These pseudotyped viruses were used in a neutralizing assay performed in CRFK-hACE2 cells. As the receptor for SARS-CoV-2 entry, ACE2 overexpression is considered a mechanism by which cell lines can be produced that display “high infectability”. In the converse, a cell line with minimal or lower ACE2 expression can be considered to display “low infectability”.

Specifically, mock-medium or serially diluted (5-fold) antibody Fab was mixed with the same amount of the pseudotyped virus carrying SARS-CoV-2 wild-type (WT) and incubated at 37° C. for 1 h. Then, the mixtures were transduced into CRFK-hACE or CRFK-hDDP4 cells in the presence of polybrene (Santa Cruz Biotech, Santa Cruz, CA) (10 μg/mL). Following incubation of the transduced cells at 37° C. for 48 h, lysis buffer was added, and the RLU were measured.

A summary of pseudovirus neutralization of identified antibodies is set forth in Table E3. The cow ultralong CDR3 antibodies are highly potent and neutralize variant strains, with a half maximal inhibition at concentrations less than 1-5 ng/mL for some antibodies. In general, the ultralong CDR3 antibodies exhibited more potent neutralization than the antibodies with a standard CDR3 length.

TABLE E3
Pseudovirus Neutralization of Exemplary
Candidate SARS CoV-2 Antibodies

| Name | IC50 (ng/mL) |
|---|---|
| RBD A2 | 69 |
| RBD C6 | 278 |
| RBD F4 | 39.7 |
| R4C1 | 520 |
| R2C3 (R5C1) | 2.12-3.2 |
| SKD | 0.05-0.33 |
| SKM | 0.07-0.29 |
| R2G3 | >1 |
| R2F12 | 0.15-2 |
| SR3A3 | 1.45-160 |

### Example 6: Bacterial Expression and Purification of CDR3-Knob Only Antibodies

A system was developed to express and purify CDR3-knobs, which are small peptide sequences of 20-50 amino acids with 1-6 disulfide bonds derived from an ultralong CDR3 cow antibody as described above. The expression system included fusion with the bacterial chaperone TrxA (UniProt POAA25; SEQ ID NO:193 and encoding the protein set forth in SEQ ID NO:194). CDR3-knobs as well as trxA-CDR-knob fusions were tested for spike and RBD binding.

### A. TrxA-CDR3-Knob Fusion and CDR3-Knob Expression and Purification

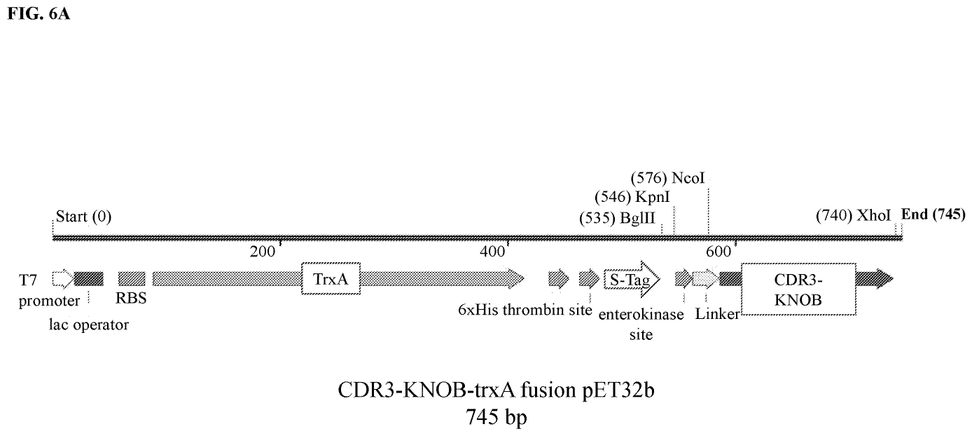

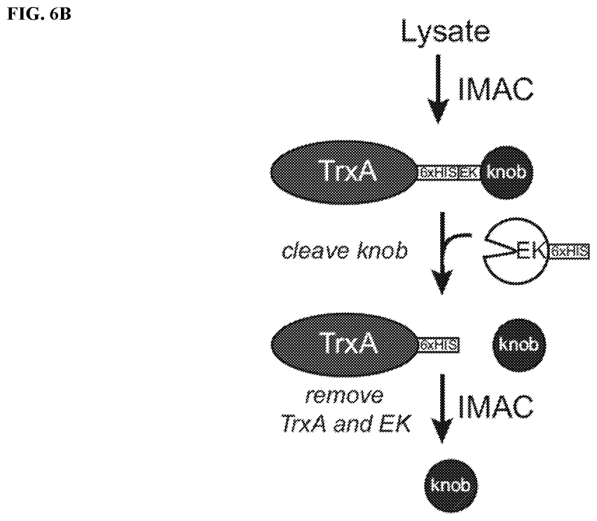

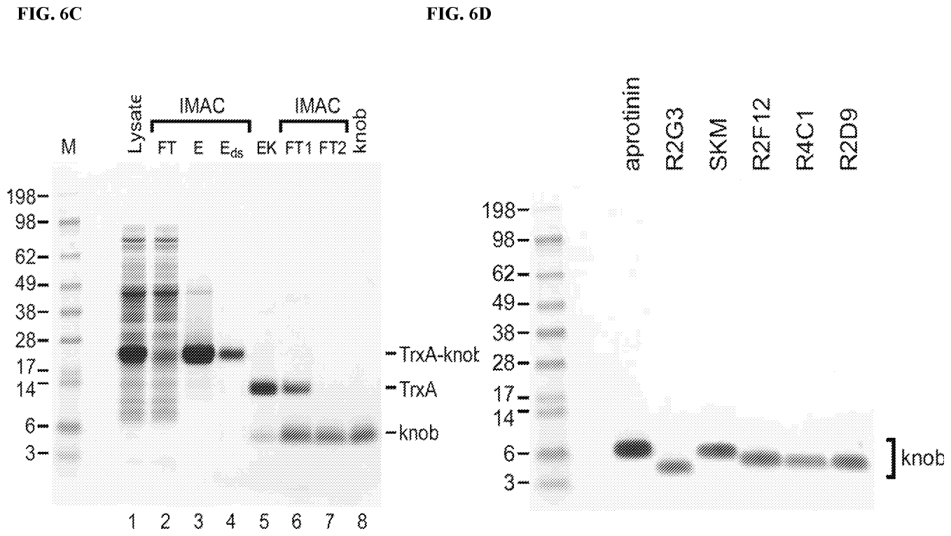

CDR3-knobs from candidate ultralong CDR3 antibodies described in Examples 2-5 were cloned into pET32b vectors (EMD-Millipore) as KpnI-XhoI (or NcoI-XhoI as appropriate) fragments (FIG. 6A), and transformed into Origami 2 DE3 bacteria, and expressed as described below. These CDR3-knobs had sequences set forth in SEQ ID NO: 60-67, and encoded by a DNA sequence set forth in SEQ ID NO: 52-59, respectively.

A trxA-CDR3-knob fusion clone was grown overnight at 37° C. in 20 mL of 2×TY/50 μg/mL carbenicillin/10 μg/mL tetracycline/2% glucose, transferred to 200 mL of the same medium, and grown at 37° C. to an OD600 nm of approximately 1.0, after which the bacteria were spun down and resuspended in 200 mL of 2×TY/50 μg/mL carbenicillin/0.5 mM IPTG and grown overnight at 22° C. The bacteria were again pelleted, resuspended in 10 mL of Bugbuster HT (EMD-Millipore), rotated for 30 minutes at room temperature, and debris pelleted for 20 minutes at 14,000 μg at 4° C. The supernatant was added to an equilibrated Talon resin column (1 mL resin TaKaRa), rotated at 4° C. for 2 hours, washed with five column volumes wash buffer (5 mM imidazole), then 1 column volume wash buffer (10 mM imidazole), eluted with 2.5 mL of 300 mM imidazole elution buffer, and then buffer exchanged to PBS/saline with a PD10 spin column (GE Healthcare). The trxA-CDR3-knob was adjusted to 50 mM Tris pH 7.4, 150 mM NaCl, and 2.5 mM CaCl₂) (lx enterokinase (EK) reaction buffer), and 400u recombinant his-tagged Enterokinase (Genscript) was added and incubated overnight at room temperature. Digested trxA and enterokinase were removed by incubation on a fresh equilibrated Talon resin column (1.2 mL resin) for 2 hours at 4° C., and purified CDR-knob was collected in the flowthrough. Again, the sample was buffer exchanged to saline/PBS. In some cases, endotoxin removal may be carried out by anion exchange chromatography prior to use or testing, such as testing in a viral neutralization assay. CDR3-knobs cloned and expressed in E. coli as independent domains are set forth in SEQ ID NO: 60-67.

The stepwise purification is depicted in FIG. 6B. As shown in FIG. 6C, stepwise purification, as monitored by SDS-PAGE, efficiently purified both trxA-CDR3-knob fuion proteins as well as soluble CDR3-knobs from E. coli lysates. FIG. 6D depicts an exemplary SDS-PAGE gel of several purified ultralong CDR H3 knob peptides. The samples were treated with reducing agent DTT, which in some aspects is sufficient to break disulfide bonds. The similarly sized protein aprotinin was included as a size control.

IMAC-purified trxA-CDR3-knob fusion Spike or RBD binding

In order to assess CDR3-knob binding as trxA fusions, prior to enterokinase cleavage from trxA, half-area Costar ELISA plates were coated overnight at 4° C. with serial dilutions of IMAC purified trxA-knob fusions from 25 μL of trxA fusion in 50 μl/well PBS. RBD-binding clones R2G3, R2F12, SKM, and SKD (nucleic acid sequences set forth in SEQ ID NO: 52, SEQ ID NO: 54, SEQ ID NO: 56, and SEQ ID NO: 57, respectively; and amino acid sequences set forth in SEQ ID NO: 60, SEQ ID NO: 62, SEQ ID NO: 64, and SEQ ID NO: 65, respectively), and spike-binding clone R4C1 (nucleic acid sequence set forth in SEQ ID NO: 55, and amino acid sequence set forth in SEQ ID NO: 63), were tested.

Plates were then blocked for 1 hour at room temperature with 100 μL/well of 2% milk powder/PBS, and then washed twice with 100 μL/well of PBS. Approximately 50 μL/well of 1 μg/mL Wuhan-Hu-1 spike protein in 2% milk powder/PBS was incubated for 1 hour, and wells were then washed three times with 100 μl/well of PBS. To detect bound spike protein, 1 μg/mL of full length IgG chimeric ultralong CDR3 was added, either anti-RBD R2G3 IgG1 (for R4C1), or anti-R4C1 IgG1 antibody (for R2F12, R2G3, SKD and SKM fusions), in 2% milk powder/PBS, incubated for 1 hour, and then wells were washed three times with 100 μL/well of PBS. Bound IgG was then detected by incubation with 1:5000 diluted anti-human IgG-Fc-HRP conjugate in 2% milk powder/PBS for 1 hour, and wells were then washed three times with 100 μL/well of PBS. The plate was then washed and developed for 5-10 minutes at room temperature with 50 μL/well TMB (3,3′, 5,5′-Tetramethylbenzidine) substrate buffer (Thermofisher). The reaction was stopped with 100 μL/well of 0.5N H₂SO₄ and read at 450 nm.

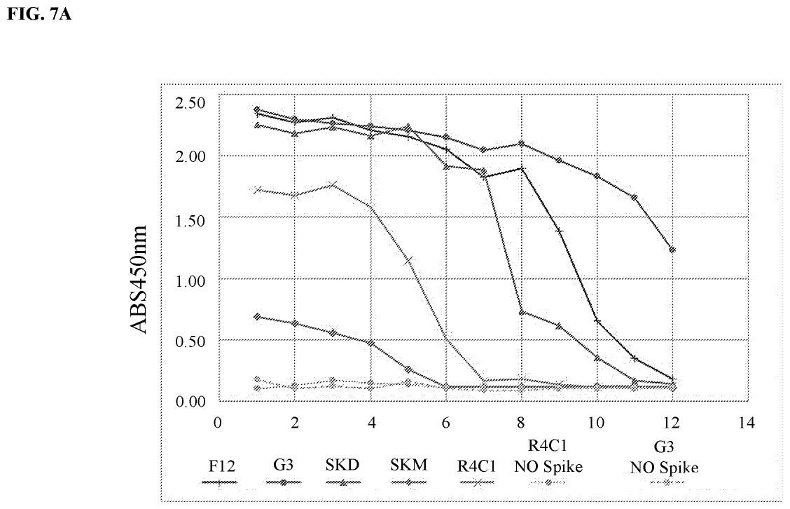

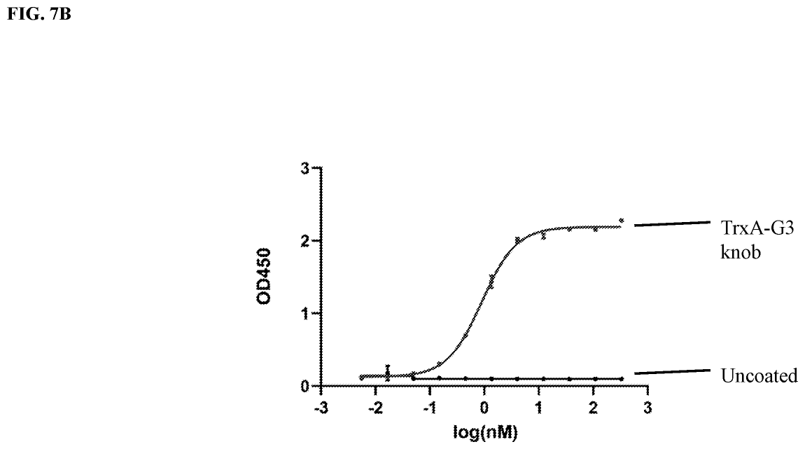

As shown in FIG. 7A (in which R2F12 is denoted as “F12”, and R2G3 is denoted as “G3”), the tested trxA-knob fusion proteins showed spike protein binding. Control conditions in which fusion proteins R3C1 and R2G3 were incubated in the absence of spike protein (denoted “R3C1 NO Spike” and “G3 NO Spike”) did not show binding. Binding for the TrxA-R2G3 fusion protein is also shown separately in FIG. 7B, relative to uncoated plates.

### B. Purified R2G3 CDR3-Knob Binding to Wuhan-Hu-1 RBD

Binding of purified R2G3 CDR3-knob (after enterokinase cleavage from trxA as described above) to RBD was evaluated by ELISA. The nucleic acid sequence encoding R2G3 CDR3-knob is set forth in SEQ ID NO: 52, and the amino acid sequence set forth in SEQ ID NO: 60.

Wells in a half-area Costar ELISA plate (Corning) were coated, in duplicate, with 50 L/well of purified CDR3-knob diluted 2-fold from 84-0.082031 nM in PBS. The plate was incubated at 37° C. for 1 hour, then blocked with 180 L/well of 2% milk powder/TBS/0.1% Tween20 at room temperature for 2 hours. Next, biotinylated RBD was diluted to 0.5 ng/L in 2% milk/TBS/0.1% Tween20, and 50 L/well was added to coated/uncoated wells. After 1 hour at room temperature, wells were washed four times with 180 L/well of TBS/0.1% Tween20, and bound biotinylated RBD was detected with 50 L/well of streptavidin-HRP (Invitrogen) diluted 1:5000 in 2% milk/TBS/0.1% Tween20 for 30 minutes at room temperature. The wells were then washed five times with 180 L/well TBS/0.1% Tween20 before addition of 50 L/well TMB (3,3′, 5,5′-Tetramethylbenzidine) substrate buffer (Thermo Scientific). After 1-2 minutes at room temperature, the reaction was stopped with 50 L/well 1N H₂SO₄, and OD 450 nm values were recorded. The average OD450 of uncoated wells was subtracted from the OD450 in each coated well. Background-subtracted OD450 values were plotted in GraphPad Prism (GraphPad Software LLC) against Log(CDR3-knob nM).

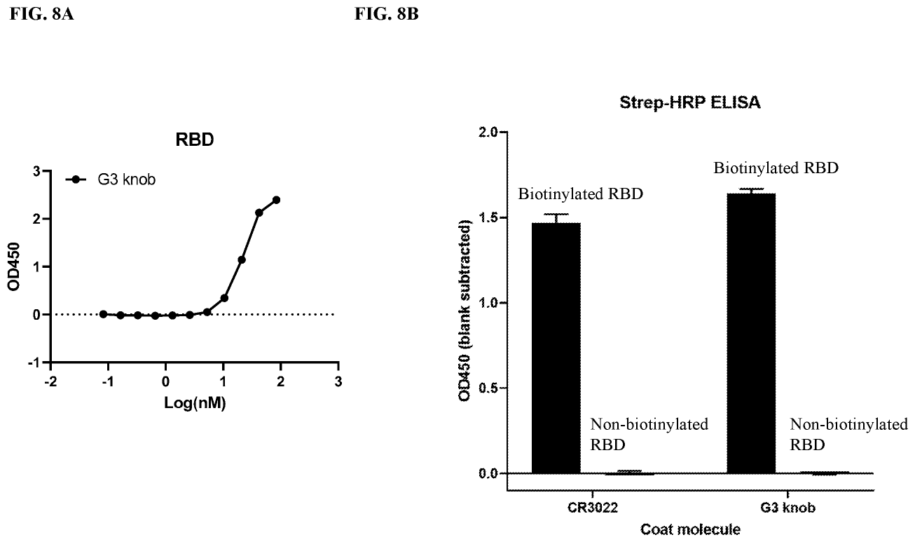

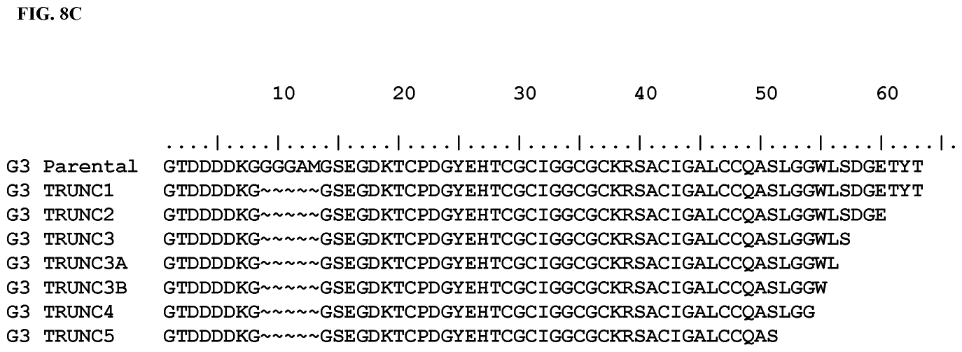

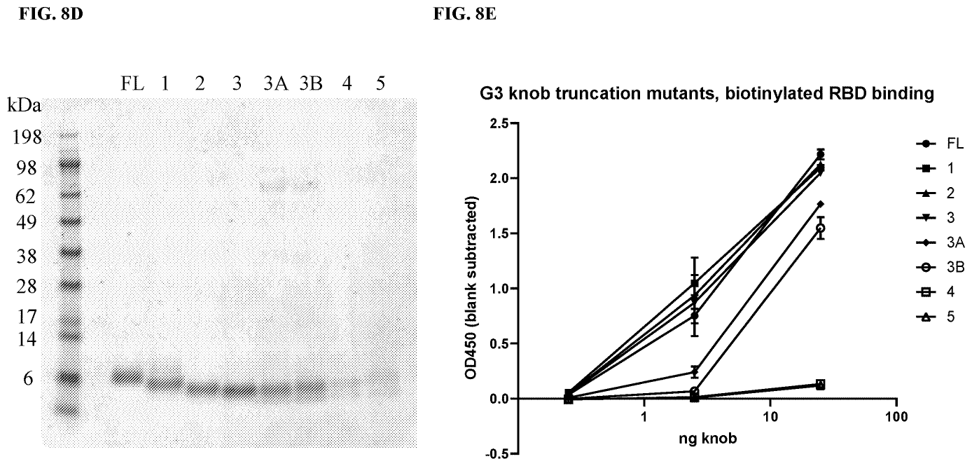

As shown in FIG. 8A, the soluble R2G3 knob showed binding to the RBD. As shown in FIG. 8B, soluble R2G3 knob binding was increased relative to that of a reference anti-spike protein antibody, CR3022.

### C. Binding of Truncated R2G3 CDR3-Knobs to Wuhan-Hu-1 RBD

Truncated R2G3 CDR3-knobs were cloned and produced as described above using pET32b vectors encoding an R2G3 truncated mutant followed by an enterokinase cleavage site. Amino acid sequences of the truncated R2G3 mutants are shown in FIG. 8C. As shown in FIG. 8D, Truncations 1-3 showed compact bands following enterokinase cleavage and gel electrophoresis (0.75 μg of truncated knob protein per lane, 250 mM DTT).

The truncated R2G3 CDR3-knobs were also tested for RBD binding as described above. As shown in FIG. 8E, Truncations 1-3 had preserved RBD binding ability, whereas Truncations 4 and 5 lacked RBD binding.

### D. Defining the Minimal CDR3-Knob C-Terminal Requirement

In order to define the C-terminal requirements (i.e., C-terminal minimal sequence) of a prototypical CDR3-knob, a series of R2G3 truncations were cloned into pET32b and expressed and purified as described in Example 6 above. These truncations were as set forth in Table E4 below.

TABLE E4
Exemplary R2G3 Truncations

| CLONE | Mature amino acid sequence after Enterokinase cleavage |
|---|---|
| G3 Parental | GGGGAMGSEGDKTCPDGYEHTCGCIGGCGCKRSACIGALCCQASLGG |
| SEQ ID ON: | WLSDGETYT |
| 86 | |
| G3 TRUNC1 | ~~~~~GGSEGDKTCPDGYEHTCGCIGGCGCKRSACIGALCCQASLGGWL |
| SEQ ID ON: | SDGETYT |
| 87 | |
| G3 TRUNC2 | ~~~~~GGSEGDKTCPDGYEHTCGCIGGCGCKRSACIGALCCQASLGGWL |
| SEQ ID ON: | SDGE |
| 88 | |
| G3 TRUNC3 | ~~~~~GGSEGDKTCPDGYEHTCGCIGGCGCKRSACIGALCCQASLGGWL |
| SEQ ID ON: | S |
| 89 | |
| G3 TRUNC3A | ~~~~~GGSEGDKTCPDGYEHTCGCIGGCGCKRSACIGALCCQASLGGWL |
| SEQ ID ON: | |
| 90 | |
| G3 TRUNC3B | ~~~~~GGSEGDKTCPDGYEHTCGCIGGCGCKRSACIGALCCQASLGGW |
| SEQ ID ON: | |
| 91 | |
| G3 TRUNC4 | ~~~~~GGSEGDKTCPDGYEHTCGCIGGCGCKRSACIGALCCQASLGG |
| SEQ ID ON: | |
| 92 | |
| G3 TRUNC5 | ~~~~~GGSEGDKTCPDGYEHTCGCIGGCGCKRSACIGALCCQAS |
| SEQ ID ON: | |
| 93 | |

The quality of expressed material was assessed by SDS-PAGE and RBD ELISA as described in Example 6D above. Only Truncations 4 (G3 TRUNC4) and 5 (G3 TRUNC5) were observed to exhibit no RBD binding capability. Truncations 3A (G3 TRUNC3A) and 3B (G3 TRUNC3B) demonstrated reduced binding in an ELISA and increased band diffuseness in SDS-PAGE as depicted in FIG. 8A. ELISAs performed with truncations 1-3 yielded no observed loss in binding activity relative to parental R2G3 CDR3-knob as shown in FIG. 8B. These data support that a minimum of at least 9 amino acids is required after the last non-canonical Cys residue for R2G3 binding.

### E. CDR3-Knob Purification by Size Exclusion Chromatography

Size exclusion chromatography (SEC) was used to resolve if soluble CDR3-knobs that were purified following bacterial expression were present in multiple forms. Soluble R4C1 and R2G3 knobs were produced as described above and subjected to SEC.

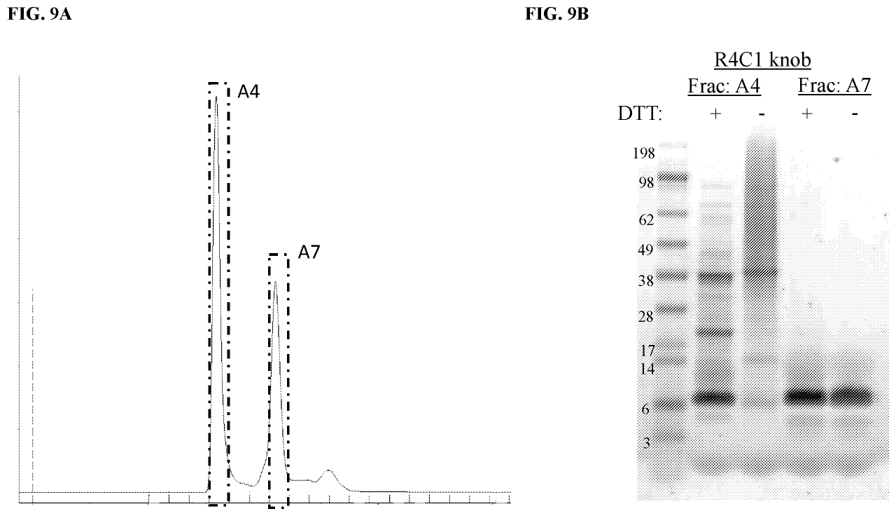

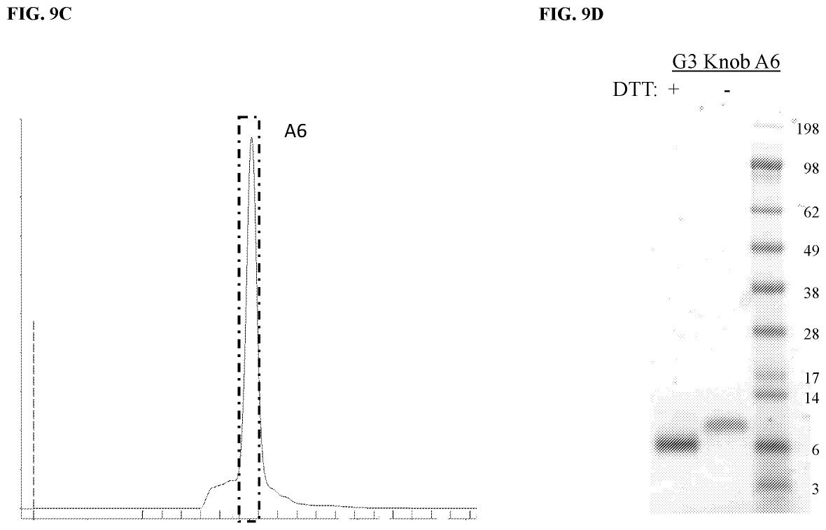

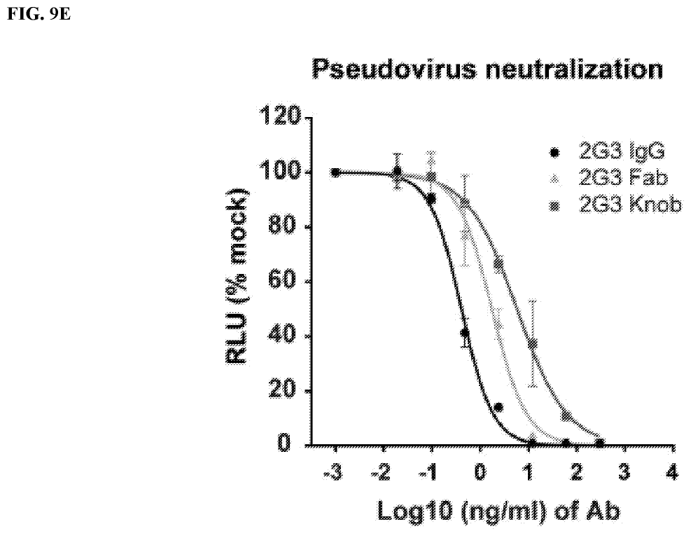

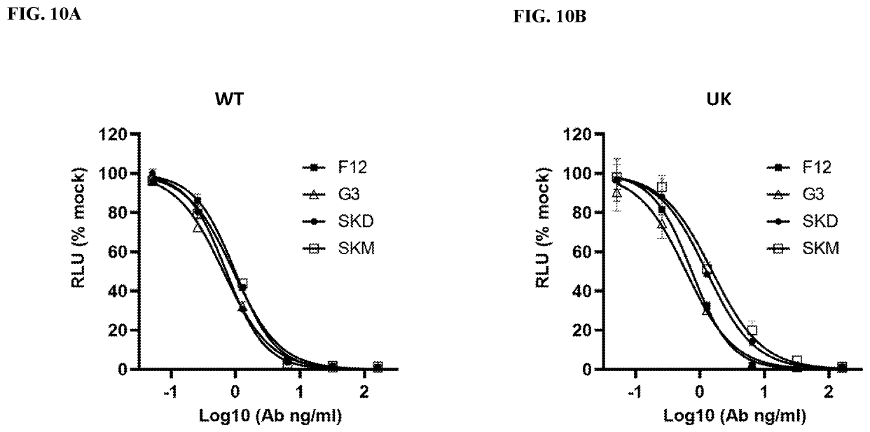

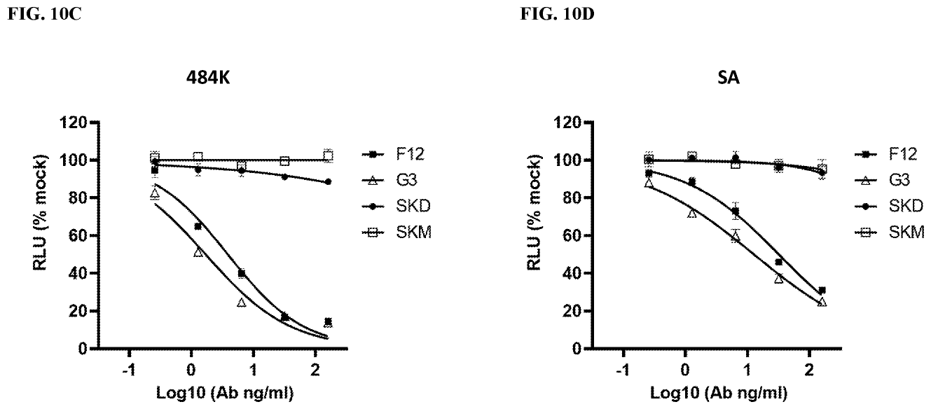

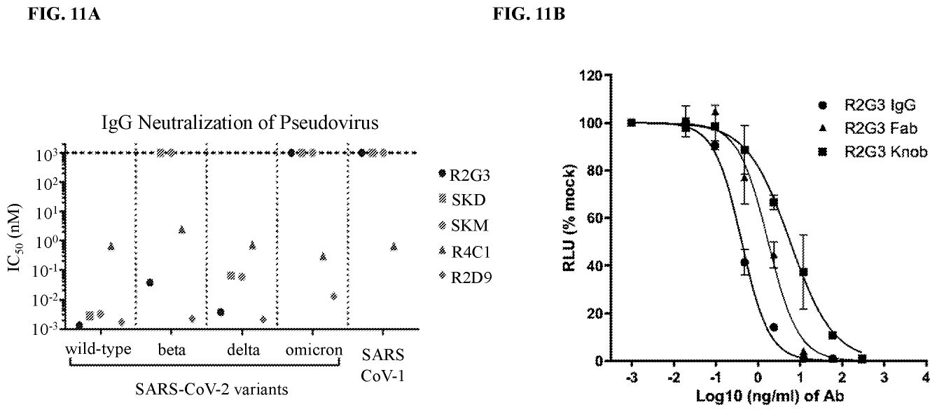

As shown in FIG. 9A, SEC revealed at least two distinct elution fractions (fractions A4 and A7) for purified R4C1 knobs, indicating that purified R4C1 knobs were present in multiple forms following bacterial expression. Gel electrophoresis was performed on fractions A4 and A7. As shown in FIG. 9B, fraction A4 contained a larger soluble aggregate as well as smaller, active soluble CDR3-knobs. Fraction A7 contained only the smaller, active soluble CDR3-knobs.

As shown in FIG. 9C, SEC revealed only one distinct elution fraction (fraction A6) for purified R2G3 knobs (fraction A6). This result was corroborated by gel electrophoresis performed on fraction A6 (FIG. 9D).

### Example 7: Comparison of SARS-CoV 2 Virus Neutralization of Chimeric Fab Ultralong CDR3 and CDR3-Knob

To assess virus neutralization of a CDR3-knob only antibody, assays to assess neutralization of pseudovirus or live WT SARS-CoV2 virus were carried out. In this example, purified R2G3 CDR3-knob (“G3-Knob”) or a Fab of the chimeric R2G3 ultralong CDR3 antibody (“G3-Fab”), or a full length IgG chimeric R2G3 ultralong CDR3 antibody (“G3”) were tested, as indicated.

A pseudovirus luciferase assay (PLSA) substantially as described in Example 5 was performed. Virus neutralization was assessed against pseudotyped virus carrying SARS-CoV-2 (Wuhan-Hu-1) wild-type (WT) spike protein, or the S variants (E484K/N507Y; B.1.1.7 or “UK” variant; and K417N/E484K/N501Y; B.1.351 or “SA” variant). Mock-medium or serially diluted (5-fold) antibody G3-Knob, G3-Fab or G3 was mixed with the same amount of the pseudotyped virus carrying SARS-CoV-2 wild-type (WT), the S variants (484K, B.1.1.7 and B.1.351) and incubated at 37° C. for 1 h. Then, the mixtures were transduced into CRFK-hACE or CRFK-hDDP4 cells in the presence of polybrene (Santa Cruz Biotech, Santa Cruz, CA) (10 μg/mL). As the receptor for SARS-CoV-2 entry, ACE2 overexpression is considered a mechanism by which cell lines can be produced that display “high infectability”. In the converse, a cell line with minimal or lower ACE2 expression can be considered to display “low infectability”.

Following incubation of the transduced cells at 37° C. for 48 h, lysis buffer was added, and the RLU were measured. Inhibition curves of serial dilutions of each antibody, G3-Fab or G3-Knob, against mock treatment were generated, and the 50% effective concentration (EC50) values were determined by GraphPad Prism software using a variable slope (GraphPad, La Jolla, CA). The results are summarized in Table E5.

To assess neutralizing activity against live SARS-CoV-2, selected antibodies of G3, G3-Fab or G3-Knob were investigated for their neutralizing activity against the replication of SARS-CoV-2 or B.1.17 or B.1.351 variants in Vero E6 cells. Briefly, 50-100 plaque forming units of SARS-CoV-2 hCoV/USA-WA1/2020 (wild type), SARS-CoV-2 hCoV-19/England/204820464/2020 (B.1.1.7 variant), or SARS-CoV-2 hCoV-19/South Africa/KRISP-EC-K005321/2020 (B.1.351 variants) were mixed with mock-medium or serially diluted (5-fold) G3-Fab or G3-Knob. Following incubation at 37° C. for 1 h, the mixtures were inoculated to confluent Vero E6 cells in 24 well plates. After 2 hr incubation, medium containing agar (1% final concentration) and neutral red was added to the cells. After 48-72 hr, plaques in each well were counted. The EC50 values were determined as described above and shown in Table E5 below.

In a similar study, a pseudovirus infection assay was carried out on parental SARS-CoV-2 pseudovirus comparing 2G3 IgG, Fab fragment, and knob. As shown in FIG. 9E, the Fab reproducibly had 3-5× greater potency than the knob fragment.

Together, the results shown in Table E5 and FIG. 9E demonstrate that the exemplary cow ultralong CDR3 R2G3, in either a standard IgG Fab format or as a CDR3-knob only format, exhibited potent neutralizing activity against WT SARS-CoV-2 as well as the tested variants. The cow ultralong CDR3 antibody is highly potent and neutralizes variant strains, with a half maximal inhibition at concentrations less than 1-5 ng/mL, depending on the antibody format. Remarkably, despite being a short sequence of only 51 amino acids in length, the CDR3-knob only antibody retained subnanomolar potency. Due to the small size of the CDR3-knob antibodies, this example supports utility of the CDR3-knob antibodies as novel therapeutic antibody candidates for an inhalation formulation for respiratory targets, including other viruses, bacteria, other infectious diseases, asthma or lung cancer.

TABLE E5
Neutralization pseudovirus and live virus for R2-G3 IgG, Fab and CDR3-knob

| | Pseudotype | | Pseudotype | | SARS-CoV-2 VeroE6 | | | SARS-CoV-2 VeroE6 | | |
|---|---|---|---|---|---|---|---|---|---|---|
| | EC50 (ng/mL) | | EC50 (pM) | | EC50 (ng/ml) | | | EC50 (pM) | | |
| | | SA | | SA | | UK | SA | | UK | SA |
| | WT | variant | WT | variant | WT | variant | variant | WT | variant | variant |
| G3 | 0.59 | 5.28 | 3.55 | 32.2 | 0.5 | 0.51 | 2.6 | 3.15 | 3.2 | 19.14 |
| G3 | 1.23 | 1.19 | 22.8 | 22.39 | 3.67 | 1.35 | 5.03 | 72.58 | 26.19 | 103.31 |
| Fab | | | | | | | | | | |
| G3 | 5.49 | 52.23 | 904.5 | 8830.1 | 0.92 | 1.94 | 333.28 | 712 | 535 | 49,968 |
| Knob | | | | | | | | | | |

### A. SARS CoV-2 Variants

In a further assessment of virus neutralization of ultralong CDR3 antibodies, assays to assess neutralization of live WT SARS-CoV2 virus or several variant SARS CoV-2 viruses were carried out. In this example, full length IgG chimeric ultralong CDR3 antibodies F12, G3, SKD, and SKM were tested, as indicated.

A pseudovirus luciferase assay (PLSA) substantially as described in Example 5 was performed. Virus neutralization was assessed against pseudotyped virus carrying SARS-CoV-2 (Wuhan-Hu-1) wild-type (WT) spike protein, the S variants (E484K/N507Y; B.1.1.7 or “UK” variant; and K417N/E484K/N501Y; B.1.351 or “SA” variant) or 484K. Mock-medium or serially diluted (5-fold) antibody was mixed with the same amount of the pseudotyped virus carrying SARS-CoV-2 wild-type (WT), the S variants (484K, B.1.1.7 and B.1.351) and incubated at 37° C. for 1 h. Then, the mixtures were transduced into Vero, CRFK-hACE or CRFK-hDDP4 cells in the presence of polybrene (Santa Cruz Biotech, Santa Cruz, CA) (10 μg/mL). As the receptor for SARS-CoV-2 entry, ACE2 overexpression is considered a mechanism by which cell lines can be produced that display “high infectability”. In the converse, a cell line with minimal or lower ACE2 expression can be considered to display “low infectability”.

Following incubation of the transduced cells at 37° C. for 48 h, lysis buffer was added, and the RLU were measured. As shown in FIG. 10A-10D, each exemplary ultralong CDR3 antibody exhibited activity against more than one variant SARS CoV-2 S protein. Inhibition curves of serial dilutions of each antibody against mock treatment were generated, and the 50% effective concentration (EC50) values were determined by GraphPad Prism software using a variable slope (GraphPad, La Jolla, CA). The results are summarized in Table E6.

TABLE E6
Neutralization pseudovirus and live virus
for Exemplary Ultralong CDR3 Antibodies

EC₅₀ (pM)

| | WT | UK | 484K | SA |
|---|---|---|---|---|
| F12 | 6.15 | 4.64 | 24.19 | 200.85 |
| G3 | 4.03 | 3.79 | 10.60 | 80.235 |
| SKD | 4.47 | 7.71 | >1000 | >1000 |
| SKM | 5.77 | 9.74 | >1000 | >1000 |

Together, the results shown in Table E5 demonstrate that the exemplary cow ultralong CDR3 antibodies, F12, G3, SKD, and SKM, exhibited potent neutralizing activity against WT SARS-CoV-2 as well as the tested variants. The cow ultralong CDR3 antibody is highly potent and neutralizes variant strains, with a half maximal inhibition at concentrations less than 1-5 ng/mL, depending on the antibody format. Remarkably, despite being a short sequence of only 51 amino acids in length, the CDR3-knob only antibody retained subnanomolar potency. Due to the small size of the CDR3-knob antibodies, this examples supports utility of the CDR3-knob antibodies as novel therapeutic antibody candidates for an inhalation formulation for respiratory targets, including other viruses, bacteria, other infectious diseases, asthma or lung cancer.

### Example 8: SARS CoV-1 Cross Reactivity

To assess possible cross reactivity and broad neutralization of exemplary Ultralong CDR3 antibodies, assays to assess neutralization of pseudovirus were carried out. In this example, exemplary R4C1 and R2D9 ultralong CDR3 antibodies were tested, as indicated.

A pseudovirus luciferase assay (PLSA) substantially as described in Example 5 was performed. Virus neutralization was assessed against pseudotyped virus carrying SARS-CoV-2 (Wuhan-Hu-1) wild-type (WT) spike protein, the S protein of a SARS-CoV-1 virus, or a VSV-G control. Mock-medium or serially diluted (5-fold) antibody G3-Knob, G3-Fab or G3 was mixed with the same amount of the pseudotyped virus carrying SARS-CoV-2 wild-type (WT), SARS-CoV-1 wild-type, or VSV-G, and incubated at 37° C. for 1 h. Then, the mixtures were transduced into cells in the presence of polybrene (Santa Cruz Biotech, Santa Cruz, CA) (10 μg/mL).

Following incubation of the transduced cells at 37° C. for 48 h, lysis buffer was added, and percent neutralization were measured. Inhibition curves of serial dilutions of each antibody against mock treatment were generated, and the maximum percent neutralization (MPN), i.e. the percent at which the neutralization curve plateaus for those viruses neutralized, were determined by GraphPad Prism software using a variable slope (GraphPad, La Jolla, CA).

FIG. 11A shows the IC50 values of different IgG antibodies against pseudoviruses from various coronavirus strains. Note that R4C1 and R2D9 maintain activity against the omicron variant of SARS-CoV-2. All of the antibodies exhibit subnanomolar potency, with several in the low picomolar range.

### Example 9: Neutralization of Live Variant Virus

To assess additional cross reactivity and potential broad neutralization of exemplary antibodies, assays to assess neutralization of pseudovirus in addition to live virus were carried out. In this example, exemplary SKM, SKD, R4C1 (IgG, Fab, and Knob), G3 (IgG, Fab, and Knob) and R2D9 (IgG and knob) as described above were tested as indicated.

A pseudovirus luciferase assay (PLSA) substantially as described in Example 5 was performed. Virus neutralization was assessed against pseudotyped virus carrying SARS-CoV-2 (Wuhan-Hu-1) wild-type (WT) spike protein, the S protein of a SARS-CoV-2 beta lineage virus, or a SARS-CoV-2 delta lineage virus. Mock-medium or serially diluted (5-fold) antibody, knob, or fab was mixed with the same amount of the pseudotyped virus carrying SARS-CoV-2 spike protein, and incubated at 37° C. for 1 h. Then, the mixtures were transduced into cells in the presence of polybrene (Santa Cruz Biotech, Santa Cruz, CA) (10 μg/ml). Following incubation of the transduced cells at 37° C. for 48 h, lysis buffer was added, and the RLU were measured.

Neutralization was also assayed using live virus in BSL-3 conditions. Similarly as described above, serially diluted (5-fold) antibody, knob, or fab was mixed with the same amount of wildtype SARS-CoV-2 virus (Wuhan-Hu-1), or either of an alpha (United Kingdom) or beta (South Africa) lineage variant, and incubated at 37° C. for 1 h. The cells were washed, and then plaque forming units (PFU) measured following incubation of the cells at 37° C. for 48 h.

In experiments with pseudo- or live virus, percent neutralization were measured. Inhibition curves of serial dilutions of each antibody against mock treatment were generated, and the maximum percent neutralization (MPN), i.e. the percent at which the neutralization curve plateaus for those viruses neutralized, were determined by GraphPad Prism software using a variable slope (GraphPad, La Jolla, CA). For example, results for exemplary antibody candidate R2G3 (IgG, Fab, and Knob) are shown in FIG. 11B. The results are summarized in Table E7 in ng/mL, with standard deviations of three independent replicates to the right.

TABLE E7
Neutralization of Pseudovirus and Live Virus by Ultralong CDR H3 IgG,
Fabs, and Knobs Against Different SARS-CoV-2 Strains

| | Pseudovirus | | | Live SARS CoV-2 | | |
|---|---|---|---|---|---|---|
| | WT | Beta | Delta | WT | Alpha | Beta |
| SKM | R0.48 ± 0.02 | R > 1000 | R9.05 ± 1.20 | R3.07 ± 1.57 | S | |
| (IgG) | | | | | | |
| SSKD | 0.41 ± 0.01 | N > 1000 | N9.85 ± 1.90 | 04.87 ± 3.65 | 2 | N |
| (IgG) | | | | | | |
| R4C1 | 99.93 ± 31.08 | 375.15 ± 71.63 | 109.8 ± 43.3 | | | |
| (IgG) | | | | | | |
| R4C1 | 184.25 ± 37.12 | 377.95 ± 120.1 | 244.5 ± 67.74 | | | |
| (Fab) | | | | | | |
| R4C1 | 641.95 ± 84.22 | 1024.9 ± 297.1 | 401.3 ± 97.30 | | | |
| (knob) | | | | | | |
| SG3 | 0.20 ± 0.03 | 5.70 ± 0.14 | 0.56 ± 0.09 | >0.50 ± 0.05 | 0.51 ± 0.04 | 3.08 ± 1.73 |
| (IgG) | | | | | | |

### Example 10: Bi- and Multispecific Antibodies with Ultralong CDR3s

Knobs derived from bovine ultralong CDRH3 antibodies are expressed as fusion proteins or as part of dimeric or multimeric molecules, creating bivalent, bispecific, multivalent, or multispecific proteins (FIG. 12). Two or more knobs are expressed as a fusion protein, for example with a flexible linker (e.g., Gly-Gly-Gly-Ser, or the like) between the C-terminus of one knob and the N-terminus of another knob. Additionally, bispecific molecules are made wherein one knob is in its wild-type conformation as a bovine, or humanized bovine, VH region and expressed with a light chain as an IgG, while a second knob is fused to the C-terminus of the heavy chain constant region. In this situation, the two VH regions are identical and have the specificity of knob 1, but the C-terminus has a new specificity as determined by knob 2.

In another approach, ‘knobs into holes’ technology is employed where two heavy chains are co-expressed where one heavy chain contains a VH region with one knob (knob 1) within its CDRH3 and a second heavy chain has a VH region with a second knob within its CDRH3 (knob 2). The two heavy chains also differ by having constant region mutations such that only the heterologous heavy chains effectively pair with one another to form a dimer. In this case, the homodimers are not formed to an appreciable extent. Such ‘knobs-into-holes’ mutations include T22Y (on one chain) and Y86T (on the other chain) in the CH3 domain of Fc.

DNA vectors encoding such molecules are generated by standard molecular biology techniques and expressed and purified as described above in previous Examples. Additionally, individual knobs are chemically covalently linked together using small molecule linkers, or polyethylene glycol (PEG) linkers, including heterobifunctional or heteromultifunctional linkers (e.g., Pierce). In this case, individual knobs are expressed and purified and then added together in the presence of linker and the appropriate reaction conditions to covalently couple the linkers to the knob proteins. Amine, carboxyl, maleimide, NHS ester, and hydrazide chemistries are commonly used in these cross-linking approaches. Furthermore, the knobs are used in the context of a nanoparticle to provide specificity or activity to the nanoparticle. In this regard, the nanoparticle can be a protein-based nanoparticle, including particles formed from viral proteins, albumin nanoparticles, and the like. The nanoparticles can also be derived from non-protein molecules including lipids (e.g., lipoparticles), carbohydrates, etc.

### Example 11: Bioinformatic Identification of Bovine Ultralong CDR H3 Knob Domain Ends

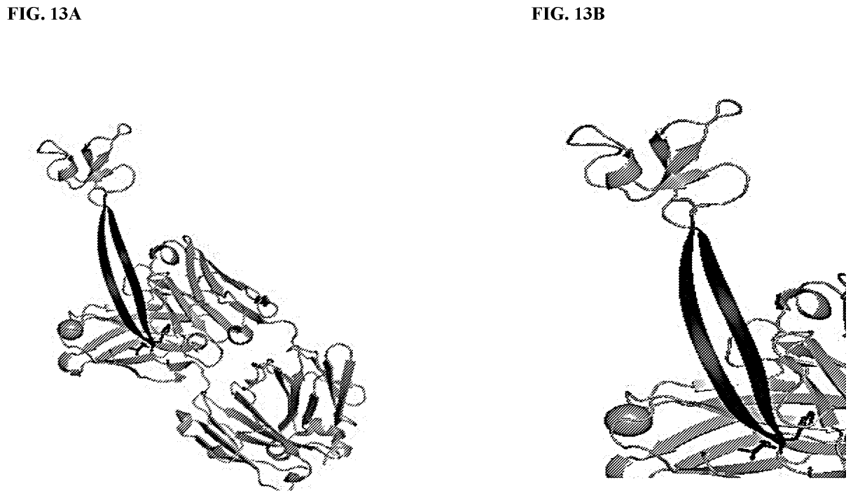

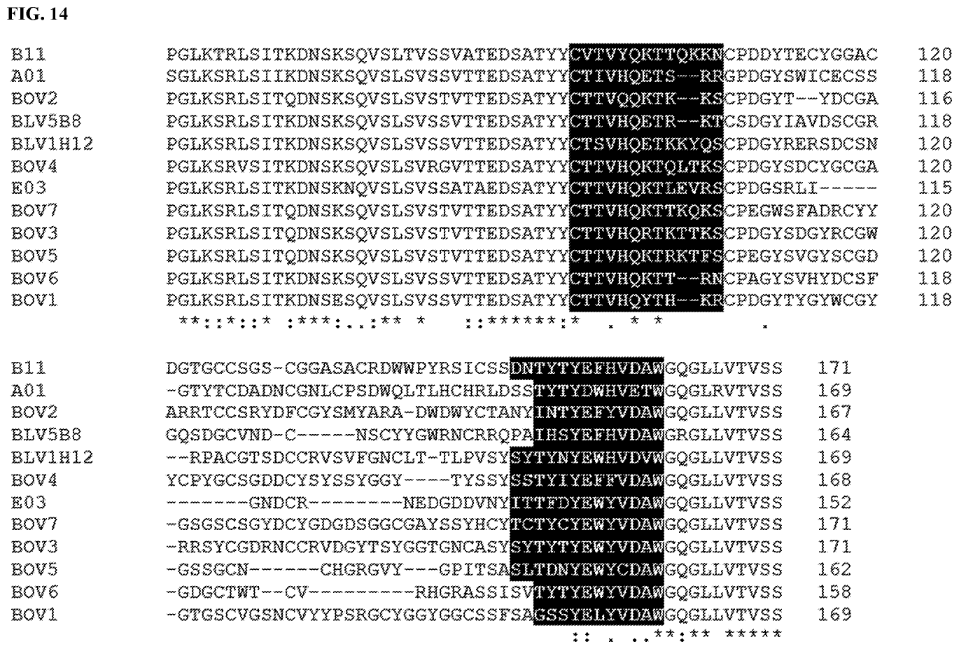

An algorithm was developed to identify bovine ultralong CDR H3 knob domain boundaries by amino acid sequence. By sequence, the bovine ultralong CDR H3 region ranges from “the third residue following the conserved cysteine in framework 3 to the residue immediately preceding the conserved tryptophan in framework 4” (Wang et al. Cell 2013, 153(6):1379-1393). Structurally, the knob domain is defined as the small disulfide-rich domain located upon the distal end of the anti-parallel β-ribbon stalk domain (FIGS. 13A and 13B).

Crystal structures of exemplary bovine ultralong antibodies (Table E8) were analyzed in conjunction with sequences (FIG. 14) to formulate a precise definition of the knob boundaries by both sequence and structure. In the analysis, the first residue of the knob domain was defined as the first conserved D_{H} cysteine, or other residue at this position in rare exceptions such as A01, preceding the conserved “PDG” motif. For the purpose of locating the final knob domain residue, the stalk domain was then also defined. By crystal structure analysis, symmetry was observed in the length of the ascending and descending stalk β-ribbon strands. The conserved framework 3 cysteine, preceding the first CDR H3 residue (Wang et al. 2013) by 3 amino acid positions, is located proximal to the base of the ascending stalk strand and is situated directly across from the conserved framework 4 tryptophan which is one residue downstream of the final CDR H3 residue (Wang et al. 2013). In the analysis, the first ascending stalk residue was defined as the conserved framework 3 cysteine and the final descending stalk residue was defined as the conserved framework 4 tryptophan. The C-terminal knob boundary position was located by subtracting the number of ascending stalk residues from the framework 4 tryptophan position (Table E8).

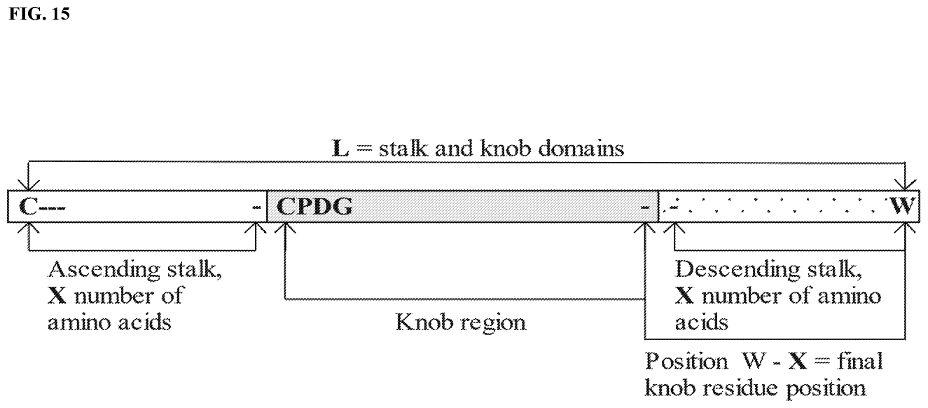

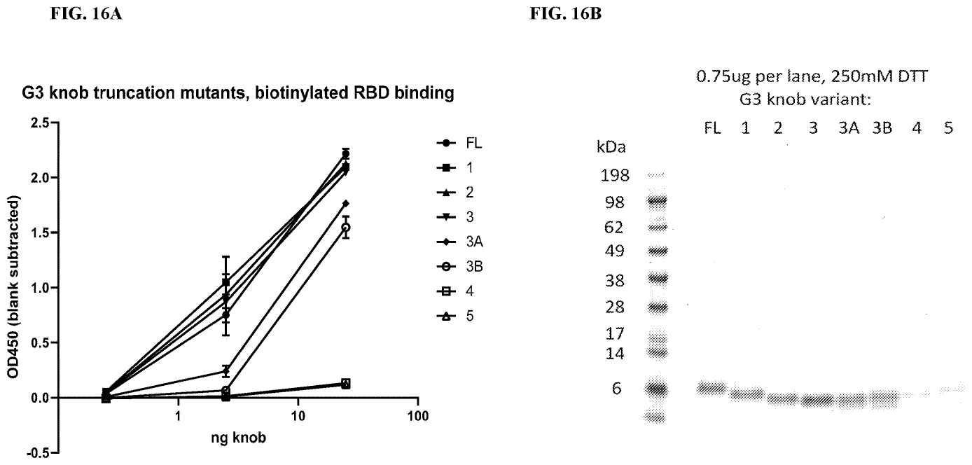

In summary, our algorithm (below) defines the knob region N-terminal boundary as the first D_{H} cysteine in the “CPDG” motif and the C-terminal boundary as the position located by subtracting number of ascending stalk residues from the framework 4 tryptophan position (FIG. 15). The algorithm serves as a general rule that can be applied to bovine ultralong CDR H3 antibody sequences.

In summary, our algorithm (below) defines the knob region N-terminal boundary as the first D_{H} cysteine in the “CPDG” motif and the C-terminal boundary as the position located by subtracting number of ascending stalk residues from the framework 4 tryptophan position (FIG. 15). The algorithm serves as a general rule that can be applied to bovine ultralong CDR H3 antibody sequences.

The algorithm is described as follows: L=number of amino acids encompassing stalk and knob domains, starting at canonical framework 3 cysteine and ending at canonical framework 4 tryptophan. X=number of amino acids, starting at the framework 3 canonical cysteine that defines the ascending stalk, and ending at the amino acid preceding the conserved first D region cysteine in the “CPDG” motif.

Position of conserved framework 4 tryptophan −X=knob boundary position (C-terminal end); Number of residues in the knob (K)=L−2X; K position=(X+1) to (X+K)

TABLE E8

| | Length | Number of | Number of | |
|---|---|---|---|---|
| | encompassing | residues | amino acids in | |
| | stalk and knob | in knob | each stalk | |
| Antibody | domains (L) | domain (K) | strand (X) | PDB ID |
| A01 | 65 | 43 | 11 | 5ilt |
| B11 | 67 | 41 | 13 | 5ihu |
| BLV1H12 | 65 | 39 | 13 | 4k3d |
| BLV5B8 | 60 | 38 | 11 | 4k3e |
| E03 | 48 | 22 | 13 | 5ijv |
| BOV1 | 65 | 43 | 11 | 6e8v |
| BOV2 | 63 | 41 | 11 | 6e9g |
| BOV3 | 67 | 41 | 13 | 6e9h |
| BOV4 | 64 | 38 | 13 | 6e9i |
| BOV5 | 58 | 32 | 13 | 6e9k |
| BOV6 | 54 | 32 | 11 | 6e9q |
| BOV7 | 67 | 41 | 13 | 6e9u |

Bovine ultralong antibodies with published crystal structures that were analyzed, with X number of amino acids in the ascending and descending strands. Total number of amino acids

comprising the stalk and knob domain (L) and knob domain alone (K) for each antibody are also noted.

### Example 12: Defining the Minimal CDR3-Knob C-Terminus and Minimal CDR3-Knob N-Terminus

The algorithm described in Example 11 was validated experimentally by expressing and testing C-terminal truncations (subsection A below) and N-terminal truncations (subsection B below) of a stalk and knob region from an antibody with an unknown structure. In some cases, 1, 2, 3, 4 or 5 amino acids may be added to the knob ends for improved expression or stability.

### A. Defining Minimal CDR3-Knob C-Terminus

In order to define the C-terminal requirements of a prototypical CDR3-knob, a series of R2G3 truncations were cloned into pET32b and expressed as described in Example 6 above. The quality of expressed material was assessed by SDS-PAGE and RBD ELISA also as described in Example 6. Exemplary tested R2G3 truncations are set forth below in Table E9, each truncation was made with a reduced Terminal linker.

TABLE E9
R2G3 CDR3-knob truncations-C-Terminus

| CLONE | Mature amino acid sequence after Enterokinase cleavage |
|---|---|
| G3 Parental | GGGGAMGSEGDKTCPDGYEHTCGCIGGCGCKRSACIGALCCQASLG |
| SEQ ID NO: 86 | GWLSDGETYT |
| G3 TRUNC1 | ~~~~~GGSEGDKTCPDGYEHTCGCIGGCGCKRSACIGALCCQASLGG |
| SEQ ID NO: 87 | WLSDGETYT |
| G3 TRUNC2 | ~~~~~GGSEGDKTCPDGYEHTCGCIGGCGCKRSACIGALCCQASLGG |
| SEQ ID NO: 88 | WLSDGE |
| G3 TRUNC3 | ~~~~~GGSEGDKTCPDGYEHTCGCIGGCGCKRSACIGALCCQASLGG |
| SEQ ID NO: 89 | WLS |
| G3 TRUNC3A | ~~~~~GGSEGDKTCPDGYEHTCGCIGGCGCKRSACIGALCCQASLGG |
| SEQ ID NO: 90 | WL |
| G3 TRUNC3B | ~~~~~GGSEGDKTCPDGYEHTCGCIGGCGCKRSACIGALCCQASLGG |
| SEQ ID NO: 91 | W |
| G3 TRUNC4 | ~~~~~GGSEGDKTCPDGYEHTCGCIGGCGCKRSACIGALCCQASLGG |
| SEQ ID NO: 92 | |
| G3 TRUNC5 | ~~~~~GGSEGDKTCPDGYEHTCGCIGGCGCKRSACIGALCCQAS |
| SEQ ID NO: 93 | |

As shown in FIG. 16A, only Truncations 4 and 5 resulted in no observed RBD binding. Truncations 3A and 3B demonstrated reduced binding in ELISA and increased band diffuseness in SDS-PAGE (FIG. 16B). Truncations 1-3 had no loss in binding activity relative to parental R2G3 CDR3-knob. Taken together, these results support a minimum of 9 amino acids after the last non-canonical Cys residue for R2G3 binding.

### B. Defining the Minimal CDR3-Knob N-Terminus

Similarly as described in Example 11, a series of R2G3 truncations were cloned into pET32b to define the N-terminal requirements of a prototypical CDR3-knob and expressed as described in Example 6 above. The quality of expressed material was assessed by SDS-PAGE and RBD ELISA as described in Example 6. Exemplary tested R2G3 truncations are set forth below in Table E10.

TABLE E10
R2G3 CDR3-knob truncations-N-Terminus

| CLONE | Mature amino acid sequence after Enterokinase cleavage |
|---|---|
| G3 Parental | GGSEGDKTCPDGYEHTCGCIGGCGCKRSACIGALCCQASLGGWLSD |
| SEQ ID NO: | |
| 131 | |
| G3 NTRUNC1 | GGS~GDKTCPDGYEHTCGCIGGCGCKRSACIGALCCQASLGGWLSD |
| SEQ ID NO: | |
| 132 | |
| G3 NTRUNC2 | GGS~~DKTCPDGYEHTCGCIGGCGCKRSACIGALCCQASLGGWLSD |
| SEQ ID NO: | |
| 133 | |
| G3 NTRUNC3 | GGS~~~KTCPDGYEHTCGCIGGCGCKRSACIGALCCQASLGGWLSD |
| SEQ ID NO: | |
| 134 | |
| G3 NTRUNC4 | GGS~~~~TCPDGYEHTCGCIGGCGCKRSACIGALCCQASLGGWLSD |
| SEQ ID NO: | |
| 135 | |
| G3 NTRUNC5 | GGS~~~~~CPDGYEHTCGCIGGCGCKRSACIGALCCQASLGGWLSD |
| SEQ ID NO: | |
| 136 | |

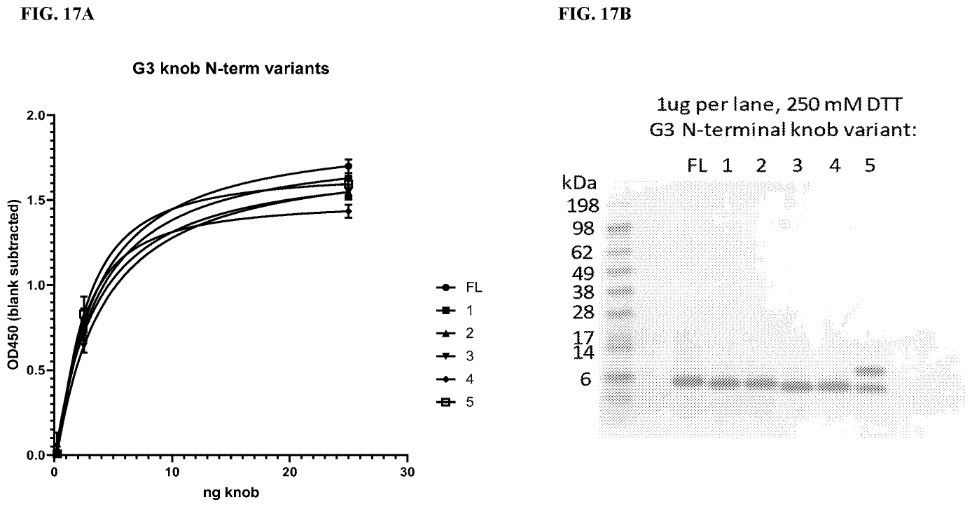

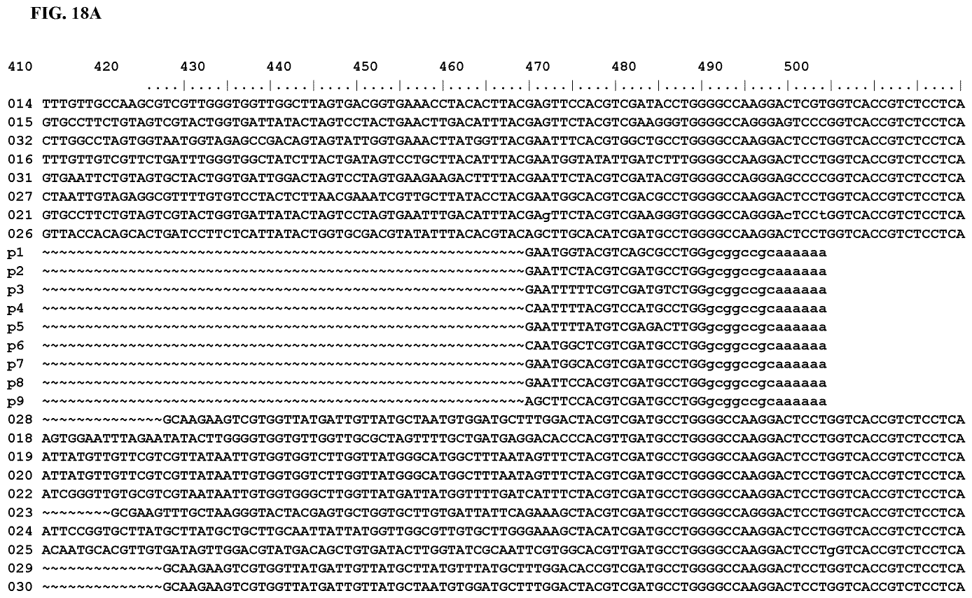

Each of the exemplary N-terminal truncation tested was observed to display similar binding profiles to biotinylated RBD by ELISA and band diffuseness in SDS-PAGE (FIGS. 17A and 17B, respectively). It was noted that truncation 5 resulted in two bands via SDS-PAGE, however this did not correlate with any reduction in binding activity. These results suggest that none of the amino acids deleted in these exemplary truncated R2G3 sequences are part of the knob domain.

### Example 13: Selective Amplification of Ultralong CDR3-Knob Domains

Ultralong CDR3-knob domains were selectively amplified from a cow VH template library. The cow VH template library was prepared substantially as described in Example 2.

Specifically, RNA was isolated from 5×10⁶-10⁷ bovine PBMCs using an RNAeasy kit (Qiagen). Immune cow antibody CDR3-knob repertoires were obtained through cDNA synthesis from 5 μg total RNA using Superscript IV First-Strand cDNA synthesis kit (ThermoFisher), followed by PCR amplification. To generate the VH template library, the cDNA template for CDR3-knobs was synthesized using a pool of IgM (SEQ ID NO: 4), IgA (SEQ ID NO: 5), and IgG-specific (SEQ ID NO: 3 and 6) primers.

Primary stalk-knob CDR3 were amplified from 1^{st} strand cDNA with IgHV1-7 family specific primers specific for either side of the stalk domain of the CDR3 region. Primary stalk-knob CDR3 were amplified using a pool of primers containing all of the primers set forth in SEQ ID NO: 8-11 as well as one of the primers set forth in SEQ ID NO: 122-130. The amplified sequences were then analyzed for the prevalence of ultralong CDR3-knob domains using gel electrophoresis with a 2% agarose gel.

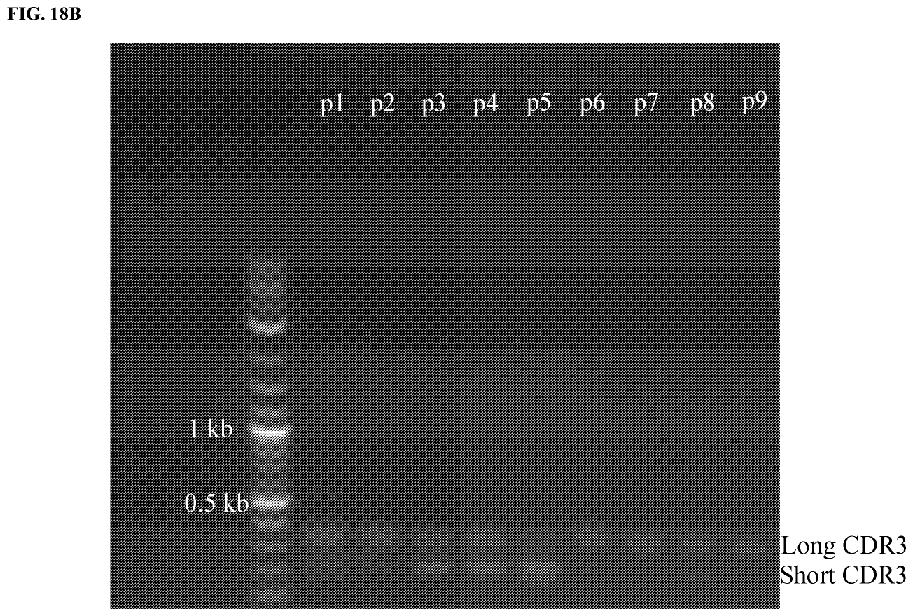

An alignment of the primers set forth in SEQ ID NO: 122-130 (primers p1-p9) to sequences of exemplary standard short CDR3 antibodies (antibodies 028-030) and ultralong CDR3 antibodies (antibodies 01-026) is shown in FIG. 18A. Sequence identifiers (SEQ ID NO) of the sequences shown in FIG. 18A are shown in Table E11.

TABLE E11
Sequence Identifiers (SEQ ID NO)
for Sequences Shown in FIG. 18A

| SEQUENCE | SEQ ID NO |
|---|---|
| 014 | 137 |
| 015 | 138 |
| 032 | 139 |
| 016 | 140 |
| 031 | 141 |
| 027 | 142 |
| 021 | 143 |
| 026 | 144 |
| p1 | 122 |
| p2 | 123 |
| p3 | 124 |
| p4 | 125 |
| p5 | 126 |
| p6 | 127 |
| p7 | 128 |
| p8 | 129 |
| p9 | 130 |
| 028 | 145 |
| 018 | 146 |
| 019 | 147 |
| 020 | 148 |
| 022 | 149 |
| 023 | 150 |
| 024 | 151 |
| 025 | 152 |
| 029 | 153 |
| 030 | 154 |

Results of gel electrophoresis indicated that amplification with the pools of primers containing the primers set forth in SEQ ID NO: 123, 127, and 128 resulted in enrichment for ultralong CDR3-knob domains (FIG. 18B), especially with annealing between 65-68° C. Specifically, while two bands were apparent for PCR products obtained using some of the primers, indicating the amplification of standard short as well as ultralong CDR3-knob domains, only one band corresponding to sequences of ultralong CDR3-knob domains (expected PCR product size of approximately 300-350 bp) was obtained using the primers set forth in SEQ ID NO: 123, 127, and 128.

A stalk-knob CDR3 library was constructed from DNA amplified using the primers set forth in SEQ ID NO: 8-11, 123, 127, and 128. The library was constructed substantially as described in Example 2 and was selected against Spike protein for two rounds of selection as described in Example 3. Over 90% of screened clones were Spike-binding clones, and all binding clones were ultralong CDR3 antibodies.

These results indicate that ultralong CDR3-knob domains can be selectively amplified from a VH template library using particular primers specific for the stalk domain of the CDR3 region.

### Example 14: Generation of Knob 2×NNK Phage Display library and Selection of High Affinity Stable Phage

Examples 7 and 10 above demonstrate that CDR3-knob only antibodies retain robust biological antibody as exemplified by virus neutralization, including at a subnanomolar potency. However, the results demonstrated that the CDR3-knob only antibody can, in some cases, have poorer neutralization potency than the Fab formats. Without wishing to be bound by theory, it is contemplated that some activity may be lost due to removal of the β-ribbon “stalk”—including the ascending (stalk A) and descending (stalk B) residues located upstream and downstream of the knob in the CDR3 (e.g. see Example 11)—which could serve to “clamp” the knob N- and C-termini.

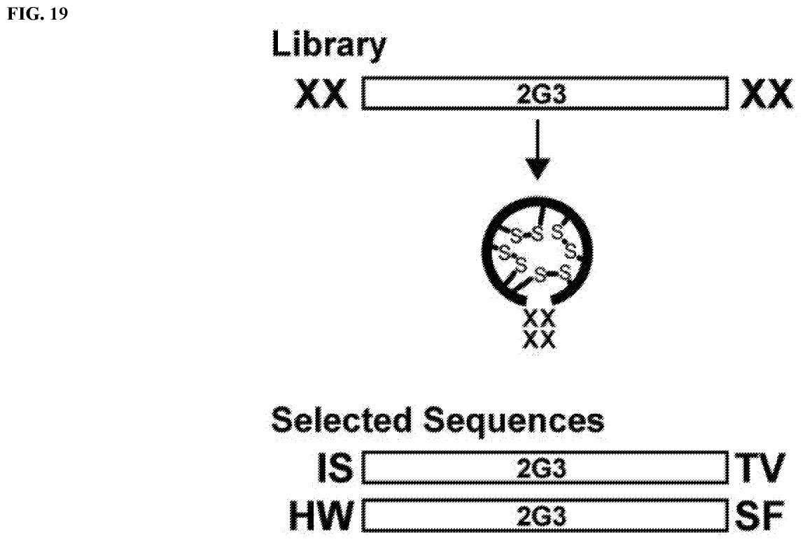

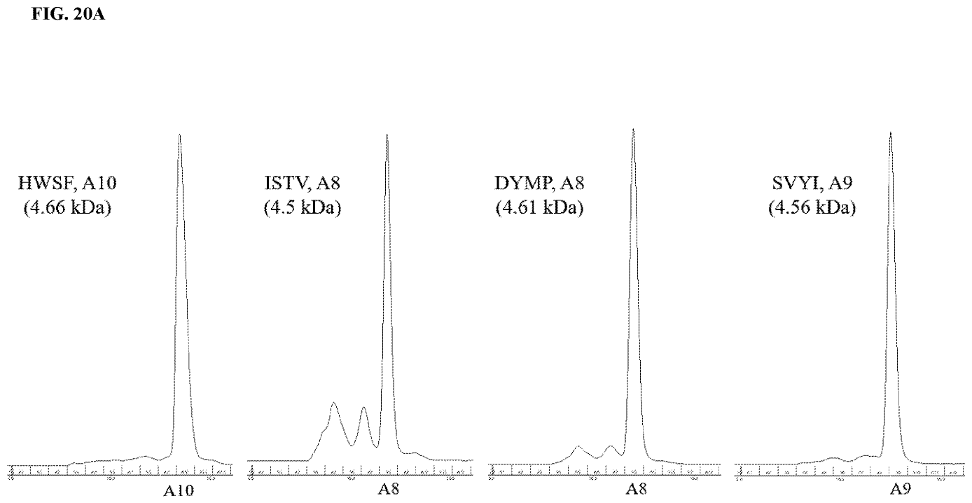

To identify if adding amino acid residues to the N- and C-termini could improve stability and binding activity of a CD3-knob only antibody, the exemplary CD3-knob only antibody R2G3 having the minimal knob sequence TCPDGYEHTCGCIGGCGCKRSACIGALCCQASLGGWLSD (see SEQ ID NO:155) was used as a template to generate a library with 2×NNK on both N- and C-terminal ends of the CD3-knob only antibody (see FIG. 19). A nucleic acid sequence encoding the R2G3 CD3-knob only antibody was cloned into pTAU1 pIII fusion phage display vector such that the cloned R2G3 sequence was flanked by a 5′ NcoI site (GCCATGGCC) and a 3′ Not1 site (GCGGCCGCA) and fused to pIIII via a 6×His tag, and used as a template to generate a phage display library. Sequences were selected based on their stability to heat and proteases, and their ability to compete with R2G3 IgG or R2G3 CD3-knob only antibody (without N- or C-terminal sequences) for antigen binding as described below.

### A. Generation of 2×NNK Knob Phage Display Libraries

R2G3 knob sequence (SEQ ID NO: 155) from the pTAU1_G3Knob template vector were amplified using NNK forward (SEQ ID NO:156) and NNK reverse primers (SEQ ID NO:157) to generate R2G3 knobs with randomized residues at the N- and C-terminus. The amplified products were subjected to 2 hours of digestion with the NcoI and NotI (NEB) and subcloned into pTAU1 pIIII fusion phage display vector by ligation overnight with T4 DNA ligase at 16° C. Final libraries were obtained by electroporation of electrocompetent TG1 cells (Lucigen) with the purified ligation products. Amplification by PCR of select colonies showed that the clones had the correct size of 431 bp. The size of the library was about 2.87×10⁸ clones with a theoretical diversity of 1.6×10⁸.

### B. Screening of Phage Display Libraries and Selection of High Affinity Stable Phage

The randomized phage display libraries described above were subjected to multiple rounds of phage display selections against SARS CoV-2 “South African” B.1.351 variant Spike protein as described in Example 3 above. Table E12 describes the selection strategies that were used.

TABLE E12

| Strategy | Description |
|---|---|
| 1 | Binding to SARS CoV-2 target proteins, no other |
| | selection pressure - not moving forward after round 1. |
| 2 | Competition with R2G3 knob |
| 3 | Pre-treatment with trypsin-and/or chymotrypsin-agarose |
| 4 | Mixing equal amounts of phage from strategy 2 and strategy 3 |
| 5 | Competition with R2G3 IgG |
| 6 | Heat at 65° C. + competition with R2G3 knob |

Table E13A-E13C set forth the input and outputs for selections using the above strategies. For each selection, input phage particles from the library were subjected to heating at 65 RC, exposed to trypsin and chymotrypsin agarose beads, and/or competed for binding to SARS-CoV-2 wild-type spike protein with the R2G3 IgG or R2G3 CDR3-knob only antibody. The selections were carried out sequentially with increased selection pressure. The phage particles from the pre-treatment were then added to 1 mL 4% milk powder dissolved in PBS, and made up to 2 mL total volume with PBS, and then added to tubes with the spike protein. Phage were eluted from the tubes with trimethylamine elution. Remaining “tube bound” phage after trimethylamine elution were also monitored since some phage was leftover on the tube walls after elution. For tube bound conditions, TG1 cells are added directly into tubes for the leftover phage to infect.

TABLE E13A

| Round of | | | Output |
|---|---|---|---|
| selection | Strategy number | Input # | # |
| Round 1 | 1 (no competition/ | 2.4 × 10¹² | 8.86 × 10⁷ |
| | treatment) | | |
| | 2 (competition with | | 7 × 10⁶ |
| | R2G3 knob) | | |
| | 3 (pre-treatment with | | 2.87 × 10⁷ |
| | trypsin-agarose) | | |
| | 2 (competition with | 8 × 10¹² | 1.45 × 10⁷ |
| | R2G3 knob) | | |
| Round 2 | 3 (treatment with trypsin | 4 × 10¹⁴ | 1.4 × 10⁸ |
| | and chymotrypsin) | | |
| | 4 (phage from strategy 2 | 4 × 10¹² (#2) + 2 × | 10⁶ |
| | and 3 combined & | 10¹⁴ (#3) | |
| | trypsin + chymotrypsin | | |
| | mixture) | | |

TABLE E13B

| Phage from selection 4 in Table 2 (competition | | Output # | |
|---|---|---|---|
| & trypsin + chymotrypsin mixture in round 2) - | | (in elution | Output # |
| Round 3 | Input # | buffer) | (tube bound) |
| 5 (competition with G3 IgG) | 5.1 × 10¹² | 8 × 10⁵ | ~5000 |
| 6 (65° C. + competition with G3 knob) | | 1.5 × 10⁶ | 1.4 × 10⁴ |

TABLE E13C

| | | Output # | |
|---|---|---|---|
| Phage from selection 2 in Table 2 (competition | | (in elution | Output # |
| with G3 knob in round 1&2) Round 3 | Input # | buffer) | (tube bound) |
| 5 (competition with G3 IgG) | 2 × 10¹² | 1.1 × 10⁶ | >400 |
| 6 (65° C. + competition with G3 knob) | | 3.1 × 10⁶ | 4200 |

The results indicated that combined treatment in round 2 potentially selected for high affinity/stable phage for binding to spike protein. Further, results in round 3 indicated that competition with R2G3 IgG resulted in higher selection pressure than heating.

Phage were selected that were (i) stable to heat, (ii) resistant to proteases, and (iii) compete with wild type R2G3 for antigen binding. Positive clones from the selection screening were identified and sequenced. Several sequences were identified that survived this selection scheme. Exemplary sequences identified following selection by scheme in Table E13B are depicted in Table E14A and exemplary sequences identified following selection by scheme in Table E13C are depicted in Table E141B. Amino acids with “Q” indicate phage with TAG stop codon providing a growth advantage; these sequences were not chosen for further study. Likewise, sequences with extra cysteines also were not chosen for further study. From the results in Tables E14A and Tables E14, N- and C-terminal sequences DY-MP (e.g. G3-C, SEQ ID NO: 160), SV-YI (e.g. G3-E, SEQ ID NO: 162), LV-IP (e.g. G3-G), HW-SF (e.g. G3-R, SEQ ID NO: 164 and FIG. 19), GM-RS (e.g. G3-Z, SEQ ID NO: 182), IS-TV (e.g. G3-AA, SEQ ID NO: 183 and FIG. 19) were identified as residues that conferred improved stability to the knob sequence.

TABLE E14A
Selected sequences from #4 Phage (competition or
trypsin&chymotrypsin mixture selected in round 2)-Round 3

| | | SEQ ID |
|---|---|---|
| Name | Sequence | NO |
| | 5. Competition with G3 IgG | |
| G3-A | TITCPDGYEHTCGCIGGCGCKRSACIGALCCQASLGGWLSDQS | 158 |
| G3-B | CKTCPDGYEHTCGCIGGCGCKRSACIGALCCQASLGGWLSDQH | 159 |
| | 6. 65C heat + competition with G3 knob-tube bound | |
| G3-C | DYTCPDGYEHTCGCIGGCGCKRSACIGALCCQASLGGWLSDMP | 160 |
| G3-D | LQTCPDGYEHTCGCIGGCGCKRSACIGALCCQASLGGWLSDPP | 161 |
| G3-E | SVTCPDGYEHTCGCIGGCGCKRSACIGALCCQASLGGWLSDYI | 162 |
| G3-F | NGTCPDGYEHTCGCIGGCGCKRSACIGALCCQASLGGWLSDMQ | 163 |
| G3-G | LVTCPDGYEHTCGCIGGCGCKRSACIGALCCQASLGGWLSDIP | 164 |
| | 6. 65C heat + competition with G3 knob-in elution | |
| G3-H | QGTCPDGYEHTCGCIGGCGCKRSACIGALCCQASLGGWLSDCQ | 165 |
| G3-I | LSTCPDGYEHTCGCIGGCGCKRSACIGALCCQASLGGWLSDQV | 166 |
| G3-K | MITCPDGYEHTCGCIGGCGCKRSACIGALCCQASLGGWLSDQV | 167 |
| G3-L | LVTCPDGYEHTCGCIGGCGCKRSACIGALCCQASLGGWLSDRQ | 168 |
| G3-M | QPTCPDGYEHTCGCIGGCGCKRSACIGALCCQASLGGWLSDKQ | 169 |

TABLE E14B
Selected sequences from #2 Phage (competition with G3 knob
selected in round 1 & 2)-Round 3

| | | SEQ ID |
|---|---|---|
| Name | Sequence | NO |
| | 5. Competition with G3 IgG in elution | |
| G3-N | QQTCPDGYEHTCGCIGGCGCKRSACIGALCCQASLGGWLSDYL | 170 |
| G3-O | AQTCPDGYEHTCGCIGGCGCKRSACIGALCCQASLGGWLSDYH | 171 |
| G3-P | HLTCPDGYEHTCGCIGGCGCKRSACIGALCCQASLGGWLSDQQ | 172 |
| G3-Q | PQTCPDGYEHTCGCIGGCGCKRSACIGALCCQASLGGWLSDQF | 173 |
| G3-R | HWTCPDGYEHTCGCIGGCGCKRSACIGALCCQASLGGWLSDSF | 174 |
| G3-S | SCTCPDGYEHTCGCIGGCGCKRSACIGALCCQASLGGWLSDQP | 175 |
| G3-T | PFTCPDGYEHTCGCIGGCGCKRSACIGALCCQASLGGWLSDQH | 176 |
| G3-U | WMTCPDGYEHTCGCIGGCGCKRSACIGALCCQASLGGWLSDQQ | 177 |
| | 6. 65C heat + competition with G3 knob-tube bound | |
| G3-V | SCTCPDGYEHTCGCIGGCGCKRSACIGALCCQASLGGWLSDDS | 178 |
| G3-W | LSTCPDGYEHTCGCIGGCGCKRSACIGALCCQASLGGWLSDVQ | 179 |
| G3-X | QLTCPDGYEHTCGCIGGCGCKRSACIGALCCQASLGGWLSDYV | 190 |
| G3-Y | LVTCPDGYEHTCGCIGGCGCKRSACIGALCCQASLGGWLSDQQ | 181 |
| | 6. 65C heat + competition with G3 knob-in elution | |
| G3-Z | GMTCPDGYEHTCGCIGGCGCKRSACIGALCCQASLGGWLSDRS | 182 |
| G3-AA | ISTCPDGYEHTCGCIGGCGCKRSACIGALCCQASLGGWLSDTV | 183 |
| G3-BB | SQTCPDGYEHTCGCIGGCGCKRSACIGALCCQASLGGWLSDHL | 184 |
| G3-CC | CDTCPDGYEHTCGCIGGCGCKRSACIGALCCQASLGGWLSDQR | 185 |
| G3-DD | SLTCPDGYEHTCGCIGGCGCKRSACIGALCCQASLGGWLSDQR | 186 |

### Example 15: Biochemical Characterization and Activity of Modified CDR3-Knob Domains

Modified G3 Knobs selected in Example 14 above were further characterized. In addition, exemplary N- and C-terminal overhang modifications were also introduced to other knob sequences to assess if the modifications were transplantable to other knob sequences to improve stability and activity. Specifically, the N- and C-terminal overhang modifications HW-SF or IS-TV were introduced to the minimal CD3-knob only antibody R4C1 sequence NCPDGYIYSSNTASGYDCGVWICRRVGSAFCSRTGDYTSP (SEQ ID NO:187) to generate the modified R4C1 sequence set forth in SEQ ID NO:188 or 189, and to the minimal CD3-knob antibody sequence R2D9 TCPNGYNWFDRCCSWDGTCGDGCCSNRAWPSGNGRADSSIGE (SEQ ID NO:190) to generate the modified R2D9 sequence set forth in SEQ ID NO:191 or 192.

The selected or generated knob sequences were cloned into the pET32b vector (FIG. 6A) as described in Example 6 to produce a trxA-CDR3-knob fusion clone. The CDR3-knobs were purified as described in Example 6, and characterized by size exclusion chromatography and by gel electrophoresis or for activity in a neutralization assay.

### A. Biochemical Characterization

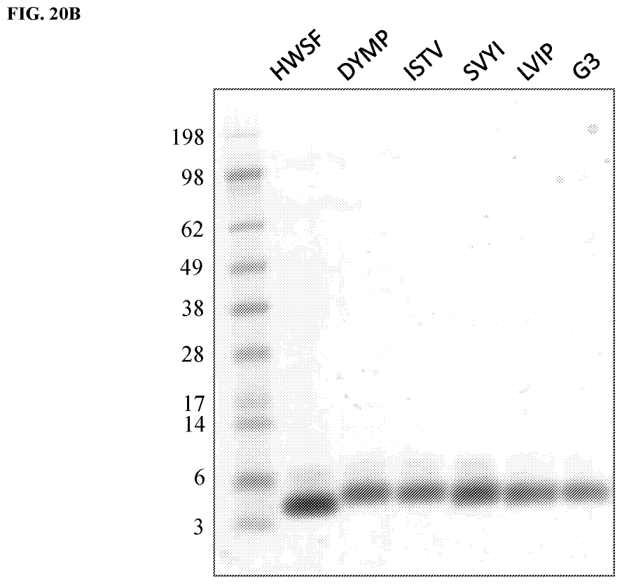

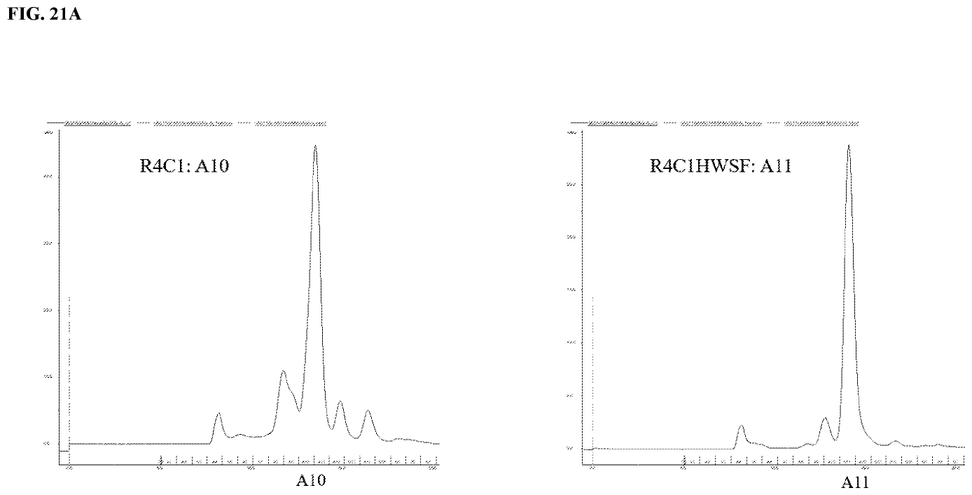

Size exclusion chromatography (SEC) was used to characterize the purified CDR3-antibody knobs. As shown in FIG. 20A, size exclusion chromatography revealed at least three distinct elution fractions (fractions A8, A9 and A10) for purified 2×NNK R2G3 knobs, indicating that the amino acid changes introduced into the 2×NNK ends affected the size of R2G3 knobs. As shown in FIG. 20B, analysis by gel electrophoresis under reducing conditions show that the modification HW-SF makes the G3 knob more compact than the other N- and C-terminal overhangs.

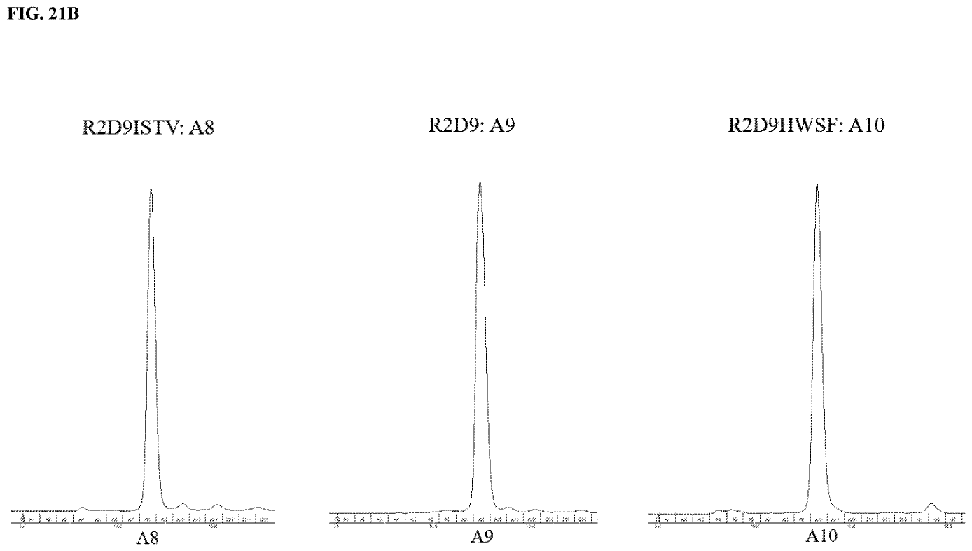

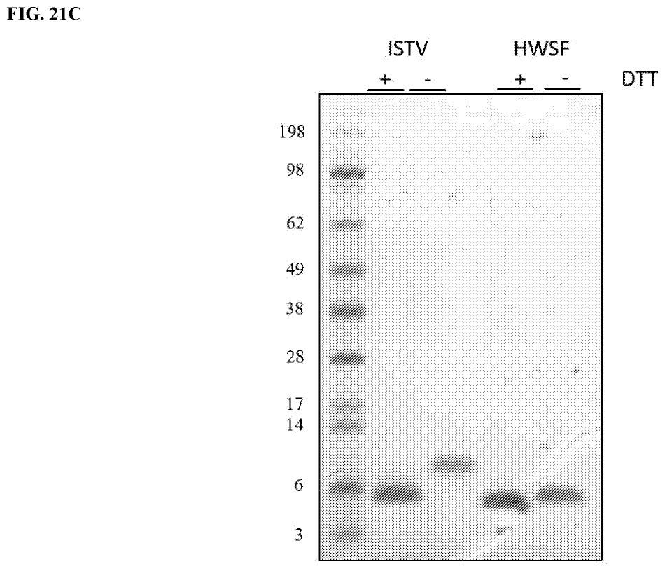

SEC analysis of R4C1 or R2D9 CD3-only antibody knobs, or HWSF or ISTV modified versions thereof, showed that the N- and C-terminal overhang sequences affected the size of the knob sequences. In particular, the N- and C-terminal HW-SF overhang similarly made the alternative knob sequences more compact, as shown in FIG. 21A (R4C1 or modified knobs) and FIG. 21B (R2D9 or modified knobs). As shown in FIG. 21C, analysis by gel electrophoresis under reducing conditions (with and without DTT) further confirmed that modification of R2D9 with the N- and C-terminal HW-SF overhang makes the G3 more compact than modification with the IS-TV overhang.

### B. Activity

G3 purified knob sequence (SEQ ID NO:155) or HW-SF G3 overhang-modified G3 knob sequence (SEQ ID NO:164) or IS-TV overhang-modified G3 knob sequence (SEQ ID NO:183) were tested in a pseudovirus neutralization assay substantially as described in Examples 5 and 7 for their ability to neutralize infection of cells with a SARS CoV-2 pseudovirus carrying SARS-CoV-2 (Wuhan-Hu-1) wild-type (WT) spike protein. For comparison, pseudovirus neutralization of the Fab of the R2G3 CDR3 antibody (“G3-Fab”) or R2G3 IgG also was assessed in the same assay.

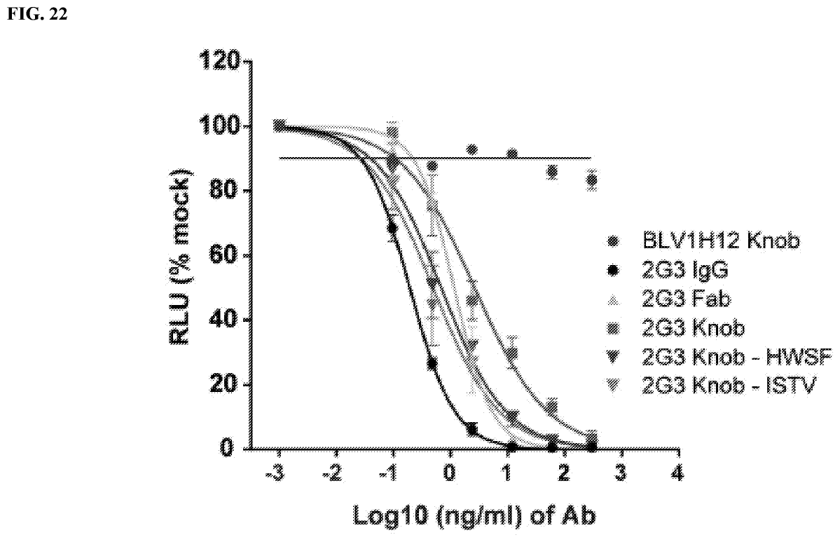

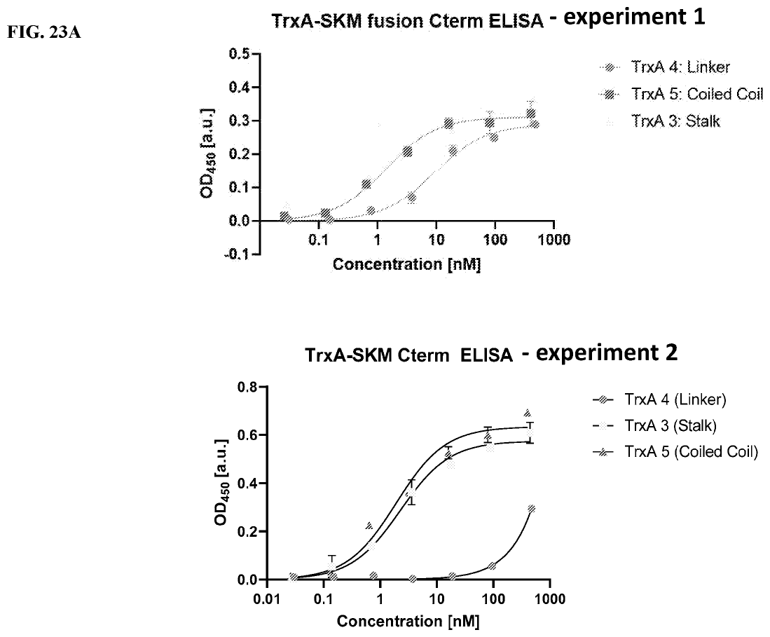

As shown in FIG. 22, the two modified sequences had improved potency in the pseudovirus neutralization assay in comparison to the parental G3 purified knob. The results surprisingly showed that the modified sequences were as equally potent as the 2G3 Fab for neutralization of SARS CoV-2 pseudovirus carrying wild-type (WT) spike protein. Table E15 also demonstrates the potent neutralization properties of the modified knob sequences against pseudotyped virus carrying SARS-CoV-2 (Wuhan-Hu-1) wild-type (WT) spike protein, or variants from the beta and delta lineages.

TABLE E15
Virus Neutralization

| | Pseudovirus (nM) | | |
|---|---|---|---|
| Antibody | Wild-type | Beta | Delta |
| 2G3 IgG | 0.0013 ± 0.0002 | 0.0380 ± 0.0009 | 0.0037 ± 0.0006 |
| 2G3 Fab | 0.026 ± 0.004 | 0.043 ± 0.02 | 1.61 ± 0.98 |
| 2G3 Knob | 1.3 ± 0.3 | 15.65 ± 2.61 | 71.3 ± 37.16 |
| 2G3 knob | 0.12 ± 0.03 | 20.87 ± 7.95 | 55.97 ± 7.02 |
| ISTV | | | |
| 2G3 knob | 0.15 ± 0.03 | 7.82 ± 1.59 | 21.17 ± 22.92 |
| HWSF | | | |

Together these results establish that highly active peptide knobs can be produced, and their activities optimized through mutation of their interacting N- and C-termini. These results are consistent with a finding that the selected N- and C-terminal overhang modifications are transplantable to other knob sequences and exhibit similar improvements in stability and activity.

### Example 16: Comparison of Additional Modified N- and C-Terminal “Clamp” Knob Constructs

Additional strategies to modify the N- and C-termini of knob sequences were assessed to compare impacts on knob expression and activity. For these strategies, the N- and C-termini of knob sequences were modified by retaining a portion of the ascending and descending stalk sequences of the CDR3 (stalk-knob-stalk), adding an N- and C-terminal coiled coil (CC) domain (CC-knob-CC), or adding an N- and C-terminal GGS linker (GGS-knob-GGS). For the coiled-coil strategy, the sequence of the ascending peptide with linkers at each end is GGSGAKLAALKAKLAALKGGGGS (SEQ ID NO:202) and the sequence of the descending peptide with linkers at each end is GGGGSELAALEAELAALEAGGSG (SEQ ID NO:203), see e.g. Zhang et al. Angew Chem Int Ed Engl. 2014; 53:132-135. The experiments were carried out with the exemplary SARs-CoV-2 binding knob SKM set forth in SEQ ID NO:198.

The generated knob sequences were cloned into the pET32b vector (FIG. 6A) as either (1) a TrxA C-terminal fusion substantially as described in Example 6 to produce a trxA-CDR3-knob fusion clone; or (2) as a TrxA loop engineered fusion. For the TrxA loop engineered fusions, the knob peptide fusions were engineered to be displayed by an active site loop of TrxA. TrxA has three active site loops that are involved in interactions with its substrates: the catalytic loop corresponding to residues 31-35 of SEQ ID NO:194, a first binding loop corresponding to residues 74-76 of SEQ ID NO:194 and a second binding loop corresponding to residues 91-93 of SEQ ID NO:194. In an exemplary strategy, the fusion knob peptides were engineered into the last loop of TrxA between Val-92 and Gly-93 of the sequence set forth in SEQ ID NO:194.

Table E16A summarizes the TrxA engineered loop fusion constructs that were generated. In some cases, a C-terminal linker (e.g. GG or TG) can be added after the C-terminal TrxA, which can correspond to an encoded restriction site sequence (e.g. EcoRI or BamHI) used for cloning. In some cases, a final C-terminal tag sequence also can be included, such as to facilitate purification of the fusion protein. An exemplary tag is QETFSDLWKLLPEN (SEQ ID NO:208), which is a p53-transactivation domain (tad) tag recognized by anti-p53 antibody DO1 (e.g. Cat. No. GTX70214 GeneTex, San Antonio Texas), and allowed specific detection of knobs only. Alternatively, a 6×His tag (SEQ ID NO: 217) can be used.

TABLE E16A
TrxA engineered loop fusion constructs

| | N- | N- | | C- | | | |
|---|---|---|---|---|---|---|---|
| | terminal | terminal | | terminal | | | |
| | portion | knob | Knob | knob | C- | | |
| | of TrxA | overhang | peptide | overhang | terminal | Trx A fusion | |
| | SEQ ID | SEQ ID | SEQ ID | SEQ ID | portion of | | Amino |
| Name | NO | NO | NO | NO | TrxA | Nucleotide | acid |
| TrxA1: | 195 | Stalk A | STM | Stalk B | 199 | 200 | 201 |
| TrxA Loop SKS- | | (e.g. 196) | knob | (e.g. 197) | | | |
| STM | | | (e.g. SEQ | | | | |
| | | | ID | | | | |
| | | | NO:198) | | | | |
| TrxA2: | 195 | GGS | STM | GGT | 199 | 206 | 207 |
| TrxA_Loop_GKG- | | | knob | | | | |
| SKM | | | (e.g. SEQ | | | | |
| | | | ID | | | | |
| | | | NO:198) | | | | |
| TrxA6: | 195 | 202 | STM | 203 | 199 | 204 | 205 |
| TrxA_Loop_CKC- | | | knob | | | | |
| SKM | | | (e.g. SEQ | | | | |
| | | | ID | | | | |
| | | | NO:198) | | | | |

Table E16B summarizes the TrxA C-terminal knob fusion constructs that were generated. In some cases, GS linkers (e.g. GGS or GS) can be added on either side of the Flag/enterokinase cleavage site. In some cases, a C-terminal linker (e.g. GGS) can be added after the C-terminal overhang sequence. In some cases, a final C-terminal tag sequence also can be included, such as to facilitate purification of the fusion protein. An exemplary tag is QETFSDLWKLLPEN (SEQ ID NO:208), which is a p53-transactivation domain (tad) tag recognized by anti-p53 antibody DO1 (e.g. Cat. No. GTX70214 GeneTex, San Antonio Texas). Alternatively, a 6×His tag (SEQ ID NO: 217) can be used.

TABLE E16B
TrxA C-terminal knob fusion constructs

| | | | N- | | C- | | |
|---|---|---|---|---|---|---|---|
| | | | terminal | | terminal | | |
| | | | knob | Knob | knob | | |
| | TrxA | FLAG/ | overhang | peptide | overhang | TrxA fusion | |
| | SEQ | Enterokinase | SEQ ID | SEQ ID | SEQ ID | | Amino |
| Name | ID NO | site | NO | NO | NO | Nucleotide | acid |
| TrxA3: | 194 | 209 | Stalk A | STM | Stalk B | 211 | 212 |
| TrxA_Cterm_SKS- | | (e.g. 210 with | (e.g. 196) | knob | (e.g. 197) | | |
| SKM | | GS linkers) | | (e.g. | | | |
| | | | | SEQ ID | | | |
| | | | | NO: 198) | | | |
| TrxA4: | 194 | 209 | GGS | STM | GTG | 215 | 216 |
| TrxA_Cterm_GKG- | | (e.g. 210 with | | knob | | | |
| SKM | | GS linkers) | | (e.g. | | | |
| | | | | SEQ ID | | | |
| | | | | NO: 198) | | | |
| TrxA5: | 194 | 209 | 202 | STM | 203 | 213 | 214 |
| TrxA_Cterm_CKC- | | (e.g. 210 with | | knob | | | |
| SKM | | GS linkers) | | (e.g. | | | |
| | | | | SEQ ID | | | |
| | | | | NO: 198) | | | |

Nucleotide sequences encoding the Trx A fusion constructs in Tables 16A and 16B, cloned into pET32b vector (EMD-Millipore), were transformed into Origami 2 DE3 bacteria. The bacteria were grown substantially as described in Example 6. Supernatant was collected and were purified using 6×His tag over cobalt resin. For TrxA C-terminal knob fusion constructs containing an Enterokinase cleavage site, trxA-CDR3-knob was adjusted to 50 mM Tris pH 7.4, 150 mM NaCl, and 2.5 mM CaCl₂) (1× enterokinase (EK) reaction buffer), and 400u recombinant his-tagged Enterokinase (Genscript) was added and incubated overnight at room temperature. Digested trxA and enterokinase were removed, and purified CDR-knob was collected in the flowthrough.

Tables E17A and E17B summarize the yield of protein expressed by the above strategies in two separate experiments. Yield was determined using nanodrop at 280 nm based on extinction coefficient and molecular weight. As shown, the coiled coil constructs have the highest level of expressions, whether the knob is expressed by display on an engineered TrxA loop or as a C-terminal fusion with enterokinase cleavage.

TABLE E17A

| Construct | Yield [mg/L] |
|---|---|
| TrxA2: TrxA-SKM Loop Linker | 0.62 |
| TrxA4: TrxA-SKM Cterm Linker | 0.85 |
| TrxA6: TrxA-SKM Loop Coiled Coil | 8.2 |
| TrxA5: TrxA-SKM Cterm Coiled Coil | 10.8 |
| TrxA1: TrxA-SKM Loop Stalk | 7.2 |
| TrxA3: TrxA-SKM Cterm-Stalk | 2.5 |

TABLE E17B

| Construct | Yield [mg/L] |
|---|---|
| TrxA2: TrxA-SKM Loop Linker | 0.78 |
| TrxA4: TrxA-SKM Cterm Linker | 0.46 |
| TrxA6: TrxA-SKM Loop Coiled Coil | 9.5 |
| TrxA5: TrxA-SKM Cterm Coiled Coil | 21.8 |
| TrxA1: TrxA-SKM Loop Stalk | 4.8 |
| TrxA3: TrxA-SKM Cterm-Stalk | 5.5 |

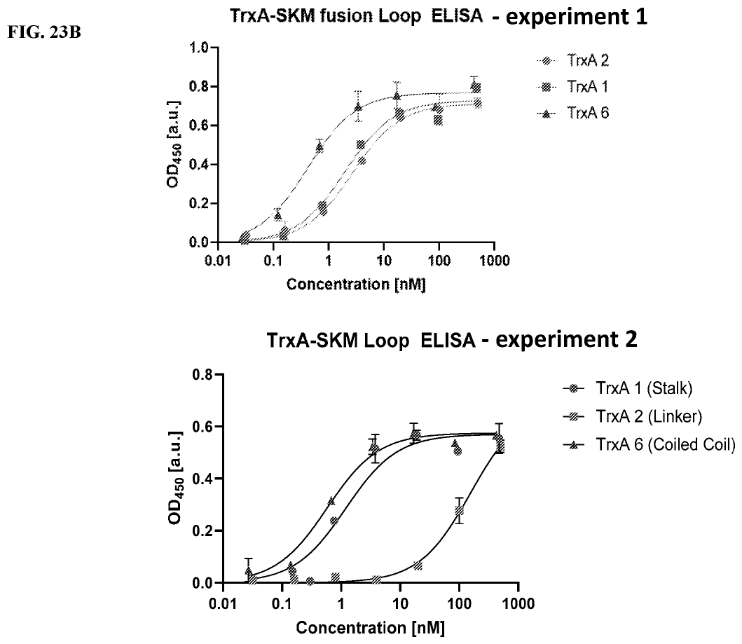

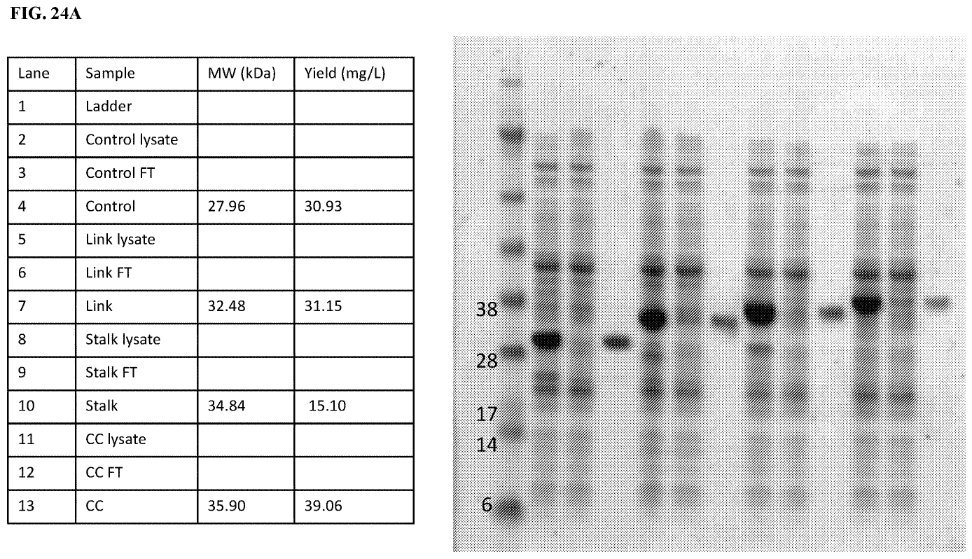

Binding of the purified proteins to recombinant stabilized spike protein derived from the wild-type (WT) Wuhan-Hu-1 strain was assessed by ELISA substantially as described in Example 4. OD 450 nm values were determined. Results are depicted in FIG. 23A for the TrxA-SKM C-terminal fusions from two different experiments and in FIG. 23B for the TrxA-SKM Loop fusion constructs. The maximum (max) OD450 as well as the IC50 for binding the spike protein are depicted in Tables 18A (TrxA-SKM C-terminal fusions) and 18B (TrxA-SKM Loop fusion constructs) for each experiment. The results further confirm improved binding activity of the coiled-coil domain constructs compared to the other constructs. The results further demonstrate that expression with an N- and C-terminal GS linker did not consistently produce active protein.

TABLE 18A
TrxA-SKM C-terminal fusions

| | | | |
|---|---|---|---|
| | TrxA-SKM GKG- | TrxA-SKM CKC- | TrxA-SKM SKS- |
| Parameter | Cterm (4) | Cterm (5) | Cterm (3) |

Experiment 1

| OD_{450, max} | 0.2892 | 0.3116 | 0.3222 |
|---|---|---|---|
| [a.u.] | | | |
| IC₅₀ [nM] | 9.174 | 1.364 | 1.369 |
| R² | 0.9890 | 0.9837 | 0.9477 |
| | TrxA-SKM GKG- | TrxA-SKM SKS- | TrxA-SKM CKC- |
| Parameter | Cterm (4) | Cterm (3) | Cterm (5) |

Experiment 2

| | | | |
|---|---|---|---|
| OD_{450, max} | 2.6 × 10¹² | 0.5749 | 0.6343 |
| [a.u.] | | | |
| IC₅₀ [nM] | Unstable | 2.280 | 1.917 |
| R² | 0.9924 | 0.9814 | 0.9714 |

TABLE 18B
TrxA-SKM Loop fusion constructs

| | | | |
|---|---|---|---|
| | TrxA-SKM GKG- | TrxA-SKM CKC- | TrxA-SKM SKS- |
| Parameter | Cterm (2) | Cterm (1) | Cterm (6) |

Experiment 1

| OD_{450, max} | 0.7133 | 0.7283 | 0.7681 |
|---|---|---|---|
| [a.u.] | | | |
| IC₅₀ [nM] | 2.691 | 1.971 | 0.4183 |
| R² | 0.9901 | 0.9820 | 0.9808 |
| | TrxA-SKM GKG- | TrxA-SKM SKS- | TrxA-SKM CKC- |
| Parameter | Cterm (2) | Cterm (1) | Cterm (6) |

Experiment 2

| | | | |
|---|---|---|---|
| OD_{450, max} | 0.69 | 0.5704 | 0.5748 |
| [a.u.] | | | |
| IC₅₀ [nM] | 156.3 | 1.197 | 0.5815 |
| R² | 0.9901 | 0.9267 | 0.9775 |

### Example 17: Generation and Binding of Knob Fusion Constructs

Additional strategies to modify expression of knob sequences were assessed to compare impacts on protein yield, stability, and activity. In these strategies, knob fusions constructs were generated that were linked to sfGFP (superfolded GFP) or an immunoglobulin Fc domain. Additional TrxA-fusion constructs also were assessed.

### A. Protein Expression

sfGFP (superfolded GFP)-knob and Fc-knob loop fusions were generated as described below.

sfGFP-knob fusions were expressed in electrocompetent Origami E. coli cells as modified pET32b (+) plasmids encoding the sequence for the loop engineered sfGFP proteins as set forth in Table E19A. The loop engineered sfGFP proteins were designed to comprise a G3 knob (i.e., R2G3 knob as set forth in SEQ ID NO: 291) into the D174-G175 loop domain of the sfGFP (SEQ ID NO: 321) to allow for a detectable label with the small high-affinity knob peptide.

TABLE E19A
sfGFP Loop Constructs

| | N-terminal | | C-terminal | Full |
|---|---|---|---|---|
| | Knob Overhang | Knob Peptide | Knob Overhang | construct |
| | SEQ ID NO | SEQ ID NO | SEQ ID NO | SEQ ID NO |
| sfGFP control | N/A | N/A | N/A | 321 |
| sfGFP-link-G3 | GGGS | G3 | GGS | 322 |
| | | (e.g., 291) | (e.g., 109) | |
| sfGFP-stalk-G3 | Stalk A | G3 | Stalk B | 323 |
| | (e.g. 196) | (e.g., 291) | (e.g. 197) | |
| sfGFP-coiled | 202 | G3 | 203 | 324 |
| coil-G3 | | (e.g., 291) | | |

Fc-knob loop fusions as set forth in Table E19B below were similarly expressed in electrocompetent Origami E. coli cells as modified pET32b (+) plasmids encoding the human immunoglobulin G1 (IgG1) constant region with the G3 knob domain (i.e., R2G3 knob as set forth in SEQ ID NO: 291) engineered into the SKAKGQPREP loop of IgG1 Fc (e.g., defined by S122-P131 of SEQ ID NO: 332). The encoded protein in some cases also comprised a signal sequence, such as the IL-2 signal sequence set forth in SEQ ID NO: 333. Without wishing to be bound by theory, it is considered that fusion to Fc can increase protein expression and facilitate purification via increased stability.

TABLE E19B
Fc-G3 Constructs

| | N-terminal | | C-terminal | Full |
|---|---|---|---|---|
| | Knob Overhang | Knob Peptide | Knob Overhang | construct |
| | SEQ ID NO | SEQ ID NO | SEQ ID NO | SEQ ID NO |
| Fc-G3 coiled | 202 | G3 | 203 | 325 |
| coil (cc) | | (e.g., 291) | | |
| Fc-G3 link | GGGS | G3 | GGS | 326 |
| | | (e.g., 291) | (e.g., 109) | |

sfGFP-kob loop constructs were inoculated into a 10 mL starter culture of 2×YT growth medium containing 2% glucose, 10 μg/mL tetracycline, and 50 μg/mL carbenicillin. The starter culture was grown at 37° C. overnight with shaking and added to 100 mL of fresh 2×YT growth medium containing 2% glucose, 10 μg/mL tetracycline, and 50 μg/mL carbenicillin then incubated with shaking until the OD600 of the culture was between 0.5-0.8, at which point the cells were pelleted by centrifugation at 10,000×g for 10 minutes and resuspended in an expression media made from 100 mL of fresh 2×YT broth, 50 μg/mL carbenicillin, and 100 μM IPTG. The expression culture was incubated at 22° C. with shaking for 5 hours and then pelleted by centrifugation at 10,000×g for 10 minutes and stored at −20° C. overnight. Pelleted cells were resuspended in Bug Buster® HT lysis buffer (Millipore Sigma) and incubated at room temperature for 20 minutes on a rotating mixer. The lysis mixture was spun at 16,000×g for 20 minutes. The supernatant was saved and applied to column IMAC purification using Talon® Metal Affinity Resin (Takara Bio), which was first equilibrated with 6 column volumes of a 50 mM NaPO4 pH 8.0, 300 mM NaCl, and 10 mM Imidazole buffer. The lysate supernatant was allowed to bind to the column at 4° C. for two hours on a rotating mixer. The column was drained and washed with 6 CV of the equilibration buffer. Bound protein was eluted by applying 1 CV of buffers with 50 mM NaPO4 pH 8.0, 300 mM NaCl, and increasing concentrations of Imidazole, from 25 mM to 500 mM. The eluted fractions containing protein were spin concentrated and buffer exchanged into 1×PBS pH 7.4 (Corning) using Amicon Ultra-15 10K MWCO centrifuge filters (Millipore Sigma), according to manufacturer's specifications.

Fc-knob loop fusions were similarly inoculated as described above, including transient transfection of the constructs as set forth in Table E19B. Cells were shaken at 37° C. for 5 days in the presence of 8% CO2. The cell suspension was separated by centrifugation at 300 rpm for 5 min, followed by filtration of the supernatant with a 0.22 μm filter. The supernatant was concentrated using a 30 kDa MWCO spin filtration unit (Pierce) and loaded onto a Protein A-Sepharose column (Cytiva), which was previously equilibrated with 6 CV 20 mM NaPO4 pH 7.0. The supernatant was allowed to bind to the column at 4° C. for two hours on a rotating mixer. The column was drained and washed with 6 CV of the equilibration buffer. Bound protein was eluted by applying 2 CV 0.1 M Glycine-HCl pH 2.7 and collected into 0.2 CV of 1M Tris buffer pH 8.0. The eluted protein was spin concentrated and buffer exchanged into 1×PBS pH 7.4 (Corning) using Amicon Ultra-15 30K MWCO centrifuge filters (Millipore Sigma), according to manufacturer's specifications.

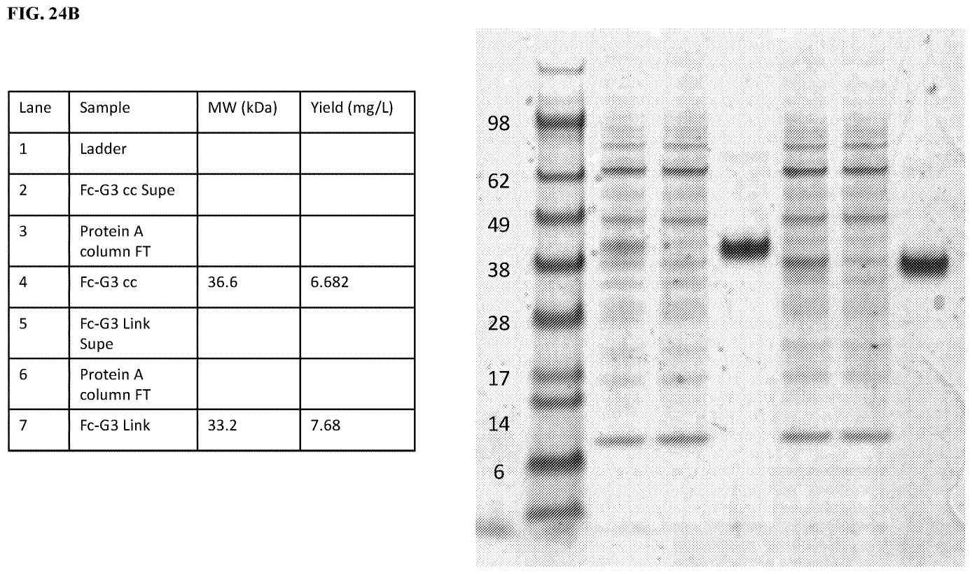

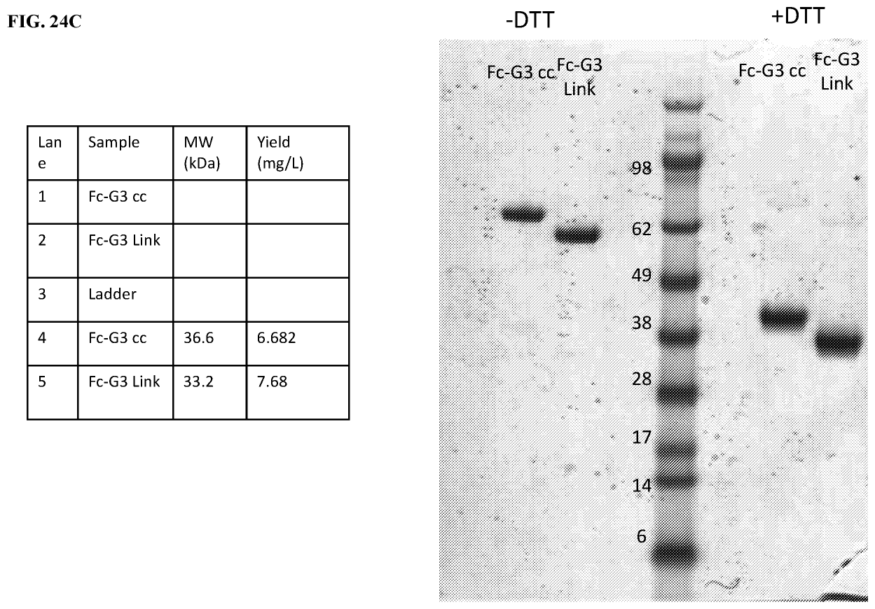

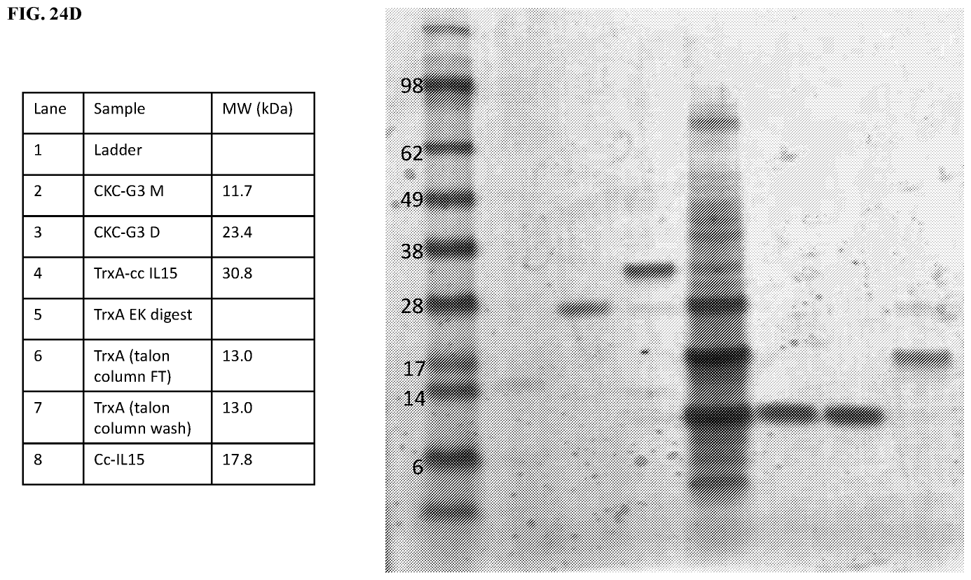

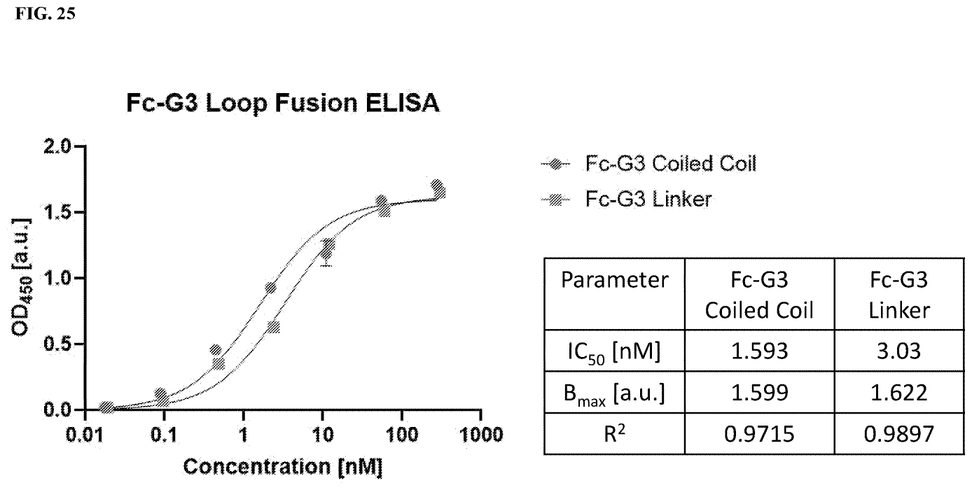

Samples were then quantified using A280 measured on a NanodropOne spectrophotometer (Thermo Fisher Scientific) and resolved by SDS-Page and stained with InstantBlue Coomassie Protein Stain (Abcam) as shown in FIG. 24A (sfGFP Loop Constructs) and FIG. 24B (Fc-G3 Constructs).

The Fc-G3 constructs were also resolved by SDS-Page in the presence and absence of DTT in FIG. 24C. The Fc-G3 construct Fc-G3 coiled coil (i.e., CKC-G3) was observed to form dimers (i.e., G3-D) as well as monomers (i.e., G3-M) with the addition of DTT as shown in FIG. 24D.

Additional strategies to modify fusion protein sequences were also assessed, including strategies similar to those described in Example 16 to generate TrxA fusion constructs. In these strategies, either knob peptide fusion constructs or constructs using IL-15 instead of a knob peptide (e.g., G3) were generated with a coiled-coil motif (CXC). The IL-15 constructs included a coiled coil (cc) IL-15 peptide that was generated from human IL-15 (SEQ ID NO: 320) with coil-coiled motifs, as well as a construct in which the coiled coil IL-15 was fused at the c-terminus of the TrxA chaperone (i.e., TrxA_Cterm-ccIL15). Finally, a TrxA with flag as described in Example 16 was also assessed. The generated constructs are set forth in Table E19C. A purified CKC-G3 protein monomer product (CKC-G3 (M)) was generated using Size Exclusion Chromatography on FPLC (Fast Pressure Liquid Chromatography).

TABLE E19C
Additional Constructs

| | |
|---|---|
| CKC-G3 (M) | SEQ ID NO: 327 |
| TrxA_Cterm-ccIL15 | SEQ ID NO: 328 |
| TrxA + flag | SEQ ID NO: 329 |
| cc-IL15 | SEQ ID NO: 330 |

Nucleotide sequences encoding the fusion constructs set forth in Table E19C above were cloned into an expression vector and transformed into Origami 2 DE3 bacteria. The bacteria were grown substantially as described in Example 6. Supernatant was collected and were purified as previously described. The proteins were assessed by SDS-PAGE as shown in FIG. 24D. Quantification data similarly showed improved expression for the coiled coil constructs (TrxA coiled coil, sfGFP coiled coil, and TrxA coiled coil IL15).

### B. Binding Activity

Binding for constructs generated above were assessed via Enzyme-linked Immunosorbent Assay (ELISA).

For Fc fusion constructs, high-binding, clear bottom, flat 96-well plates (Corning 3690) were coated with 50 ng soluble WT SARS-CoV-2 RBD (NCBI accession number: NC_045512.2) in 1×PBS pH 7.4 (Corning). For sfGFP fusion constructs, high binding, flat bottom, black opaque 96-well plates were coated with 50 ng soluble WT SARS-CoV-2 RBD (NCBI accession number: NC_045512.2) in 1×PBS pH 7.4 (Corning).TrxA fusion constructs described in Table E19C were also assessed using a high-binding, clear bottom, flat 96-well plates (Corning 3690) were coated with 50 ng soluble WT SARS-CoV-2 RBD (NCBI accession number: NC_045512.2) in 1×PBS pH 7.4 (Corning).

Plates were incubated at 4° C. overnight and then blocked with 2% milk (Marvel, dried skim milk) in Tris buffered saline pH 7.4 containing 0.1% Tween-20 (TBST) at room temperature for 2 hours. Serially diluted loop engineered samples and controls as described above were added to the wells in 2% milk/TBST and incubated at room temperature for 1 hour. Plates were then washed four times in TBST, then Anti-p53 (pantropic) Mouse mAb (EMD Millipore Corp. OP43-100UG) diluted 1:5000 in 2% milk in TBST was added to the wells. Plates were incubated at room temperature for 30 minutes, washed four times in TBST, then Goat pAb anti-mouse IgG-HRP (Abcam ab205719) diluted 1:5000 in 2% milk in TBST was added to the wells. Plates were again incubated at room temperature for 30 minutes and washed five times with TBST. Plates were then developed by adding 50 μL TMB substrate solution (Thermo Scientific) per well and incubated at room temperature for 30 seconds. The HRP-TMB reaction was terminated by the addition of 50 μL 1.0N H2SO4 per well. The optical density at 450 nm was read on a microplate reader (Spectramax M2, Molecular Devices). Antigen-binding curves and IC50 values were generated and calculated using a non-linear regression model of one site-specific binding in GraphPad Prism 9.3.1 (GraphPad Software Inc., San Diego, CA.).

The results of the ELISA shown in FIG. 25 show that the Fc-G3 coiled coil construct has a lower IC₅₀ and Bₘₐₓ than the Fc-G3 linker.

These results support that the coiled coil motif stabilizes the knob peptide when expressed as a fusion construct, resulting in higher yields as well as improved binding.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID | Name Sequence | |
| 1 | CAGGCCGTCCTGAACCAGCCAAGCAGCGTCTCCGGGTCTCT | BLV1H12 Light |
| | GGGGCAGCGGGTCTCAATCACCTGTAGCGGGTCTTCCTCCA | chain DNA |
| | ATGTCGGCAACGGCTACGTGTCTTGGTATCAGCTGATCCCTG | |
| | GCAGTGCCCCACGAACCCTGATCTACGGCGACACATCCAGA | |
| | GCTTCTGGGGTCCCCGATCGGTTCTCAGGGAGCAGATCCGG | |
| | AAACACAGCTACTCTGACCATCAGCTCCCTCCAGGCTGAGG | |
| | ACGAAGCAGATTATTTCTGCGCGTCTGCCGAGGACTCTAGTT | |
| | CAAATGCCGTGTTTGGAAGCGGCACCACACTGACAGTCCTA | |
| 2 | QAVLNQPSSVSGSLGQRVSITCSGSSSNVGNGYVSWYQLIPGS | BLV1H12 variable |
| | APRTLIYGDTSRASGVPDRFSGSRSGNTATLTISSLQAEDEADY | Light chain |
| | FCASAEDSSSNAVFGSGTTLTVL | |
| 3 | CCGCTCTTCAGGGCACCCGAGTTCC | igGCDNA2REV |
| 4 | CTGACTGTGCTGTTGTTGAACTTCC | igMCDNA2REV |
| 5 | GACACGCTGTCGCCATTCTGGTTCC | igACDNA2REV |
| 6 | CGGGCACGGTCACCATGCTGCTGAGAGAGTAG | igGCDNA1.7REV |
| 7 | TTACCTGCGGCCGCTGAGGAGACGGTGACCAGGAGTCCAAC | BOVVHFR4REV |
| | TGGAGCTCCATCAAG | |
| 8 | CAGCCGGCCATGGCCACATACTACAGTACTACTGTACACC | BOVSTALKFOR1 |
| 9 | CAGCCGGCCATGGCCACATACTACAGTACTACTGTATACC | BOVSTALKFOR2 |
| 10 | CAGCCGGCCATGGCCACATACTACAGTACTACTGTGCTCC | BOVSTALKFOR3 |
| 11 | CAGCCGGCCATGGCCACATACTACAGTGGTACTGTGCACC | BOVSTALKFOR4 |
| 12 | AAAAAGCCATGGTGCAGGTGCAGCTGCGGGAGTCGGG | BOVVHNCOFOR2 |
| | | NotI restriction |
| | | enzyme site |
| | | (bold/underline) |
| 13 | TTACCTCTCGAGTGAGGAGACGGTGACCAGGAGTCC | BOVVHFR4XHOREV |
| | | Xho I restriction |
| | | enzyme site |
| | | (bold/underline) |
| 14 | CAGGTGCAGCTGCGGGAGTCGGGCCCCAGCCTGGTGAAGCC | R2G3 |
| | CTCACAGACCCTCTCGCTCACCTGCGCGGCCTCTGGATTCTC | |
| | ATTGAGCGACAAGGCTGTAGGCTGGGTCCGCCGGGCTCCAG | |
| | GGAAGGCGCTGGAGTGGCTCGGTAGTATAGACACTGGTGGA | |
| | AGCACAGGCTATAACCCAGGCCTGAAATCCCGGCTCAGCAT | |
| | CACCAAGGACAACTCCAAGAGCCAAGTCTCCCTGTCAATTA | |
| | GCAGCGTAACGTCTGAGGACTCGGCCACATACTACTGTGCA | |
| | ACTGTACACCAGAAAACAGCTGAAGGAGACAAAACGTGTC | |
| | CTGATGGTTACGAGCATACTTGTGGTTGCATTGGGGGTTGTG | |
| | GTTGCAAAAGGTCTGCCTGTATAGGTGCACTTTGTTGCCAAG | |
| | CGTCGTTGGGTGGTTGGCTTAGTGACGGTGAAACCTACACTT | |
| | ACGAGTTCCACGTCGATACCTGGGGCCAAGGACTCGTGGTC | |
| | ACCGTCTCCTCA | |
| 15 | CAGGTGCAGCTGCGGGAGTCGGGCCCCAGCCTGGTGAAGCC | SR3A3 |
| | CTCACAGACCCTCTCCCTCACCTGCACAATCTCTGGATTCTC | |
| | ATTGAGTAGCTATGCTGTACTCTGGGTCCGCCAGGCTCCAG | |
| | GGAAGCCGCTGGAGTGGCTCGGTAGTATAGACACTGCGGAA | |
| | AACACAGGCTATAACCCAGGCCTGAAATCCCGGCTCAGCAT | |
| | CACCAAGGACAACTCCAAGAGCCAAGTCTCTCTGTCAGTGA | |
| | GCAGCGTGACAACCGAGGACTCGGCCACATACTACTGTGCT | |
| | ACTGTACACCAGAAAACGCGAAAAGAAAAAAATTGTCCTG | |
| | ATGGCTATATCTATAGTTCTAATATCACTAGCGGTTTTGATT | |
| | GTGGTGTCTGGATTTGTCGTCGCGTCGGTAGTGCCTTCTGTA | |
| | GTCGTACTGGTGATTATACTAGTCCTACTGAACTTGACATTT | |
| | ACGAGTTCTACGTCGAAGGGTGGGGCCAGGGAGTCCCGGTC | |
| | ACCGTCTCCTCA | |
| 16 | CAGGTGCAGCTGCGGGAGTCGGGCCCCAGCCTGGTGAAGCC | R2F12 |
| | CTCACAGACCCTCTCCCTCACCTGCACGGTCTCTGGATTCTC | |
| | ATTGAGCGACAAGGCTGTAGGCTGGGTCCGCCGGGCTCCAG | |
| | GGAAGGCGCTGGAGTGGCTCGGTAGTATAGACACTGGTGGA | |
| | ATGACAGGCTATAACCCAGGCCTGAAATCCCGGCTCAGCAT | |
| | CACCAAGGACAACTCCAAAAGCCAAGTCTCTCTATCAGTGA | |
| | ATAGCGTGACAACTGAGGACTCGGCCACGTACTACTGTGCC | |
| | ACTGTAGACCAGAAAACGAAAAATGCTTGCCCTGATGATTT | |
| | CGATTATCGTTGTTCGTGTATCGGTGGTTGTGGCTGCGCCCG | |
| | TAAAGGATGCGTTGGTCCTCTTTGTTGTCGTTCTGATTTGGG | |
| | TGGCTATCTTACTGATAGTCCTGCTTACATTTACGAATGGTA | |
| | TATTGATCTTTGGGGCCAAGGACTCCTGGTCACCGTCTCCTC | |
| | A | |
| 17 | CAGGTGCAGCTGCGGGAGTCGGGCCCCAGCCTGGTGAAGCC | R5A3 |
| | GTCACAGACCCTCTCGCTCACCTGCACGGCCTCTGGATTCTC | |
| | ATTGAGCGACAAGGCTGTAGGCTGGGTCCGCCAGGCTCCAG | |
| | GGAAGGCGCTGGAGTGGCTCGGTAGTATAGACACTGGTGGA | |
| | AGCACAGGCTATAACCCAGGCCTGAAATCCCGGCTCAGCAT | |
| | CACCAAGGACAACTCCAAGAGCCAAGTCTCTCTGTCAGTGA | |
| | GCAGCGTGACAACTGAGGACTCGGCCACATACTACTGTACT | |
| | ACTGTGCACTGTAGTGATGGTGGTTATGTTGAGGCGGGTTTT | |
| | GGTTGTTGGCCTTGGGATTATGGTTATCCTTACGTCGATGCC | |
| | TGGGGCCAAGGACTCCTGGTCACCGTCTCCTCA | |
| 18 | CAGGTGCAGCTGCGGGAGTCGGGCCCCAGCCTGGTGAAGCC | R4G11 |
| | CTCACAGACCCTCTCCCTCACCTGCACGGTCTCTGGATTCTC | |
| | ATTGAGCAGCTATGGTATAACCTGGGTCCGCCAGGCTCCAG | |
| | GGAAGGCGCTGGAGTGCCTCGGTAGTATAAGCAGTGGTGGA | |
| | ACCACAGACTACAACCCAGCCCTGAAATCCCGGCTCAGCAT | |
| | CACCAAGGACAACTCCAAGAGCCAAGTCTCTCTGTCAGTGA | |
| | GCAGCGTGACACCTGAGGACACGGCCACATACTACTGTTCG | |
| | AAGTGGAATTTAGAATATACTTGGGGTGGTGTTGGTTGCGC | |
| | TAGTTTTGCTGATGAGGACACCCACGTTGATGCCTGGGGCC | |
| | AAGGACTCCTGGTCACCGTCTCCTCA | |
| 19 | CAGGTGCAGCTGCGGGAGTCGGGCCCCAGCCTGATGAAGCC | R4G3 |
| | CTCACAGACCCTCTCCCTCACCTGCACGGTCTCTGGGTTCTC | |
| | ATTGAGCGACTATGCTGTAGGCTGGGTCCGCCAGGCCCCAG | |
| | GGAAGGCGCTGGAGTGGCTCGGTGGTATAGACACTGGTGGA | |
| | AGCACAGGCTATAACCCAGGCCTGGAATCCCGGCTCAGCAT | |
| | CACCAAGGACAACTCCAAGAGCCAAGTCTCTCTGTCAGTGA | |
| | GCAGCGTGACAACTGAGGACTCGGCCACATACTACTGTACT | |
| | ACTGTGGTCCTTTGTTATTTTAATTATGTTGTTCGTCGTTATA | |
| | ATTGTGGTGGTCTTGGTTATGGGCATGGCTTTAATAGTTTCT | |
| | ACGTCGATGCCTGGGGCCAAGGACTCCTGGTCACCGTCTCC | |
| | TCA | |
| 20 | CAGGTGCAGCTGCGGGAGTCGGGCCCCAGCCTGGTGAAGCC | R4E5 |
| | CTCACAGACCCTCTCCCTCACCTGCACGACCTCTGGATTCTC | |
| | ACTGAGAAACTATGCTGTAGGCTGGGTCCGCCAGGCTCCGG | |
| | GGAAGGCGCTGGAGTGGCTCGGTGGTATAGACACTGGTGGA | |
| | AGCACAGGCTATAACCCAGGCCTGGAATCCCGGCTCAGCAT | |
| | CACCAAGGACAACTCCAAGAGCCAAGTCTCTCTGTCAGTGA | |
| | GCAGCGTGACAACTGAGGACTCGGCCACATACTACTGTACT | |
| | ACTGTGGTCCTTTGTTATTTTAATTATGTTGTTCGTCGTTATA | |
| | ATTGTGGTGGTCTTGGTTATGGGCATGGCTTTAATAGTTTCT | |
| | ACGTCGATGCCTGGGGCCAAGGACTCCTGGTCACCGTCTCC | |
| | TCA | |
| 21 | CAGGTGCAGCTGCGGGAGTCGGGCCCCAGCCTGGTGAAGCC | R4C1 |
| | GTCACAGACCCTCTCGCTCACCTGCACGGCCTCTGGATTCTC | |
| | ATTGAGCGATAAGGCTGTAGGCTGGGTCCGCCAGGCTCCAG | |
| | GGAAGCCGCTGGAGTGGCTCGGTAGTATAGACACTGCGGAA | |
| | AACACAGGCTATAACCCAGGCCTGAAATCTCGGCTCAGCAT | |
| | CACCAAGGACAACTCCAAGAGCCAAGTCTCTCTGTCAGTGA | |
| | GCAGCGTGACAACTGAGGACTCGGCCACATACTACTGTGCT | |
| | ACTGTACACCAGAAAACGCGAAAAGAAAAAAATTGTCCTG | |
| | ATGGCTATATCTATAGTTCTAATACCGCcAGCGGTTATGATT | |
| | GTGGTGTCTGGATTTGTCGTCGCGTCGGTAGTGCCTTCTGTA | |
| | GTCGTACTGGTGATTATACTAGTCCTAGTGAATTTGACATTT | |
| | ACGAgTTCTACGTCGAAGGGTGGGGCCAGGGAcTCCtGGTCA | |
| | CCGTCTCCTCA | |
| 22 | CAGGTGCAGCTGCGGGAGTCGGGCCCCAGCCTGGTGAAGCC | R4A10 |
| | CTCACAGACCCTCTCCCTCACCTGCACGACCTCTGGATTCTC | |
| | ATTGAGCGACTATGCTGTAGGCTGGGTCCGCCAGGCTCCAG | |
| | GGAAGGCGCTGGAGTGGCTCGGTGGTATAGACACTGGTGGA | |
| | AGCACAGGCTATAACCCAGGCCTGAAATCCCGGCTCAGCAT | |
| | CACCAAGGACAACTCCAAGAGTCAAGTCTCTCTGTCAGTGA | |
| | GCAGCGTGACAACTGAGGATTCGGCCACATACTACTGTACT | |
| | GCCGTGGTCCTCTGTTATTACAATCGGGTTGTGCGTCGTAAT | |
| | AATTGTGGTGGGCTTGGTTATGATTATGGTTTTGATCATTTC | |
| | TACGTCGATGCCTGGGGCCAAGGACTCCTGGTCACCGTCTC | |
| | CTCA | |
| 23 | CAGGTGCAGCTGCGGGAGTCGGGCCCCAGCCTGGTGAAGCC | R2G1 |
| | CTCACAGACCCTCTCCCTCACCTGCACGGTCTCTGGATTCTC | |
| | ATTGAGCAACTATGCTGTAGGCTGGGTCCGCCAGGCTCCAG | |
| | GGAAGGCGCTGGAGTGCCTCGGTGATGTAGACAGTAGTGGA | |
| | GGCACAGCCTATAACCCAGCCCTGAAATCCCGGTTCATCAT | |
| | CGCCAAGGACAACTCCAAGAACCAAGTCTCTCTGTCAGTCC | |
| | GCAGCGTGACACCTGAGGACACGGCCACATACTACTGTGCG | |
| | AAGTTTGCTAAGGGTACTACGAGTGCTGGTGCTTGTGATTAT | |
| | TCAGAAAGCTACGTCGATGCCTGGGGCCAGGGACTCCTGGT | |
| | CACCGTCTCCTCA | |
| 24 | CAGGTGCAGCTGCGGGAGTCGGGCCCCAGCCTGGTGAAGCC | R2D6 |
| | CTCACAGACCCTCTCCCTCACCTGCACGACCTCTGGATTCTC | |
| | ACTGAGCAGCTATGCTGTAGGCTGGGTCCGCCAGGCTCCGG | |
| | GGAAGGCGCTGGAGTGGGTTGGTGATATAGATTATGTCGGA | |
| | AACACAGACTATAACCCAGCCCTGAAATCCCGGCTCAGCAT | |
| | CACCAAGGACAACTCCAAGAGCCAAGTCTCTCTGGTAGTGA | |
| | GCAGCGTGACAGCTGAGGACGCGGCCACATACTACTGTGCG | |
| | AAATATTCCGGTGCTTATGCTTATGCTGCTTGCAATTATTAT | |
| | GGTTGGCGTTGTGCTTGGGAAAGCTACATCGATGCCTGGGG | |
| | CCAAGGACTCCTGGTCACCGTCTCCTCA | |
| 25 | CAGGTGCAGCTGCGGGAGTCGGGCCCCAGCCTGGTGAAGCC | R2B1 |
| | CTCACAGACCCTCTCCCTCACCTGCACGGTCTCTGGATTTTC | |
| | ATTAAGCGATAATAATGTAGGCTGGGTCCGCCAGGCTCCAG | |
| | GAAAGGCGCTGGAGTGGCTCGGTGTAATGCATAATGATGGG | |
| | AACAAAGGCTATAACCCAGCCCTGAAATCCCGGCTCAGCAT | |
| | CACCAAGGACAGCTCCAAGAGCCAAGTCTCTCTATCACTAA | |
| | GCAGCGTGACAAGTGAGGACACGGCCACATACTACTGTACA | |
| | AGAGACAATGCACGTTGTGATAGTTGGACGTATGACAGCTG | |
| | TGATACTTGGTATCGCAATTCGTGGCACGTTGATGCCTGGGG | |
| | CCAAGGACTCCTgGTCACCGTCTCCTCA | |
| 26 | CAGGTGCAGCTGCGCGAGTCGGGCCCCAGCCTGGTGAAGCC | SKM-BLV1H12 |
| | GTCACAGACCCTCTCGCTCACCTGCACGGCCTCTGGATTCTC | |
| | ATTGAGCGACAAGGCTGTAGGCTGGGTCCGCCAGGCTCCAG | |
| | GGAAGGCGCTGGAGTGGCTCGGTAGTATAGACACTGGTGGA | |
| | AACACAGGCTATAACCCAGGCCTGAAATCCCGGCTCAGCAT | |
| | CACCAAGGACAACTCCAAGAGTCAAGTCTCTCTGTCAGTGA | |
| | GCAGCGTGACAACTGAGGACTCGGCCACATACTACTGTACT | |
| | ACTGTGCACCAAGAGACCTTACGTAGTTGTCCTGATGGTTAT | |
| | ATTGATAATTCTGGATGCACGGCTGATTGGGGTTGTGCAGCT | |
| | CTTGATTGTTGGCGGCGTCGTTTTGGTTACCACAGCACTGAT | |
| | CCTTCTCATTATACTGGTGCGACGTATATTTACACGTACAGC | |
| | TTGCACATCGATGCCTGGGGCCAAGGACTCCTGGTCACCGT | |
| | CTCCTCA | |
| 27 | CAGGTGCAGCTGCGCGAGTCGGGCCCCAGCCTGGTGAAGCC | SKD-BLV1H12 |
| | GTCACAGACCCTCTCGCTCACCTGCACGGCCTCTGGATTCTC | |
| | ATTGAGCGACAAGGCTGTAGGCTGGGTCCGCCAGGCTCCAG | |
| | GGAAGGCGCTGGAGTGGCTCGGTAGTATAGACACTGGTGGA | |
| | AACACAGGCTATAACCCAGGCCTGAAATCCCGGCTCAGCAT | |
| | CACCAAGGACAACTCCAAGAGTCAAGTCTCTCTGTCAGTGA | |
| | GCAGCGTGACAACTGAGGACTCGGCCACATACTACTGTACT | |
| | ACTGTGCACCAGCGTACAAGCGAAAAAAGAAGTTGTCCTGG | |
| | CGGTAGTAGTAGACGTTATCCTAGTGGCGCCAGTTGTGACG | |
| | TTAGTGGGGGCGCTTGTGCGTGTTATGTTTCTAATTGTAGAG | |
| | GCGTTTTGTGTCCTACTCTTAACGAAATCGTTGCTTATACCT | |
| | ACGAATGGCACGTCGACGCCTGGGGCCAAGGACTCCTGGTC | |
| | ACCGTCTCCTCA | |
| 28 | CAGGTGCAGCTGCGGGAGTCGGGCCCCAGCCTGGTGAAGCC | RBD F4 |
| | CTCACAGACCCTCTCCCTCACCTGCACGGTCTCTGGATTCTC | |
| | ATTGAGCAGCAATGGTGTGGTCTGGGTCCGCCAGGCTCCAG | |
| | GGAAGGCGCTGGAGTGGCTCGGTGATATATGCAGTACTGGA | |
| | GGCACAAGCTTTAACCCAGCCCTGAAATCCCGGCTCAGCAT | |
| | CGCCAAGGACAACTCCAAGAGCCAAGTCTCTCTGTCAGTGA | |
| | GAAGCGTGACACCTGAGGACACGGCCACATATTACTGTGCA | |
| | AGAAGTCGTGGTTATGATTGTTATGCTAATGTGGATGCTTTG | |
| | GACTACGTCGATGCCTGGGGCCAAGGACTCCTGGTCACCGT | |
| | CTCCTCA | |
| 29 | CAGGTGCAGCTGCGGGAGTCGGGCCCCAGCCTGGTGAAGCC | RBD C6 |
| | CTCACAGACCCTCTCCCTCACCTGCACGGTCTCTGGATTCTC | |
| | ATTGAGCAGCAATGGTGTAGTCTGGGTCCGCCAGGCTCCAG | |
| | GGAGACCACTGGAGTGGCTCGGTGATATATGCAGTAATGGA | |
| | GGCACAAGCTTTAACCCAGCCCTGAAATCCCGGCTCAGCAT | |
| | CGCCAAGGACAACTCCGAGAGCCAAGTCTCTCTGACCGTGA | |
| | GAAGCGTGACACCTGAGGACACAGCCACATATTACTGTGCA | |
| | AGAAGTCGTGGTTATGATTGTTATGCTTATGTTTATGCTTTG | |
| | GACACCGTCGATGCCTGGGGCCAAGGACTCCTGGTCACCGT | |
| | CTCCTCA | |
| 30 | CAGGTGCAGCTGCGGGAGTCGGGCCCCAGCCTGGTGAAGCC | RBD A2 |
| | CCTACAGATCCTCTCCCTCACCTGCACGGTCTCTGGATTCTC | |
| | ATTGAGCAGCAATGGTGTGGTCTGGGTCCGCCAGGCTCCAG | |
| | GGAAGGCGCTGGAGTGGCTCGGTGATATATGCAGTACTGGA | |
| | GGCACAAGCTTTAACCCAGCCCTGAAATCCCGGCTCAGCAT | |
| | CGCCAAGGACAACTCCAAGAGCCAAGTCTCTCTGTCAGTGA | |
| | GAAGCGTGACACCTGAGGACACGGCCACATATTACTGTGCA | |
| | AGAAGTCGTGGTTATGATTGTTATGCTAATGTGGATGCTTTG | |
| | GACTACGTCGATGCCTGGGGCCAAGGACTCCTGGTCACCGT | |
| | CTCCTCA | |
| 31 | CAGGTGCAGCTGCGGGAGTCGGGCCCCAGCCTGGTGAAGCC | SA-R2C3 |
| | GTCACAGACCCTCTCGCTCACCTGCACGGCCTCTGGATTCTC | |
| | ATTGAGCGATAAGCCTGTAGGCTGGGTCCGCCAGGCTCCAG | |
| | GGAAGCCACTGGAGTGGCTCGGTAGTATAGACACTGCGGAA | |
| | AACACAGGCTATAACCCAGGCCTGAAATCTCGGCTCAGCAT | |
| | CACCAAGGACAACTCCAAGAGCCAAGTCTCTCTGTCACTGA | |
| | GCAGCGTGACGACTGAGGACTCGGCCACATACTACTGTGCT | |
| | ACTGTACACCAGAAAACGCGGAAGGAAAAAAGTTGTCCTG | |
| | ATGGCTATCTCTATAGTTCTAATACCGGCCGCGGTTATGATT | |
| | GTGGTGTCTGGACTTGTCGTCGCGTCGGTGGTGAATTCTGTA | |
| | GTGCTACTGGTGATTGGACTAGTCCTAGTGAAGAAGACTTTT | |
| | ACGAATTCTACGTCGATACGTGGGGCCAGGGAGCCCCGGTC | |
| | ACCGTCTCCTCA | |
| 32 | CAGGTGCAGCTGCGGGAGTCGGGCCCCAGCCTGGTGAAGCC | SA-R2D9 |
| | GTCACAGACCCTCTCGCTCACCTGCACGGCCTCTGGATTCTC | |
| | ATTAAGCGACAAGGCTATTGGCTGGGTCCGCCAGGCTCCAG | |
| | GGAAGGCGCTGGAGTGGCTCGGTAGTATAGACACCCGTGGA | |
| | AACACAGGCTATAACCCAGGCCTGAAATCCCGACTCAGCAT | |
| | CACCAAGGACAGCTCCAAGAGCCAAGTCTCTCTGTCAGTGA | |
| | ACAGCGTGACAACTGAAGACTCGGCCACGTACCTCTGTGCT | |
| | ATTGTGCAGCAGATCACACACAAAACTTGTCCTAATGGTTA | |
| | CAATTGGTTTGATCGTTGTTGTTCTTGGGATGGTACCTGTGG | |
| | TGATGGTTGTTGCAGTAATCGTGCTTGGCCTAGTGGTAATGG | |
| | TAGAGCCGACAGTAGTATTGGTGAAACTTATGGTTACGAAT | |
| | TTCACGTGGCTGCCTGGGGCCAAGGACTCCTGGTCACCGTCT | |
| | CCTCA | |
| 33 | QVQLRESGPSLVKPSQTLSLTCAASGFSLSDKAVGWVRRAPGK | R2G3 |
| | ALEWLGSIDTGGSTGYNPGLKSRLSITKDNSKSQVSLSISSVTSE | |
| | DSATYYCATVHQKTAEGDKTCPDGYEHTCGCIGGCGCKRSAC | |
| | IGALCCQASLGGWLSDGETYTYEFHVDTWGQGLVVTVSS | |
| 34 | QVQLRESGPSLVKPSQTLSLTCTISGFSLSSYAVLWVRQAPGKP | SR3A3 |
| | LEWLGSIDTAENTGYNPGLKSRLSITKDNSKSQVSLSVSSVTTE | |
| | DSATYYCATVHQKTRKEKNCPDGYIYSSNITSGFDCGVWICRR | |
| | VGSAFCSRTGDYTSPTELDIYEFYVEGWGQGVPVTVSS | |
| 35 | QVQLRESGPSLVKPSQTLSLTCTVSGFSLSDKAVGWVRRAPGK | R2F12 |
| | ALEWLGSIDTGGMTGYNPGLKSRLSITKDNSKSQVSLSVNSVT | |
| | TEDSATYYCATVDQKTKNACPDDFDYRCSCIGGCGCARKGCV | |
| | GPLCCRSDLGGYLTDSPAYIYEWYIDLWGQGLLVTVSS | |
| 36 | QVQLRESGPSLVKPSQTLSLTCTASGFSLSDKAVGWVRQAPGK | R5A3 |
| | ALEWLGSIDTGGSTGYNPGLKSRLSITKDNSKSQVSLSVSSVTT | |
| | EDSATYYCTTVHCSDGGYVEAGFGCWPWDYGYPYVDAWGQ | |
| | GLLVTVSS | |
| 37 | QVQLRESGPSLVKPSQTLSLTCTVSGFSLSSYGITWVRQAPGKA | R4G11 |
| | LECLGSISSGGTTDYNPALKSRLSITKDNSKSQVSLSVSSVTPED | |
| | TATYYCSKWNLEYTWGGVGCASFADEDTHVDAWGQGLLVT | |
| | VSS | |
| 38 | QVQLRESGPSLMKPSQTLSLTCTVSGFSLSDYAVGWVRQAPGK | R4G3 |
| | ALEWLGGIDTGGSTGYNPGLESRLSITKDNSKSQVSLSVSSVTT | |
| | EDSATYYCTTVVLCYFNYVVRRYNCGGLGYGHGFNSFYVDA | |
| | WGQGLLVTVSS | |
| 39 | QVQLRESGPSLVKPSQTLSLTCTTSGFSLRNYAVGWVRQAPGK | R4E5 |
| | ALEWLGGIDTGGSTGYNPGLESRLSITKDNSKSQVSLSVSSVTT | |
| | EDSATYYCTTVVLCYFNYVVRRYNCGGLGYGHGFNSFYVDA | |
| | WGQGLLVTVSS | |
| 40 | QVQLRESGPSLVKPSQTLSLTCTASGFSLSDKAVGWVRQAPGK | R4C1 |
| | PLEWLGSIDTAENTGYNPGLKSRLSITKDNSKSQVSLSVSSVTT | |
| | EDSATYYCATVHQKTRKEKNCPDGYIYSSNTASGYDCGVWIC | |
| | RRVGSAFCSRTGDYTSPSEFDIYEFYVEGWGQGLLVTVSS | |
| 41 | QVQLRESGPSLVKPSQTLSLTCTTSGFSLSDYAVGWVRQAPGK | R4A10 |
| | ALEWLGGIDTGGSTGYNPGLKSRLSITKDNSKSQVSLSVSSVTT | |
| | EDSATYYCTAVVLCYYNRVVRRNNCGGLGYDYGFDHFYVDA | |
| | WGQGLLVTVSS | |
| 42 | QVQLRESGPSLVKPSQTLSLTCTVSGFSLSNYAVGWVRQAPGK | R2G1 |
| | ALECLGDVDSSGGTAYNPALKSRFIIAKDNSKNQVSLSVRSVTP | |
| | EDTATYYCAKFAKGTTSAGACDYSESYVDAWGQGLLVTVSS | |
| 43 | QVQLRESGPSLVKPSQTLSLTCTTSGFSLSSYAVGWVRQAPGK | R2D6 |
| | ALEWVGDIDYVGNTDYNPALKSRLSITKDNSKSQVSLVVSSVT | |
| | AEDAATYYCAKYSGAYAYAACNYYGWRCAWESYIDAWGQG | |
| | LLVTVSS | |
| 44 | QVQLRESGPSLVKPSQTLSLTCTVSGFSLSDNNVGWVRQAPGK | R2B1 |
| | ALEWLGVMHNDGNKGYNPALKSRLSITKDSSKSQVSLSLSSVT | |
| | SEDTATYYCTRDNARCDSWTYDSCDTWYRNSWHVDAWGQG | |
| | LLVTVSS | |
| 45 | QVQLRESGPSLVKPSQTLSLTCTASGFSLSDKAVGWVRQAPGK | SKM-BLV1H12 |
| | ALEWLGSIDTGGNTGYNPGLKSRLSITKDNSKSQVSLSVSSVTT | |
| | EDSATYYCTTVHQETLRSCPDGYIDNSGCTADWGCAALDCWR | |
| | RRFGYHSTDPSHYTGATYIYTYSLHIDAWGQGLLVTVSS | |
| 46 | QVQLRESGPSLVKPSQTLSLTCTASGFSLSDKAVGWVRQAPGK | SKD-BLV1H12 |
| | ALEWLGSIDTGGNTGYNPGLKSRLSITKDNSKSQVSLSVSSVTT | |
| | EDSATYYCTTVHQRTSEKRSCPGGSSRRYPSGASCDVSGGACA | |
| | CYVSNCRGVLCPTLNEIVAYTYEWHVDAWGQGLLVTVSS | |
| 47 | QVQLRESGPSLVKPSQTLSLTCTVSGFSLSSNGVVWVRQAPGK | RBD F4 |
| | ALEWLGDICSTGGTSFNPALKSRLSIAKDNSKSQVSLSVRSVTP | |
| | EDTATYYCARSRGYDCYANVDALDYVDAWGQGLLVTVSS | |
| 48 | QVQLRESGPSLVKPSQTLSLTCTVSGFSLSSNGVVWVRQAPGR | RBD C6 |
| | PLEWLGDICSNGGTSFNPALKSRLSIAKDNSESQVSLTVRSVTP | |
| | EDTATYYCARSRGYDCYAYVYALDTVDAWGQGLLVTVSS | |
| 49 | QVQLRESGPSLVKPLQILSLTCTVSGFSLSSNGVVWVRQAPGK | RBD A2 |
| | ALEWLGDICSTGGTSFNPALKSRLSIAKDNSKSQVSLSVRSVTP | |
| | EDTATYYCARSRGYDCYANVDALDYVDAWGQGLLVTVSS | |
| 50 | QVQLRESGPSLVKPSQTLSLTCTASGFSLSDKPVGWVRQAPGK | SA-R2C3 |
| | PLEWLGSIDTAENTGYNPGLKSRLSITKDNSKSQVSLSLSSVTT | |
| | EDSATYYCATVHQKTRKEKSCPDGYLYSSNTGRGYDCGVWT | |
| | CRRVGGEFCSATGDWTSPSEEDFYEFYVDTWGQGAPVTVSS | |
| 51 | QVQLRESGPSLVKPSQTLSLTCTASGFSLSDKAIGWVRQAPGK | SA-R2D9 |
| | ALEWLGSIDTRGNTGYNPGLKSRLSITKDSSKSQVSLSVNSVTT | |
| | EDSATYLCAIVQQITHKTCPNGYNWFDRCCSWDGTCGDGCCS | |
| | NRAWPSGNGRADSSIGETYGYEFHVAAWGQGLLVTVSS | |
| 52 | GAAGGAGACAAAACGTGTCCTGATGGTTACGAGCATACTTG | R2G3 |
| | TGGTTGCATTGGGGGTTGTGGTTGCAAAAGGTCTGCCTGTAT | |
| | AGGTGCACTTTGTTGCCAAGCGTCGTTGGGTGGTTGGCTTAG | |
| | TGACGGTGAAACCTACACT | |
| 53 | AAAGAAAAAAATTGTCCTGATGGCTATATCTATAGTTCTAA | SR3A3 |
| | TATCACTAGCGGTTTTGATTGTGGTGTCTGGATTTGTCGTCG | |
| | CGTCGGTAGTGCCTTCTGTAGTCGTACTGGTGATTATACTAG | |
| | TCCTACTGAACTTGACATTTACGAGTTC | |
| 54 | AAAACGAAAAATGCTTGCCCTGATGATTTCGATTATCGTTGT | R2F12 |
| | TCGTGTATCGGTGGTTGTGGCTGCGCCCGTAAAGGATGCGTT | |
| | GGTCCTCTTTGTTGTCGTTCTGATTTGGGTGGCTATCTTACTG | |
| | ATAGTCCTGCTTACATTTACGAA | |
| 55 | AAAGAAAAAAATTGTCCTGATGGCTATATCTATAGTTCTAA | R4C1 |
| | TACCGCCAGCGGTTATGATTGTGGTGTCTGGATTTGTCGTCG | |
| | CGTCGGTAGTGCCTTCTGTAGTCGTACTGGTGATTATACTAG | |
| | TCCTAGTGAATTTGACATTTAC | |
| 56 | CTGCGTAGTTGTCCTGATGGTTATATTGATAATTCTGGATGC | SKM-BLV1H12 |
| | ACGGCTGATTGGGGTTGTGCAGCTCTTGATTGTTGGCGGCGT | |
| | CGTTTTGGTTACCACAGCACTGATCCTTCTCATTATACTGGT | |
| | GCGACGTATATTTACACGTAC | |
| 57 | AGCGAAAAAAGAAGTTGTCCTGGCGGTAGTAGTAGACGTTA | SKD-BLV1H12 |
| | TCCTAGTGGCGCCAGTTGTGACGTTAGTGGGGGCGCTTGTG | |
| | CGTGTTATGTTTCTAATTGTAGAGGCGTTTTGTGTCCTACTCT | |
| | TAACGAAATCGTTGCTTATACCTAC | |
| 58 | CGGAAGGAAAAAAGTTGTCCTGATGGCTATCTCTATAGTTC | SA-R2C3 |
| | TAATACCGGCCGCGGTTATGATTGTGGTGTCTGGACTTGTCG | |
| | TCGCGTCGGTGGTGAATTCTGTAGTGCTACTGGTGATTGGAC | |
| | TAGTCCTAGTGAAGAAGACTTTTACGAATTC | |
| 59 | ATCACACACAAAACTTGTCCTAATGGTTACAATTGGTTTGAT | SA-R2D9 |
| | CGTTGTTGTTCTTGGGATGGTACCTGTGGTGATGGTTGTTGC | |
| | AGTAATCGTGCTTGGCCTAGTGGTAATGGTAGAGCCGACAG | |
| | TAGTATTGGTGAAACTTATGGTTACGAATTT | |
| 60 | EGDKTCPDGYEHTCGCIGGCGCKRSACIGALCCQASLGGWLS | R2G3 |
| | DGETYTYEF | |
| 61 | KEKNCPDGYIYSSNITSGFDCGVWICRRVGSAFCSRTGDYTSPT | SR3A3 |
| | ELDIYEF | |
| 62 | KTKNACPDDFDYRCSCIGGCGCARKGCVGPLCCRSDLGGYLT | R2F12 |
| | DSPAYIYE | |
| 63 | RKEKNCPDGYIYSSNTASGYDCGVWICRRVGSAFCSRTGDYTS | R4C1 |
| | PSEFDIY | |
| 64 | LRSCPDGYIDNSGCTADWGCAALDCWRRRFGYHSTDPSHYTG | SKM-BLV1H12 |
| | ATYIYTY | |
| 65 | SEKRSCPGGSSRRYPSGASCDVSGGACACYVSNCRGVLCPTLN | SKD-BLV1H12 |
| | EIVAYTY | |
| 66 | RKEKSCPDGYLYSSNTGRGYDCGVWTCRRVGGEFCSATGDW | SA-R2C3 |
| | TSPSEEDFYEF | |
| 67 | ITHKTCPNGYNWFDRCCSWDGTCGDGCCSNRAWPSGNGRAD | SA-R2D9 |
| | SSIGETYGYEF | |
| 68 | CTTVHQRTSEKRSCPGGSSRRYPSGASCDVSGGACACYVSNCR | SKD |
| | GVLCPTLNEIVAYTYEWHVDAWGQGLLVTVSS | |
| 69 | CTTVHQETLRSCPDGYIDNSGCTADWGCAALDCWRRRFGYHS | SKM |
| | TDPSHYTGATYIYTYSLHIDAWGQGLLVTVSS | |
| 70 | CATVHQKTRKEKNCPDGYIYSSNTASGYDCGVWICRRVGSAF | R4C1 |
| | CSRTGDYTSPSEFDIYEFYVEGWGQGLLVTVSS | |
| 71 | CATVHQKTRKEKSCPDGYLYSSNTGRGYDCGVWTCRRVGGE | R5C1 |
| | FCSATGDWTSPSEEDFYEFYVDTWGQGLLVTVSS | |
| 72 | CATVHQKTRKEKNCPDGYIYSSNITSGFDCGVWICRRVGSAFC | SR3A3 |
| | SRTGDYTSPTELDIYEFYVEGWGQGVPVTVSS | |
| 73 | CATVDQKTKNACPDDFDYRCSCIGGCGCARKGCVGPLCCRSD | RR2F12 |
| | LGGYLTDSPAYIYEWYIDLWGQGLLVTVSS | |
| 74 | CATVHQKTAEGDKTCPDGYEHTCGCIGGCGCKRSACIGALCC | RR2G3 |
| | QASLGGWLSDGETYTYEFHVDTWGQGLVVTVSS | |
| 75 | CTTVHQSCPDGYSYGYGCGYGYGCSGYDCYGYGGYGGYGGY | Germ |
| | GYSSYSYSYTYEYYVDAWGQGLLVTVSS | |
| 76 | MFVFLVLLPLVSSQCVNLTTRTQLPPAYTNSFTRGVYYPDKVF | WT Wuhan-Hu-1 S |
| | RSSVLHSTQDLFLPFFSNVTWFHAIHVSGTNGTKRFDNPVLPFN | protein |
| | DGVYFASTEKSNIIRGWIFGTTLDSKTQSLLIVNNATNVVIKVC | NCBI Reference |
| | EFQFCNDPFLGVYYHKNNKSWMESEFRVYSSANNCTFEYVSQ | Sequence: |
| | PFLMDLEGKQGNFKNLREFVFKNIDGYFKIYSKHTPINLVRDLP | YP_009724390.1 |
| | QGFSALEPLVDLPIGINITRFQTLLALHRSYLTPGDSSSGWTAGA | (RBD shown in bold, |
| | AAYYVGYLQPRTFLLKYNENGTITDAVDCALDPLSETKCTLKS | intravirion in |
| | FTVEKGIYQTSNFRVQPTESIVRFPNITNLCPFGEVFNATRFA | underline) |
| | SVYAWNRKRISNCVADYSVLYNSASFSTFKCYGVSPTKLND | |
| | LCFTNVYADSFVIRGDEVRQIAPGQTGKIADYNYKLPDDFT | |
| | GCVIAWNSNNLDSKVGGNYNYLYRLFRKSNLKPFERDISTE | |
| | IYQAGSTPCNGVEGFNCYFPLQSYGFQPTNGVGYQPYRVV | |
| | VLSFELLHAPATVCGPKKSTNLVKNKCVNFNFNGLTGTGVL | |
| | TESNKKFLPFQQFGRDIADTTDAVRDPQTLEILDITPCSFGGVSV | |
| | ITPGTNTSNQVAVLYQDVNCTEVPVAIHADQLTPTWRVYSTGS | |
| | NVFQTRAGCLIGAEHVNNSYECDIPIGAGICASYQTQTNSPRRA | |
| | RSVASQSIIAYTMSLGAENSVAYSNNSIAIPTNFTISVTTEILPVS | |
| | MTKTSVDCTMYICGDSTECSNLLLQYGSFCTQLNRALTGIAVE | |
| | QDKNTQEVFAQVKQIYKTPPIKDFGGFNFSQILPDPSKPSKRSFI | |
| | EDLLFNKVTLADAGFIKQYGDCLGDIAARDLICAQKFNGLTVL | |
| | PPLLTDEMIAQYTSALLAGTITSGWTFGAGAALQIPFAMQMAY | |
| | RFNGIGVTQNVLYENQKLIANQFNSAIGKIQDSLSSTASALGKL | |
| | QDVVNQNAQALNTLVKQLSSNFGAISSVLNDILSRLDKVEAEV | |
| | QIDRLITGRLQSLQTYVTQQLIRAAEIRASANLAATKMSECVLG | |
| | QSKRVDFCGKGYHLMSFPQSAPHGVVFLHVTYVPAQEKNFTT | |
| | APAICHDGKAHFPREGVFVSNGTHWFVTQRNFYEPQIITTDNTF | |
| | VSGNCDVVIGIVNNTVYDPLQPELDSFKEELDKYFKNHTSPDV | |
| | DLGDISGINASVVNIQKEIDRLNEVAKNLNESLIDLQELKYEQYI | |
| | KWPWYIWLGFIAGLIAIVMVTIMLCCMTSCCSCLKGCCSCGSC | |
| | CKFDEDDSEPVLKGVKLHYT | |
| 77 | RVQPTESIVRFPNITNLCPFGEVENATRFASVYAWNRKRISNCV | Wuhan-Hu-1 S |
| | ADYSVLYNSASFSTFKCYGVSPTKLNDLCFTNVYADSFVIRGD | protein |
| | EVRQIAPGQTGKIADYNYKLPDDFTGCVIAWNSNNLDSKVGG | NCBI Reference |
| | NYNYLYRLFRKSNLKPFERDISTEIYQAGSTPCNGVEGENCYFP | Sequence: |
| | LQSYGFQPTNGVGYQPYRVVVLSFELLHAPATVCGPKKSTNLV | YP_009724390.1 |
| | KNKCVNF | RBD AA 319-541 |
| 78 | MFVFLVLLPLVSSQCVNLTTRTQLPPAYTNSFTRGVYYPDKVF | Wuhan-Hu-1 S |
| | RSSVLHSTQDLFLPFFSNVTWFHAIHVSGTNGTKRFDNPVLPFN | protein with furin |
| | DGVYFASTEKSNIIRGWIFGTTLDSKTQSLLIVNNATNVVIKVC | site removed |
| | EFQFCNDPFLGVYYHKNNKSWMESEFRVYSSANNCTFEYVSQ | (AA685-686) and |
| | PFLMDLEGKQGNFKNLREFVFKNIDGYFKIYSKHTPINLVRDLP | K986P and V987P |
| | QGFSALEPLVDLPIGINITRFQTLLALHRSYLTPGDSSSGWTAGA | stabilizing mutations |
| | AAYYVGYLQPRTFLLKYNENGTITDAVDCALDPLSETKCTLKS | (bold) |
| | FTVEKGIYQTSNFRVQPTESIVRFPNITNLCPFGEVENATRFASV | Extracellular domain |
| | YAWNRKRISNCVADYSVLYNSASFSTFKCYGVSPTKLNDLCFT | only (AA1233-1273 |
| | NVYADSFVIRGDEVRQIAPGQTGKIADYNYKLPDDFTGCVIAW | removed) |
| | NSNNLDSKVGGNYNYLYRLFRKSNLKPFERDISTEIYQAGSTPC | NCBI Reference |
| | NGVEGFNCYFPLQSYGFQPTNGVGYQPYRVVVLSFELLHAPAT | Sequence: |
| | VCGPKKSTNLVKNKCVNFNFNGLTGTGVLTESNKKFLPFQQFG | YP_009724390.1 |
| | RDIADTTDAVRDPQTLEILDITPCSFGGVSVITPGTNTSNQVAVL | |
| | YQDVNCTEVPVAIHADQLTPTWRVYSTGSNVFQTRAGCLIGAE | |
| | HVNNSYECDIPIGAGICASYQTQTNSPRRAVASQSIIAYTMSLG | |
| | AENSVAYSNNSIAIPTNFTISVTTEILPVSMTKTSVDCTMYICGD | |
| | STECSNLLLQYGSFCTQLNRALTGIAVEQDKNTQEVFAQVKQI | |
| | YKTPPIKDFGGFNFSQILPDPSKPSKRSFIEDLLFNKVTLADAGFI | |
| | KQYGDCLGDIAARDLICAQKFNGLTVLPPLLTDEMIAQYTSAL | |
| | LAGTITSGWTFGAGAALQIPFAMQMAYRFNGIGVTQNVLYEN | |
| | QKLIANQFNSAIGKIQDSLSSTASALGKLQDVVNQNAQALNTL | |
| | VKQLSSNFGAISSVLNDILSRLDPPEAEVQIDRLITGRLQSLQTY | |
| | VTQQLIRAAEIRASANLAATKMSECVLGQSKRVDFCGKGYHL | |
| | MSFPQSAPHGVVFLHVTYVPAQEKNFTTAPAICHDGKAHFPRE | |
| | GVFVSNGTHWFVTQRNFYEPQIITTDNTFVSGNCDVVIGIVNNT | |
| | VYDPLQPELDSFKEELDKYFKNHTSPDVDLGDISGINASVVNIQ | |
| | KEIDRLNEVAKNLNESLIDLQELKYEQYIKWPWYIWLGFIAGLI | |
| | AIVMVTI | |
| 79 | MFVFLVLLPLVSSQCVNFTTRTQLPPAYTNSFTRGVYYPDKVF | 7LYN |
| | RSSVLHSTQDLFLPFFSNVTWFHAIHVSGTNGTKRFANPVLPFN | South African |
| | DGVYFASTEKSNIIRGWIFGTTLDSKTQSLLIVNNATNVVIKVC | (B.1.351) SARS- |
| | EFQFCNDPFLGVYYHKNNKSWMESEFRVYSSANNCTFEYVSQ | CoV-2 spike protein |
| | PFLMDLEGKQGNFKNLREFVFKNIDGYFKIYSKHTPINLVRGLP | variant (S-GSAS- |
| | QGFSALEPLVDLPIGINITRFQTLLALHISYLTPGDSSSGWTAGA | B.1.351) |
| | AAYYVGYLQPRTFLLKYNENGTITDAVDCALDPLSETKCTLKS | |
| | FTVEKGIYQTSNFRVQPTESIVRFPNITNLCPFGEVENATRFASV | |
| | YAWNRKRISNCVADYSVLYNSASFSTFKCYGVSPTKLNDLCFT | |
| | NVYADSFVIRGDEVRQIAPGQTGNIADYNYKLPDDFTGCVIAW | |
| | NSNNLDSKVGGNYNYLYRLFRKSNLKPFERDISTEIYQAGSTPC | |
| | NGVKGFNCYFPLQSYGFQPTYGVGYQPYRVVVLSFELLHAPA | |
| | TVCGPKKSTNLVKNKCVNFNFNGLTGTGVLTESNKKFLPFQQF | |
| | GRDIADTTDAVRDPQTLEILDITPCSFGGVSVITPGTNTSNQVA | |
| | VLYQGVNCTEVPVAIHADQLTPTWRVYSTGSNVFQTRAGCLIG | |
| | AEHVNNSYECDIPIGAGICASYQTQTNSPGSASSVASQSIIAYTM | |
| | SLGVENSVAYSNNSIAIPTNFTISVTTEILPVSMTKTSVDCTMYI | |
| | CGDSTECSNLLLQYGSFCTQLNRALTGIAVEQDKNTQEVFAQV | |
| | KQIYKTPPIKDFGGFNFSQILPDPSKPSKRSFIEDLLFNKVTLAD | |
| | AGFIKQYGDCLGDIAARDLICAQKFNGLTVLPPLLTDEMIAQYT | |
| | SALLAGTITSGWTFGAGAALQIPFAMQMAYRENGIGVTQNVL | |
| | YENQKLIANQFNSAIGKIQDSLSSTASALGKLQDVVNQNAQAL | |
| | NTLVKQLSSNFGAISSVLNDILSRLDKVEAEVQIDRLITGRLQSL | |
| | QTYVTQQLIRAAEIRASANLAATKMSECVLGQSKRVDFCGKG | |
| | YHLMSFPQSAPHGVVFLHVTYVPAQEKNFTTAPAICHDGKAHF | |
| | PREGVFVSNGTHWFVTQRNFYEPQIITTDNTFVSGNCDVVIGIV | |
| | NNTVYDPLQPELDSFKEELDKYFKNHTSPDVDLGDISGINASV | |
| | VNIQKEIDRLNEVAKNLNESLIDLQELGKYEQGSGYIPEAPRDG | |
| | QAYVRKDGEWVLLSTFLGRSLEVLFQGPGHHHHHHHHSAWS | |
| | HPQFEKGGGSGGGGSGGSAWSHPQFEK | |
| 80 | MFVFLVLLPLVSSQCVNFTTRTQLPPAYTNSFTRGVYYPDKVF | 7LYN |
| | RSSVLHSTQDLFLPFFSNVTWFHAIHVSGTNGTKRFANPVLPFN | South African |
| | DGVYFASTEKSNIIRGWIFGTTLDSKTQSLLIVNNATNVVIKVC | (B.1.351) SARS- |
| | EFQFCNDPFLGVYYHKNNKSWMESEFRVYSSANNCTFEYVSQ | CoV-2 spike protein |
| | PFLMDLEGKQGNFKNLREFVFKNIDGYFKIYSKHTPINLVRGLP | variant (S-GSAS- |
| | QGFSALEPLVDLPIGINITRFQTLLALHISYLTPGDSSSGWTAGA | B.1.351) |
| | AAYYVGYLQPRTFLLKYNENGTITDAVDCALDPLSETKCTLKS | with furin site |
| | FTVEKGIYQTSNFRVQPTESIVRFPNITNLCPFGEVFNATRFASV | removed and K986P |
| | YAWNRKRISNCVADYSVLYNSASFSTFKCYGVSPTKLNDLCFT | and V987P |
| | NVYADSFVIRGDEVRQIAPGQTGNIADYNYKLPDDFTGCVIAW | stabilizing mutations |
| | NSNNLDSKVGGNYNYLYRLFRKSNLKPFERDISTEIYQAGSTPC | (bold) |
| | NGVKGFNCYFPLQSYGFQPTYGVGYQPYRVVVLSFELLHAPA | Extracellular domain |
| | TVCGPKKSTNLVKNKCVNFNFNGLTGTGVLTESNKKFLPFQQF | only |
| | GRDIADTTDAVRDPQTLEILDITPCSFGGVSVITPGTNTSNQVA | |
| | VLYQGVNCTEVPVAIHADQLTPTWRVYSTGSNVFQTRAGCLIG | |
| | AEHVNNSYECDIPIGAGICASYQTQTNSPGSASSVASQSIIAYTM | |
| | SLGVENSVAYSNNSIAIPTNFTISVTTEILPVSMTKTSVDCTMYI | |
| | CGDSTECSNLLLQYGSFCTQLNRALTGIAVEQDKNTQEVFAQV | |
| | KQIYKTPPIKDFGGFNFSQILPDPSKPSKRSFIEDLLFNKVTLAD | |
| | AGFIKQYGDCLGDIAARDLICAQKFNGLTVLPPLLTDEMIAQYT | |
| | SALLAGTITSGWTFGAGAALQIPFAMQMAYRENGIGVTQNVL | |
| | YENQKLIANQFNSAIGKIQDSLSSTASALGKLQDVVNQNAQAL | |
| | NTLVKQLSSNFGAISSVLNDILSRLDPPEAEVQIDRLITGRLQSL | |
| | QTYVTQQLIRAAEIRASANLAATKMSECVLGQSKRVDFCGKG | |
| | YHLMSFPQSAPHGVVFLHVTYVPAQEKNFTTAPAICHDGKAHF | |
| | PREGVFVSNGTHWFVTQRNFYEPQIITTDNTFVSGNCDVVIGIV | |
| | NNTVYDPLQPELDSFKEELDKYFKNHTSPDVDLGDISGINASV | |
| | VNIQKEIDRLNEVAKNLNESLIDLQELGKYEQGSGYIPEAPRDG | |
| | QAYVRKDGEWVLLSTFLGRSLEVLFQGPGHHHHHHHHSAWS | |
| | HPQFEKGGGSGGGGSGGSAWSHPQFEK | |
| 81 | QVQLRESGPSLVKPSQTLSLTCTVSGFSLSDKAVGWVRQAPGK | IgHV1-7 V gene |
| | ALEWLGGIDTGGSTGYNPGLKSRLSITKDNSKSQVSLSVSSVTT | |
| | EDSATYYCTTVHQ | |
| 82 | SCPDGYSYGYGCGYGYGCSGYDCYGYGGYGGYGGYGYSSYS | IDHD8-2 |
| | YSYTYEY | |
| 83 | YVDAWGQGLLVTVSS | IGHJ2-4 |
| 84 | TGCAGGTGCAGCTGCGGGAGTCGGG | Minimal |
| | | BOVVHNCOFOR2 |
| | | primer |
| 85 | TGAGGAGACGGTGACCAGGAGTCC | Minimal |
| | | BOVVHFR4XHOREV |
| | | primer |
| 86 | GGGGAMGSEGDKTCPDGYEHTCGCIGGCGCKRSACIGALCCQ | R2G3 Parental |
| | ASLGGWLSDGETYT | |
| 87 | GGSEGDKTCPDGYEHTCGCIGGCGCKRSACIGALCCQASLGG | R2G3 TRUNC1 |
| | WLSDGETYT | |
| 88 | GGSEGDKTCPDGYEHTCGCIGGCGCKRSACIGALCCQASLGG | R2G3 TRUNC2 |
| | WLSDGE | |
| 89 | GGSEGDKTCPDGYEHTCGCIGGCGCKRSACIGALCCQASLGG | R2G3 TRUNC3 |
| | WLS | |
| 90 | GGSEGDKTCPDGYEHTCGCIGGCGCKRSACIGALCCQASLGG | R2G3 TRUNC3A |
| | WL | |
| 91 | GGSEGDKTCPDGYEHTCGCIGGCGCKRSACIGALCCQASLGG | R2G3 TRUNC3B |
| | W | |
| 92 | GGSEGDKTCPDGYEHTCGCIGGCGCKRSACIGALCCQASLGG | R2G3 TRUNC4 |
| 93 | GGSEGDKTCPDGYEHTCGCIGGCGCKRSACIGALCCQAS | R2G3 TRUNC5 |
| 94 | GlyGlyGlyGlySerGlyGlyGlyGlySerGlyGlyGlyGlySer | Flexible Linker |
| 95 | GGVCPKILQRCRRDSDSPGACICRGNGYCGSGSD | Mcoti-I |
| 96 | GGVCPKILKKCRRDSDSPGACICRGNGYCGSGSD | Mcoti-II |
| 97 | ERACPRILKKCRRDSDSPGACICRGNGYCG | Mcoti-III |
| 98 | CTTVHQ | Base of Stalk A |
| 99 | CATVHQ | Base of Stalk A |
| 100 | CAIVQQ | Base of Stalk A |
| 101 | CATVDQ | Base of Stalk A |
| 102 | YX₁YX₂Y | Stalk B |
| | | X1 and X2 are any |
| | | amino acid |
| 103 | CX₂TVX₅Q | Ascending Stalk |
| | | Domain |
| | | X₂ and X₅ are any |
| | | amino acid |
| 104 | CX₂TVX₅Q | Ascending Stalk |
| | | Domain |
| | | X₂ is Ser, Thr, Gly, |
| | | Asn, Ala, or Pro, and |
| | | X₅ is His, Gln, Arg, |
| | | Lys, Gly, Thr, Tyr, |
| | | Phe, Trp, Met, Ile, |
| | | Val, or Leu |
| 105 | CX₂TVX₅Q | Ascending Stalk |
| | | Domain |
| | | X₂ is Ser, Ala, or |
| | | Thr, and X₅ is His or |
| | | Tyr |
| 106 | DDDDK | Enterokinase |
| | | Cleavage Tag |
| 107 | QAVLNQPSSVSGSLGQKVTISCSGSSSNIGNNYVSWYQQLPGT | Humanized |
| | APKLLIYGDTKRPSGIPDRESGSKSGTSATLGITGLQTGDEADY | BLV1H12 Variable |
| | YCASAEDSSSNAVFGSGTTLTVLGQP | Light |
| 108 | GGGGAMGS | Flexible Linker |
| 109 | GGS | Flexible Linker |
| 110 | QAVLNQPSSVSGSLGQRVSITCSGSSSNVGNGYVSWYQLIPGS | BLV5B8 Variable |
| | APRTLIYGDTSRASGVPDRESGSRSGNTATLTISSLQAEDEADY | Light Region |
| | FCASAEDSSSNAVFGSGTTLTVLGQP | |
| 111 | QVQLREWGAGLLKPSETLSLTCAVYGGSFSDKYWSWIRQPPG | Humanized V1 |
| | KGLEWIGSINHSGSTNYNPSLKSRVTISVDTSKNQFSLKLSSVT | Region |
| | AADTAVYY | |
| 112 | WGQGLLVTVSS | V2 Region |
| 113 | QAVLNQPSSVSGSLGQRVSITCSGSSSNVGNGYVSWYQLIPGS | BLV1H12 Light |
| | APRTLIYGDTSRASGVPDRFSGSRSGNTATLTISSLQAEDEADY | Chain |
| | FCASAEDSSSNAVFGSGTTLTVLGQPKSPPSVTLFPPSTEELNGN | |
| | KATLVCLISDFYPGSVTVVWKADGSTITRNVETTRASKQSNSK | |
| | YAASSYLSLTSSDWKSKGSYSCEVTHEGSTVTKTVKPSECS | |
| 114 | QAVLNQPSSVSGSLGQKVTISCSGSSSNIGNNYVSWYQQLPGT | B15 Humanized |
| | APKLLIYGDTKRPSGIPDRESGSKSGTSATLGITGLQTGDEADY | Light Chain |
| | YCASAEDSSSNAVFGSGTTLTVLGQPKAAPSVTLFPPSSEELQA | |
| | NKATLVCLISDFYPGAVTVAWKADSSPVKAGVETTTPSKQSNN | |
| | KYAASSYLSLTPEQWKSHRSYSCQVTHEGSTVEKTVAPTECS | |
| 115 | QAVLNQPSSVSGSLGQRVSITCSGSSSNVGNGYVSWYQLIPGS | BLV5B8 light chain |
| | APRTLIYGDTSRASGVPDRESGSRSGNTATLTISSLQAEDEADY | |
| | FCASAEDSSSNAVFGSGTTLTVLGQPKSPPSVTLFPPSTEELNGN | |
| | KATLVCLISDFYPGSVTVVWKADGSTITRNVETTRASKQSNSK | |
| | YAASSYLSLTSSDWKSKGSYSCEVTHEGSTVTKTVKPSECS | |
| 116 | QSVLTQPPSVSAAPGQKVTISCSGSSSNIGNNYVSWYQQLPGTA | human VL1-51 |
| | PKLLIYDNNKRPSGIPDRFSGSKSGTSATLGITGLQTGDEADYY | |
| | CASAEDSSSNAVFGSGTTLTVLGQPKAAPSVTLFPPSSEELQAN | |
| | KATLVCLISDFYPGAVTVAWKADSSPVKAGVETTTPSKQSNNK | |
| | YAASSYLSLTPEQWKSHRSYSCQVTHEGSTVEKTVAPTECS | |
| 117 | QSVLTQPPSASGTPGQRVTISCSGSSSNIGSNYVYWYQQLPGTA | Human germline |
| | PKLLIYRNNQRPSGVPDRFSGSKSGTSASLAISGLRSEDEADYY | light chain variable |
| | CAAWDDSLSG | region sequence |
| | | VL1-47 |
| 118 | QSVLTQPPSVSGAPGQRVTISCTGSSSNIGAGYDVHWYQQLPG | Human germline |
| | TAPKLLIYGNSNRPSGVPDRFSGSKSGTSASLAITGLQAEDEAD | light chain variable |
| | YYCQSYDSSLSG | region sequence |
| | | VL1-40*1 |
| 119 | QSVLTQPPSVSAAPGQKVTISCSGSSSNIGNNYVSWYQQLPGTA | Human germline |
| | PKLLIYDNNKRPSGIPDRFSGSKSGTSATLGITGLQTGDEADYY | light chain variable |
| | CGTWDSSLSA | region sequence |
| | | VL1-51*01 |
| 120 | QSALTQPPSVSGSPGQSVTISCTGTSSDVGSYNRVSWYQQPPGT | Human germline |
| | APKLMIYEVSNRPSGVPDRFSGSKSGNTASLTISGLQAEDEADY | light chain variable |
| | YCSSYTSSSTF | region sequence |
| | | VL2-18*02 |
| 121 | ttacctgcggccgctgaggagacggtgaccaggagtcc | BOVVHFR4REV |
| 122 | ttttttgeggccgcccaggcgctgacgtaccattc | ULp1 |
| 123 | ttttttgcggccgcccaggcatcgacgtagaattc | Ulp2 |
| 124 | ttttttgcggccgcccagacatcgacgaaaaattc | Ulp3 |
| 125 | ttttttgcggccgcccaggcatggacgtaaaattg | Ulp4 |
| 126 | ttttttgeggccgcccaagtctcgacataaaattc | Ulp5 |
| 127 | ttttttgcggccgcccaggcatcgacgagccattg | Ulp6 |
| 128 | ttttttgcggccgcccaggcatcgacgtgccattc | Ulp7 |
| 129 | ttttttgcggccgcccaggcatcgacgtggaattc | Ulp8 |
| 130 | ttttttgcggccgcccaggcatcgacgtggaagct | Ulp9 |
| 131 | GGSEGDKTCPDGYEHTCGCIGGCGCKRSACIGALCCQASLGG | G3 Parental |
| | WLSD | |
| 132 | GGSGDKTCPDGYEHTCGCIGGCGCKRSACIGALCCQASLGGW | G3 NTRUNC1 |
| | LSD | |
| 133 | GGSDKTCPDGYEHTCGCIGGCGCKRSACIGALCCQASLGGWL | G3 NTRUNC2 |
| | SD | |
| 134 | GGSKTCPDGYEHTCGCIGGCGCKRSACIGALCCQASLGGWLS | G3 NTRUNC3 |
| | D | |
| 135 | GGSTCPDGYEHTCGCIGGCGCKRSACIGALCCQASLGGWLSD | G3 NTRUNC4 |
| 136 | GGSCPDGYEHTCGCIGGCGCKRSACIGALCCQASLGGWLSD | G3 NTRUNC5 |
| 137 | tttgttgccaagcgtcgttgggtggttggcttagtgacggtgaaacctacacttacga | Ultralong CD3 |
| | gttccacgtcgatacctggggccaaggactcgtggtcaccgtctcctca | Antibody 014 |
| 138 | gtgccttctgtagtcgtactggtgattatactagtcctactgaacttgacatttacga | Ultralong CD3 |
| | gttctacgtcgaagggtggggccagggagtcccggtcaccgtctcctca | Antibody 015 |
| 139 | cttggcctagtggtaatggtagagccgacagtagtattggtgaaacttatggttacga | Ultralong CD3 |
| | atttcacgtggctgcctggggccaaggactcctggtcaccgtctcctca | Antibody 032 |
| 140 | tttgttgtcgttctgatttgggtggctatcttactgatagtcctgcttacatttacga | Ultralong CD3 |
| | atggtatattgatctttggggccaaggactcctggtcaccgtctcctca | Antibody 016 |
| 141 | gtgaattctgtagtgctactggtgattggactagtcctagtgaagaagacttttacga | Ultralong CD3 |
| | attctacgtcgatacgtggggccagggagccccggtcaccgtctcctca | Antibody 031 |
| 142 | ctaattgtagaggcgttttgtgtcctactcttaacgaaatcgttgcttatacctacga | Ultralong CD3 |
| | atggcacgtcgacgcctggggccaaggactcctggtcaccgtctcctca | Antibody 027 |
| 143 | gtgccttctgtagtcgtactggtgattatactagtcctagtgaatttgacatttacga | Ultralong CD3 |
| | gttctacgtcgaagggtggggccagggactcctggtcaccgtctcctca | Antibody 021 |
| 144 | gttaccacagcactgatccttctcattatactggtgcgacgtatatttacacgtacag | Ultralong CD3 |
| | cttgcacatcgatgcctggggccaaggactcctggtcaccgtctcctca | Antibody 026 |
| 145 | gcaagaagtcgtggttatgattgttatgctaatgtggatgctttggactacgtcgatg | Standard Short |
| | cctggggccaaggactcctggtcaccgtctcctca | CDR3 Antibody 028 |
| 146 | agtggaatttagaatatacttggggtggtgttggttgcgctagttttgctgatgagga | Ultralong CD3 |
| | cacccacgttgatgcctggggccaaggactcctggtcaccgtctcctca | Antibody 018 |
| 147 | attatgttgttcgtcgttataattgtggtggtcttggttatgggcatggctttaatag | Ultralong CD3 |
| | tttctacgtcgatgcctggggccaaggactcctggtcaccgtctcctca | Antibody 019 |
| 148 | attatgttgttcgtcgttataattgtggtggtcttggttatgggcatggctttaatag | Ultralong CD3 |
| | tttctacgtcgatgcctggggccaaggactcctggtcaccgtctcctca | Antibody 020 |
| 149 | atcgggttgtgcgtcgtaataattgtggtgggcttggttatgattatggttttgatca | Ultralong CD3 |
| | tttctacgtcgatgcctggggccaaggactcctggtcaccgtctcctca | Antibody 022 |
| 150 | gcgaagtttgctaagggtactacgagtgctggtgcttgtgattattcagaaagctacg | Ultralong CD3 |
| | tcgatgcctggggccagggactcctggtcaccgtctcctca | Antibody 023 |
| 151 | attccggtgcttatgcttatgctgcttgcaattattatggttggcgttgtgcttggga | Ultralong CD3 |
| | aagctacatcgatgcctggggccaaggactcctggtcaccgt | Antibody 024 |
| 152 | acaatgcacgttgtgatagttggacgtatgacagctgtgatacttggtatcgcaattc | Ultralong CD3 |
| | agtggcacgttgtgcctggggccaaggactcctggtcaccgtctcctca | Antibody 025 |
| 153 | gcaagaagtcgtggttatgattgttatgcttatgtttatgctttggacaccgtcgatg | Standard Short |
| | cctggggccaaggactcctggtcaccgtctcctca | CDR3 Antibody 029 |
| 154 | gcaagaagtcgtggttatgattgttatgctaatgtggatgctttggactacgtcgatg | Standard Short |
| | cctggggccaaggactcctggtcaccgtctcctca | CDR3 Antibody 030 |
| 155 | TCPDGYEHTCGCIGGCGCKRSACIGALCCQASLGGWLSD | R2G3 |
| 156 | GGAACCCATGGCCGGCNNKNNKACGTGTCCTGATGGTTAC | Forward primer |
| 157 | GGAACGCGGCCGCMNNMNNGTCACTAAGCCAACCACC | Reverse primer |
| 158 | TITCPDGYEHTCGCIGGCGCKRSACIGALCCQASLGGWLSDQS | G3-A knob |
| 159 | CKTCPDGYEHTCGCIGGCGCKRSACIGALCCQASLGGWLSDQ | G3-B knob |
| | H | |
| 160 | DYTCPDGYEHTCGCIGGCGCKRSACIGALCCQASLGGWLSDM | G3-C knob |
| | P | |
| 161 | LQTCPDGYEHTCGCIGGCGCKRSACIGALCCQASLGGWLSDPP | G3-D knob |
| 162 | SVTCPDGYEHTCGCIGGCGCKRSACIGALCCQASLGGWLSDYI | G3-E knob |
| 163 | NGTCPDGYEHTCGCIGGCGCKRSACIGALCCQASLGGWLSDM | G3-F knob |
| | Q | |
| 164 | LVTCPDGYEHTCGCIGGCGCKRSACIGALCCQASLGGWLSDIP | G3-G knob |
| 165 | QGTCPDGYEHTCGCIGGCGCKRSACIGALCCQASLGGWLSDC | G3-H knob |
| | Q | |
| 166 | LSTCPDGYEHTCGCIGGCGCKRSACIGALCCQASLGGWLSDQV | G3-I knob |
| 167 | MITCPDGYEHTCGCIGGCGCKRSACIGALCCQASLGGWLSDQV | G3-K knob |
| 168 | LVTCPDGYEHTCGCIGGCGCKRSACIGALCCQASLGGWLSDR | G3-L knob |
| | Q | |
| 169 | QPTCPDGYEHTCGCIGGCGCKRSACIGALCCQASLGGWLSDK | G3-M knob |
| | Q | |
| 170 | QQTCPDGYEHTCGCIGGCGCKRSACIGALCCQASLGGWLSDY | G3-N |
| | L | |
| 171 | AQTCPDGYEHTCGCIGGCGCKRSACIGALCCQASLGGWLSDY | G3-O |
| | H | |
| 172 | HLTCPDGYEHTCGCIGGCGCKRSACIGALCCQASLGGWLSDQ | G3-P |
| | Q | |
| 173 | PQTCPDGYEHTCGCIGGCGCKRSACIGALCCQASLGGWLSDQF | G3-Q |
| 174 | HWTCPDGYEHTCGCIGGCGCKRSACIGALCCQASLGGWLSDS | G3-R |
| | F | |
| 175 | SCTCPDGYEHTCGCIGGCGCKRSACIGALCCQASLGGWLSDQP | G3-S |
| 176 | PFTCPDGYEHTCGCIGGCGCKRSACIGALCCQASLGGWLSDQH | G3-T |
| 177 | WMTCPDGYEHTCGCIGGCGCKRSACIGALCCQASLGGWLSDQ | G3-U |
| | Q | |
| 178 | SCTCPDGYEHTCGCIGGCGCKRSACIGALCCQASLGGWLSDDS | G3-V |
| 179 | LSTCPDGYEHTCGCIGGCGCKRSACIGALCCQASLGGWLSDVQ | G3-W |
| 180 | QLTCPDGYEHTCGCIGGCGCKRSACIGALCCQASLGGWLSDY | G3-X |
| | V | |
| 181 | LVTCPDGYEHTCGCIGGCGCKRSACIGALCCQASLGGWLSDQ | G3-Y |
| | Q | |
| 182 | GMTCPDGYEHTCGCIGGCGCKRSACIGALCCQASLGGWLSDR | G3-Z |
| | S | |
| 183 | ISTCPDGYEHTCGCIGGCGCKRSACIGALCCQASLGGWLSDTV | G3-AA |
| 184 | SQTCPDGYEHTCGCIGGCGCKRSACIGALCCQASLGGWLSDHL | G3-BB |
| 185 | CDTCPDGYEHTCGCIGGCGCKRSACIGALCCQASLGGWLSDQ | G3-CC |
| | R | |
| 186 | SLTCPDGYEHTCGCIGGCGCKRSACIGALCCQASLGGWLSDQR | G3-DD |
| 187 | NCPDGYIYSSNTASGYDCGVWICRRVGSAFCSRTGDYTSP | R4C1 |
| 188 | HWNCPDGYIYSSNTASGYDCGVWICRRVGSAFCSRTGDYTSPS | HW-SF modified |
| | F | R4C1 |
| 189 | ISNCPDGYIYSSNTASGYDCGVWICRRVGSAFCSRTGDYTSPTV | IS-TV modified |
| | | R4C1 |
| 190 | TCPNGYNWFDRCCSWDGTCGDGCCSNRAWPSGNGRADSSIGE | R2D9 |
| 191 | HWTCPNGYNWFDRCCSWDGTCGDGCCSNRAWPSGNGRADSS | HW-SF modified |
| | IGESF | R2D9 |
| 192 | ISTCPNGYNWFDRCCSWDGTCGDGCCSNRAWPSGNGRADSSI | IS-TV modified |
| | GETV | R2D9 |
| 193 | ATGAGCGATAAAATTATTCACCTGACTGACGACAGTTTTGACACGGA | TrxA |
| | TGTACTCAAAGCGGACGGGGCGATCCTCGTCGATTTCTGGGCAGAGT | |
| | GGTGCGGTCCGTGCAAAATGATCGCCCCGATTCTGGATGAAATCGCT | |
| | GACGAATATCAGGGCAAACTGACCGTTGCAAAACTGAACATCGATCA | |
| | AAACCCTGGCACTGCGCCGAAATATGGCATCCGTGGTATCCCGACTC | |
| | TGCTGCTGTTCAAAAACGGTGAAGTGGCGGCAACCAAAGTGGGIGCA | |
| | CTGTCTAAAGGTCAGTTGAAAGAGTTCCTCGACGCTAACCTGGCC | |
| 194 | MSDKIIHLTDDSFDTDVLKADGAILVDFWAEWCGPCKMIAPILDEIA | TrxA |
| | DEYQGKLTVAKLNIDQNPGTAPKYGIRGIPTLLLFKNGEVAATKVGA | |
| | LSKGQLKEFLDANLA | |
| 195 | MSDKIIHLTDDSFDTDVLKADGAILVDFWAEWCGPCKMIAPILDEIA | TrxA N-terminal |
| | DEYQGKLTVAKLNIDQNPGTAPKYGIRGIPTLLLFKNGEVAATKV | portion before last |
| | | engineered loop |
| 196 | HQETLR | SKM stalk A |
| 197 | YTYSLH | SKM stalk B |
| 198 | SCPDGYIDNSGCTADWGCAALDCWRRRFGYHSTDP SHYTGATYI | SKM knob |
| 199 | GALSKGQLKEFLDANLA | TrxA C-terminal |
| | | portion after last |
| | | engineered loop |
| 200 | ATGAGCGATAAAATTATTCACCTGACTGACGACAGTTTTGACACGGA | TrxA Loop SKS- |
| | TGTACTCAAAGCGGACGGGGCGATCCTCGTCGATTTCTGGGCAGAGT | STM |
| | GGTGCGGTCCGTGCAAAATGATCGCCCCGATTCTGGATGAAATCGCT | Nucleotide |
| | GACGAATATCAGGGCAAACTGACCGTTGCAAAACTGAACATCGATCA | |
| | AAACCCTGGCACTGCGCCGAAATATGGCATCCGTGGTATCCCGACTC | |
| | TGCTGCTGTTCAAAAACGGTGAAGTGGCGGCAACCAAAGTGCACCAA | |
| | GAGACCTTACGTAGTTGTCCTGATGGTTATATTGATAATTCTGGATG | |
| | CACGGCTGATTGGGGTTGTGCAGCTCTTGATTGTTGGCGGCGTCGTT | |
| | TTGGTTACCACAGCACTGATCCTTCTCATTATACTGGTGCGACGTAT | |
| | ATTTACACGTACAGCTTGCACGGTGCACTGTCTAAAGGTCAGTTGAA | |
| | AGAGTTCCTCGACGCTAACCTGGCCGGTGGCCAGGAAACCTTTAGCG | |
| | ATCTGTGGAAACTGCTGCCGGAAAATctcgag | |
| 201 | MSDKIIHLTDDSFDTDVLKADGAILVDFWAEWCGPCKMIAPILDEIA | TrxA Loop SKS- |
| | DEYQGKLTVAKLNIDQNPGTAPKYGIRGIPTLLLFKNGEVAATKVHQ | STM |
| | ETLRSCPDGYIDNSGCTADWGCAALDCWRRRFGYHSTDPSHYTGATY | Amino acid |
| | IYTYSLHGALSKGQLKEFLDANLAGGQETFSDLWKLLPENLE | |
| 202 | GGSGAKLAALKAKLAALKGGGGS | Coiled-coil |
| | | ascending |
| 203 | GGGGSELAALEAELAALEAGGSG | Coiled-coil |
| | | descending |
| 204 | TCTAGAAATAATTTTGTTTAACTTTAAGAAGGAGATATACAT | TrxA_Loop_CKC- |
| | ATGAGCGATAAAATTATTCACCTGACTGACGACAGTTTTGA | SKM |
| | CACGGATGTACTCAAAGCGGACGGGGCGATCCTCGTCGATT | nucleotide |
| | TCTGGGCAGAGTGGTGCGGTCCGTGCAAAATGATCGCCCCG | |
| | ATTCTGGATGAAATCGCTGACGAATATCAGGGCAAACTGAC | |
| | CGTTGCAAAACTGAACATCGATCAAAACCCTGGCACTGCGC | |
| | CGAAATATGGCATCCGTGGTATCCCGACTCTGCTGCTGTTCA | |
| | AAAACGGTGAAGTGGCGGCAACCAAAGTGGGCGGCAGCGG | |
| | CGCGAAACTGGCGGCGCTGAAAGCGAAACTGGCGGCGCTG | |
| | AAAGGCGGCGGCGGCAGCAGTTGTCCTGATGGTTATATTGA | |
| | TAATTCTGGATGCACGGCTGATTGGGGTTGTGCAGCTCTTGA | |
| | TTGTTGGCGGCGTCGTTTTGGTTACCACAGCACTGATCCTTC | |
| | TCATTATACTGGTGCGACGTATATTGGCGGCGGCGGCAGCG | |
| | AACTGGCGGCGCTGGAAGCGGAACTGGCGGCGCTGGAAGC | |
| | GGGCGGCAGCGGCGGTGCACTGTCTAAAGGTCAGTTGAAAG | |
| | AGTTCCTCGACGCTAACCTGGCCGGTGGCCAGGAAACCTTT | |
| | AGCGATCTGTGGAAACTGCTGCCGGAAAATctcgag | |
| 205 | MSDKIIHLTDDSFDTDVLKADGAILVDFWAEWCGPCKMIAPIL | TrxA_Loop_CKC- |
| | DEIADEYQGKLTVAKLNIDQNPGTAPKYGIRGIPTLLLFKNGEV | SKM |
| | AATKVGGSGAKLAALKAKLAALKGGGGSSCPDGYIDNSGCTA | Amino acid |
| | DWGCAALDCWRRRFGYHSTDPSHYTGATYIGGGGSELAALEA | |
| | ELAALEAGGSGGALSKGQLKEFLDANLAGGQETFSDLWKLLP | |
| | ENLE | |
| 206 | TCTAGAAATAATTTTGTTTAACTTTAAGAAGGAGATATACAT | TrxA_Loop_GKG- |
| | ATGAGCGATAAAATTATTCACCTGACTGACGACAGTTTTGA | SKM |
| | CACGGATGTACTCAAAGCGGACGGGGCGATCCTCGTCGATT | Nucleotide |
| | TCTGGGCAGAGTGGTGCGGTCCGTGCAAAATGATCGCCCCG | |
| | ATTCTGGATGAAATCGCTGACGAATATCAGGGCAAACTGAC | |
| | CGTTGCAAAACTGAACATCGATCAAAACCCTGGCACTGCGC | |
| | CGAAATATGGCATCCGTGGTATCCCGACTCTGCTGCTGTTCA | |
| | AAAACGGTGAAGTGGCGGCAACCAAAGTGGGCGGCGGATC | |
| | CAGTTGTCCTGATGGTTATATTGATAATTCTGGATGCACGGC | |
| | TGATTGGGGTTGTGCAGCTCTTGATTGTTGGCGGCGTCGTTT | |
| | TGGTTACCACAGCACTGATCCTTCTCATTATACTGGTGCGAC | |
| | GTATATTGGCGGTACCGGTGCACTGTCTAAAGGTCAGTTGA | |
| | AAGAGTTCCTCGACGCTAACCTGGCCGGTGGCCAGGAAACC | |
| | TTTAGCGATCTGTGGAAACTGCTGCCGGAAAATctcgag | |
| 207 | MSDKIIHLTDDSFDTDVLKADGAILVDFWAEWCGPCKMIAPIL | TrxA_Loop_GKG- |
| | DEIADEYQGKLTVAKLNIDQNPGTAPKYGIRGIPTLLLFKNGEV | SKM |
| | AATKVGGGSSCPDGYIDNSGCTADWGCAALDCWRRRFGYHS | Amino acid |
| | TDPSHYTGATYIGGTGALSKGQLKEFLDANLAGGQETFSDLW | |
| | KLLPENLE | |
| 208 | QETFSDLWKLLPEN | D01 tag |
| 209 | DYKDDDDK | Flag/Enterokinase |
| | | site |
| 210 | GGSDYKDDDDKGS | Flag/Enterokinase |
| | | site with GS linkers |
| 211 | TCTAGAAATAATTTTGTTTAACTTTAAGAAGGAGATATACAT | TrxA_Cterm_SKS- |
| | ATGAGCGATAAAATTATTCACCTGACTGACGACAGTTTTGA | SKM |
| | CACGGATGTACTCAAAGCGGACGGGGCGATCCTCGTCGATT | nucleotide |
| | TCTGGGCAGAGTGGTGCGGTCCGTGCAAAATGATCGCCCCG | |
| | ATTCTGGATGAAATCGCTGACGAATATCAGGGCAAACTGAC | |
| | CGTTGCAAAACTGAACATCGATCAAAACCCTGGCACTGCGC | |
| | CGAAATATGGCATCCGTGGTATCCCGACTCTGCTGCTGTTCA | |
| | AAAACGGTGAAGTGGCGGCAACCAAAGTGGGTGCACTGTCT | |
| | AAAGGTCAGTTGAAAGAGTTCCTCGACGCTAACCTGGCCGG | |
| | CGGATCCGATTATAAAGATGACGACGATAAAGGCAGCCACC | |
| | AAGAGACCTTACGTAGTTGTCCTGATGGTTATATTGATAATT | |
| | CTGGATGCACGGCTGATTGGGGTTGTGCAGCTCTTGATTGTT | |
| | GGCGGCGTCGTTTTGGTTACCACAGCACTGATCCTTCTCATT | |
| | ATACTGGTGCGACGTATATTTACACGTACAGCTTGCACGGC | |
| | GGTAGCCAGGAAACCTTTAGCGATCTGTGGAAACTGCTGCC | |
| | GGAAAATctcgag | |
| 212 | MSDKIIHLTDDSFDTDVLKADGAILVDFWAEWCGPCKMIAPIL | TrxA_Cterm_SKS- |
| | DEIADEYQGKLTVAKLNIDQNPGTAPKYGIRGIPTLLLFKNGEV | SKM |
| | AATKVGALSKGQLKEFLDANLAGGSDYKDDDDKGSHQETLRS | Amino acid |
| | CPDGYIDNSGCTADWGCAALDCWRRRFGYHSTDPSHYTGATY | |
| | IYTYSLHGGSQETFSDLWKLLPENLE | |
| 213 | TCTAGAAATAATTTTGTTTAACTTTAAGAAGGAGATATACAT | TrxA_Cterm_CKC- |
| | ATGAGCGATAAAATTATTCACCTGACTGACGACAGTTTTGA | SKM |
| | CACGGATGTACTCAAAGCGGACGGGGCGATCCTCGTCGATT | Nucleotide |
| | TCTGGGCAGAGTGGTGCGGTCCGTGCAAAATGATCGCCCCG | |
| | ATTCTGGATGAAATCGCTGACGAATATCAGGGCAAACTGAC | |
| | CGTTGCAAAACTGAACATCGATCAAAACCCTGGCACTGCGC | |
| | CGAAATATGGCATCCGTGGTATCCCGACTCTGCTGCTGTTCA | |
| | AAAACGGTGAAGTGGCGGCAACCAAAGTGGGTGCACTGTCT | |
| | AAAGGTCAGTTGAAAGAGTTCCTCGACGCTAACCTGGCCGG | |
| | CGGATCCGATTATAAAGATGACGACGATAAAGGCAGCGGC | |
| | GGCAGCGGCGCGAAACTGGCGGCGCTGAAAGCGAAACTGG | |
| | CGGCGCTGAAAGGCGGCGGCGGCAGCAGTTGTCCTGATGGT | |
| | TATATTGATAATTCTGGATGCACGGCTGATTGGGGTTGTGCA | |
| | GCTCTTGATTGTTGGCGGCGTCGTTTTGGTTACCACAGCACT | |
| | GATCCTTCTCATTATACTGGTGCGACGTATATTGGCGGCGGC | |
| | GGCAGCGAACTGGCGGCGCTGGAAGCGGAACTGGCGGCGC | |
| | TGGAAGCGGGCGGCAGCGGCGGCGGTAGCCAGGAAACCTT | |
| | TAGCGATCTGTGGAAACTGCTGCCGGAAAATctcgag | |
| 214 | MSDKIIHLTDDSFDTDVLKADGAILVDFWAEWCGPCKMIAPIL | TrxA_Cterm_CKC- |
| | DEIADEYQGKLTVAKLNIDQNPGTAPKYGIRGIPTLLLFKNGEV | SKM |
| | AATKVGALSKGQLKEFLDANLAGGSDYKDDDDKGSGGSGAK | Nucleotide |
| | LAALKAKLAALKGGGGSSCPDGYIDNSGCTADWGCAALDCW | |
| | RRRFGYHSTDPSHYTGATYIGGGGSELAALEAELAALEAGGSG | |
| | GGSQETFSDLWKLLPENLE | |
| 215 | TCTAGAAATAATTTTGTTTAACTTTAAGAAGGAGATATACAT | TrxA_Cterm_GKG- |
| | ATGAGCGATAAAATTATTCACCTGACTGACGACAGTTTTGA | SKM |
| | CACGGATGTACTCAAAGCGGACGGGGCGATCCTCGTCGATT | Nucleotide |
| | TCTGGGCAGAGTGGTGCGGTCCGTGCAAAATGATCGCCCCG | |
| | ATTCTGGATGAAATCGCTGACGAATATCAGGGCAAACTGAC | |
| | CGTTGCAAAACTGAACATCGATCAAAACCCTGGCACTGCGC | |
| | CGAAATATGGCATCCGTGGTATCCCGACTCTGCTGCTGTTCA | |
| | AAAACGGTGAAGTGGCGGCAACCAAAGTGGGTGCACTGTCT | |
| | AAAGGTCAGTTGAAAGAGTTCCTCGACGCTAACCTGGCCGG | |
| | CGGTAGCGATTATAAAGATGACGACGATAAAGGCGGATCCA | |
| | GTTGTCCTGATGGTTATATTGATAATTCTGGATGCACGGCTG | |
| | ATTGGGGTTGTGCAGCTCTTGATTGTTGGCGGCGTCGTTTTG | |
| | GTTACCACAGCACTGATCCTTCTCATTATACTGGTGCGACGT | |
| | ATATTGGCACCGGTCAGGAAACCTTTAGCGATCTGTGGAAA | |
| | CTGCTGCCGGAAAATctcgag | |
| 216 | MSDKIIHLTDDSFDTDVLKADGAILVDFWAEWCGPCKMIAPIL | TrxA_Cterm_GKG- |
| | DEIADEYQGKLTVAKLNIDQNPGTAPKYGIRGIPTLLLFKNGEV | SKM |
| | AATKVGALSKGQLKEFLDANLAGGSDYKDDDDKGGSSCPDG | Amino acid |
| | YIDNSGCTADWGCAALDCWRRR | |
| | FGYHSTDPSHYTGATYIGTGQETFSDLWKLLPENLE | |
| 217 | HHHHHH | 6xHis |
| 218 | I-E[D]-G-R-X1, where X1 can be any | Factor Xa cleavage |
| | amino acid except arginine and proline. | site |
| 219 | X4-X3-P-R/K-X1′-X2′, | Thrombin cleavage |
| | where X4 and X3 are hydrophobic amino acid and X1′, X2′ | site |
| | are non-acidic amino acids | |
| 220 | LVPRGS | Thrombin cleavage |
| | | site |
| 221 | LVPRGF | Thrombin cleavage |
| | | site |
| 222 | MYPRGN | Thrombin cleavage |
| | | site |
| 223 | E-X-X-Y-X-Q-S | TEV protease |
| | | cleavage site, X can |
| | | be any amino acid |
| 224 | YX₁YX₂X₃ | Stalk B motif, X₁, X₂, |
| | | X₃ is any amino acid |
| 225 | YX₁YX₂F | Stalk B motif, X₁ and |
| | | X₂, is any amino acid |
| 226 | YX₁YX₂Y | Stalk B motif, X1 and |
| | | X₂, is any amino acid |
| 227 | CPDGYIYSSNTASGYDCGVWICRRVGSAFCSRTGDYTSPS | 4C1 knob |
| 228 | NCPDGYIYSSNTASGYDCGVWICRRVGSAFCSRTGDYTSPS | 4C1 knob |
| 229 | CPDGYLYSSNTGRGYDCGVWTCRRVGGEFCSATGDWTSPS | R2C3 (R5C1) knob |
| 230 | SCPDGYLYSSNTGRGYDCGVWTCRRVGGEFCSATGDWTSPS | R2C3 (R5C1) knob |
| 231 | CPGGSSRRYPSGASCDVSGGACACYVSNCRGVLCPTLNEI | SKD knob |
| 232 | SCPGGSSRRYPSGASCDVSGGACACYVSNCRGVLCPTLNEI | SKD knob |
| 233 | CPDGYIDNSGCTADWGCAALDCWRRRFGYHSTDPSHYTGATY | SKM knob |
| | I | |
| 234 | CPDGYEHTCGCIGGCGCKRSACIGALCCQASLGGWLSD | 2G3 knob |
| 235 | CPDDFDYRCSCIGGCGCARKGCVGPLCCRSDLGGYLTDSPA | R2F12 knob |
| 236 | ACPDDFDYRCSCIGGCGCARKGCVGPLCCRSDLGGYLTDSPA | R2F12 knob |
| 237 | CPDGYIYSSNITSGFDCGVWICRRVGSAFCSRTGDYTSPT | SR3A3 knob |
| 238 | NCPDGYIYSSNITSGFDCGVWICRRVGSAFCSRTGDYTSPT | SR3A3 knob |
| 239 | CPNGYNWFDRCCSWDGTCGDGCCSNRAWPSGNGRADSSIG | R2D9 knob |
| 240 | TCPNGYNWFDRCCSWDGTCGDGCCSNRAWPSGNGRADSSIG | R2D9 knob |
| 241 | RKEKNCPDGYIYSSNTASGYDCGVWICRRVGSAFCSRTGDYTS | R4C1 knob |
| | PSEFDIYEFY | |
| 242 | RKEKNCPDGYIYSSNTASGYDCGVWICRRVGSAFCSRTGDYTS | R4C1 knob |
| 243 | RKEKNCPDGYIYSSNTASGYDCGVWICRRVGSAFCSRTGDYTS | R4C1 knob |
| | P | |
| 244 | RKEKNCPDGYIYSSNTASGYDCGVWICRRVGSAFCSRTGDYTS | R4C1 knob |
| | PS | |
| 245 | RKEKNCPDGYIYSSNTASGYDCGVWICRRVGSAFCSRTGDYTS | R4C1 knob |
| | PSE | |
| 246 | RKEKNCPDGYIYSSNTASGYDCGVWICRRVGSAFCSRTGDYTS | R4C1 knob |
| | PSEF | |
| 247 | RKEKNCPDGYIYSSNTASGYDCGVWICRRVGSAFCSRTGDYTS | R4C1 knob |
| | PSEFD | |
| 248 | RKEKNCPDGYIYSSNTASGYDCGVWICRRVGSAFCSRTGDYTS | R4C1 knob |
| | PSEFDI | |
| 249 | RKEKNCPDGYIYSSNTASGYDCGVWICRRVGSAFCSRTGDYTS | R4C1 knob |
| | PSEFDIYE | |
| 250 | RKEKNCPDGYIYSSNTASGYDCGVWICRRVGSAFCSRTGDYTS | R4C1 knob |
| | PSEFDIYEF | |
| 251 | RKEKSCPDGYLYSSNTGRGYDCGVWTCRRVGGEFCSATGDW | R2C3 knob |
| | TS | |
| 252 | RKEKSCPDGYLYSSNTGRGYDCGVWTCRRVGGEFCSATGDW | R2C3 knob |
| | TSP | |
| 253 | RKEKSCPDGYLYSSNTGRGYDCGVWTCRRVGGEFCSATGDW | R2C3 knob |
| | TSPS | |
| 254 | RKEKSCPDGYLYSSNTGRGYDCGVWTCRRVGGEFCSATGDW | R2C3 knob |
| | TSPSE | |
| 255 | RKEKSCPDGYLYSSNTGRGYDCGVWTCRRVGGEFCSATGDW | R2C3 knob |
| | TSPSEE | |
| 256 | RKEKSCPDGYLYSSNTGRGYDCGVWTCRRVGGEFCSATGDW | R2C3 knob |
| | TSPSEED | |
| 257 | RKEKSCPDGYLYSSNTGRGYDCGVWTCRRVGGEFCSATGDW | R2C3 knob |
| | TSPSEEDF | |
| 258 | RKEKSCPDGYLYSSNTGRGYDCGVWTCRRVGGEFCSATGDW | R2C3 knob |
| | TSPSEEDFY | |
| 259 | RKEKSCPDGYLYSSNTGRGYDCGVWTCRRVGGEFCSATGDW | R2C3 knob |
| | TSPSEEDFYE | |
| 260 | SEKRSCPGGSSRRYPSGASCDVSGGACACYVSNCRGVLCPTLN | SKD knob |
| | EIVAYTYEWHVD | |
| 261 | SEKRSCPGGSSRRYPSGASCDVSGGACACYVSNCRGVLCPTLN | SKD knob |
| | EI | |
| 262 | SEKRSCPGGSSRRYPSGASCDVSGGACACYVSNCRGVLCPTLN | SKD knob |
| | EIV | |
| 263 | SEKRSCPGGSSRRYPSGASCDVSGGACACYVSNCRGVLCPTLN | SKD knob |
| | EIVA | |
| 264 | SEKRSCPGGSSRRYPSGASCDVSGGACACYVSNCRGVLCPTLN | SKD knob |
| | EIVAY | |
| 265 | SEKRSCPGGSSRRYPSGASCDVSGGACACYVSNCRGVLCPTLN | SKD knob |
| | EIVAYT | |
| 266 | SEKRSCPGGSSRRYPSGASCDVSGGACACYVSNCRGVLCPTLN | SKD knob |
| | EIVAYTYE | |
| 267 | SEKRSCPGGSSRRYPSGASCDVSGGACACYVSNCRGVLCPTLN | SKD knob |
| | EIVAYTYEW | |
| 268 | SEKRSCPGGSSRRYPSGASCDVSGGACACYVSNCRGVLCPTLN | SKD knob |
| | EIVAYTYEWH | |
| 269 | SEKRSCPGGSSRRYPSGASCDVSGGACACYVSNCRGVLCPTLN | SKD knob |
| | EIVAYTYEWHV | |
| 270 | LRSCPDGYIDNSGCTADWGCAALDCWRRRFGYHSTDPSHYTG | SKM knob |
| | ATYIYTYSHID | |
| 271 | LRSCPDGYIDNSGCTADWGCAALDCWRRRFGYHSTDPSHYTG | SKM knob |
| | AT | |
| 272 | LRSCPDGYIDNSGCTADWGCAALDCWRRRFGYHSTDPSHYTG | SKM knob |
| | ATY | |
| 273 | LRSCPDGYIDNSGCTADWGCAALDCWRRRFGYHSTDPSHYTG | SKM knob |
| | ATYI | |
| 274 | LRSCPDGYIDNSGCTADWGCAALDCWRRRFGYHSTDPSHYTG | SKM knob |
| | ATYIY | |
| 275 | LRSCPDGYIDNSGCTADWGCAALDCWRRRFGYHSTDPSHYTG | SKM knob |
| | ATYIYT | |
| 276 | LRSCPDGYIDNSGCTADWGCAALDCWRRRFGYHSTDPSHYTG | SKM knob |
| | ATYIYTYS | |
| 277 | LRSCPDGYIDNSGCTADWGCAALDCWRRRFGYHSTDPSHYTG | SKM knob |
| | ATYIYTYS | |
| 278 | LRSCPDGYIDNSGCTADWGCAALDCWRRRFGYHSTDPSHYTG | SKM knob |
| | ATYIYTYSH | |
| 279 | LRSCPDGYIDNSGCTADWGCAALDCWRRRFGYHSTDPSHYTG | SKM knob |
| | ATYIYTYSHI | |
| 280 | EGDKTCPDGYEHTCGCIGGCGCKRSACIGALCCQASLGGWLS | R2G3 knob |
| 281 | EGDKTCPDGYEHTCGCIGGCGCKRSACIGALCCQASLGGWLS | R2G3 knob |
| | D | |
| 282 | EGDKTCPDGYEHTCGCIGGCGCKRSACIGALCCQASLGGWLS | R2G3 knob |
| | DG | |
| 283 | EGDKTCPDGYEHTCGCIGGCGCKRSACIGALCCQASLGGWLS | R2G3 knob |
| | DGE | |
| 284 | EGDKTCPDGYEHTCGCIGGCGCKRSACIGALCCQASLGGWLS | R2G3 knob |
| | DGET | |
| 285 | EGDKTCPDGYEHTCGCIGGCGCKRSACIGALCCQASLGGWLS | R2G3 knob |
| | DGETY | |
| 286 | EGDKTCPDGYEHTCGCIGGCGCKRSACIGALCCQASLGGWLS | R2G3 knob |
| | DGETYT | |
| 287 | EGDKTCPDGYEHTCGCIGGCGCKRSACIGALCCQASLGGWLS | R2G3 knob |
| | DGETYTY | |
| 288 | EGDKTCPDGYEHTCGCIGGCGCKRSACIGALCCQASLGGWLS | R2G3 knob |
| | DGETYTYE | |
| 289 | GDKTCPDGYEHTCGCIGGCGCKRSACIGALCCQASLGGWLSD | R2G3 knob |
| 290 | DKTCPDGYEHTCGCIGGCGCKRSACIGALCCQASLGGWLSD | R2G3 knob |
| 291 | KTCPDGYEHTCGCIGGCGCKRSACIGALCCQASLGGWLSD | R2G3 knob |
| 292 | KTKNACPDDFDYRCSCIGGCGCARKGCVGPLCCRSDLGGYLT | R2F12 knob |
| | DSPAYIYEWY | |
| 293 | KTKNACPDDFDYRCSCIGGCGCARKGCVGPLCCRSDLGGYLT | R2F12 knob |
| | DS | |
| 294 | KTKNACPDDFDYRCSCIGGCGCARKGCVGPLCCRSDLGGYLT | R2F12 knob |
| | DSP | |
| 295 | KTKNACPDDFDYRCSCIGGCGCARKGCVGPLCCRSDLGGYLT | R2F12 knob |
| | DSPA | |
| 296 | KTKNACPDDFDYRCSCIGGCGCARKGCVGPLCCRSDLGGYLT | R2F12 knob |
| | DSPAY | |
| 297 | KTKNACPDDFDYRCSCIGGCGCARKGCVGPLCCRSDLGGYLT | R2F12 knob |
| | DSPAYI | |
| 298 | KTKNACPDDFDYRCSCIGGCGCARKGCVGPLCCRSDLGGYLT | R2F12 knob |
| | DSPAYIY | |
| 299 | KTKNACPDDFDYRCSCIGGCGCARKGCVGPLCCRSDLGGYLT | R2F12 knob |
| | DSPAYIYEW | |
| 300 | KEKNCPDGYIYSSNITSGFDCGVWICRRVGSAFCSRTGDYTS | SR3A3 knob |
| 301 | KEKNCPDGYIYSSNITSGFDCGVWICRRVGSAFCSRTGDYTSP | SR3A3 knob |
| 302 | KEKNCPDGYIYSSNITSGFDCGVWICRRVGSAFCSRTGDYTSPT | SR3A3 knob |
| 303 | KEKNCPDGYIYSSNITSGFDCGVWICRRVGSAFCSRTGDYTSPT | SR3A3 knob |
| | E | |
| 304 | KEKNCPDGYIYSSNITSGFDCGVWICRRVGSAFCSRTGDYTSPT | SR3A3 knob |
| | EL | |
| 305 | KEKNCPDGYIYSSNITSGFDCGVWICRRVGSAFCSRTGDYTSPT | SR3A3 knob |
| | ELD | |
| 306 | KEKNCPDGYIYSSNITSGFDCGVWICRRVGSAFCSRTGDYTSPT | SR3A3 knob |
| | ELDI | |
| 307 | KEKNCPDGYIYSSNITSGFDCGVWICRRVGSAFCSRTGDYTSPT | SR3A3 knob |
| | ELDIY | |
| 308 | KEKNCPDGYIYSSNITSGFDCGVWICRRVGSAFCSRTGDYTSPT | SR3A3 knob |
| | ELDIYE | |
| 309 | ITHKTCPNGYNWFDRCCSWDGTCGDGCCSNRAWPSGNGRAD | R2D9 knob |
| | SS | |
| 310 | ITHKTCPNGYNWFDRCCSWDGTCGDGCCSNRAWPSGNGRAD | R2D9 knob |
| | SSI | |
| 311 | ITHKTCPNGYNWFDRCCSWDGTCGDGCCSNRAWPSGNGRAD | R2D9 knob |
| | SSIG | |
| 312 | ITHKTCPNGYNWFDRCCSWDGTCGDGCCSNRAWPSGNGRAD | R2D9 knob |
| | SSIGE | |
| 313 | ITHKTCPNGYNWFDRCCSWDGTCGDGCCSNRAWPSGNGRAD | R2D9 knob |
| | SSIGET | |
| 314 | ITHKTCPNGYNWFDRCCSWDGTCGDGCCSNRAWPSGNGRAD | R2D9 knob |
| | SSIGETY | |
| 315 | ITHKTCPNGYNWFDRCCSWDGTCGDGCCSNRAWPSGNGRAD | R2D9 knob |
| | SSIGETYG | |
| 316 | ITHKTCPNGYNWFDRCCSWDGTCGDGCCSNRAWPSGNGRAD | R2D9 knob |
| | SSIGETYGY | |
| 317 | ITHKTCPNGYNWFDRCCSWDGTCGDGCCSNRAWPSGNGRAD | R2D9 knob |
| | SSIGETYGYE | |
| 318 | HQETLRSCPDGYIDNSGCTADWGCAALDCWRRRFGYHSTDPS | SKM knob |
| | HYTGATYIYTYSLH | |
| 319 | APTSSSTKKTQLQLEHLLLDLQMILNGINNYKNPKLTRMLTFKF | IL2 |
| | YMPKKATELKHLQCLEEELKPLEEVLNLAQSKNFHLRPRDLIS | |
| | NINVIVLELKGSETTFMCEYADETATIVEFLNRWITFCQSIISTLT | |
| 320 | NWVNVISDLKKIEDLIQSMHIDATLYTESDVHPSCKVTAMKCF | IL-15 |
| | LLELQVISLESGDASIHDTVENLIILANNSLSSNGNVTESGCKEC | |
| | EELEEKNIKEFLQSFVHIVQMFINTS | |
| 321 | MSKGEELFTGVVPILVELDGDVNGHKFSVRGEGEGDATNGKL | sfGFP control |
| | TLKFICTTGKLPVPWPTLVTTLTYGVQCFSRYPDHMKRHDFFK | |
| | SAMPEGYVQERTISFKDDGTYKTRAEVKFEGDTLVNRIELKGI | |
| | DFKEDGNILGHKLEYNFNSHNVYITADKQKNGIKANFKIRHNV | |
| | EDGSVQLADHYQQNTPIGDGPVLLPDNHYLSTQSVLSKDPNEK | |
| | RDHMVLLEFVTAAGITHGMDELYKGGLEHHHHHH | |
| 322 | MSKGEELFTGVVPILVELDGDVNGHKFSVRGEGEGDATNGKL | sfGFP-link-G3 |
| | TLKFICTTGKLPVPWPTLVTTLTYGVQCFSRYPDHMKRHDFFK | |
| | SAMPEGYVQERTISFKDDGTYKTRAEVKFEGDTLVNRIELKGI | |
| | DFKEDGNILGHKLEYNFNSHNVYITADKQKNGIKANFKIRHNV | |
| | EDGGGSKTCPDGYEHTCGCIGGCGCKRSACIGALCCQASLGG | |
| | WLSDGGGSGSVQLADHYQQNTPIGDGPVLLPDNHYLSTQSVL | |
| | SKDPNEKRDHMVLLEFVTAAGITHGMDELYKGGLEHHHHHH | |
| 323 | MSKGEELFTGVVPILVELDGDVNGHKFSVRGEGEGDATNGKL | sfGFP-stalk-G3 |
| | TLKFICTTGKLPVPWPTLVTTLTYGVQCFSRYPDHMKRHDFFK | |
| | SAMPEGYVQERTISFKDDGTYKTRAEVKFEGDTLVNRIELKGI | |
| | DFKEDGNILGHKLEYNFNSHNVYITADKQKNGIKANFKIRHNV | |
| | EDCATVHQKTAEGDKTCPDGYEHTCGCIGGCGCKRSACIGAL | |
| | CCQASLGGWLSDGETYTYEFHVDTWGSVQLADHYQQNTPIGD | |
| | GPVLLPDNHYLSTQSVLSKDPNEKRDHMVLLEFVTAAGITHG | |
| | MDELYKGGLEHHHHHH | |
| 324 | MSKGEELFTGVVPILVELDGDVNGHKFSVRGEGEGDATNGKL | sfGFP-coiled coil- |
| | TLKFICTTGKLPVPWPTLVTTLTYGVQCFSRYPDHMKRHDFFK | G3 |
| | SAMPEGYVQERTISFKDDGTYKTRAEVKFEGDTLVNRIELKGI | |
| | DFKEDGNILGHKLEYNFNSHNVYITADKQKNGIKANFKIRHNV | |
| | EDGGSGAKLAALKAKLAALKGGGGSKTCPDGYEHTCGCIGGC | |
| | GCKRSACIGALCCQASLGGWLSDGGGGSELAALEAELAALEA | |
| | GGSGGSVQLADHYQQNTPIGDGPVLLPDNHYLSTQSVLSKDPN | |
| | EKRDHMVLLEFVTAAGITHGMDELYKGGSLEHHHHHH | |
| 325 | MRRMQLLLLIALSLALVTNSGGGGSEPKSCDKTHTCPPCPAPE | Fc-G3 coiled coil |
| | LLGGPSVFLFPPKPKDTLMISRTPEVTCVVVDVSHEDPEVKFN | (cc) |
| | WYVDGVEVHNAKTKPREEQYNSTYRVVSVLTVLHQDWLNGK | |
| | EYKCKVSNKALPAPIEKTISKAKGGGGSAKLAALKAKLAALK | |
| | GGGGSKTCPDGYEHTCGCIGGCGCKRSACIGALCCQASLGGW | |
| | LSDGGGGSELAALEAELAALEAGGGSQPREPQVYTLPPSRDEL | |
| | TKNQVSLTCLVKGFYPSDIAVEWESNGQPENNYKTTPPVLDSD | |
| | GSFFLYSKLTVDKSRWQQGNVFSCSVMHEALHNHYTQKSLSL | |
| | SPGK | |
| 326 | MRRMQLLLLIALSLALVTNSGGGGSEPKSCDKTHTCPPCPAPE | Fc-G3-link |
| | LLGGPSVFLFPPKPKDTLMISRTPEVTCVVVDVSHEDPEVKFN | |
| | WYVDGVEVHNAKTKPREEQYNSTYRVVSVLTVLHQDWLNGK | |
| | EYKCKVSNKALPAPIEKTISKAKGGGGSKTCPDGYEHTCGCIG | |
| | GCGCKRSACIGALCCQASLGGWLSDGGGSQPREPQVYTLPPSR | |
| | DELTKNQVSLTCLVKGFYPSDIAVEWESNGQPENNYKTTPPVL | |
| | DSDGSFFLYSKLTVDKSRWQQGNVFSCSVMHEALHNHYTQKS | |
| | LSLSPGK | |
| 327 | MGGSGAKLAALKAKLAALKGGGGSDYKDDDDKGGGSKTCP | CKC-G3 |
| | DGYEHTCGCIGGCGCKRSACIGALCCQASLGGWLSDGGGSDY | |
| | KDDDDKGGGSSELAALEAELAALEAGGSGGGSLEHHHHHH | |
| 328 | MSDKIIHLTDDSFDTDVLKADGAILVDFWAEWCGPCKMIAPIL | TrxA_Cterm- |
| | DEIADEYQGKLTVAKLNIDQNPGTAPKYGIRGIPTLLLFKNGEV | cchIL15 |
| | AATKVGALSKGQLKEFLDANLAGGSDYKDDDDKGGSGAKLA | |
| | ALKAKLAALKGGGGSNWVNVISDLKKIEDLIQSMHIDATLYTE | |
| | SDVHPSCKVTAMKCFLLELQVISLESGDASIHDTVENLIILANNS | |
| | LSSNGNVTESGCKECEELEEKNIKEFLQSFVHIVQMFINTSGGG | |
| | GSELAALEAELAALEAGGSGLEHHHHHH | |
| 329 | MSDKIIHLTDDSFDTDVLKADGAILVDFWAEWCGPCKMIAPIL | TrxA + flag |
| | DEIADEYQGKLTVAKLNIDQNPGTAPKYGIRGIPTLLLFKNGEV | |
| | AATKVGALSKGQLKEFLDANLAGGSDYKDDDDK | |
| 330 | GGSGAKLAALKAKLAALKGGGGSNWVNVISDLKKIEDLIQSM | ccIL15 |
| | HIDATLYTESDVHPSCKVTAMKCFLLELQVISLESGDASIHDTV | |
| | ENLIILANNSLSSNGNVTESGCKECEELEEKNIKEFLQSFVHIVQ | |
| | MFINTSGGGGSELAALEAELAALEAGGSGLEHHHHHH | |
| 331 | MDAMKRGLCCVLLLCGAVFVSPSQEIHARFRRGARGGSGAKL | cc-hIL15-TEV |
| | AALKAKLAALKGGGGSNWVNVISDLKKIEDLIQSMHIDATLYT | |
| | ESDVHPSCKVTAMKCFLLELQVISLESGDASIHDTVENLIILAN | |
| | NSLSSNGNVTESGCKECEELEEKNIKEFLQSFVHIVQMFINTSG | |
| | GGGSELAALEAELAALEAGGSGENLYFQSAGHHHHHH | |
| 332 | EPKSCDKTHTCPPCPAPELLGGPSVFLFPPKPKDTLMISRTPEVT | IgG Fc |
| | CVVVDVSHEDPEVKFNWYVDGVEVHNAKTKPREEQYNSTYR | |
| | VVSVLTVLHQDWLNGKEYKCKVSNKALPAPIEKTISKAKGQP | |
| | REPQVYTLPPSRDELTKNQVSLTCLVKGFYPSDIAVEWESNGQ | |
| | PENNYKTTPPVLDSDGSFFLYSKLTVDKSRWQQGNVFSCSVM | |
| | HEALHNHYTQKSLSLSPGK | |
| 333 | MRRMQLLLLIALSLALVINS | IL-2 signal |

What is claimed is:

1. A modified fusion polypeptide comprising the formula N-terminus to C-terminus: Y1-[polypeptide]-Y2, wherein the polypeptide is a protein in which the distance between the N-termini and C-termini is no more than 10 Angstroms and wherein Y1 and Y2 are amino acid peptide sequences that interact with one another when in proximity.

2. The modified fusion polypeptide of claim 1, wherein Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif.

3. The modified fusion polypeptide of claim 2, wherein the Y1 and Y2 are HW and SF, respectively.

4. The modified fusion polypeptide of claim 2, wherein Y1 and Y2 are IS and TV, respectively.

5. The modified fusion polypeptide of claim 2, wherein Y1 and Y2 are sequences that are each 14-30 amino acids in length comprising a heptad repeat, wherein Y1 and Y2 are able to interact to form an anti-parallel coiled-coil motif.

6. The modified fusion polypeptide of claim 2 and 5, wherein Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203.

7. The modified fusion polypeptide of claim 2, 5 or 6, wherein the Y1 is set forth in SEQ ID NO:202 and Y2 is set forth in SEQ ID NO:203, respectively.

8. The modified fusion polypeptide of any of claims 1-7, wherein the distance between the N- and C-termini is no more than 5, 6, 7, 8 or 9 Angstroms.

9. The modified fusion polypeptide of any of claims 1-8, wherein the distance between the N- and C-termini is between 2 and 10 Angstroms.

10. The modified fusion polypeptide of any of claims 1-9, wherein the distance between the N- and C-termini is between 2 and 8 Angstroms.

11. The modified fusion polypeptide of any of claims 1-10, wherein the polypeptide is selected from a cysteine motif peptide or a cytokine, wherein the cysteine motif binding peptide is 20-50 amino acids in length in which 2-12 amino acids are cysteine residues able to form 1-6 disulfide bonds.

12. The modified fusion polypeptide of claim 11, wherein the cysteine motif binding peptide is a knob peptide from an ultralong CDR3 of a cow antibody.

13. The modified fusion polypeptide of claim 11 or claim 12, wherein the cysteine motif binding peptide does not comprise an ascending or descending stalk domain or a contiguous portion thereof of more than 3 amino acids in length from the ultralong CDR3 of the cow antibody.

14. The modified fusion polypeptide of any of claims 11-13, wherein the cysteine motif binding peptide is 22 to 43 amino acids in length.

15. The modified fusion polypeptide of any of claims 11-14, wherein the cysteine motif binding peptide comprises at least 4 cysteine residues.

16. The modified fusion polypeptide of any of claims 11-15, wherein the cysteine motif binding peptide contains 4 cysteine residues, 6 cysteine residues or 8 cysteine residues.

17. The modified fusion polypeptide of any of claims 11-16, wherein the cysteine motif binding peptide is able to form at least 2 disulfide bonds.

18. The modified fusion polypeptide of any of claims 11-17, wherein the cysteine motif binding peptide is able to form 2 disulfide bonds, 3 disulfide bonds or 4 disulfide bonds.

19. The modified fusion polypeptide of any of claim 11-18, wherein the cysteine motif binding peptides binds to a target antigen that is a nonvirulent bacteria, a virus, a viral protein, an immunomodulatory protein, a cancer antigen, a human IgG, or a recombinant protein thereof.

20. The modified fusion polypeptide of claim 19, wherein the target antigen is a coronavirus, a coronavirus pseudovirus, a recombinant coronavirus Spike protein, or a receptor-binding domain (RBD) of a coronavirus Spike protein.

21. The modified fusion polypeptide of claim 20, wherein the coronavirus is selected from the group consisting of 229E, NL63, OC43, HKU1, MERS-CoV, SARS-CoV, and SARS-CoV2.

22. The modified fusion polypeptide of claim 20 or claim 21, wherein the coronavirus is a SARS-CoV2 selected from Wuhan-Hu-1 isolate, B.1.351 South African variant, B.1.1.7 UK variant, a beta variant, a delta variant, or an omicron variant.

23. The modified fusion polypeptide of any of claims 11-22, wherein the cysteine motif binding peptide is set forth in any one of SEQ ID NOS: 155, 198, and 227-240.

24. The modified polypeptide of any of claims 1-18, wherein the cytokine is IL-2 or IL-15.

25. A fusion protein, comprising a modified fusion polypeptide of any of claims 1-24 and a moiety selected from a half-life extending moiety or a detectable moiety.

26. The fusion protein of claim 25, wherein the half-life extending moiety is an immunoglobulin Fc.

27. The fusion protein of claim 25, wherein the detectable moiety is a fluorescent protein.

28. The fusion protein of claim 27, wherein the fluorescent protein is a GFP, optionally sfGFP.

29. The fusion protein of any of claims 25-28, wherein the modified fusion polypeptide is inserted within the half-life extending moiety or detectable moiety.

30. The fusion protein of any of claims 25-29, wherein the modified fusion polypeptide is inserted within a loop of the half-life extending moiety or detectable moiety.

31. A nucleic acid encoding a modified fusion polypeptide of any of claims 1-24 or the fusion protein of any of claims 25-30.

32. An expression vector comprising the nucleic acid molecule of claim 31.

33. A composition comprising the modified fusion polypeptide of any of claims 1-24 or the fusion protein of any of claims 25-30.

34. The composition of claim 33 that is a pharmaceutical composition comprising a pharmaceutically acceptable excipient.

35. A method of producing a modified fusion polypeptide, the method comprising adding an N-terminus sequence (Y1) and a C-terminus sequence (Y2) to a polypeptide, wherein the polypeptide is a protein in which the distance between the N-termini and C-termini is no more than 10 Angstroms and wherein the modified fusion polypeptide has the formula N-terminus to C-terminus: Y1-[polypeptide]-Y2, wherein Y1 and Y2 are amino acid peptide sequences that interact with one another when in proximity.

36. The method of claim 35, wherein Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif.

37. The method of claim 35 or claim 36, wherein the polypeptide is selected from a cysteine motif peptide or a cytokine, wherein the cysteine motif binding peptide is 20-50 amino acids in length in which 2-12 amino acids are cysteine residues able to form 1-6 disulfide bonds.

38. The method of claim 37, wherein the cytokine is IL-2 or IL-15.

39. A method of producing a modified binding peptide, the method comprising:
(a) obtaining a cysteine motif binding peptide of 20-50 amino acids in length in which 2-12 amino acids are cysteine residues able to form 1-6 disulfide bonds; and
(b) modifying the binding peptide by adding an N-terminus sequence (Y1) and a C-terminus sequence (Y2) to the cysteine motif binding peptide, wherein the modified binding peptide has the formula N-terminus to C-terminus: Y1-[cysteine motif binding peptide]-Y2, wherein Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif.

40. The method of any of claims 35-39, wherein the Y1 and Y2 are HW and SF, respectively.

41. The method of any of claims 35-39, wherein Y1 and Y2 are IS and TV, respectively.

42. The method of any of claims 35-39, wherein Y1 and Y2 are sequences that are each 14-30 amino acids in length comprising a heptad repeat, wherein Y1 and Y2 are able to interact to form an anti-parallel coiled-coil motif.

43. The method of any of claims 35-39 and 42, wherein Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203.

44. The method of any of claims 35-39, 42 and 43, wherein the Y1 is set forth in SEQ ID NO:202 and Y2 is set forth in SEQ ID NO:203, respectively.

45. The method of any of claims 37 and 39-44, wherein the cysteine motif binding peptide is a knob peptide from an ultralong CDR3 of a cow antibody.

46. The method of claim 45, wherein the cysteine motif binding peptide does not comprise an ascending or descending stalk domain or a contiguous portion thereof of more than 3 amino acids in length from the ultralong CDR3 of the cow antibody.

47. The method of any of claims 37 and 39-46, wherein the cysteine motif binding peptide is 22 to 43 amino acids in length.

48. The method of any of claims 37 and 39-47, wherein the cysteine motif binding peptide comprises at least 4 cysteine residues.

49. The method of any of claims 37 and 39-48, wherein the cysteine motif binding peptide contains 4 cysteine residues, 6 cysteine residues or 8 cysteine residues.

50. The method of any of claims 37 and 39-49, wherein the cysteine motif binding peptide is able to form at least 2 disulfide bonds.

51. The method of any of claims 37 and 39-50, wherein the cysteine motif binding peptide is able to form 2 disulfide bonds, 3 disulfide bonds or 4 disulfide bonds.

52. The method of any of claims 37 and 39-51, wherein the cysteine motif binding peptide is identified by a method comprising:
(1) immunizing a cow with a target antigen or a sequence portion comprising an epitope thereof;
(2) identifying a knob peptide sequence from an antibody variable heavy chain (VH) sequence from peripheral blood mononuclear cells (PBMCs) from the immunized cow, wherein the knob peptide is a sequence between the ascending and descending stalk sequences of an ultralong CDR3, wherein the ultralong CDR3 is 40 to 70 amino acids in length, and wherein the knob peptide is a cysteine motif binding peptide of 20-50 amino acids in length in which 2-12 amino acids are cysteine residues able to form 1-6 disulfide bonds.

53. The method of claim 52, wherein the knob peptide is identified from the VH sequence by an algorithm comprising:
identifying the conserved cysteine in framework 3 and the conserved tryptophan in framework 4; and
determining the sequence of the knob, in which:
the knob has the amino acid sequence length K;
the sequence begins at position X+1 and ends at X+K; and
K=L−2X; wherein L is the number of amino acids in an amino acid sequence starting at the conserved cysteine in framework 3 and ending at the conserved tryptophan in framework 4, and X is the number of amino acids from the first cysteine in framework 3 to the first conserved cysteine encoded by the D_{H} region in CDR H3.

54. The method of claim 52 or claim 53, wherein the cysteine motif binding peptide is extended by one, two, three, four, or five amino acids at the N and/or C termini of the ultralong CDR3 compared to the determined knob sequence.

55. The method of any of claims 52-54, wherein identifying the knob peptide comprises:
(a) amplifying sequences encoding a plurality of variable heavy (VH) regions of the IgHV1-7 family from a VH chain complementary DNA (cDNA) template library prepared from RNA isolated from the PBMCs from the immunized cow;
(b) constructing a plurality of replicable expression vectors for the plurality of VH regions, wherein each replicable expression vector comprises a nucleic acid sequence encoding a single chain variable fragment (scFv) comprising an amplified VH region joined to a variable lambda light (VL) region selected from the group consisting of VL regions of BLV1H12, BLV5D3, BLV8C11, BF1H1, BLV5B8, and F18, or a humanized variant thereof;
(c) transforming suitable host cells with the plurality of replicable expression vectors under conditions suitable to produce amplified display particles; and
(d) collecting the amplified display particles, wherein the amplified display particles comprise display particles displaying a fusion protein comprising an scFv; and
(e) contacting the amplified display particles with a target antigen under conditions to allow binding of a display particle to the target antigen; and
(f) selecting display particles comprising an antibody that binds to the target antigen by separating the display particles that bind from those that do not; and
(g) sequencing the fusion gene in the selected display particles to identify the antibody with a VH sequence that comprises or is suspected of comprising an ultralong CDR3.

56. The method of claim 55, wherein the VL region is the BLV1H12 VL region.

57. The method of claim 56, wherein the BLV1H12 lambda VL region is set forth in SEQ ID NO: 2.

58. The method of claim 55, wherein the BLV1H12 lambda VL region is a humanized variant of the lambda VL region of BLV1H12.

59. The method of claim 58, wherein the humanized variant comprises one or more of amino acid replacements S2A, T5N, P8S, A12G, A13S, and P14L based on Kabat numbering, amino acid replacements I29V and N32G in the CDR1 region, and/or amino acid substitution of DNN to GDT in the CDR2 region.

60. The method of any of claim 58 or claim 59, wherein the humanized variant comprises the sequence set forth in SEQ ID NO: 107.

61. The method of any of claims 55-60, wherein the cDNA template library is prepared from RNA isolated from peripheral blood mononuclear cells (PBMCs) from an immunized cow.

62. The method of any of claims 55-61, wherein the plurality of VH regions of the IgHV1-7 family from the cDNA template library are amplified with a forward primer comprising the sequence set forth in SEQ ID NO: 84 and a reverse primer comprising the sequence set forth in SEQ ID NO: 85.

63. The method of any of claims 55-62, wherein prior to the constructing, the method further comprises performing a size separation on the sequences encoding the plurality of amplified VH regions to enrich for VH regions with an ultralong CDR3, optionally wherein the size separation is by gel electrophoresis.

64. The method of claim 63, wherein the size separation comprises separating sequences of, of about, or greater than 550 base pairs in length from the sequences encoding the plurality of amplified VH regions, wherein the sequences of, of about, or greater than 550 base pairs in length comprise sequences encoding VH regions with an ultralong CDR3.

65. The method of any of claims 52-54, wherein identifying the knob peptide sequence comprises amplification from a variable heavy chain cDNA template library from the immunized cow using primers specific for either side of the stalk domain of a cow ultralong CDR3 region.

66. The method of any of claims 52-54 and 65, wherein identifying the knob peptide comprises:
(a) amplifying sequences encoding a plurality of CDR3-knob only antibodies from a cow antibody variable heavy (VH) chain complementary DNA (cDNA) template library with forward and reverse primers specific for the ascending and descending stalk domains of a cow ultralong CDR3 region;
(b) constructing a plurality of replicable expression vectors for the plurality of CDR3-knob only antibodies, wherein each replicable expression vector comprises a nucleic acid sequence encoding an amplified CDR3 knob;
(c) transforming suitable host cells with the plurality of replicable expression vectors under conditions suitable to produce amplified display particles;
(d) collecting the amplified display particles, wherein the amplified display particles comprise display particles displaying a fusion protein comprising an amplified CDR3 knob;
(e) contacting the amplified display particles with a target antigen under conditions to allow binding of a display particle to the target antigen;
(f) selecting display particles comprising a CDR3-knob only antibody that binds to the target antigen by separating the display particles that bind from those that do not; and
(g) sequencing the fusion gene in the selected display particles to identify the CDR3-knob antibody.

67. The method of claim 65 or claim 66, wherein the cDNA template library is prepared from RNA isolated from peripheral blood mononuclear cells (PBMCs) from an immunized cow.

68. The method of any of claims 65-67, wherein the primers are a pool of primers that comprise or consist of any of the sequences set forth in SEQ ID NO: 7-11 and 121-130, optionally comprise or consist of any of the sequences set forth in SEQ ID NO: 123, 127, and 128.

69. The method of any of claims 55-68, wherein the amplified display particles are phage display particles.

70. The method of any of claims 39-69, wherein the cysteine motif binding peptide binds to a target antigen.

71. The method of any of claims 52-70, wherein the target antigen is a nonvirulent bacteria, a virus, a viral protein, an immunomodulatory protein, a cancer antigen, a human IgG, or a recombinant protein thereof.

72. The method of any of claims 52-71, wherein the target antigen is a coronavirus, a coronavirus pseudovirus, a recombinant coronavirus Spike protein, or a receptor-binding domain (RBD) of a coronavirus Spike protein.

73. The method of claim 72, wherein the coronavirus is selected from the group consisting of 229E, NL63, OC43, HKU1, MERS-CoV, SARS-CoV, and SARS-CoV2.

74. The method of claim 72 or claim 73, wherein the coronavirus is a SARS-CoV2 selected from Wuhan-Hu-1 isolate, B.1.351 South African variant, B.1.1.7 UK variant, a beta variant, a delta variant, or a omicron variant.

75. The method of any of claims 35-74, wherein Y1 and Y2 are added by synthetic methods or by recombinant DNA techniques.

76. A modified binding peptide produced by the methods of any of claims 35-75.

77. A nucleic acid molecule encoding a modified binding peptide produced by the methods of any of claims 35-75.

78. A method for producing a soluble binding peptide, comprising:
(a) transforming E. coli with an expression vector encoding a fusion protein comprising a binding peptide and thioredoxin A (TrxA) bacterial chaperone set forth in SEQ ID NO:194 or a sequence of amino acids that exhibits at least 85%, 86%, 87%, 88%, 89%, 90%, 91%, 92%, 92%, 94%, 95%, 96%, 97%, 98%, 99% sequence identity to SEQ ID NO:194, wherein the binding peptide is a cysteine modified binding peptide of 20-50 amino acids in length in which 2-12 amino acids are cysteine residues able to form 1-6 disulfide bonds; and;
(b) culturing the bacteria under conditions permissive of expression of the fusion protein; and
(c) isolating the fusion protein from supernatant of a bacterial cell lysate.

79. The method of claim 78, wherein the cysteine modified binding peptide binding peptide comprises a knob peptide from an ultralong CDR3 of a cow antibody.

80. The method of claim 78 or claim 79, wherein the cysteine modified binding peptide is set forth in any one of SEQ ID NOS: 60, 61, 62, 63, 64, 65, 66, 67, 68, 155, 198 and 227-317.

81. A method for producing a soluble ultralong CDR3 knob, comprising:
(a) transforming E. coli with an expression vector encoding a fusion protein comprising a binding peptide and a bacterial chaperone, wherein the binding peptide is a modified binding peptide that has the formula N-terminus to C-terminus: Y1-[cysteine motif binding peptide]-Y2, wherein the cysteine motif binding peptide is a peptide sequence of 20-50 amino acids with a cysteine motif comprising 2-12 cysteine residues able to form 1-6 disulfide bonds and Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif;
(b) culturing the bacteria under conditions permissive of expression of the fusion protein; and
(c) isolating the fusion protein from supernatant of a bacterial cell lysate.

82. The method of claim 81, wherein the Y1 and Y2 are HW and SF, respectively.

83. The method of claim 81, wherein Y1 and Y2 are IS and TV, respectively.

84. The method of claim 81, wherein Y1 and Y2 are sequences that are each 14-30 amino acids in length comprising a heptad repeat, wherein Y1 and Y2 are able to interact to form an anti-parallel coiled-coil motif.

85. The method of claim 81 or claim 84, wherein Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203.

86. The method of claim 81, 84 or 85, wherein the Y1 is set forth in SEQ ID NO:202 and Y2 is set forth in SEQ ID NO:203, respectively.

87. The method of any of claims 81-86, wherein the cysteine motif binding peptide is a knob peptide from an ultralong CDR3 of a cow antibody.

88. The method of claim 87, wherein the cysteine motif binding peptide does not comprise an ascending or descending stalk domain or a contiguous portion thereof of more than 3 amino acids in length from the ultralong CDR3 of the cow antibody.

89. The method of any of claims 81-88, wherein the cysteine motif binding peptide is 22 to 43 amino acids in length.

90. The method of any of claims 81-89, wherein the cysteine motif binding peptide comprises at least 4 cysteine residues.

91. The method of any of claims 81-90, wherein the cysteine motif binding peptide contains 4 cysteine residues, 6 cysteine residues or 8 cysteine residues.

92. The method of any of claims 81-91, wherein the cysteine motif binding peptide is able to form at least 2 disulfide bonds.

93. The method of any of claims 81-92, wherein the cysteine motif binding peptide is able to form 2 disulfide bonds, 3 disulfide bonds or 4 disulfide bonds.

94. A method for producing a soluble binding peptide, comprising:
(a) transforming E. coli with an expression vector encoding a fusion protein comprising a binding peptide and a bacterial chaperone, wherein the binding peptide is set forth in any one of SEQ ID NOS: 60, 61, 62, 63, 64, 65, 66, 67, 68, 155, 198 and 227-317 and;
(b) culturing the bacteria under conditions permissive of expression of the fusion protein;
(c) isolating the fusion protein from supernatant of a bacterial cell lysate.

95. The method of claims 78-94, wherein the binding peptide is set forth in any of SEQ ID NOS: 155, 198, and 227-240.

96. The method of any of claims 81-95, wherein the bacterial chaperone is thioredoxin A (TrxA).

97. The method of claim 96, wherein TrxA has the sequence set forth in SEQ ID NO:194 or a sequence of amino acids that exhibits at least 85%, 86%, 87%, 88%, 89%, 90%, 91%, 92%, 92%, 94%, 95%, 96%, 97%, 98%, 99% sequence identity to SEQ ID NO:194.

98. The method of any of claims 78-80, 96 and 97, wherein TrxA has the sequence set forth in SEQ ID NO:194.

99. The method of any of claims 78-98, wherein the binding peptide and bacterial chaperone are joined by a cleavable linker.

100. The method of any of claims 78-99, wherein the binding peptide is C-terminal to the bacterial chaperone.

101. The method of claim 99 or 100, wherein the method further comprises (d) cleaving the cleavable linker of the fusion protein, thereby producing a soluble binding peptide comprising 1-6 disulfide bonds free of the bacterial chaperone.

102. The method of any of claims 99-101, wherein the cleavable linker comprises a cleavage site selected from:
(i) an enterokinase cleavage site, optionally comprising the amino acid sequence set forth by DDDDK (SEQ ID NO:106);
(ii) a Factor Xa cleavage site, optionally comprising the amino acid sequence set forth by SEQ ID NO: 218 or I-E/D-G-R;
(iii) a thrombin cleavage site, optionally comprising the amino acid sequence set forth by any one of SEQ ID NOS: 219-222, more optionally set forth by SEQ ID NO: 220; or
(iv) a TEV protease cleavage site set forth by SEQ ID NO: 223 or SEQ ID NO:224.

103. The method of claim 101 or claim 102, wherein cleaving the cleavable linker comprises contacting the fusion protein with the protease that recognizes the cleavage site.

104. The method of any of claims 99-103, wherein the cleavable linker is a enterokinase cleavable linker comprising the amino acid sequence DDDDK (SEQ ID NO:106).

105. The method of claim 104, wherein the enterokinase cleavable linker comprises the sequence DYKDDDK (SEQ ID NO: 209) or GGSDYKDDDDKGS (SEQ ID NO:210).

106. The method of any of claims 101-105, wherein cleaving the cleavable linker comprises contacting the fusion protein with an enterokinase.

107. The method of any of claims 101-106, further comprising removing the bacterial chaperone from the solution comprising the soluble modified binding peptide.

108. The method of any of claims 101-107, further comprising removing the protease, optionally the enterokinase, from the solution comprising the soluble modified binding peptide.

109. The method of any of claims 78-99, wherein the binding is peptide is engineered into a loop of the bacterial chaperone.

110. The method of claim 109, wherein the bacterial chaperone is TrxA and the loop is selected the catalytic loop corresponding to residues 31-35 of SEQ ID NO: 194, the first binding loop corresponding to residues 74-76 of SEQ ID NO:194 or the second binding loop corresponding to residues 91-93 of SEQ ID NO:194.

111. The method of claim 109 or claim 110, wherein the bacterial chaperone is TrxA and the loop is the second binding loop corresponding to residues 91-93 of SEQ ID NO:194, optionally wherein the modified binding peptide is engineered between Val-92 and Gly-93 of the sequence set forth in SEQ ID NO:194.

112. The method of any of claims 109-111, wherein the binding peptide is engineered into the loop between a first and second cleavable linker positioned on the N-terminus and C-terminus of the binding polypeptide, respectively.

113. The method of claim 112, wherein the first and second cleavable linker are the same.

114. The method of claim 112 or claim 113, wherein the first and second cleavable linker comprises a cleavage site selected from:
(i) a enterokinase cleavage site, optionally comprising the amino acid sequence set forth by DDDDK (SEQ ID NO:106);
(ii) a Factor Xa cleavage site, optionally comprising the amino acid sequence set forth by SEQ ID NO: 218 or I-E/D-G-R;
(iii) a thrombin cleavage site, optionally comprising the amino acid sequence set forth by any one of SEQ ID NOS: 219-222, more optionally set forth by SEQ ID NO: 220; or
(iv) a TEV protease cleavage site set forth by SEQ ID NO: 223 or SEQ ID NO:224.

115. A fusion protein comprising a modified binding peptide and a bacterial chaperone joined by a cleavable linker, wherein the modified binding peptide has the formula N-terminus to C-terminus: Y1-[cysteine motif binding peptide]-Y2, wherein:
the cysteine motif binding peptide is 20-50 amino acids in length in which 2-12 amino acids are cysteine residues able to form 1-6 disulfide bonds; and
Y1 and Y2 are amino acid peptide sequences that interact with one another when in proximity.

116. The fusion protein of claim 115, wherein Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif.

117. The fusion protein of claim 115 or claim 116, wherein the Y1 and Y2 are HW and SF, respectively.

118. The fusion protein of claim 115 or claim 116, wherein Y1 and Y2 are IS and TV, respectively.

119. The fusion protein of claim 115 or claim 116, wherein Y1 and Y2 are sequences that are each 14-30 amino acids in length comprising a heptad repeat, wherein Y1 and Y2 are able to interact to form an anti-parallel coiled-coil motif.

120. The fusion protein of claim 115 or 116, wherein Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203.

121. The fusion protein of claim 115, 116 or 120, wherein the Y1 is set forth in SEQ ID NO:202 and Y2 is set forth in SEQ ID NO:203, respectively.

122. The fusion protein of any of claims 115-121, wherein the cysteine motif binding peptide is a knob peptide from an ultralong CDR3 of a cow antibody.

123. The fusion protein of claim 122, wherein the cysteine motif binding peptide does not comprise an ascending or descending stalk domain or a contiguous portion thereof of more than 3 amino acids in length from the ultralong CDR3 of the cow antibody.

124. The fusion protein of any of claims 115-123, wherein the cysteine motif binding peptide is 22 to 43 amino acids in length.

125. The fusion protein of any of claims 115-124, wherein the cysteine motif binding peptide comprises at least 4 cysteine residues.

126. The fusion protein of any of claims 115-125, wherein the cysteine motif binding peptide contains 4 cysteine residues, 6 cysteine residues or 8 cysteine residues.

127. The fusion protein of any of claims 115-126, wherein the cysteine motif binding peptide is able to form at least 2 disulfide bonds.

128. The fusion protein of any of claims 115-127, wherein the cysteine motif binding peptide is able to form 2 disulfide bonds, 3 disulfide bonds or 4 disulfide bonds.

129. The fusion protein of any of claims 115-128, wherein the cysteine motif binding peptides binds to a target antigen that is a nonvirulent bacteria, a virus, a viral protein, an immunomodulatory protein, a cancer antigen, a human IgG, or a recombinant protein thereof.

130. The fusion protein of any of claims 115-129, wherein the target antigen is a coronavirus, a coronavirus pseudovirus, a recombinant coronavirus Spike protein, or a receptor-binding domain (RBD) of a coronavirus Spike protein.

131. The fusion protein of claim 130, wherein the coronavirus is selected from the group consisting of 229E, NL63, OC43, HKU1, MERS-CoV, SARS-CoV, and SARS-CoV2.

132. The fusion protein of claim 130 or claim 131, wherein the coronavirus is a SARS-CoV2 selected from Wuhan-Hu-1 isolate, B.1.351 South African variant, B.1.1.7 UK variant, a beta variant, a delta variant, or a omicron variant.

133. The fusion protein of any of claims 115-132, wherein the cysteine motif binding peptide is set forth in any one of SEQ ID NOS: 155, 198, and 227-240.

134. The fusion protein of any of claims 115-133, wherein the cleavable linker comprises a cleavage site selected from:
(i) a enterokinase cleavage site, optionally comprising the amino acid sequence set forth by DDDDK (SEQ ID NO:106);
(ii) a Factor Xa cleavage site, optionally comprising the amino acid sequence set forth by SEQ ID NO: 218 or I-E/D-G-R;
(iii) a thrombin cleavage site, optionally comprising the amino acid sequence set forth by any one of SEQ ID NOS: 219-222, more optionally set forth by SEQ ID NO: 220; or
(iv) a TEV protease cleavage site set forth by SEQ ID NO: 223 or SEQ ID NO:224.

135. The fusion protein of any of claims 115-134, wherein the cleavable linker is a enterokinase cleavable linker comprising the amino acid sequence DDDDK (SEQ ID NO:106).

136. The fusion protein of claim 135, wherein the enterokinase cleavable linker comprises the sequence DYKDDDK (SEQ ID NO: 209) or GGSDYKDDDDKGS (SEQ ID NO:210).

137. The fusion protein of any of claims 115-136, wherein the bacterial chaperone is thioredoxin A (TrxA).

138. The fusion protein of claim 137, wherein TrxA has the sequence set forth in SEQ ID NO:194 or a sequence of amino acids that exhibits at least 85%, 86%, 87%, 88%, 89%, 90%, 91%, 92%, 92%, 94%, 95%, 96%, 97%, 98%, 99% sequence identity to SEQ ID NO:194.

139. A soluble modified binding peptide produced by the method of any of claims 78-114.

140. A soluble peptide comprising a modified binding peptide that is disulfide-bonded, wherein the modified binding peptide has the formula N-terminus to C-terminus: Y1-[cysteine motif binding peptide]-Y2, wherein:
the cysteine motif binding peptide is 20-50 amino acids in length in which 2-12 amino acids are cysteine residues and wherein the soluble peptide contains 1 to 6 disulfide bonds; and
Y1 and Y2 are amino acid peptide sequences that interact with one another when in proximity.

141. The soluble peptide of claim 140, wherein Y1 and Y2 are characterized by:
(i) Y1 and Y2 are selected from (i) HW and SF, respectively; (ii) IS and TV, respectively; (iii) DY and MP, respectively; (iv) LV and IP, respectively; or (v) SV and YI, respectively; or
(ii) Y1 and Y2 are sequences that are each 14-30 amino acids in length comprising a heptad repeat, wherein Y1 and Y2 are able to interact to form an anti-parallel coiled-coil motif.

142. The soluble peptide of claim 140 or claim 141, wherein the Y1 and Y2 are HW and SF, respectively.

143. The soluble peptide of claim 140 or claim 141, wherein Y1 and Y2 are IS and TV, respectively.

144. The soluble peptide of claim 140 or claim 141, wherein Y1 and Y2 are sequences that are each 14-30 amino acids in length comprising a heptad repeat, wherein Y1 and Y2 are able to interact to form an anti-parallel coiled-coil motif.

145. The soluble peptide of claim 140 or claim 141, wherein Y1 is the sequence set forth in SEQ ID NO:202 or a sequence having no or about 1, 2, 3 or 4 amino acid substitutions or deletions from SEQ ID NO:202, and Y2 is the sequence set forth in SEQ ID NO:203 or a sequence having no or about 1, 2, 3 or 4 amino acid substitutions or deletions from SEQ ID NO:203.

146. The soluble peptide of claim 140, 141 or 145, wherein the Y1 is set forth in SEQ ID NO:202 and Y2 is set forth in SEQ ID NO:203, respectively.

147. The soluble peptide of any of claims 140-146, wherein the cysteine motif binding peptide is a knob peptide from an ultralong CDR3 of a cow antibody.

148. The soluble peptide of claim 147, wherein the cysteine motif binding peptide does not comprise an ascending or descending stalk domain or a contiguous portion thereof of more than 3 amino acids in length from the ultralong CDR3 of the cow antibody.

149. The soluble peptide of any of claims 140-148, wherein the cysteine motif binding peptide is 22 to 43 amino acids in length.

150. The soluble peptide of any of claims 140-149, wherein the cysteine motif binding peptide comprises at least 4 cysteine residues.

151. The soluble peptide of any of claims 140-150, wherein the cysteine motif binding peptide contains 4 cysteine residues, 6 cysteine residues or 8 cysteine residues.

152. The soluble peptide of any of claims 140-151, wherein the soluble peptide has at least 2 disulfide bonds.

153. The soluble peptide of any of claims 140-152, wherein the soluble peptide has 2 disulfide bonds, 3 disulfide bonds or 4 disulfide bonds.

154. The soluble peptide of any of claims 140-153, wherein the soluble peptide binds to a target antigen that is a nonvirulent bacteria, a virus, a viral protein, an immunomodulatory protein, a cancer antigen, a human IgG, or a recombinant protein thereof.

155. The soluble peptide of any of claims 140-154, wherein the target antigen is a coronavirus, a coronavirus pseudovirus, a recombinant coronavirus Spike protein, or a receptor-binding domain (RBD) of a coronavirus Spike protein.

156. The soluble peptide of claim 155, wherein the coronavirus is selected from the group consisting of 229E, NL63, OC43, HKU1, MERS-CoV, SARS-CoV, and SARS-CoV2.

157. The soluble peptide of claim 155 or claim 156, wherein the coronavirus is a SARS-CoV2 selected from Wuhan-Hu-1 isolate, B.1.351 South African variant, B.1.1.7 UK variant, a beta variant, a delta variant, or an omicron variant.

158. The soluble peptide of any of claims 140-157, wherein the cysteine motif binding peptide is set forth in any one of SEQ ID NOS: 155, 198, and 227-240.

159. A composition comprising the soluble peptide of any of claims 139-158.

160. The composition of claim 159, that is a pharmaceutical composition comprising a pharmaceutically acceptable excipient.

161. A method of administering to a subject the modified fusion polypeptide of any of claims 1-24, the fusion peptide of any of claims 25-31, the composition of claim 33 or claim 34, the modified binding peptide of any of claim 77, the soluble peptide of any of claims 139-158, or the composition of claim 159 or claim 160 for use in treating a disease or condition.

162. The method of claim 161, wherein the disease or condition is a virus infection.

163. The method of claim 162, wherein the virus infection is infection with a coronavirus.
